(19) United States
(12) Patent Application Publication
(10) Pub. No.: US 2025/0101096 A1
(43) Pub. Date: Mar. 27, 2025

(54) SHBG AND IBP4 ANTIBODIES AND METHODS USING SAME

(71) Applicant: Sera Prognostics, Inc., Salt Lake City, UT (US)

(72) Inventors: Andrew Dennis Gassman, Salt Lake City, UT (US); Jeffrey S. Flick, Salt Lake City, UT (US); John M. Peltier, Murray City, UT (US)

(21) Appl. No.: 18/730,657

(22) PCT Filed: Feb. 9, 2023
(86) PCT No.: PCT/US2023/062311

§ 371 Date: Jul. 19, 2024

Related U.S. Application Data
(60) Provisional application No. 63/308,453, filed on Feb. 9, 2022.

(51) Int. Cl. C07K 16/26; G01N 33/543; G01N 33/68; G01N 33/74

(52) U.S. Cl. CPC C07K 16/26; G01N 33/54313; G01N 33/6848; G01N 33/74; C07K 2317/24; C07K 2317/34; C07K 2317/565; C07K 2317/92; G01N 2333/4745; G01N 2333/575; G01N 2800/042; G01N 2800/321; G01N 2800/368

(57) ABSTRACT

Sequence-defined anti-SHBG and anti-IBP4 antibodies or antigen-binding fragments thereof are described. The antibodies are comprise heavy chain variable and light chain variable domains, each having sequence-specified complementarity determining regions (CDRs). Also described are nucleic acids encoding the antibodies, compositions comprising the antibodies, methods of producing the antibodies, and methods of using the antibodies and sequence-defined epitopes of the antibodies.

### CROSS-REFERENCE TO RELATED APPLICATIONS

This application claims the benefit of priority of U.S. Provisional Application No. 63/308,453, filed Feb. 9, 2022, the entire contents of which is incorporated by reference herein.

### SEQUENCE LISTING

The instant application contains a Sequence Listing, which has been submitted via Patent Center. The Sequence Listing titled 203123-025002PCT.xml, which was created on Feb. 9, 2023 and is 320,751 bytes in size, is hereby incorporated by reference it its entirety.

### FIELD OF THE INVENTION

This invention relates to anti-sex hormone-binding globulin (SHBG) antibodies and anti-insulin-like growth factor binding protein 4 (IBP4), nucleic acids and expression vectors encoding the antibodies, recombinant cells containing the vectors, and compositions comprising the antibodies. Methods of making the antibodies, and methods of using the antibodies to diagnose preterm birth or pregnancy associated complications, are also provided.

### BACKGROUND OF THE INVENTION

According to the World Health Organization, an estimated 15 million babies are born preterm (before 37 completed weeks of gestation) every year. In almost all countries with reliable data, preterm birth rates are increasing. See, World Health Organization; March of Dimes; The Partnership for Maternal, Newborn & Child Health; Save the Children, Born too soon: the global action report on preterm birth, ISBN 9789241503433(2012). An estimated 1 million babies die annually from preterm birth complications. Globally, preterm birth is the leading cause of newborn deaths (babies in the first four weeks of life) and the second leading cause of death after pneumonia in children under five years. Many survivors face a lifetime of disability, including learning disabilities and visual and hearing problems.

To prevent preterm birth in women who are less than 24 weeks pregnant with an ultrasound showing cervical opening, a surgical procedure known as cervical cerclage can be employed in which the cervix is stitched closed with strong sutures. For women less than 34 weeks pregnant and in active preterm labor, hospitalization may be necessary as well as the administration of medications to temporarily halt preterm labor and/or promote the fetal lung development. If a pregnant women is determined to be at risk for preterm birth, health care providers can implement various clinical strategies that may include preventive medications, for example, 17-α hydroxyprogesterone caproate (Makena) injections and/or vaginal progesterone gel, cervical pessaries, restrictions on sexual activity and/or other physical activities, and alterations of treatments for chronic conditions, such as diabetes and high blood pressure, that increase the risk of preterm labor.

There is a great need to identify and provide women at risk for preterm birth with proper antenatal care. Women identified as high-risk can be scheduled for more intensive antenatal surveillance and prophylactic interventions. Current strategies for risk assessment are based on the obstetric and medical history and clinical examination, but these strategies are only able to identify a small percentage of women who are at risk for preterm delivery. Prior history of spontaneous preterm birth (sPTB) is currently the single strongest predictor of subsequent preterm birth (PTB). After one prior sPTB the probability of a second PTB is 30-50%. Other maternal risk factors include: black race, low maternal body-mass index, and short cervical length. Amniotic fluid, cervicovaginal fluid, and serum biomarker studies to predict sPTB suggest that multiple molecular pathways are aberrant in women who ultimately deliver preterm. Reliable early identification of risk for preterm birth would enable planning appropriate monitoring and clinical management to prevent preterm delivery. Such monitoring and management might include: more frequent prenatal care visits, serial cervical length measurements, enhanced education regarding signs and symptoms of early preterm labor, lifestyle interventions for modifiable risk behaviors such as smoking cessation, cervical pessaries and progesterone treatment. Finally, reliable antenatal identification of risk for preterm birth also is crucial to cost-effective allocation of monitoring resources

Despite intense research to identify at-risk women, PTB prediction algorithms based solely on clinical and demographic factors or using measured serum or vaginal biomarkers have not resulted in clinically useful tests. More accurate methods to identify women at risk during their first pregnancy and sufficiently early in gestation are needed to allow for clinical intervention. The present disclosure addresses this need by providing SHBG and IBP4 binding agents for the detection and quantification of biomarkers indicative of a susceptibility to preterm birth.

### BRIEF SUMMARY OF THE INVENTION

This disclosure relates to anti-sex hormone-binding globulin (SHBG) antibodies and anti-insulin-like growth factor binding protein 4 (IBP4), nucleic acids, and expression vectors encoding the antibodies, recombinant cells containing the vectors, and compositions comprising the antibodies. This disclosure further relates to methods of making the antibodies, and methods of using the antibodies to diagnose or treat conditions associated with SHBG and/or IBP4.

In one aspect, this disclosure provides an anti-SHBG antibody or a fragment thereof, comprising: (i) a heavy chain, wherein the heavy chain comprises: a heavy chain complementarity determining region 1 (HCDR1) comprising an amino acid sequence selected from the group consisting of SEQ ID NOS: 11, 14, 17, 20, 52, and 115; and a heavy chain complementarity determining region 2 (HCDR2) comprising an amino acid sequence selected from the group consisting of SEQ ID NOS: 12, 15, 18, 21, 44, 47, 50, 53, 116, and 117; and a heavy chain complementarity determining region 3 (HCDR3) comprising an amino acid sequence selected from the group consisting of SEQ ID NOS: 13, 16, 19, 22, 45, 48, 51, and 54; or (ii) a light chain, wherein the light chain comprises: a light chain complementarity determining region 1 (LCDR1) comprising an amino acid sequence selected from the group consisting of SEQ ID NOS: 23, 26, 29, and 32; and a light chain complementarity determining region 2 (LCDR2) comprising an amino acid sequence selected from the group consisting of SEQ ID NOS: 24, 27, 30, and 33; and a light chain complementarity determining region 3 (LCDR3) comprising an amino acid sequence selected from the group consisting of SEQ ID NOS: 25, 31, and 34. In some embodiments, the isolated anti-SHBG antibody or the fragment thereof comprises the heavy chain. In some embodiments, the isolated anti-SHBG antibody or the fragment thereof comprises the light chain. In some embodiments, the isolated anti-SHBG antibody or the fragment thereof comprises the heavy chain and the light chain.

In some embodiments, this disclosure provides an anti-SHBG antibody or a fragment thereof, wherein the HCDR1 comprises the amino acid sequence of SEQ ID NO: 11, the HCDR2 comprises the amino acid sequence of SEQ ID NO: 12, and the HCDR3 comprises the amino acid sequence of SEQ ID NO: 13; and the light chain comprises the LCDR1 comprises the amino acid sequence of SEQ ID NO: 23, the LCDR2 comprises the amino acid sequence of SEQ ID NO: 24, and the LCDR3 comprises the amino acid sequence of SEQ ID NO: 25. In some embodiments, the HCDR1 comprises the amino acid sequence of SEQ ID NO: 11, the HCDR2 comprises the amino acid sequence of SEQ ID NO: 44, and the HCDR3 comprises the amino acid sequence of SEQ ID NO: 45; and the LCDR1 comprises the amino acid sequence of SEQ ID NO:23, the LCDR2 comprises the amino acid sequence of SEQ ID NO:24, and the LCDR3 comprises the amino acid sequence of SEQ ID NO:25. In some embodiments, the HCDR1 comprises the amino acid sequence of SEQ ID NO: 14, the HCDR2 comprises the amino acid sequence of SEQ ID NO: 15, and the HCDR3 comprises the amino acid sequence of SEQ ID NO: 16; and the LCDR1 comprises the amino acid sequence of SEQ ID NO: 26, the LCDR2 comprises the amino acid sequence of SEQ ID NO: 27, and the LCDR3 comprises the amino acid sequence of SEQ ID NO: 25. In some embodiments, the HCDR1 comprises the amino acid sequence of SEQ ID NO: 14, the HCDR2 comprises the amino acid sequence of SEQ ID NO: 47, and the HCDR3 comprises the amino acid sequence of SEQ ID NO: 48; and the LCDR1 comprises the amino acid sequence of SEQ ID NO: 26, the LCDR2 comprises the amino acid sequence of SEQ ID NO: 27, and the LCDR3 comprises the amino acid sequence of SEQ ID NO: 25. In some embodiments, the HCDR1 comprises the amino acid sequence of SEQ ID NO: 17, the HCDR2 comprises the amino acid sequence of SEQ ID NO: 18, and the HCDR3 comprises the amino acid sequence of SEQ ID NO: 19; and the LCDR1 comprises the amino acid sequence of SEQ ID NO: 29, the LCDR2 comprises the amino acid sequence of SEQ ID NO: 30, and the LCDR3 comprises the amino acid sequence of SEQ ID NO: 31 In some embodiments, the HCDR1 comprises the amino acid sequence of SEQ ID NO: 17, the HCDR2 comprises the amino acid sequence of SEQ ID NO: 50, and the HCDR3 comprises the amino acid sequence of SEQ ID NO: 51; and the LCDR1 comprises the amino acid sequence of SEQ ID NO: 29, the LCDR2 comprises the amino acid sequence of SEQ ID NO: 30, and the LCDR3 comprises the amino acid sequence of SEQ ID NO: 31. In some embodiments, the HCDR1 comprises the amino acid sequence of SEQ ID NO: 20, the HCDR2 comprises the amino acid sequence of SEQ ID NO: 21, and the HCDR3 comprises the amino acid sequence of SEQ ID NO: 22; and the LCDR1 comprises the amino acid sequence of SEQ ID NO: 32, the LCDR2 comprises the amino acid sequence of SEQ ID NO: 33, and the LCDR3 comprises the amino acid sequence of SEQ ID NO: 34. In some embodiments, the the HCDR1 comprises the amino acid sequence of SEQ ID NO: 52, the HCDR2 comprises the amino acid sequence of SEQ ID NO: 53, the HCDR3 comprises the amino acid sequence of SEQ ID NO: 54; and the LCDR1 comprises the amino acid sequence of SEQ ID NO: 32, the LCDR2 comprises the amino acid sequence of SEQ ID NO: 33, and the LCDR3 comprises the amino acid sequence of SEQ ID NO: 34. In some embodiments, the HCDR1 comprises the amino acid sequence of SEQ ID NO: 115, the HCDR2 comprises the amino acid sequence of SEQ ID NO: 116, and the HCDR3 comprises the amino acid sequence of SEQ ID NO: 19; and the LCDR1 comprises the amino acid sequence of SEQ ID NO: 29, the LCDR2 comprises the amino acid sequence of SEQ ID NO: 30, and the LCDR3 comprises the amino acid sequence of SEQ ID NO: 31 In some embodiments, the HCDR1 comprises the amino acid sequence of SEQ ID NO: 115, the HCDR2 comprises the amino acid sequence of SEQ ID NO: 116, and the HCDR3 comprises the amino acid sequence of SEQ ID NO: 51; and the LCDR1 comprises the amino acid sequence of SEQ ID NO: 29, the LCDR2 comprises the amino acid sequence of SEQ ID NO: 30, and the LCDR3 comprises the amino acid sequence of SEQ ID NO: 31.

In another aspect, this disclosure provides an isolated anti-SHBG antibody or a fragment thereof, comprising: (i) a heavy chain comprising a HCDR1 comprising amino acid sequence GYTFTGYW (SEQ ID NO: 11), a HCDR2 comprising amino acid sequence IDPSX₁X₂YT (SEQ ID NO: 112), wherein X₁ is independently any naturally occurring amino acid residue and X₂ is independently any naturally occurring amino acid residue, and a HCDR3 comprising amino acid sequence TRX₃PLVTADFX₃Y (SEQ ID NO: 113), wherein X₃ is independently any naturally occurring amino acid residue; and (ii) a light chain comprising a LCDR1 comprising amino acid sequence QNVDTN (SEQ ID NO: 23), a LCDR2 comprising amino acid sequence SAS (SEQ ID NO: 24), and a LCDR3 comprising amino acid sequence QQYDSYPY (SEQ ID NO: 25). In some embodiments, X₁ comprises threonine or aspartic acid, X₂ comprises glycine or threonine, and X₃ comprises glutamic acid or aspartic acid. In some embodidments, X₁ comprises threonine, X₂ comprises glycine, and X₃ comprises glutamic acid.

In another aspect, this disclosure provides an isolated anti-SHBG antibody or a fragment thereof, comprising: (i) a heavy chain comprising an amino acid sequence that has at least 85%, 90%, 95%, 96%, 97%, 98%, 99%, or 100% identity to SEQ ID NO: 4, or a light chain comprising an amino acid sequence that has at least 85%, 90%, 95%, 96%, 97%, 98%, 99%, or 100% identity to SEQ ID NO: 6; or (ii) a heavy chain comprising an amino acid sequence that has at least 85%, 90%, 95%, 96%, 97%, 98%, 99%, or 100% identity to SEQ ID NO: 36, or a light chain comprising an amino acid sequence that has at least 85%, 90%, 95%, 96%, 97%, 98%, 99%, or 100% identity to SEQ ID NO: 38. In some embodiments, the antibody or the fragment thereof comprises the heavy chain of SEQ ID NO: 4. In some embodiments, the antibody or the fragment thereof of claim 18, wherein the antibody or the fragment thereof comprises the light chain of SEQ ID NO: 6. In some embodiments, the antibody or the fragment thereof comprises the heavy chain of SEQ ID NO: 4 and the light chain of SEQ ID NO: 6. In some embodiments, the antibody or the fragment thereof comprises the heavy chain of SEQ ID NO: 36. In some embodiments, the antibody or the fragment thereof comprises the light chain of SEQ ID NO: 38. In some embodiments, the antibody or the fragment thereof comprises the heavy chain of SEQ ID NO: 36 and the light chain of SEQ ID NO: 38. In some embodiments, the heavy chain and the light chain form an antigen binding domain for binding to an epitope of SHBG. In some embodiments, the epitope of SHBG comprises the amino acid sequence of SEQ ID NO: 110.

In some embodiments, this disclosure provides an isolated anti-SHBG antibody or a fragment thereof, wherein the anti-SHBG antibody or the fragment thereof has a higher affinity for a biologically active form of SHBG than for a biologically inactive form of SHBG. In some embodiments, the antibody or the fragment thereof comprises a higher affinity for glycosylated SHBG than for non-glycosylated SHBG. In some embodiments, the glycosylated SHBG comprises an epitope comprising a glycosylated amino acid, wherein the glycosylated amino acid comprises a threonine at position 36. In some embodiments, the glycosylated amino acid comprises an O-linked glycan.

In another aspect, this disclosure provides an isolated anti-IBP4 antibody or a fragment thereof, comprising: (i) a heavy chain, wherein the heavy chain comprises: a heavy chain complementarity determining region 1 (HCDR1) comprising an amino acid sequence selected from the group consisting of SEQ ID NOS: 75, 78, 81, 84, 118, 244, 247, 250, 253, 256, 288, 291, 284, and 314; and a heavy chain complementarity determining region 2 (HCDR2) comprising an amino acid sequence selected from the group consisting of SEQ ID NOS: 76, 79, 82, 85, 119, 216, 218, 220, 222, 224, 245, 248, 251, 254, 257, 284, 286, 289, 292, 295, 312, 315, 317, 319, and 321; and a heavy chain complementarity determining region 3 (HCDR3) comprising an amino acid sequence selected from the group consisting of SEQ ID NOS: 77, 80, 83, 86, 217, 219, 221, 223, 225, 246, 249, 252, 255, 258, 285, 287, 290, 293, 296, 313, 316, 318, 320, 322; or (ii) a light chain, wherein the light chain comprises: a light chain complementarity determining region 1 (LCDR1) comprising an amino acid sequence selected from the group consisting of SEQ ID NOS: 87, 90, 93, 96, 226, 228, 230, 233, 272, 274, 275, 323, 325, 327, and 330; and a light chain complementarity determining region 2 (LCDR2) comprising an amino acid sequence selected from the group consisting of SEQ ID NOS: 24, 91, 94, 97, 229, 231, 234, 260, 261, 263, 273, 297, 299, 300, 302, 326, 328, and 331; and a light chain complementarity determining region 3 (LCDR3) comprising an amino acid sequence selected from the group consisting of SEQ ID NOS: 89, 95, 98, 99, 100, 101, 227, 232, 235, 259, 262, 264, 298, 301, 303, 324, 329, and 332. In some embodiments, the antibody or the fragment thereof comprises the heavy chain. In some embodiments, the antibody or the fragment thereof comprises the light chain. In some embodiments, the antibody or the fragment thereof comprises the heavy chain and the light chain.

In some embodiments, this disclosure provides an isolated anti-IBP4 antibody or a fragment thereof, wherein: (i) the HCDR1 comprises the amino acid sequence of SEQ ID NO: 75, the HCDR2 comprises the amino acid sequence of SEQ ID NO: 76, and the HCDR3 comprises the amino acid sequence of SEQ ID NO: 77; and (ii) the LCDR1 comprises the amino acid sequence of SEQ ID NO: 87, the LCDR2 comprises the amino acid sequence of SEQ ID NO: 24, and the LCDR3 comprises the amino acid sequence of SEQ ID NO: 99. In some embodiments, (i) the HCDR1 comprises the amino acid sequence of SEQ ID NO: 78, the HCDR2 comprises the amino acid sequence of SEQ ID NO: 79, and the HCDR3 comprises the amino acid sequence of SEQ ID NO: 80; and (ii) the LCDR1 comprises the amino acid sequence of SEQ ID NO: 90, the LCDR2 comprises the amino acid sequence of SEQ ID NO: 91, and the LCDR3 comprises the amino acid sequence of SEQ ID NO: 89. In some embodiments, (i) the HCDR1 comprises the amino acid sequence of SEQ ID NO: 78, the HCDR2 comprises the amino acid sequence of SEQ ID NO: 79, and the HCDR3 comprises the amino acid sequence of SEQ ID NO: 80; and (ii) the LCDR1 comprises the amino acid sequence of SEQ ID NO: 90, the LCDR2 comprises the amino acid sequence of SEQ ID NO: 91, and the LCDR3 comprises the amino acid sequence of SEQ ID NO: 99. In some embodiments, (i) the HCDR1 comprises the amino acid sequence of SEQ ID NO: 81, the HCDR2 comprises the amino acid sequence of SEQ ID NO: 82, and the HCDR3 comprises the amino acid sequence of SEQ ID NO: 83; and (ii) the LCDR1 comprises the amino acid sequence of SEQ ID NO: 93, the LCDR2 comprises the amino acid sequence of SEQ ID NO: 94, and the LCDR3 comprises the amino acid sequence of SEQ ID NO: 95. In some embodiments, (i) the HCDR1 comprises the amino acid sequence of SEQ ID NO: 81, the HCDR2 comprises the amino acid sequence of SEQ ID NO: 82, the HCDR3 comprises the amino acid sequence of SEQ ID NO: 83; and (ii) the LCDR1 comprises the amino acid sequence of SEQ ID NO: 93, the LCDR2 comprises the amino acid sequence of SEQ ID NO: 94, and the LCDR3 comprises the amino acid sequence of SEQ ID NO: 100. In some embodiments, (i) the HCDR1 comprises the amino acid sequence of SEQ ID NO: 84, the HCDR2 comprises the amino acid sequence of SEQ ID NO: 85, and the HCDR3 comprises the amino acid sequence of SEQ ID NO: 86; and (ii) the LCDR1 comprises the amino acid sequence of SEQ ID NO: 96, the LCDR2 comprises the amino acid sequence of SEQ ID NO: 97, and the LCDR3 comprises the amino acid sequence of SEQ ID NO: 98. In some embodiments, (i) the HCDR1 comprises the amino acid sequence of SEQ ID NO: 84, the HCDR2 comprises the amino acid sequence of SEQ ID NO: 85, and the HCDR3 comprises the amino acid sequence of SEQ ID NO: 86; and (ii) the LCDR1 comprises the amino acid sequence of SEQ ID NO: 96, the LCDR2 comprises the amino acid sequence of SEQ ID NO: 97, and the LCDR3 comprises the amino acid sequence of SEQ ID NO: 101. In some embodiments, (i) the HCDR1 comprises the amino acid sequence of SEQ ID NO: 118, the HCDR2 comprises the amino acid sequence of SEQ ID NO: 119, and the HCDR3 comprises the amino acid sequence of SEQ ID NO: 83; and (ii) the LCDR1 comprises the amino acid sequence of SEQ ID NO: 93, the LCDR2 comprises the amino acid sequence of SEQ ID NO: 94, and the LCDR3 comprises the amino acid sequence of SEQ ID NO: 89. In some embodiments, (i) the HCDR1 comprises the amino acid sequence of SEQ ID NO: 118, the HCDR2 comprises the amino acid sequence of SEQ ID NO: 119, and the HCDR3 comprises the amino acid sequence of SEQ ID NO: 83; and (ii) the LCDR1 comprises the amino acid sequence of SEQ ID NO: 93, the LCDR2 comprises the amino acid sequence of SEQ ID NO: 94, and the LCDR3 comprises the amino acid sequence of SEQ ID NO: 99.

In some embodiments, this disclosure provides an anti-IBP4 antibody or a fragment thereof, wherein the heavy chain and the light chain form an antigen binding domain for binding to an epitope of IBP4. In some embodiments, the epitope of IBP4 comprises the amino acid sequence of SEQ ID NO: 111.

In some embodiments, this disclosure provides an isolated anti-IBP4 antibody or a fragment thereof, wherein: (i) the HCDR1 comprises the amino acid sequence of SEQ ID NO: 75, the HCDR2 comprises the amino acid sequence of SEQ ID NO: 216, and the HCDR3 comprises the amino acid sequence of SEQ ID NO: 217; and (ii) the LCDR1 comprises the amino acid sequence of SEQ ID NO: 226, the LCDR2 comprises the amino acid sequence of SEQ ID NO: 24, and the LCDR3 comprises the amino acid sequence of SEQ ID NO: 227. In some embodiments, (i) the HCDR1 comprises the amino acid sequence of SEQ ID NO: 78, the HCDR2 comprises the amino acid sequence of SEQ ID NO: 218, and the HCDR3 comprises the amino acid sequence of SEQ ID NO: 219; and (ii) the LCDR1 comprises the amino acid sequence of SEQ ID NO: 228, the LCDR2 comprises the amino acid sequence of SEQ ID NO: 229, and the LCDR3 comprises the amino acid sequence of SEQ ID NO: 227. In some embodiments, (i) the HCDR1 comprises the amino acid sequence of SEQ ID NO: 81, the HCDR2 comprises the amino acid sequence of SEQ ID NO: 220, and the HCDR3 comprises the amino acid sequence of SEQ ID NO: 221; and (ii) the LCDR1 comprises the amino acid sequence of SEQ ID NO: 230, the LCDR2 comprises the amino acid sequence of SEQ ID NO: 231, and the LCDR3 comprises the amino acid sequence of SEQ ID NO: 232. In some embodiments, (i) the HCDR1 comprises the amino acid sequence of SEQ ID NO: 84, the HCDR2 comprises the amino acid sequence of SEQ ID NO: 223, and the HCDR3 comprises the amino acid sequence of SEQ ID NO: 223; and (ii) the LCDR1 comprises the amino acid sequence of SEQ ID NO: 233, the LCDR2 comprises the amino acid sequence of SEQ ID NO: 234, and the LCDR3 comprises the amino acid sequence of SEQ ID NO: 235. In some embodiments, (i) the HCDR1 comprises the amino acid sequence of SEQ ID NO: 118, the HCDR2 comprises the amino acid sequence of SEQ ID NO: 224, and the HCDR3 comprises the amino acid sequence of SEQ ID NO: 225; and (ii) the LCDR1 comprises the amino acid sequence of SEQ ID NO: 230, the LCDR2 comprises the amino acid sequence of SEQ ID NO: 231, and the LCDR3 comprises the amino acid sequence of SEQ ID NO: 227. In some embodiments, (i) the HCDR1 comprises the amino acid sequence of SEQ ID NO: 244, the HCDR2 comprises the amino acid sequence of SEQ ID NO: 245, and the HCDR3 comprises the amino acid sequence of SEQ ID NO: 246; and (ii) the LCDR1 comprises the amino acid sequence of SEQ ID NO: 87, the LCDR2 comprises the amino acid sequence of SEQ ID NO: 24, and the LCDR3 comprises the amino acid sequence of SEQ ID NO: 259. In some embodiments, (i) the HCDR1 comprises the amino acid sequence of SEQ ID NO: 247, the HCDR2 comprises the amino acid sequence of SEQ ID NO: 248, and the HCDR3 comprises the amino acid sequence of SEQ ID NO: 249; and (ii) the LCDR1 comprises the amino acid sequence of SEQ ID NO: 90, the LCDR2 comprises the amino acid sequence of SEQ ID NO: 260, and the LCDR3 comprises the amino acid sequence of SEQ ID NO: 259. In some embodiments, (i) the HCDR1 comprises the amino acid sequence of SEQ ID NO: 250, the HCDR2 comprises the amino acid sequence of SEQ ID NO: 251, and the HCDR3 comprises the amino acid sequence of SEQ ID NO: 252; and (ii) the LCDR1 comprises the amino acid sequence of SEQ ID NO: 93, the LCDR2 comprises the amino acid sequence of SEQ ID NO: 261, and the LCDR3 comprises the amino acid sequence of SEQ ID NO: 262. In some embodiments, (i) the HCDR1 comprises the amino acid sequence of SEQ ID NO: 253, the HCDR2 comprises the amino acid sequence of SEQ ID NO: 254, and the HCDR3 comprises the amino acid sequence of SEQ ID NO: 255; and (ii) the LCDR1 comprises the amino acid sequence of SEQ ID NO: 96, the LCDR2 comprises the amino acid sequence of SEQ ID NO: 263, and the LCDR3 comprises the amino acid sequence of SEQ ID NO: 264. In some embodiments, (i) the HCDR1 comprises the amino acid sequence of SEQ ID NO: 256, the HCDR2 comprises the amino acid sequence of SEQ ID NO: 257, and the HCDR3 comprises the amino acid sequence of SEQ ID NO: 258; and (ii) the LCDR1 comprises the amino acid sequence of SEQ ID NO: 93, the LCDR2 comprises the amino acid sequence of SEQ ID NO: 261, and the LCDR3 comprises the amino acid sequence of SEQ ID NO: 259. In some embodiments, (i) the HCDR1 comprises the amino acid sequence of SEQ ID NO: 244, the HCDR2 comprises the amino acid sequence of SEQ ID NO: 245, and the HCDR3 comprises the amino acid sequence of SEQ ID NO: 246; and (ii) the LCDR1 comprises the amino acid sequence of SEQ ID NO: 23, the LCDR2 comprises the amino acid sequence of SEQ ID NO: 24, and the LCDR3 comprises the amino acid sequence of SEQ ID NO: 259. In some embodiments, (i) the HCDR1 comprises the amino acid sequence of SEQ ID NO: 247, the HCDR2 comprises the amino acid sequence of SEQ ID NO: 248, and the HCDR3 comprises the amino acid sequence of SEQ ID NO: 249; and (ii) the LCDR1 comprises the amino acid sequence of SEQ ID NO: 272, the LCDR2 comprises the amino acid sequence of SEQ ID NO: 273, and the LCDR3 comprises the amino acid sequence of SEQ ID NO: 259. In some embodiments, (i) the HCDR1 comprises the amino acid sequence of SEQ ID NO: 250, the HCDR2 comprises the amino acid sequence of SEQ ID NO: 251, and the HCDR3 comprises the amino acid sequence of SEQ ID NO: 252; and (ii) the LCDR1 comprises the amino acid sequence of SEQ ID NO: 274, the LCDR2 comprises the amino acid sequence of SEQ ID NO: 94, and the LCDR3 comprises the amino acid sequence of SEQ ID NO: 262. In some embodiments, (i) the HCDR1 comprises the amino acid sequence of SEQ ID NO: 253, the HCDR2 comprises the amino acid sequence of SEQ ID NO: 254, and the HCDR3 comprises the amino acid sequence of SEQ ID NO: 255; and (ii) the LCDR1 comprises the amino acid sequence of SEQ ID NO: 275, the LCDR2 comprises the amino acid sequence of SEQ ID NO: 97, and the LCDR3 comprises the amino acid sequence of SEQ ID NO: 264. In some embodiments, (i) the HCDR1 comprises the amino acid sequence of SEQ ID NO: 256, the HCDR2 comprises the amino acid sequence of SEQ ID NO: 257, and the HCDR3 comprises the amino acid sequence of SEQ ID NO: 258; and (ii) the LCDR1 comprises the amino acid sequence of SEQ ID NO: 274, the LCDR2 comprises the amino acid sequence of SEQ ID NO: 94, and the LCDR3 comprises the amino acid sequence of SEQ ID NO: 259. In some embodiments, (i) the HCDR1 comprises the amino acid sequence of SEQ ID NO: 244, the HCDR2 comprises the amino acid sequence of SEQ ID NO: 284, and the HCDR3 comprises the amino acid sequence of SEQ ID NO: 285; and (ii) the LCDR1 comprises the amino acid sequence of SEQ ID NO: 87, the LCDR2 comprises the amino acid sequence of SEQ ID NO: 297, and the LCDR3 comprises the amino acid sequence of SEQ ID NO: 298. In some embodiments, (i) the HCDR1 comprises the amino acid sequence of SEQ ID NO: 247, the HCDR2 comprises the amino acid sequence of SEQ ID NO: 286, and the HCDR3 comprises the amino acid sequence of SEQ ID NO: 287; and (ii) the LCDR1 comprises the amino acid sequence of SEQ ID NO: 90, the LCDR2 comprises the amino acid sequence of SEQ ID NO: 299, and the LCDR3 comprises the amino acid sequence of SEQ ID NO: 298. In some embodiments, (i) the HCDR1 comprises the amino acid sequence of SEQ ID NO: 288, the HCDR2 comprises the amino acid sequence of SEQ ID NO: 289, and the HCDR3 comprises the amino acid sequence of SEQ ID NO: 290; and (ii) the LCDR1 comprises the amino acid sequence of SEQ ID NO: 93, the LCDR2 comprises the amino acid sequence of SEQ ID NO: 300, and the LCDR3 comprises the amino acid sequence of SEQ ID NO: 301. In some embodiments, (i) the HCDR1 comprises the amino acid sequence of SEQ ID NO: 291, the HCDR2 comprises the amino acid sequence of SEQ ID NO: 292, and the HCDR3 comprises the amino acid sequence of SEQ ID NO: 293; and (ii) the LCDR1 comprises the amino acid sequence of SEQ ID NO: 96, the LCDR2 comprises the amino acid sequence of SEQ ID NO: 302, and the LCDR3 comprises the amino acid sequence of SEQ ID NO: 303. In some embodiments, (i) the HCDR1 comprises the amino acid sequence of SEQ ID NO: 294, the HCDR2 comprises the amino acid sequence of SEQ ID NO: 295, and the HCDR3 comprises the amino acid sequence of SEQ ID NO: 296; and (ii) the LCDR1 comprises the amino acid sequence of SEQ ID NO: 93, the LCDR2 comprises the amino acid sequence of SEQ ID NO: 300, and the LCDR3 comprises the amino acid sequence of SEQ ID NO: 298. In some embodiments, (i) the HCDR1 comprises the amino acid sequence of SEQ ID NO: 244, the HCDR2 comprises the amino acid sequence of SEQ ID NO: 312, and the HCDR3 comprises the amino acid sequence of SEQ ID NO: 313; and (ii) the LCDR1 comprises the amino acid sequence of SEQ ID NO: 323, the LCDR2 comprises the amino acid sequence of SEQ ID NO: 24, and the LCDR3 comprises the amino acid sequence of SEQ ID NO: 324. In some embodiments, (i) the HCDR1 comprises the amino acid sequence of SEQ ID NO: 314, the HCDR2 comprises the amino acid sequence of SEQ ID NO: 315, and the HCDR3 comprises the amino acid sequence of SEQ ID NO: 316; and (ii) the LCDR1 comprises the amino acid sequence of SEQ ID NO: 325, the LCDR2 comprises the amino acid sequence of SEQ ID NO: 326, and the LCDR3 comprises the amino acid sequence of SEQ ID NO: 324. In some embodiments, (i) the HCDR1 comprises the amino acid sequence of SEQ ID NO: 288, the HCDR2 comprises the amino acid sequence of SEQ ID NO: 317, and the HCDR3 comprises the amino acid sequence of SEQ ID NO: 318; and (ii) the LCDR1 comprises the amino acid sequence of SEQ ID NO: 327, the LCDR2 comprises the amino acid sequence of SEQ ID NO: 328, and the LCDR3 comprises the amino acid sequence of SEQ ID NO: 329. In some embodiments, (i) the HCDR1 comprises the amino acid sequence of SEQ ID NO: 291, the HCDR2 comprises the amino acid sequence of SEQ ID NO: 319, and the HCDR3 comprises the amino acid sequence of SEQ ID NO: 320; and (ii) the LCDR1 comprises the amino acid sequence of SEQ ID NO: 330, the LCDR2 comprises the amino acid sequence of SEQ ID NO: 331, and the LCDR3 comprises the amino acid sequence of SEQ ID NO: 332. In some embodiments, (i) the HCDR1 comprises the amino acid sequence of SEQ ID NO: 294, the HCDR2 comprises the amino acid sequence of SEQ ID NO: 321, and the HCDR3 comprises the amino acid sequence of SEQ ID NO: 322; and (ii) the LCDR1 comprises the amino acid sequence of SEQ ID NO: 327, the LCDR2 comprises the amino acid sequence of SEQ ID NO: 328, and the LCDR3 comprises the amino acid sequence of SEQ ID NO: 324.

In another aspect, this disclosure provides an isolated anti-IBP4 antibody or a fragment thereof, comprising: (i) a HCDR1 comprising amino acid sequence GYTFTDYY (SEQ ID NO: 75), a HCDR2 comprising amino acid sequence INPNNGGS (SEQ ID NO: 76), and a HCDR3 comprising amino acid sequence ARWDGGFDY (SEQ ID NO: 77); and (ii) a LCDR1 comprising amino acid sequence QNVGTN (SEQ ID NO: 87), a LCDR2 comprising amino acid sequence SAS (SEQ ID NO: 24), and a LCDR3 comprising amino acid sequence QQYNX₁YPLT (SEQ ID NO: 114) wherein X₁ is any naturally occurring amino acid residue. In further embodiments, X₁ comprises isoleucine or serine.

In another aspect, this disclosure provides an isolated anti-IBP4 antibody or a fragment thereof, comprising (i) a heavy chain comprising a HCDR1 comprising amino acid sequence GYTFTYX₁ (SEQ ID NO: 354), a HCDR2 comprising amino acid sequence IX₂X₃X₄X₅GGX₆ (SEQ ID NO: 355), and a HCDR3 comprising amino acid sequence X₇RX₈X₉X₁₀X₁₁X₁₂X₁₃X₁₄X₁₅X₁₆X₁₇DX₁₈ (SEQ ID NO: 356); and (ii) a light chain comprising a LCDR1 comprising amino acid sequence QNVX₁₉X₂₀X₂₁ (SEQ ID NO: 357), a LCDR2 comprising amino acid sequence SX₂₂S (SEQ ID NO: 358), and a LCDR3 comprising amino acid sequence QQYX₂₃X₂₄YPLX₂₅ (SEQ ID NO: 359), wherein each of X₁-X₅, X₇, X₁₂-X₁₄, X₁₅-X₂₀, X₂₂, and X₂₄—X₂₅ are independently any naturally occurring amino acid residue, wherein X₆ is independently any naturally occurring amino acid residue except isoleucine, wherein X₈ is independently a deletion or any naturally occurring amino acid residue except asparagine, wherein X₉ is independently any naturally occurring amino acid residue except tyrosine, wherein X₁₀ is independently a deletion or any naturally occurring amino acid residue, wherein X₁₁ is independently a deletion or any naturally occurring amino acid residue except isoleucine, wherein X₁₅ is independently a deletion or any naturally occurring amino acid residue, wherein X₁₆ is independently a deletion or any naturally occurring amino acid residue except alanine, wherein X₁₇ is independently any naturally occurring amino acid residue except valine, wherein X₂₁ is independently any naturally occurring amino acid residue except alanine, and wherein X₂₃ is independently any naturally occurring amino acid residue except serine. In some embodiments, X₁ comprises tyrosine or glutamic acid; X₂ comprises asparagine or aspartic acid; X₃ comprises proline or threonine; X₄ comprises glutamic acid or asparagine; X₅ comprises threonine or asparagine; X₆ comprises alanine or proline; X₇ comprises alanine or threonine; X₈ comprises alanine; X₉ comprises tryptophan or arginine; X₁₀ comprises glycine; Xii comprises tryptophan or serine; X₁₂ comprises aspartic acid or arginine or serine; X₁₃ comprises arginine or serine or glycine; X₁₄ comprises tyrosine or glycine or valine; X₁₅ comprises tyrosine; X₁₆ comprises tyrosine; X₁₇ comprises phenylalanine; X₁₈ comprises tyrosine or asparagine; X₁₉ comprises glycine or aspartic acid; X₂₀ comprises threonine or isoleucine; X₂₁ comprises aspartic acid or asparagine; X₂₂ comprises alanine or threonine; X₂₃ comprises asparagine or aspartic acid; X₂₄ comprises isoleucine or serine or threonine; X₂₅ comprises alanine or threonine, or a combination thereof.

In another aspect, this disclosure provides an isolated anti-IBP4 antibody or a fragment thereof, comprising: (i) a heavy chain comprising a HCDR1 comprising amino acid sequence GYTFTDYY (SEQ ID NO: 75), a HCDR2 comprising amino acid sequence INPNNGGX₁(SEQ ID NO: 360), and a HCDR3 comprising amino acid sequence ARWX₂X₃DX₄X₅FDY (SEQ ID NO: 361); and (ii) a light chain comprising a LCDR1 comprising amino acid sequence QNVGX₆X₇(SEQ ID NO: 362), a LCDR2 comprising amino acid sequence SAS (SEQ ID NO: 24), and a LCDR3 comprising amino acid sequence QQYNX₈YPLX₉ (SEQ ID NO: 363), wherein X₁ is independently any naturally occurring amino acid residue except isoleucine, wherein each of X₂ and X₃ are independently a deletion or any naturally occurring amino acid residue, wherein X₄ is independently any naturally occurring amino acid residue except serine, wherein each of X₅, X₆, X₈, and X₉ are independently any naturally occurring amino acid residue, and wherein X₇ is independently any naturally occurring amino acid residue except alanine. In some embodiments, X₁ comprises alanine or serine; X₂ comprises glycine; X₃ comprises tryptophan; X₄ comprises arginine or glycine; X₅ comprises tyrosine or glycine; X₆ comprises isoleucine or threonine; X₇ comprises aspartic acid or asparagine; X₈ comprises serine or isoleucine; X₉ comprises alanine or threonine; or a combination thereof.

In another aspect, this disclosure provides an isolated anti-IBP4 antibody or a fragment thereof, comprising: (i) a heavy chain comprising a HCDR1 comprising amino acid sequence GYTFTDYE (SEQ ID NO: 244), a HCDR2 comprising amino acid sequence IDX₁ETGGX₂ (SEQ ID NO: 364), and a HCDR3 comprising amino acid sequence TRARGSX₃SVYYFDX₄ (SEQ ID NO: 365); and (ii) a light chain comprising a LCDR1 comprising amino acid sequence QNVX₅TN (SEQ ID NO: 366), a LCDR2 comprising amino acid sequence SX₆S (SEQ ID NO: 367), and a LCDR3 comprising amino acid sequence QQYDX₇YPLT (SEQ ID NO: 368), wherein each of X₁, X₃, X₄, X₅, X₆, and X₇ are independently any naturally occurring amino acid residue, and wherein X₂ is independently any naturally occurring amino acid residue except isoleucine. In some embodiments, X₁ comprises proline or threonine; X₂ comprises proline or alanine; X₃ comprises serine or arginine; X₄ comprises tyrosine or asparagine; X₅ comprises glycine or aspartic acid; X₆ comprises alanine or threonine; X₇ comprises serine or threonine; or a combination thereof.

In another aspect, this disclosure provides an isolated anti-IBP4 antibody or a fragment thereof, comprising: (i) a heavy chain comprising an amino acid sequence that has at least 85%, 90%, 95%, 96%, 97%, 98%, or 99% identity to SEQ ID NO: 68, or a light chain comprising an amino acid sequence that has at least 85%, 90%, 95%, 96%, 97%, 98%, or 99% identity to SEQ ID NO: 70; (ii) a heavy chain comprising an amino acid sequence that has at least 85%, 90%, 95%, 96%, 97%, 98%, or 99% identity to SEQ ID NO: 103, or a light chain comprising an amino acid sequence that has at least 85%, 90%, 95%, 96%, 97%, 98%, or 99% identity to SEQ ID NO: 105; (iii) a heavy chain comprising an amino acid sequence that has at least 85%, 90%, 95%, 96%, 97%, 98%, or 99% identity to SEQ ID NO: 209, or a light chain comprising an amino acid sequence that has at least 85%, 90%, 95%, 96%, 97%, 98%, or 99% identity to SEQ ID NO: 211; (iv) a heavy chain comprising an amino acid sequence that has at least 85%, 90%, 95%, 96%, 97%, 98%, or 99% identity to SEQ ID NO: 237, or a light chain comprising an amino acid sequence that has at least 85%, 90%, 95%, 96%, 97%, 98%, or 99% identity to SEQ ID NO: 239; (v) a heavy chain comprising an amino acid sequence that has at least 85%, 90%, 95%, 96%, 97%, 98%, or 99% identity to SEQ ID NO: 266, or a light chain comprising an amino acid sequence that has at least 85%, 90%, 95%, 96%, 97%, 98%, or 99% identity to SEQ ID NO: 268; (vi) a heavy chain comprising an amino acid sequence that has at least 85%, 90%, 95%, 96%, 97%, 98%, or 99% identity to SEQ ID NO: 277, or a light chain comprising an amino acid sequence that has at least 85%, 90%, 95%, 96%, 97%, 98%, or 99% identity to SEQ ID NO: 279; or (vii) a heavy chain comprising an amino acid sequence that has at least 85%, 90%, 95%, 96%, 97%, 98%, or 99% identity to SEQ ID NO: 305, or a light chain comprising an amino acid sequence that has at least 85%, 90%, 95%, 96%, 97%, 98%, or 99% identity to SEQ ID NO: 307.

In some embodiments, the antibody or fragment thereof comprises the heavy chain of SEQ ID NO: 68. In some embodiments, the antibody or fragment thereof comprises the light chain of SEQ ID NO: 70. In some embodiments, the antibody or fragment thereof comprises the heavy chain of SEQ ID NO: 68 and the light chain of SEQ ID NO: 70. In some embodiments, the antibody or fragment thereof comprises the heavy chain of SEQ ID NO: 103. In some embodiments, the antibody or fragment thereof comprises the light chain of SEQ ID NO: 105. In some embodiments, the antibody or fragment thereof comprises the heavy chain of SEQ ID NO: 103 and the light chain of SEQ ID NO: 105. In some embodiments, the antibody or fragment thereof comprises the heavy chain of SEQ ID NO: 209. In some embodiments, the antibody or fragment thereof comprises the light chain of SEQ ID NO: 211. In some embodiments, the antibody or fragment thereof comprises the heavy chain of SEQ ID NO: 209 and the light chain of SEQ ID NO: 211. In some embodiments, the antibody or fragment thereof comprises the heavy chain of SEQ ID NO: 237. In some embodiments, the antibody or fragment thereof comprises the light chain of SEQ ID NO: 239. In some embodiments, the antibody or fragment thereof comprises the heavy chain of SEQ ID NO: 237 and the light chain of SEQ ID NO: 239.

In some embodiments, the antibody or fragment thereof comprises the heavy chain of SEQ ID NO: 266. In some embodiments, the antibody or fragment thereof comprises the light chain of SEQ ID NO: 268. In some embodiments, the antibody or fragment thereof comprises the heavy chain of SEQ ID NO: 266 and the light chain of SEQ ID NO: 268. In some embodiments, the antibody or fragment thereof comprises the heavy chain of SEQ ID NO: 277. In some embodiments, the antibody or fragment thereof comprises the light chain of SEQ ID NO: 279 In some embodiments, the antibody or fragment thereof comprises the heavy chain of SEQ ID NO: 277 and the light chain of SEQ ID NO: 279. In some embodiments, the antibody or fragment thereof comprises the heavy chain of SEQ ID NO: 305. In some embodiments, the antibody or fragment thereof comprises the light chain of SEQ ID NO: 307. In some embodiments, the antibody or fragment thereof comprises the heavy chain of SEQ ID NO: 305 and the light chain of SEQ ID NO: 307.

In another aspect, this disclosure provides an isolated anti-IBP4 antibody or a fragment thereof, comprising: (i) a heavy chain comprising an amino acid sequence that has at least 85%, 90%, 95%, 96%, 97%, 98%, or 99% identity to SEQ ID NO: 123, or a light chain comprising an amino acid sequence that has at least 85%, 90%, 95%, 96%, 97%, 98%, or 99% identity to SEQ ID NO: 70; (ii) a heavy chain comprising an amino acid sequence that has at least 85%, 90%, 95%, 96%, 97%, 98%, or 99% identity to SEQ ID NO: 128, or a light chain comprising an amino acid sequence that has at least 85%, 90%, 95%, 96%, 97%, 98%, or 99% identity to SEQ ID NO: 105; (iii) a heavy chain comprising an amino acid sequence that has at least 85%, 90%, 95%, 96%, 97%, 98%, or 99% identity to SEQ ID NO: 333, or a light chain comprising an amino acid sequence that has at least 85%, 90%, 95%, 96%, 97%, 98%, or 99% identity to SEQ ID NO: 211; (iv) a heavy chain comprising an amino acid sequence that has at least 85%, 90%, 95%, 96%, 97%, 98%, or 99% identity to SEQ ID NO: 335, or a light chain comprising an amino acid sequence that has at least 85%, 90%, 95%, 96%, 97%, 98%, or 99% identity to SEQ ID NO: 239; (v) a heavy chain comprising an amino acid sequence that has at least 85%, 90%, 95%, 96%, 97%, 98%, or 99% identity to SEQ ID NO: 337, or a light chain comprising an amino acid sequence that has at least 85%, 90%, 95%, 96%, 97%, 98%, or 99% identity to SEQ ID NO: 268; (vi) a heavy chain comprising an amino acid sequence that has at least 85%, 90%, 95%, 96%, 97%, 98%, or 99% identity to SEQ ID NO: 339, or a light chain comprising an amino acid sequence that has at least 85%, 90%, 95%, 96%, 97%, 98%, or 99% identity to SEQ ID NO: 279; or (vii) a heavy chain comprising an amino acid sequence that has at least 85%, 90%, 95%, 96%, 97%, 98%, or 99% identity to SEQ ID NO: 341, or a light chain comprising an amino acid sequence that has at least 85%, 90%, 95%, 96%, 97%, 98%, or 99% identity to SEQ ID NO: 307.

In some embodiments, the antibody or fragment thereof comprises the heavy chain of SEQ ID NO: 123. In some embodiments, the antibody or fragment thereof comprises the light chain of SEQ ID NO: 70. In some embodiments, the antibody or fragment thereof comprises the heavy chain of SEQ ID NO: 123 and the light chain of SEQ ID NO: 70. In some embodiments, the antibody or fragment thereof comprises the heavy chain of SEQ ID NO: 128. In some embodiments, the antibody or fragment thereof comprises the light chain of SEQ ID NO: 105. In some embodiments, the antibody or fragment thereof comprises the heavy chain of SEQ ID NO: 128 and the light chain of SEQ ID NO: 105. In some embodiments, the antibody or fragment thereof comprises the heavy chain of SEQ ID NO: 333. In some embodiments, the antibody or fragment thereof comprises the light chain of SEQ ID NO: 211. In some embodiments, the antibody or fragment thereof comprises the heavy chain of SEQ ID NO: 333 and the light chain of SEQ ID NO: 211. In some embodiments, the antibody or fragment thereof comprises the heavy chain of SEQ ID NO: 335. In some embodiments, the antibody or fragment thereof comprises the light chain of SEQ ID NO: 239. In some embodiments, the antibody or fragment thereof comprises the heavy chain of SEQ ID NO: 335 and the light chain of SEQ ID NO: 239.

In some embodiments, the antibody or fragment thereof comprises the heavy chain of SEQ ID NO: 337. In some embodiments, the antibody or fragment thereof comprises the light chain of SEQ ID NO: 268. In some embodiments, the antibody or fragment thereof comprises the heavy chain of SEQ ID NO: 337 and the light chain of SEQ ID NO: 268. In some embodiments, the antibody or fragment thereof comprises the heavy chain of SEQ ID NO: 339. In some embodiments, the antibody or fragment thereof comprises the light chain of SEQ ID NO: 279 In some embodiments, the antibody or fragment thereof comprises the heavy chain of SEQ ID NO: 339 and the light chain of SEQ ID NO: 279. In some embodiments, the antibody or fragment thereof comprises the heavy chain of SEQ ID NO: 341. In some embodiments, the antibody or fragment thereof comprises the light chain of SEQ ID NO: 307. In some embodiments, the antibody or fragment thereof comprises the heavy chain of SEQ ID NO: 341 and the light chain of SEQ ID NO: 307.

In some embodiments, this disclosure provided an anti-SHBG antibody or a fragment thereof, or an anti-IBP4 antibody or a fragment thereof, wherein the antibody or the fragment thereof is a FAB, Fab′, F(ab′)₂, Fv, scFv, (scFv)₂, single chain antibody molecule, dual variable region antibody, single variable region antibody, linear antibody, or a V region formed from antibody fragments. In some embodiments, the antibody or the fragment thereof is humanized, partially humanized, chimeric, or chimeric humanized. In some embodiments, the antibody or the fragment thereof is conjugated or recombinantly fused to a diagnostic or detectable agent. In some embodiments, the antibody or fragment thereof is bound to a solid support. In some embodiments, the solid support comprises a bead.

In another aspect, this disclosure provides an anti-SHBG or anti-IBP4 antibody or fragment thereof, wherein the anti-SHBG or anti-IBP4 antibody or fragment thereof binds to its respective antigen with an affinity measured by a dissociation constant (K_{D}) of about 1×10⁻⁷ M or less, 1×10⁻⁸ M or less, 1×10⁻⁹ M or less, 1×10⁻¹⁰ M or less, 2×10⁻⁷ M or less, 2×10⁻⁸ M or less, 2×10⁻⁹ M or less, 2×10⁻¹⁰ M or less, 5×10⁻⁷ M or less, 5×10⁻⁸ M or less, 5×10⁻⁹ M or less, or 5×10⁻¹⁰ M or less.

In another aspect, this disclosure provides a nucleic acid molecule encoding a heavy chain or a light chain of anti-SHBG antibody or a fragment thereof, or an anti-IBP4 antibody or a fragment thereof. In some embodiments, the nucleic acid molecule encodes a heavy chain of an anti-SHBG antibody or a fragment thereof or an anti-IBP4 antibody or a fragment thereof. In some embodiments, the nucleic acid molecule is an isolated nucleic acid molecule. In some embodiments, the nucleic acid molecule encodes a light chain of an anti-SHBG antibody or a fragment thereof or an anti-IBP4 antibody or a fragment thereof. In some embodiments, the nucleic acid molecule is an isolated nucleic acid molecule. In some embodiments, the nucleic acid molecule encodes a heavy chain and a light chain of an anti-SHBG antibody or a fragment thereof or an anti-IBP4 antibody or a fragment thereof.

In some embodiments, the nucleic acid molecule encoding the heavy chain has at least 85%, 90%, 95%, 96%, 97%, 98%, or 99% identity to at least one of SEQ ID NOS: 3, 35, 200, or 202; or the nucleic acid molecule encoding the light chain has at least 85%, 90%, 95%, 96%, 97%, 98%, or 99% identity to SEQ ID NO: 5, 37, 201, or 203. In some embodiments, the nucleic acid molecule encoding the heavy chain has at least 85%, 90%, 95%, 96%, 97%, 98%, or 99% identity to at least one of SEQ ID NOS: 67, 102, 204,206, 208, 236, 265, 276, 304, 334, 336, 338, 340, or 342; or the nucleic acid molecule encoding the light chain has at least 85%, 90%, 95%, 96%, 97%, 98%, or 99% identity to SEQ ID NO: 69, 104, 205,207, 210, 238, 267, 278 or 306. In some embodiments, this disclosure provides a vector comprising the nucleic acid molecule. In some embodiments, this disclosure provides an expression vector comprising the nucleic acid molecule. In some embodiments, this disclosure provides a cell comprising the nucleic acid molecule, vector, or the expression vector.

In another aspect, this disclosure provides a method for detecting one or more biomarkers in a biological sample comprising: (i) contacting a biological sample comprising one or more biomarkers to an anti-SHBG antibody or a fragment thereof, or to an IBP4 antibody or a fragment therein, and (ii) detecting binding of the antibody or the fragment thereof to the one or more biomarkers. In some embodiments, detecting the binding comprises a proteomics workflow that comprises mass spectrometry quantification. In some embodiments, the one or more biomarkers comprises SHBG, and the antibody or the fragment thereof is an anti-SHBG antibody or fragment thereof. In some embodiments, the one or more biomarkers comprises IBP4, and the antibody or the fragment thereof is an anti-IBP4 antibody or fragment thereof. In some embodiments, the one or more biomarkers comprises SHBG and IBP4, and wherein the antibody the antibody or the fragment thereof comprises an anti-SHBG antibody or a fragment thereof and an anti-IBP4 antibody or a fragment thereof. In some embodiments, the biological sample comprises blood or serum. In some embodiments, the detecting comprises separating the one or more biomarkers bound to the antibody or antibody fragment thereof from the biological sample. In some embodiments, the detecting comprises an immunoassay selected from the group consisting of sandwich immunoassay, immunohistochemistry, enzyme-linked immunosorbent assay (ELISA), Western blotting, nephelometry, turbidimetry, immunoradiometric assay, lateral flow, and immunehisto/cyto-chemistry.

In another aspect, this disclosure provides a method for assessing or predicting a health status of a subject, the method comprising: (i) obtaining a biological sample comprising one or more biomarkers from a subject; (ii) contacting the antibody or the fragment thereof of an anti-SHBG antibody or fragment thereof or an anti-IBP4 antibody or a fragment thereof to the biological sample under conditions suitable for the antibody or the fragment thereof to bind to the one or more biomarkers; and (iii) detecting the binding of the antibody or the fragment thereof to the one or more biomarker, wherein detecting the binding of the antibody to the one or more biomarkers indicates the presence of the one or more biomarkers in the biological sample and not detecting the binding of the antibody to the one or more biomarkers indicates the absence of the one or more biomarkers in the biological sample, and where the presence or absence of the one or more biomarker in the biological sample is used to assess or predict the health status of the subject. In some embodiments, further comprises quantitating an amount of the one or more biomarkers bound to the antibody or the fragment thereof in the biological sample, wherein the amount of the one or more biomarker is used to assess or predict the health status of the subject. In some embodiments, the subject is a pregnant female. In some embodiments, the health status comprises any one of abnormal glucola, gestational diabetes, hypertension, preeclampsia, intrauterine growth restriction, stillbirth, fetal growth restriction, HELLP syndrome, oligohyramnios, chorioamnionitis, chorioamnionitis, placental previa, placental acreta, abruption, abruptio placenta, placental hemorrhage, preterm premature rupture of membranes, preterm labor, unfavorable cervix, postterm pregnancy, cholelithiasis, or uterine over distention. In some embodiments, the detecting comprises separating the one or more biomarkers from the biological sample with the antibody or antibody fragment thereof. In some embodiments, detecting the binding comprises a proteomics workflow that comprises mass spectrometry quantification. In some embodiments, detecting comprises an immunoassay selected from the group consisting of sandwich immunoassay, immunohistochemistry, enzyme-linked immunosorbent assay (ELISA), Western blotting, nephelometry, turbidimetry, immunoradiometric assay, lateral flow, and immunohisto/cyto-chemistry. In some embodiments, the one or more biomarkers comprises SHBG, and wherein the antibody or the fragment thereof is an anti-SHBG antibody or fragment thereof as disclosed herein herein. In some embodiments, the one or more biomarkers comprises IBP4, and wherein the antibody or the fragment thereof is an anti-IBP4 antibody or fragment thereof. In some embodiments, the one or more biomarkers comprises SHBG and IBP4, and wherein the antibody or the fragment thereof comprises an anti-SHBG antibody or fragment thereof an anti-IBP4 antibody or fragment thereof as disclosed herein.

In another aspect, this disclosure provides a method of treating a subject comprising: administering an effective amount of an anti-SHBG antibody or fragment thereof or an anti-IBP4 antibody or fragment thereof as disclosed herein to the subject. In some embodiments, the subject is afflicted with a SHBG-associated disease, disorder, or condition, and wherein the subject is administered an anti-SHBG antibody or fragment thereof. In some embodiments, the SHBG-associated disease, disorder, or condition comprises abnormal glucola, gestational diabetes, hypertension, preeclampsia, intrauterine growth restriction, stillbirth, fetal growth restriction, HELLP syndrome, oligohyramnios, chorioamnionitis, chorioamnionitis, placental previa, placental acreta, abruption, abruptio placenta, placental hemorrhage, preterm premature rupture of membranes, preterm labor, unfavorable cervix, postterm pregnancy, cholelithiasis, placental dysfunction, premature cervical shortening, microbial dysbiosis, or uterine over distention. In some embodiments, the subject is afflicted with a IBP4-associated disease, disorder, or condition, and wherein the subject is administered an anti-IBP4 antibody or fragment thereof as disclosed herein. In some embodiments, the IBP4-associated disease, disorder, or condition comprises abnormal glucola, gestational diabetes, hypertension, preeclampsia, intrauterine growth restriction, stillbirth, fetal growth restriction, HELLP syndrome, oligohyramnios, chorioamnionitis, chorioamnionitis, placental previa, placental acreta, abruption, abruptio placenta, placental hemorrhage, preterm premature rupture of membranes, preterm labor, unfavorable cervix, postterm pregnancy, cholelithiasis, placental dysfunction, premature cervical shortening, microbial dysbiosis, or uterine over distention.

In another aspect, this disclosure provides a composition comprising: an anti-SHBG antibody or the fragment thereof or an anti-IBP4 antibody or fragment thereof; and a carrier. In some embodiments, the carrier comprises a solid support. In some embodiments, he solid support comprises a bead. In some embodiments, the carrier comprises a pharmaceutically acceptable carrier.

In another aspect, this disclosure provides a kit comprising: an anti-SHBG antibody or fragment thereof, or an anti-IBP4 antibody or fragment thereof; one or more ancillary reagents; and packaging for the same. In some embodiments, the anti-SHBG antibody or fragment thereof or the anti-IBP4 antibody or fragment thereof is attached to a solid support. In some embodiments, the solid support comprises a bead. In some embodiments, the kit comprises the anti-IBP4 antibody or the fragment thereof and the anti-SHBG antibody or the fragment thereof.

### BRIEF DESCRIPTION OF THE DRAWINGS

FIGS. 1A-1B show sequence alignments of heavy chain variable regions and light chain variable regions of anti-SHBG antibodies designated as 4G10.F8 and 13C9.E5. Boundaries of exemplary complementarity-determining regions (CDRs) are in bold. FIG. 1A shows the sequence alignment of heavy chain variable regions of anti-SHBG antibodies 4G10.F8 (SEQ ID NO: 8) and 13C9.E5 (SEQ ID NO: 40). FIG. 1B shows the sequence alignments of light chain variable regions of anti-SHBG antibodies 4G10.F8 (SEQ ID NO: 10) and 13C9.E5 (SEQ ID NO: 42).

FIGS. 2A-2B show sequence alignments of heavy chain variable regions and light chain variable regions of anti-IBP4 antibodies designated as 5B3.G9 and 7D6.G4. Boundaries of exemplary CDRs are in bold. FIG. 2A shows the sequence alignment of heavy chain variable regions of anti-IBP4 antibodies 7D6.G4 (SEQ ID NO: 107) and 5B3.G9 (SEQ ID NO: 72). FIG. 2B shows the sequence alignment of the light chain variable regions of anti-IBP4 antibodies 5B3.G9 (SEQ ID NO: 74) and 7D6.G4 (SEQ ID NO: 109).

FIGS. 3A and 3B show gel images of western blots demonstrating that anti-SHBG antibodies described herein bind to human SHBG protein. The gel images demonstrate that anti-SHBG antibodies designated variant 4G10.F8 and variant 13C9.E5 (FIGS. 3A and 3B, respectively) produce a strong signal against human serum containing SHB4 protein. Signals corresponding to the anti-SHBG antibodies are indicated by black arrows.

FIG. 4 shows Biocore data of exemplary anti-SHBG antibody clones. An exemplary clone, SHBG variant 4G10.F8, is labeled with a black arrow.

FIG. 5 shows data from a pooled peptide competition assay. The top panel shows a line graph demonstrating that a peptide pool of peptides 1+2+3 competes with variant 4G10.F8 and variant 13C9.E5 for binding with a target epitope. The y axis shows unblocked signal. The x axis shows concentrations of antibodies assayed. The lower panel shows a heatmap of the data from the line graph in the upper panel.

FIG. 6 shows data from single peptide competition assays. The top panel shows a line graph of the single peptide competition assays, with the y axis showing unblocked signal and the x axis showing concentration of antibodies assayed. The lower panel shows a heatmap of the data from the competition assay.

FIG. 7 shows an amino acid sequence (SEQ ID NO: 120) of a SHBG target epitope mapped against blocking peptides 1+2+3. The shaded amino acid represents a signal sequence that is not present in mature SHBG protein. Peptides 1+2+3 map against the underlined portion of the amino acid sequence.

FIGS. 8A and 8B show data from electrochemiluminescence immunoassays (ECLIA) characterizing the impact of SHBG glycosylation on antibody binding. FIG. 8A shows the percent signal remaining in an ECLIA assay in which anti-SHBG antibody variant 13C9.E5 was used to bind SHBG serum from pregnant and non-pregnant females. FIG. 8B shows samples treated with a de-glycosylation enzyme (wells labeled with “+”) produce a lower signal than cells not treated with de-glycosylation enzyme (e.g., compare wells 1 and 2).

FIG. 9 shows an amino acid sequence of a SHBG epitope (SEQ ID NO: 121). A glycosylation site of the SHBG epitope is labeled with a black arrow at position T36. Additional sites for glyscolsylation are identified by an underlined “N”. The underlined portion of the epitope is recognized by SHBG variant antibodies 4G10.F8, and 13C9.E5.

FIG. 10 shows a gel image of a western blot confirming anti-IBP4 antibody clones variant 5B3.G9 and variant 7D6.G4 bind human IBP4 protein. Bind to human IBP4 protein. Signals corresponding to the anti-IBP4 variant antibodies 5B3.G9 and 7D6.G4 are indicated by the black arrows. Additional candidate antibodies assayed are indicated above their respective lanes.

FIGS. 11A and 11B shows data from direct competitive binding assays involving electrochemiluminescence immunoassay (ECLIA) performed with anti-IBP4 variant antibody clones 5B3.G9 and 7D6.G4. FIG. 11A illustrates that the signal of clone variant 5B3.G9 decreases with increased concentration of variant 7D6.G4. FIG. 11B illustrates that the signal of variant 7D6.G4 decreases with increased concentration of antibody clone variant 5B3.G9.

FIG. 12 shows data from ECLIA competitive binding assays that map the epitope of variant 5B3.G9 and variant 7D6.G4 antibody clones (SEQ ID NOs: 344-353, 369).

FIGS. 13A-13B show correlation plots of IBP4 response ratios (RR) as measured by alternating current mass spectrometry (AC-MS) and an antibody enrichment assay referred herein as CLIA. FIG. 13A shows data from beads coupled to IBP4-5B3.G9 antibodies for AC-MS. FIG. 13B shows beads coupled to IBP4-7D6.G4 antibodies for AC-MS. IBP4 response ratios from each AC-MS assay were plotted against the IBP4 RR data from the CLIA assay.

FIGS. 14A-14F show correlation plots of anti-SHBG antibody response ratios (RR) from AC-MS assays and antibody enrichment assays referred to as CLIA. Data from assays with beads coupled to anti-SHBG antibodies are shown in: FIG. 14A: variant 13C9.E5; FIG. 14B: 4G10.F8; FIG. 14C: 16A6.A2; FIG. 14D: 1G7.C5; FIG. 14E: 11C6.A8; and FIG. 14F: SHBG-6001 control anti-SHBG antibody. SHBG response ratios from AC-MS assay were plotted against the response ratios from CLIA assay.

FIG. 15 shows AC-MS data from IBP4 and SHBG bead combinations.

FIG. 16 shows the Direct ECLIA data generated for the five additional anti-IBP4 antibodies (1H9.B6, 5C11.A7, 7G11.H4, 3D4.E9, and 1G3.A3) compared with 5B3.G9, demonstrating their similar affinity to 5B3.G9 as a reference.

FIG. 17 shows the Competition Direct ECLIA Data demonstrating each of the respective anti-IBP4 antibodies' ability to block 5B3.G9 from binding to its epitope, therefore suggesting they bind to the same or similar epitopes.

FIGS. 18A-18C show correlation plots comparing AC-MS and CLIA IBP4 Response Ratios (RRs). FIG. 18A shows M-PVA beads coupled to the 1H9.B6 anti-IBP4 antibody for AC-MS. FIG. 18B shows beads coupled to the 5C11.A7 anti-IBP4 antibody for AC-MS. FIG. 18C shows beads coupled to the 7G11.H4 anti-IBP4 antibody for AC-MS. IBP4 response ratios from each AC-MS assay were plotted against the IBP4 RR data from the CLIA assay.

FIG. 19 shows the heavy chain CLUSTAL O (1.2.4) full-length multiple sequence alignment for the 1G3.A3 (SEQ ID NO: 305), 1H9.B6 (SEQ ID NO: 209), 3D4.E9 (SEQ ID NO: 277), 5C11.A7 (SEQ ID NO: 237), 7G11.H4 (SEQ ID NO: 266), 7D6.G4 (SEQ ID NO: 103), and 5B3.G9 (SEQ ID NO: 68) anti-IBP4 antibodies.

FIG. 20 shows the light chain CLUSTAL O (1.2.4) full-length multiple sequence alignment for the 1G3.A3 (SEQ ID NO: 307), 1H9.B6 (SEQ ID NO: 70), 3D4.E9 (SEQ ID NO: 279), 5C11.A7 (SEQ ID NO: 239), 7G11.H4 (SEQ ID NO: 268), 7D6.G4 (SEQ ID NO: 105), and 5B3.G9 (SEQ ID NO: 70) anti-IBP4 antibodies.

FIGS. 21A-21B show the heavy chain and light chain IMGT consensus CDRs (complementarity determining regions) for the anti-IBP4 antibodies 1H9.B6, 5C11.A7, 7G11.H4, 5B3.G9, 7D6.G4, 3D4.E9, and 1G3.A3. The CDR nomenclature is shown below the sequences. FIG. 21A shows the heavy chain IMGT consensus CDRs. Sequences for each CDRH1, CDRH2, and CDRH3 are shown in the left, center, and right columns, respectively (SEQ ID NOs: 275, 216 and 217 for 1H9.B6; SEQ ID NOs: 244, 245, and 246 for 5C11.A7 and 7G11.H4; SEQ ID NOs: 75, 216 and 77 for 5B3.G9 and 7D6.G4; SEQ ID NOs: 244, 284 and 285 for 3D4.E9; and SEQ ID NOs. 244, 312 and 313 for 1G3.A3). FIG. 21B shows the light chain IMGT consensus CDRs. Sequences for each CDRL1, CDRL2, and CDRL3 are shown in the left, center, and right columns, respectively (SEQ ID NOs. 226, 24 and 227 for 1H9.B6; SEQ ID NOs. 87, 24 and 259 for 5C11.A7; SEQ ID NOs. 23, 24 and 259 for 7G11.H4; SEQ ID NOs. 87, 24 and 89 for 5B3.G9; SEQ ID NOs. 87, 24 and 99 for 7D6.G4; SEQ ID NOs. 87, 297 and 298 for 3D4.E9; and SEQ ID NOs. 323, 24 and 324 for 1G3.A3).

FIGS. 22A-22B show the heavy chain variable regions and light chain variable regions for the anti-IBP4 antibodies 1G3.A3, 1H9.B6, 3D4.E9, 5C11.A7, 7G11.H4, 7D6.G4, and 5B3.G9. Bold and underlined residues indicate less favorable residues from the 1G3.A3 clone according to the alignment of these antibodies. FIG. 22A shows the heavy chain variable regions for 1G3.A3 (SEQ ID NO: 309), 1H9.B6 (SEQ ID NO: 213), 3D4.E9 (SEQ ID NO: 281), 5C11.A7 (SEQ ID NO: 241), 7G11.H4 (SEQ ID NO: 241), 7D6.G4 (SEQ ID NO: 107), and 5B3.G9 (SEQ ID NO: 72). FIG. 22B shows the light chain variable regions for 1G3.A3 (SEQ ID NO: 311), 1H9.B6 (SEQ ID NO: 215), 3D4.E9 (SEQ ID NO: 283), 5C11.A7 (SEQ ID NO: 243), 7G11.H4 (SEQ ID NO: 271), 7D6.G4 (SEQ ID NO: 109), and 5B3.G9 (SEQ ID NO: 74).

FIGS. 23A-23B show the CLUSTAL O (1.2.4) multiple sequence alignments for the heavy chain variable regions for 2 groups of anti-IBP4 antibodies: 1H9.B6, 7D6.G4, 5B3.G9 and 1G3.A3 in one group (Group 1), and 3D4.E9, 5C11.A7, 7G11.H4, and 1G3.A3 in another group (Group 2). HCDRs for each alignment and group are highlighted, and are the same as those in FIGS. 21A-21B. Bold and underlined residues indicate less favorable residues from the 1G3.A3 clone according to the alignment of these antibodies. CDR nomenclature is shown below the sequence alignment for each group. FIG. 23A shows the sequence alignment for the heavy chain variable regions for Group 1: 1G3.A3 (SEQ ID NO: 309), 1H9.B6 (SEQ ID NO: 213), 7D6.G4 (SEQ ID NO: 107), and 5B3.G9 (SEQ ID NO: 72). FIG. 23B shows the sequence alignment for the heavy chain variable regions for Group 2: 1G3.A3 (SEQ ID NO: 309), 3D4.E9 (SEQ ID NO: 281), 5C11.A7 (SEQ ID NO: 241), and 7G11.H4 (SEQ ID NO: 241).

FIGS. 24A-24B show the CLUSTAL O (1.2.4) multiple sequence alignments for the light chain variable regions for 2 groups of anti-IBP4 antibodies: 1H9.B6, 7D6.G4, 5B3.G9 and 1G3.A3 in one group (Group 1), and 3D4.E9, 5C11.A7, 7G11.H4, and 1G3.A3 in another group (Group 2). LCDRs for each alignment and group are highlighted, and are the same as those in FIGS. 21A-21B. Bold and underlined residues indicate less favorable residues from the 1G3.A3 clone according to the alignment of these antibodies. CDR nomenclature is shown below the sequence alignment for each group. FIG. 24A shows the sequence alignment for the light chain variable regions for Group 1: 1G3.A3 (SEQ ID NO: 311), 1H9.B6 (SEQ ID NO: 215), 7D6.G4 (SEQ ID NO: 109), and 5B3.G9 (SEQ ID NO: 74). FIG. 24B shows the sequence alignment for the light chain variable regions for Group 2: 1G3.A3 (SEQ ID NO: 311), 3D4.E9 (SEQ ID NO: 283), 5C11.A7 (SEQ ID NO: 243), and 7G11.H4 (SEQ ID NO: 271).

### DETAILED DESCRIPTION OF THE INVENTION

Discussion of documents, acts, materials, devices, articles or the like which has been included in the present specification is for the purpose of providing context for aspects of this disclosure. Such discussion is not an admission that any or all of these matters form part of the prior art with respect to any embodiments disclosed herein.

Unless defined otherwise, all technical and scientific terms used herein have the same meaning as commonly understood to one of ordinary skill in the art to which this disclosure pertains. Otherwise, certain terms used herein have the meanings as set forth in the specification.

The present disclosure provides SHBG and IBP4 binding agents. Such agents include antibodies or antigen-binding fragments thereof that bind SHBG or IBP4. Such binding agents are useful in compositions and in various methods of detecting the presense (including quantitating the amount of) or absence of SHBG and/or IBP4 in a sample, including methods for determining probability of preterm birth and preeclampsia. The binding agents described herein are also useful for treatment of certain health conditions.

### Definitions

The term “SHBG,” “sex hormone-binding protein,” “SHBG polypeptide,” and similar terms refers to a polypeptide (“polypeptide” and “protein” are used interchangeably herein) or any native SHBG from any vertebrate source, including mammals such as primates (e.g., humans, cynomolgus monkey (cyno)), dogs, and rodents (e.g., mice and rats), unless otherwise indicated. The term SHBG includes any species homolog. The term SHBG also includes any SHBG that is naturally expressed by cells or can be expressed on cells transfected with genes or cDNA encoding the polypeptide. SHBG, also known in the art as androgen binding protein (ABG) and testosterone-estrogen binding globulin (TEBG), has an N-terminal steroid binding and dimerization domain, and a C-terminal domain containing a higly-conserved consensus sequence for glycosylation.

SHBG is a steroid binding protein secreted by the liver that functions as a regulator of steroid responses. SHBG is secreted by the liver into the blood and can bind androgens and estrogens. Furthermore, in a pregnant female, SHBG has been reported in the human placenta Beyond regulating steroid responses, SHBG plays a role in the prediction of preterm birth, increased length of neonatal hospital stay and increased severity of adverse neonatal outcomes. See, Markenson et al., “Performance of a proteomic preterm delivery predictor in a large independent prospective cohort.” American Journal of Obstetrics & Gynecology, 2(3): 100140 (2020).

The term SHBG encompasses “full-length,” unprocessed SHBG, as well as any form of SHBG or any fragment thereof that results from processing in the cell, including the four known alternatively spliced isoforms of SHBG that differ in the length of the intracellular tail. The term SHBG also encompasses naturally occurring variants of SHBG, such as SNP variants, splice variants and allelic variants. Unless noted, preferably the SHBG is a human SHBG. The full-length human SHBG amino acid sequence is provided by GenBank Accession Number NP_001031:

(SEQ ID NO: 1)
MESRGPLATSRLLLLLLLLLLRHTRQGWALRPVLPTQSAHDPPAVHL
SNGPGQEPIAVMTFDLTKITKTSSSFEVRTWDPEGVIFYGDTNPKDD
WFMLGLRDGRPEIQLHNHWAQLTVGAGPRLDDGRWHQVEVKMEGDSV
LLEVDGEEVLRLRQVSGPLTSKRHPIMRIALGGLLFPASNLRLPLVP
ALDGCLRRDSWLDKQAEISASAPTSLRSCDVESNPGIFLPPGTQAEF
NLRDIPQPHAEPWAFSLDLGLKQAAGSGHLLALGTPENPSWLSLHLQ
DQKVVLSSGSGPGLDLPLVLGLPLQLKLSMSRVVLSQGSKMKALALP
PLGLAPLLNLWAKPQGRLFLGALPGEDSSTSFCLNGLWAQGQRLDVD
QALNRSHEIWTHSC PQSPGNGTDASH.

Other related SHBG polypeptides that are also encompassed by the term SHBG include fragments, derivatives (e.g., substitution, deletion, truncations, and insertion variants), fusion polypeptides, and interspecies homologs that retain SHBG activity. As those skilled in the art will appreciate, a SHBG binding agent (e.g., an antibody) described herein can bind to a SHBG polypeptide, a SHBG polypeptide fragment, a SHBG antigen, and/or a SHBG epitope. An epitope may be part of a larger SHBG antigen, which may be part of a larger SHBG polypeptide fragment, which, in turn, may be part of a larger SHBG polypeptide. SHBG may exist in a native or denatured form. SHBG polypeptides described herein may be isolated from a variety of sources, such as from human tissue types or from another source, or prepared by recombinant or synthetic methods. A SHBG polypeptide may comprise a polypeptide having the same amino acid sequence as a corresponding SHBG polypeptide derived from nature. Orthologs to the SHBG polypeptide are also well known in the art.

The term “IBP4,” “insulin-like growth factor binding protein 4,” “IBP4 polypeptide,” and similar terms refer to a polypeptide or any native IBP4 from any vertebrate source, including mammals such as primates (e.g., humans, cynomolgus monkey (cyno)), dogs, and rodents (e.g., mice and rats), unless otherwise indicated. The term IBP4 also includes any species homolog. The term IBP4 also includes any IBP4 is naturally expressed by cells or can be expressed on cells transfected with genes or cDNA encoding the polypeptide. IBP4, also known in the art as “insulin-like growth factor binding protein 4” IGFBP4), has 3 structurally distinct domains, comprising a third of the molecule, with domain 1 being the IGF-binding domain.

IBP4 is a binding protein secreted by the ovary, uterus, liver, placenta, among others that prolongs the half-life of insulin-like growth factors. Beyond regulating growth responses, IBP4 plays a role in the prediction of preterm birth, increased length of neonatal hospital stay and increased severity of adverse neonatal outcomes. See, Markenson et al., “Performance of a proteomic preterm delivery predictor in a large independent prospective cohort.” American Journal of Obstetrics & Gynecology, 2(3): 100140 (2020).

The term IBP4 encompasses “full-length,” unprocessed IBP4, as well as any form of IBP4 or any fragment thereof that results from processing in the cell, including the four known alternatively spliced isoforms of IBP4 that differ in the length of the intracellular tail. The term IBP4 also encompasses naturally occurring variants of IBP4, such as SNP variants, splice variants and allelic variants. Unless noted, preferably the IBP4 is a human IBP4. The full-length human IBP4 amino acid sequence is provided by GenBank Accession Number NP_001543:

(SEQ ID NO: 2)
MLPLCLVAALLLAAGPGPSLGDEAIHCPPCSEEKLARCRPPVGCEEL
VREPGCGCCATCALGLGMPCGVYTPRCGSGLRCYPPRGVEKPLHTLM
HGQGVCMELAEIEAIQESLQPSDKDEGDHPNNSFSPCSAHDRRCLQK
HFAKIRDRSTSGGKMKVNGAPREDARPVPQGSCQSELHRALERLAAS
QSRTHEDLYIIPIPNCDRNGNFHPKQCHPALDGQRGKCWCVDRKTGV
KLPGGLEPKGELDCHQLADSFRE.

Other related IBP4 polypeptides that are also encompassed by the term IBP4 include fragments, derivatives (e.g., substitution, deletion, truncations, and insertion variants), fusion polypeptides, and interspecies homologs that retain IBP4 activity. As those skilled in the art will appreciate, a IBP4 binding agent (e.g., an antibody) described herein can bind to a IBP4 polypeptide, a IBP4 polypeptide fragment, a IBP4 antigen, and/or a IBP4 epitope. An epitope may be part of a larger IBP4 antigen, which may be part of a larger IBP4 polypeptide fragment, which, in turn, may be part of a larger IBP4 polypeptide. IBP4 may exist in a native or denatured form. IBP4 polypeptides described herein may be isolated from a variety of sources, such as from human tissue types or from another source, or prepared by recombinant or synthetic methods. A IBP4 polypeptide may comprise a polypeptide having the same amino acid sequence as a corresponding IBP4 polypeptide derived from nature. Orthologs to the IBP4 polypeptide are also well known in the art.

The terms “treating,” “treatment,” and the like are used herein to generally mean obtaining a desired pharmacologic and/or physiologic effect. The effect may be prophylactic in terms of completely or partially preventing a disease and/or may be therapeutic in terms of a partial or complete cure for a disease and/or adverse effect attributable to the disease. Treatment as used herein covers any intervention of a disease in a subject and includes: preventing the disease from occurring in a subject which may be predisposed to the disease but has not yet been diagnosed as having it; inhibiting the disease, i.e., arresting its development; or relieving the disease, i.e., causing regression of the disease. Treatment also includes any approach for obtaining beneficial or desired results, including clinical results. Non-limiting beneficial or desired results can include one or more: alleviation or amelioration of one or more symptoms, diminishment of extent of a condition (including a disease), stabilized (i.e., not worsening) state of a condition (including disease), or delay or slowing of a condition (including disease).

It must be noted that, as used herein, the singular forms “a”, “an” and “the” include plural references, unless the content clearly dictates otherwise and are used interchangeably with “at least one” and “one or more.” Thus, for example, reference to “a binding agent” includes a mixture of two or more binding agents, and the like.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the disclosure. Such equivalents are intended to be encompassed by this disclosure.

As used herein, the term “binding agent” or a grammatical equivalent thereof refers to a molecule (e.g., antibody) with one or more antigen binding sites that binds an antigen. For example, a SHBG binding agent as described herein includes an antibody, antibody fragment, or other peptide-based molecule that binds to SHBG. As another example, an IBP4 binding agent as described herein includes an antibody, antibody fragment, or other peptide-based molecule that binds to IBP4.

As used herein, the term “antibody” is used in a broad sense and includes any “immunoglobulin,” or “Ig” molecules and specifically covers, human, humanized, composite and chimeric antibodies, and antibody fragments that are polyclonal or monoclonal antibodies (including agonist, antagonist, neutralizing antibodies, full length monoclonal antibodies), antibody compositions with polyepitopic or monoepitopic specificity, recombinantly produced antibodies, monospecific antibodies, multispecific antibodies (including bispecific antibodies), synthetic antibodies, chimeric antibodies, humanized antibodies, or human versions of antibodies having full length heavy and/or light chains. In general, antibodies are proteins or peptide chains that exhibit binding specificity to a specific antigen. Antibodies may be agonistic antibodies or antagonistic antibodies. Antibody structures are well known.

The present disclosure also includes antibody fragments (and/or polypeptides that comprise antibody fragments) that retain SHBG or IBP4 binding characteristics. Non-limiting examples of antibody fragments include antigen-binding regions and/or effector regions of the antibody, e.g., Fab, Fab′, F(ab′)₂, Fv, scFv, (scFv)₂, single chain antibody molecule, dual variable region antibody, single variable region antibody, linear antibody, V region, a multispecific antibody formed from antibody fragments, F(ab)₂, Fd, Fc, diabody, di-diabody, disulfide-linked Fvs (dsFv), single-domain antibody (e.g., nanobody) or other fragments (e.g., fragments consisting of the variable regions of the heavy and light chains that are non-covalently coupled),. In general terms, a variable (V) region domain may be any suitable arrangement of immunoglobulin heavy (VH) and/or light (VL) chain variable domains. For example, the present disclosure also includes tetrameric antibodies comprising two heavy chain and two light chain molecules, an antibody light chain monomer, and an antibody heavy chain monomer. Thus, for example, the V region domain may be dimeric and contain VH-VH, VH-VL, or VL-VL dimers that bind SHBG or IBP4. If desired, the VH and VL chains may be covalently coupled either directly or through a linker to form a single chain Fv (scFv). For ease of reference, scFv proteins are referred to herein as included in the category “antibody fragments.” Another form of an antibody fragment is a peptide comprising one or more complementarity determining regions (CDRs) of an antibody. CDRs (also termed “minimal recognition units” or “hypervariable region”) can be obtained by constructing polynucleotides that encode the CDR of interest. Such polynucleotides are prepared, for example, by using the polymerase chain reaction to synthesize the variable region using mRNA of antibody-producing cells as a template (see, for example, Larrick et al., Methods: A Companion to Methods in Enzymology, 2:106 (1991); Courtenay-Luck, “Genetic Manipulation of Monoclonal Antibodies,” in Monoclonal Antibodies Production, Engineering and Clinical Application, Ritter et al. (eds.), page 166, Cambridge University Press (1995); and Ward et al., “Genetic Manipulation and Expression of Antibodies,” in Monoclonal Antibodies: Principles and Applications, Birch et al., (eds.), page 137, Wiley-Liss, Inc. (1995)). Antibody fragments may be incorporated into single domain antibodies, maxibodies, minibodies, intrabodies, diabodies, triabodies, tetrabodies, variable domains of new antigen receptors (v-NAR), and bis-single chain Fv regions (see, e.g., Hollinger and Hudson, Nature Biotechnology, 23(9):1126-1136, 2005). The binding agent, in some embodiments, contains a light chain and/or a heavy chain constant region, such as one or more constant regions, including one or more IgG1, IgG2, IgG3 and/or IgG4 constant regions. In some embodiments, antibodies can include epitope-binding fragments of any of the above. The antibodies described herein can be of any class (e.g., IgG, IgE, IgM, IgD, and IgA) or any subclass (e.g., IgG1, IgG2, IgG3, IgG4, IgA1, and IgA2) of immunoglobulin molecule. IgA and IgG are further sub-classified as the isotypes IgA1, IgA2, IgG1, IgG2, IgG3 and IgG4. Antibody light chains of vertebrate species can be assigned to one of two clearly distinct types, namely kappa and lambda, based on the amino acid sequences of their constant domains. Accordingly, the antibodies of this disclosurecan contain a kappa or lambda light chain constant domain. According to particular embodiments, the antibodies disclosed herein include heavy and/or light chain constant regions from mouse or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

As used herein, the term an “isolated antibody” refers to an antibody which is substantially free of other antibodies having different antigenic specificities (e.g., an isolated antibody that specifically binds to SHBG is substantially free of antibodies that do not bind to SHBG. In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that can be present in minor amounts. The monoclonal antibodies disclosed herein can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, an antibody that “specifically binds to SHBG” or “specifically binds to IBP4” refers to an antibody that binds to a SHBG or IBP4, preferably a human SHBG or IBP4, with a K_{D} of 1×10⁻⁷ M or less, preferably 1×10⁻⁸ M or less, more preferably 5×10⁻⁹ M or less, 1×10⁻⁹ M or less, 5×10⁻¹⁰ M or less, or 1×10⁻¹⁰ M or less. For example, an anti-SHBG antibody described herein may a K_{D} of about 1.0×10⁻⁹M to about 2.5×10⁻⁹M. An anti-IBP4 antibody described herein may have a K_{D} of about 3×10⁻⁸ M to about 8×10⁻⁸ M. The term “K_{D}” refers to the dissociation constant, which is obtained from the ratio of K_{d} to Kₐ (i.e., K_{d}/Kₐ) and is expressed as a molar concentration (M). K_{D} values for antibodies can be determined using methods in the art in view of the present disclosure. For example, the K_{D} of an antibody can be determined by using surface plasmon resonance, such as by using a biosensor system, e.g., a Biacore® system, or by using bio-layer interferometry technology, such as an Octet RED96 system. The smaller the value of the K_{D} of an antibody, the higher affinity that the antibody binds to a target antigen.

The terms “identical” or percent “identity,” in the context of two or more nucleic acids or polypeptide sequences (e.g., anti-SHBG antibodies and polynucleotides that encode them, SHBG polypeptides and SHBG polynucleotides that encode them, anti-IBP4 antibodies and polynucleotides that encode them, IBP4 polypeptides and IBP4 polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “label” refers to a molecular entity that emits a signal and can be used as a readout or measurement for detection of an analyte. Various classes of labels exist. Such labels include a fluorophore, an enzyme, a chemiluminscent moiety, a radioactive moiety, an organic dye, a small molecule, a polypeptide or functional fragment thereof. Examples of fluorophores include fluorescent dyes like phycoerytherin (PE), fluorescein isothiocyanate (FITC), tetramethylrhodamine (TRITC), BODIPY and AlexaFluor® dyes. Fluorescent dyes can also include fluorescence resonance energy transfer (FRET)-dyes or time-resolved (TR)-FRET dyes. Fluorophore labels also include fluorescent proteins such as green fluorescent protein (GFP) and cyan fluorescent protein (CFP). Examples of enzyme labels include alkaline phosphatase (AP) or horseradish peroxidase (HRP). When any of the substrates 3,3′5,5′-Tetramethylbenzidine (TMB), 3,3′-Diaminobenzidene (DAB), or 2,2′-azino-bis(3-ethylbenzothiazoline-6-sulphonic acid) (ABTS) are applied to HRP, a colored (chromogenic) or light (chemiluminescent) signal is produced. Radioactive moiety labels include carbon-14 or Tritium. Small molecule labels include biotin, resins such as agarose beads and fluorescently labeled magnetic beads, or nanoparticles such as colloidal gold. Polypeptide or functional fragment labels include Avidin, Streptavidin or NeutrAvidin which have an affinity for biotin. Polypeptide or functional fragment labels also include hemagglutinin (HA), glutathione-S-transferase (GST) or c-myc.

A label of the present disclosure can be conjugated to any of the antibodies, antigen binding fragments thereof or binding agents disclosed herein. Conjugation can include non-covalent or covalent cross-linkage as described above. In some configurations, a label conjugated to a detection probe requires an additional substrate or binding agent described above. As an example, an HRP label conjugated to an anti-SHBG antibody or antigen binding fragment thereof requires a substrate, disclosed above. Numerous other configurations for a label are known in the art. The present disclosure includes all label configurations exemplified herein and/or known in the art. In some embodiments, a label configuration can include PE conjugated to an anti-SHBG antibody, antigen binding fragment thereof or binding agent.

As used herein, the term “preterm birth” refers to delivery or birth at a gestational age less than 37 completed weeks. Other commonly used subcategories of preterm birth have been established and delineate moderately preterm (birth at 33 to 36 weeks of gestation), very preterm (birth at <33 weeks of gestation), and extremely preterm (birth at ≤28 weeks of gestation). With regard to the methods disclosed herein, those skilled in the art understand that the cut-offs that delineate preterm birth and term birth as well as the cut-offs that delineate subcategories of preterm birth can be adjusted in practicing the methods disclosed herein, for example, to maximize a particular health benefit. In various embodiments of this disclosure, cut-off that delineate preterm birth include, for example, birth at 37 weeks of gestation, ≤36 weeks of gestation, ≤35 weeks of gestation, ≤34 weeks of gestation, ≤33 weeks of gestation, ≤32 weeks of gestation, ≤30 weeks of gestation, ≤29 weeks of gestation, ≤28 weeks of gestation, ≤27 weeks of gestation, ≤26 weeks of gestation, ≤25 weeks of gestation, ≤24 weeks of gestation, ≤23 weeks of gestation or 22 weeks of gestation. In some embodiments, the cut-off delineating preterm birth is ≤35 weeks of gestation. It is further understood that such adjustments are well within the skill set of individuals considered skilled in the art and encompassed within the scope of the this disclosure. Gestational age is a proxy for the extent of fetal development and the fetus's readiness for birth. Gestational age has typically been defined as the length of time from the date of the last normal menses to the date of birth. However, obstetric measures and ultrasound estimates also can aid in estimating gestational age. Preterm births have generally been classified into two separate subgroups. One, spontaneous preterm births are those occurring subsequent to spontaneous onset of preterm labor or preterm premature rupture of membranes regardless of subsequent labor augmentation or cesarean delivery. Two, medically indicated preterm births are those occurring following induction or cesarean section for one or more conditions that the woman's caregiver determines to threaten the health or life of the mother and/or fetus and not in the presence of spontaneous initiation of labor. Also, it may be that voluntary preterm birth for non-life-threatening reasons will still be denoted as medically indicated. In some embodiments, the methods disclosed herein are directed to determining the probability for spontaneous preterm birth or medically indicated preterm birth. In some embodiments, the methods disclosed herein are directed to determining the probability for spontaneous preterm birth. In additional embodiments, the methods disclosed herein are directed to medically indicated preterm birth. In additional embodiments, the methods disclosed herein are directed to predicting gestational age at birth.

As used herein, the term “biological sample,” encompasses any sample that is taken from pregnant female and contains one or more of the biomarkers disclosed herein. Suitable samples in the context of this disclosure include, for example, blood, plasma, serum, amniotic fluid, vaginal secretions, saliva, and urine. In some embodiments, the biological sample is selected from the group consisting of whole blood, plasma, and serum. In a particular embodiment, the biological sample is serum. As will be appreciated by those skilled in the art, a biological sample can include any fraction or component of blood, without limitation, T cells, monocytes, neutrophils, erythrocytes, platelets and microvesicles such as exosomes and exosome-like vesicles. In a particular embodiment, the biological sample is serum.

As used herein, the term “diagnostic agent” or “detecteable agent” refers to a compound that can be used to detect, purify, isolate, or enrich a target, in particular a biomarker.

A “proteomics work-flow” generally encompasses one or more of the following steps: Serum samples are thawed and depleted of the 14 highest abundance proteins by immune-affinity chromatography. Depleted serum is digested with a protease, for example, trypsin, to yield peptides. The digest is subsequently fortified with a mixture of SIS peptides and then desalted and subjected to LC-MS/MS with a triple quadrupole instrument operated in MRM mode. Response ratios are formed from the area ratios of endogenous peptide peaks and the corresponding SIS peptide counterpart peaks. Those skilled in the art appreciate that other types of MS such as, for example, MALDI-TOF, or ESI-TOF, can be used in the methods of this disclosure. In addition, one skilled in the art can modify a proteomics work-flow, for example, by selecting particular reagents (such as proteases) or omitting or changing the order of certain steps, for example, it may not be necessary to immunodeplete, the SIS peptide could be added earlier or later and stable isotope labeled proteins could be used as standards instead of peptides.

As used herein, the term “mass spectrometer” refers to a device able to volatilize/ionize analytes to form gas-phase ions and determine their absolute or relative molecular masses. Suitable methods of volatilization/ionization are matrix-assisted laser desorption ionization (MALDI), electrospray, laser/light, thermal, electrical, atomized/sprayed and the like, or combinations thereof. Suitable forms of mass spectrometry include, but are not limited to, ion trap instruments, quadrupole instruments, electrostatic and magnetic sector instruments, time of flight instruments, time of flight tandem mass spectrometer (TOF MS/MS), Fourier-transform mass spectrometers, Orbitraps and hybrid instruments composed of various combinations of these types of mass analyzers. These instruments can, in turn, be interfaced with a variety of other instruments that fractionate the samples (for example, liquid chromatography or solid-phase adsorption techniques based on chemical, or biological properties) and that ionize the samples for introduction into the mass spectrometer, including matrix-assisted laser desorption (MALDI), electrospray, or nanospray ionization (ESI) or combinations thereof.

Generally, any mass spectrometric (MS) technique that can provide precise information on the mass of peptides, and preferably also on fragmentation and/or (partial) amino acid sequence of selected peptides (e.g., in tandem mass spectrometry, MS/MS; or in post source decay, TOF MS), can be used in the methods disclosed herein. Suitable peptide MS and MS/MS techniques and systems are well-known per se (see, e.g., Methods in Molecular Biology, vol. 146: “Mass Spectrometry of Proteins and Peptides”, by Chapman, ed., Humana Press 2000; Biemann 1990. Methods Enzymol 193: 455-79; or Methods in Enzymology, vol. 402: “Biological Mass Spectrometry”, by Burlingame, ed., Academic Press 2005) and can be used in practicing the methods disclosed herein. Accordingly, in some embodiments, the disclosed methods comprise performing quantitative MS to measure one or more biomarkers. Such quantitative methods can be performed in an automated (Villanueva, et al., Nature Protocols (2006) 1(2):880-891) or semi-automated format. In particular embodiments, MS can be operably linked to a liquid chromatography device (LC-MS/MS or LC-MS) or gas chromatography device (GC-MS or GC-MS/MS). Other methods useful in this context include isotope-coded affinity tag (ICAT), tandem mass tags (TMT), or stable isotope labeling by amino acids in cell culture (SILAC), followed by chromatography and MS/MS.

As used herein, the terms “multiple reaction monitoring (MRM)” or “selected reaction monitoring (SRM)” refer to an MS-based quantification method that is particularly useful for quantifying analytes that are in low abundance. In an SRM experiment, a predefined precursor ion and one or more of its fragments are selected by the two mass filters of a triple quadrupole instrument and monitored over time for precise quantification. Multiple SRM precursor and fragment ion pairs can be measured within the same experiment on the chromatographic time scale by rapidly toggling between the different precursor/fragment pairs to perform an MRM experiment. A series of transitions (precursor/fragment ion pairs) in combination with the retention time of the targeted analyte (e.g., peptide or small molecule such as chemical entity, steroid, hormone) can constitute a definitive assay. A large number of analytes can be quantified during a single LC-MS experiment. The term “scheduled,” or “dynamic” in reference to MRM or SRM, refers to a variation of the assay wherein the transitions for a particular analyte are only acquired in a time window around the expected retention time, significantly increasing the number of analytes that can be detected and quantified in a single LC-MS experiment and contributing to the selectivity of the test, as retention time is a property dependent on the physical nature of the analyte. A single analyte can also be monitored with more than one transition. Finally, included in the assay can be standards that correspond to the analytes of interest (e.g., same amino acid sequence), but differ by the inclusion of stable isotopes. Stable isotopic standards (SIS) can be incorporated into the assay at precise levels and used to quantify the corresponding unknown analyte. An additional level of specificity is contributed by the co-elution of the unknown analyte and its corresponding SIS and properties of their transitions (e.g., the similarity in the ratio of the level of two transitions of the unknown and the ratio of the two transitions of its corresponding SIS).

Mass spectrometry assays, instruments and systems suitable for biomarker peptide analysis can include, without limitation, matrix-assisted laser desorption/ionisation time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)n; ion mobility spectrometry (IMS); inductively coupled plasma mass spectrometry (ICP-MS)atmospheric pressure photoionization mass spectrometry (APPI-MS); APPI-MS/MS; and APPI-(MS)n. Peptide ion fragmentation in tandem MS (MS/MS) arrangements can be achieved using manners established in the art, such as, e.g., collision induced dissociation (CID). As described herein, detection and quantification of biomarkers by mass spectrometry can involve multiple reaction monitoring (MRM), such as described among others by Kuhn et al. Proteomics 4: 1175-86 (2004). Scheduled multiple-reaction-monitoring (Scheduled MRM) mode acquisition during LC-MS/MS analysis enhances the sensitivity and accuracy of peptide quantitation. Anderson and Hunter, Molecular and Cellular Proteomics 5(4):573 (2006). As described herein, mass spectrometry-based assays can be advantageously combined with upstream peptide or protein separation or fractionation methods, such as for example with the chromatographic and other methods described herein below. As further described herein, shotgun quantitative proteomics can be combined with SRM/MRM-based assays for high-throughput identification and verification of prognostic biomarkers of preterm birth

Antibodies that Bind to SHBG

Described herein are isolated anti-SHBG antibodies or antigen-binding fragments thereof, polypeptides and fragments thereof, nucleic acids and expression vectors encoding the antibodies, recombinant cells containing the vectors, and compositions comprising the antibodies. The antibodies disclosed herein possess one or more desirable functional properties, including, but not limited, to high-affinity binding to SHBG and specifically bind to SHBG, which as described herein provides the ability to detect, diagnosis, or treat certain conditions. The antibodies disclosed herein are likewise useful to assess a health status, or predict an adverse pregnancy event.

In one aspect, this disclosure relates to an isolated anti-SHBG antibody or antigen-binding fragment thereof. In some embodiments, the isolated anti-SHBG antibody or the fragment thereof binds to a SHBG epitope. The SHBG epitope can comprise a sequence that has at least 50%, 55%, 60%, 65%, 70%, 75%, 80%, 85%, 90%, 95%, 99%, or 100% identity to SEQ ID NO: 110. In some embodiments, the isolated anti-SHBG antibody or the fragment thereof binds to a SHBG epitope. The SHBG epitope can comprise a sequence that has at least 50%, 55%, 60%, 65%, 70%, 75%, 80%, 85%, 90%, 95%, 99%, or 100% identity to SEQ ID NO: 120. In some embodiments, the isolated anti-SHBG antibody or antigen-binding comprises a heavy chain, wherein the heavy chain comprises a heavy chain complementarity determining region 1 (HCDR1), HCDR2, and HCDR3 comprising an amino acid sequence selected from the group consisting of SEQ ID NOs:11-22, 115, and 116. In some embodiments, the isolated anti-SHBG antibody or antigen-binding comprises a light chain, wherein the light chain comprises a light chain complementarity determining region 1 (LCDR1), LCDR2, and LCDR3 comprising an amino acid sequence selected from the group consisting of SEQ ID NOs:23-34. In some embodiments, the isolated anti-SHBG antibody or antigen-binding comprises a heavy chain, wherein the heavy chain comprises a heavy chain complementarity determining region 1 (HCDR1), HCDR2, and HCDR3 and/or a light chain complementarity determining region 1 (LCDR1), LCDR2, and LCDR3 comprising any one or more amino acid sequences shown in Table 14 or in Table 16. In some embodiments, the anti-SHBG antibody provided herein comprises or consists of six CDRs. For example, in some emboidments, the anti-SHBG antibody provided herein comprises a HCDR1, HCDR2, and HCDR3, wherein the HCDRs comprise amino acid sequences selected from the group consisting of SEQ ID NOs: 11-22, 44, 45, 47, 48, 50-54, 115, or 117, and further comprises a light chain, wherein the light chain comprises a LCDR1, a LCDR2, and a LCDR3 comprising an amino acid sequence selected from the group consisting of SEQ ID NOs: 23-34. In other embodiments, the anti-SHBG antibody provided herein comprises less than the six CDRs of the anti-SHBG antibodies described herein. In some embodiments, the heavy chain is associated with the light chain to form a binding site for SHBG. In some embodiments, the antibodies provided herein comprise a heavy chain variable region. In other embodiments, the antibodies provided herein comprise a light chain variable region or VL chain. In some embodiments, the antibodies provided herein have a combination of (i) a heavy chain variable region; and/or (ii) a light chain variable region.

In some embodiments, an antibody provided herein comprises or consists of six CDRs, for example, HCDR1, HCDR2, HCDR3, LCDR1, LCDR2, and/or LCDR3 as identified in Tables 14 and 16. In some embodiments, an antibody provided herein can comprise less than six CDRs. In some embodiments, the antibody comprises or consists of one, two, three, four, or five CDRs selected from the group consisting of HCDR1, HCDR2, HCDR3, LCDR1, LCDR2, and/or LCDR3 identified in Tables 14 and 16. In some embodiments, the antibody comprises or consists of one, two, three, four, or five CDRs selected from the group consisting of HCDR1, HCDR2, HCDR3, LCDR1, LCDR2, and/or LCDR3 of the monoclonal antibody selected from the group consisting of: (a) the antibody designated 13C9.E5; (b) the antibody designated variant 13C9.E5; (c) the antibody designated 4G10.F8; or (d) the antibody designated variant 4G10.F8. Accordingly, in some embodiments, the antibody comprises or consists of one, two, three four or five CDRs of anyone of the HCDR1, HCDR2, HCDR3, LCDR1, LCDR2, and/or LCDR3 identified in Tables 14 and 16.

In some embodiments, the antibodies provided herein comprise one or more (e.g., one, two or three) HCDRs listed in Tables 14 and 16. In other embodiments, the antibodies provided herein comprise one or more (e.g., one, two or three) LCDRs listed in Tables 14 and 16. In yet other embodiments, the antibodies provided herein comprise one or more (e.g., one, two or three) HCDRs listed in Tables 14 and 16 and one or more LCDRs listed in Tables 14 and 16.

Accordingly, in certain embodiments, the antibodies comprise a variable heavy (VH) chain CDR1 having the amino acid sequence of any one of SEQ ID NOS: 11, 14, 17, 20, 52, and 115. In another embodiment, the antibodies comprise a VH CDR2 having the amino acid sequence of any one of SEQ ID NOS: 12, 15, 18, 21, 44, 47, 50, 53, 116, and 117. In another embodiment, the antibodies comprise a VH CDR3 having the amino acid sequence of any one of SEQ ID NOS: 13, 16, 19, 22, 45, 48, 51, and 54. In certain embodiments, the antibodies comprise a VH CDR1 and/or a VH CDR2 and/or a VH CDR3 independently selected from a VH CDR1, VH CDR2, VH CDR3 as depicted in any one of the amino acid sequences depicted in Table 14, and Table 16. In certain embodiments, the antibodies comprise a VL CDR1 having the amino acid sequence of any one of SEQ ID NOS: 23, 26, 29, and 32. In another embodiment, the antibodies comprise a variable light (VL) chain CDR2 having the amino acid sequence of any one of SEQ ID NOS: 24, 27, 30, and 33. In another embodiment, the antibodies comprise a VL CDR3 having the amino acid sequence of any one of SEQ ID NOS: 25, 31, and 34. In certain embodiments, the antibodies comprise a VL CDR1 and/or a VL CDR2 and/or a VL CDR3 independently selected from a VL CDR1, VL CDR2, VL CDR3 as depicted in any one of the amino acid sequences depicted in Table 14 and Table 16.

Also provided herein are antibodies comprising one or more (e.g., one, two or three) VH CDRs and one or more (e.g., one, two or three) VL CDRs listed in Table 14, and Table 16. In particular, provided herein is an antibody comprising: a VH CDR1 (SEQ ID NOS: 11, 14, 17, 20, 52, and 115) and a VL CDR1 (SEQ ID NOS: 23, 26, 29, and 32); a VH CDR1 (SEQ ID NOS: 11, 14, 17, 20, 52, and 115) and a VL CDR2 (SEQ ID NOS: 24, 27, 30, and 33); a VH CDR1 (SEQ ID NOS: 11, 14, 17, 20, 52, and 115) and a VL CDR3 (SEQ ID NOS: 25, 31, and 34); a VH CDR2 (SEQ ID NOS: 12, 15, 18, 21, 44, 47, 50, 53, 116, and 117) and a VL CDR1 (SEQ ID NOS: 23, 26, 29, and 32); a VH CDR2 (SEQ ID NOS: 12, 15, 18, 21, 44, 47, 50, 53, 116, and 117) and a VL CDR2 (SEQ ID NOS: 24, 27, 30, and 33); a VH CDR2 (SEQ ID NOS: 12, 15, 18, 21, 44, 47, 50, 53, 116, and 117) and a VL CDR3 (SEQ ID NOS: 25, 31, and 34); a VH CDR3 (SEQ ID NOS: 13, 16, 19, 22, 45, 48, 51, and 54) and a VL CDR1 (SEQ ID NOS: 23, 26, 29, and 32); a VH CDR3 (SEQ ID NOS: 13, 16, 19, 22, 45, 48, 51, and 54) and a VL CDR2 (SEQ ID NOS: 24, 27, 30, and 33); a VH CDR3 (SEQ ID NOS: 13, 16, 19, 22, 45, 48, 51, and 54) and a VL CDR3 (SEQ ID NOS: 25, 31, and 34); a VH CDR1 (SEQ ID NOS: 11, 14, 17, 20, 52, and 115), a VH CDR2 (SEQ ID NOS: 12, 15, 18, 21, 44, 47, 50, 53, 116, and 117) and a VL CDR1 (SEQ ID NOS: 23, 26, 29, and 32); a VH CDR1 (SEQ ID NOS: 11, 14, 17, 20, 52, and 115), a VH CDR2 (SEQ ID NOS: 12, 15, 18, 21, 44, 47, 50, 53, 116, and 117) and a VL CDR2 (SEQ ID NOS: 24, 27, 30, and 33); a VH CDR1 (SEQ ID NOS: 11, 14, 17, 20, 52, and 115), a VH CDR2 (SEQ ID NOS: 12, 15, 18, 21, 44, 47, 50, 53, 116, and 117) and a VL CDR3 (SEQ ID NOS: 25, 31, and 34); a VH CDR2 (SEQ ID NOS: 12, 15, 18, 21, 44, 47, 50, 53, 116, and 117), a VH CDR3 (SEQ ID NOS: 13, 16, 19, 22, 45, 48, 51, and 54) and a VL CDR1 (SEQ ID NOS: 23, 26, 29, and 32), a VH CDR2 (SEQ ID NOS: 12, 15, 18, 21, 44, 47, 50, 53, 116, and 117), a VH CDR3 (SEQ ID NOS: 13, 16, 19, 22, 45, 48, 51, and 54) and a VL CDR2 (SEQ ID NOS: 24, 27, 30, and 33); a VH CDR2 (SEQ ID NOS: 12, 15, 18, 21, 44, 47, 50, 53, 116, and 117), a VH CDR3 (SEQ ID NOS: 13, 16, 19, 22, 45, 48, 51, and 54) and a VL CDR3 (SEQ ID NOS: 25, 31, and 34); a VH CDR1 (SEQ ID NOS: 11, 14, 17, 20, 52, and 115), a VL CDR1 (SEQ ID NOS: 23, 26, 29, and 32) and a VL CDR2 (SEQ ID NOS: 24, 27, 30, and 33); a VH CDR1 (SEQ ID NOS: 11, 14, 17, 20, 52, and 115), a VL CDR1 (SEQ ID NOS: 23, 26, 29, and 32) and a VL CDR3 (SEQ ID NOS: 25, 31, and 34); a VH CDR1 (SEQ ID NOS: 11, 14, 17, 20, 52, and 115), a VL CDR2 (SEQ ID NOS: 24, 27, 30, and 33) and a VL CDR3 (SEQ ID NOS: 25, 31, and 34); a VH CDR2 (SEQ ID NOS: 12, 15, 18, 21, 44, 47, 50, 53, 116, and 117), a VL CDR1 (SEQ ID NOS: 23, 26, 29, and 32) and a VL CDR2 (SEQ ID NOS: 24, 27, 30, and 33); a VH CDR2 (SEQ ID NOS: 12, 15, 18, 21, 44, 47, 50, 53, 116, and 117), a VL CDR1 (SEQ ID NOS: 23, 26, 29, and 32) and a VL CDR3 (SEQ ID NOS: 25, 31, and 34); a VH CDR2 (SEQ ID NOS: 12, 15, 18, 21, 44, 47, 50, 53, 116, and 117), a VL CDR2 (SEQ ID NOS: 24, 27, 30, and 33) and a VL CDR3 (SEQ ID NOS: 25, 31, and 34); a VH CDR3 (SEQ ID NOS: 13, 16, 19, 22, 45, 48, 51, and 54), a VL CDR1 (SEQ ID NOS: 23, 26, 29, and 32) and a VL CDR2 (SEQ ID NOS: 24, 27, 30, and 33); a VH CDR3 (SEQ ID NOS: 13, 16, 19, 22, 45, 48, 51, and 54), a VL CDR1 (SEQ ID NOS: 23, 26, 29, and 32) and a VL CDR3 (SEQ ID NOS: 25, 31, and 34); a VH CDR3 (SEQ ID NOS: 13, 16, 19, 22, 45, 48, 51, and 54), a VL CDR2 (SEQ ID NOS: 24, 27, 30, and 33) and a VL CDR3 (SEQ ID NOS: 25, 31, and 34); a VH CDR1 (SEQ ID NOS: 11, 14, 17, 20, 52, and 115), a VH CDR2 (SEQ ID NOS: 12, 15, 18, 21, 44, 47, 50, 53, 116, and 117), a VH CDR3 (SEQ ID NOS: 13, 16, 19, 22, 45, 48, 51, and 54) and a VL CDR1 (SEQ ID NOS: 23, 26, 29, and 32); a VH CDR1 (SEQ ID NOS: 11, 14, 17, 20, 52, and 115), a VH CDR2 (SEQ ID NOS: 12, 15, 18, 21, 44, 47, 50, 53, 116, and 117), a VH CDR3 (SEQ ID NOS: 13, 16, 19, 22, 45, 48, 51, and 54) and a VL CDR2 (SEQ ID NOS: 24, 27, 30, and 33); a VH CDR1 (SEQ ID NOS: 11, 14, 17, 20, 52, and 115), a VH CDR2 (SEQ ID NOS: 12, 15, 18, 21, 44, 47, 50, 53, 116, and 117), a VH CDR3 (SEQ ID NOS: 13, 16, 19, 22, 45, 48, 51, and 54) and a VL CDR3 (SEQ ID NOS: 25, 31, and 34); a VH CDR1 (SEQ ID NOS: 11, 14, 17, 20, 52, and 115), a VH CDR2 (SEQ ID NOS: 12, 15, 18, 21, 44, 47, 50, 53, 116, and 117), a VL CDR1 (SEQ ID NOS: 23, 26, 29, and 32) and a VL CDR2 (SEQ ID NOS: 24, 27, 30, and 33); a VH CDR1 (SEQ ID NOS: 11, 14, 17, 20, 52, and 115), a VH CDR2 (SEQ ID NOS: 12, 15, 18, 21, 44, 47, 50, 53, 116, and 117), a VL CDR1 (SEQ ID NOS: 23, 26, 29, and 32) and a VL CDR3 (SEQ ID NOS: 25, 31, and 34); a VH CDR1 (SEQ ID NOS: 11, 14, 17, 20, 52, and 115), a VH CDR2 (SEQ ID NOS: 12, 15, 18, 21, 44, 47, 50, 53, 116, and 117), a VL CDR2 (SEQ ID NOS: 24, 27, 30, and 33) and a VL CDR3 (SEQ ID NOS: 25, 31, and 34); a VH CDR1 (SEQ ID NOS: 11, 14, 17, 20, 52, and 115), a VH CDR3 (SEQ ID NOS: 13, 16, 19, 22, 45, 48, 51, and 54), a VL CDR1 (SEQ ID NOS: 23, 26, 29, and 32) and a VL CDR2 (SEQ ID NOS: 24, 27, 30, and 33); a VH CDR1 (SEQ ID NOS: 11, 14, 17, 20, 52, and 115), a VH CDR3 (SEQ ID NOS: 13, 16, 19, 22, 45, 48, 51, and 54), a VL CDR1 (SEQ ID NOS: 23, 26, 29, and 32) and a VL CDR3 (SEQ ID NOS: 25, 31, and 34); a VH CDR1 (SEQ ID NOS: 11, 14, 17, 20, 52, and 115), a VH CDR3 (SEQ ID NOS: 13, 16, 19, 22, 45, 48, 51, and 54), a VL CDR2 (SEQ ID NOS: 24, 27, 30, and 33) and a VL CDR3 (SEQ ID NOS: 25, 31, and 34); a VH CDR2 (SEQ ID NOS: 12, 15, 18, 21, 44, 47, 50, 53, 116, and 117), a VH CDR3 (SEQ ID NOS: 13, 16, 19, 22, 45, 48, 51, and 54), a VL CDR1 (SEQ ID NOS: 23, 26, 29, and 32) and a VL CDR2 (SEQ ID NOS: 24, 27, 30, and 33); a VH CDR2 (SEQ ID NOS: 12, 15, 18, 21, 44, 47, 50, 53, 116, and 117), a VH CDR3 (SEQ ID NOS: 13, 16, 19, 22, 45, 48, 51, and 54), a VL CDR1 (SEQ ID NOS: 23, 26, 29, and 32) and a VL CDR3 (SEQ ID NOS: 25, 31, and 34); a VH CDR2 (SEQ ID NOS: 12, 15, 18, 21, 44, 47, 50, 53, 116, and 117), a VH CDR3 (SEQ ID NOS: 13, 16, 19, 22, 45, 48, 51, and 54), a VL CDR2 (SEQ ID NOS: 24, 27, 30, and 33) and a VL CDR3 (SEQ ID NOS: 25, 31, and 34); a VH CDR1 (SEQ ID NOS: 11, 14, 17, 20, 52, and 115), a VH CDR2 (SEQ ID NOS: 12, 15, 18, 21, 44, 47, 50, 53, 116, and 117), a VH CDR3 (SEQ ID NOS: 13, 16, 19, 22, 45, 48, 51, and 54), a VL CDR1 (SEQ ID NOS: 23, 26, 29, and 32) and a VL CDR2 (SEQ ID NOS: 24, 27, 30, and 33); a VH CDR1 (SEQ ID NOS: 11, 14, 17, 20, 52, and 115), a VH CDR2 (SEQ ID NOS: 12, 15, 18, 21, 44, 47, 50, 53, 116, and 117), a VH CDR3 (SEQ ID NOS: 13, 16, 19, 22, 45, 48, 51, and 54), a VL CDR1 (SEQ ID NOS: 23, 26, 29, and 32) and a VL CDR3 (SEQ ID NOS: 25, 31, and 34); a VH CDR1 (SEQ ID NOS: 11, 14, 17, 20, 52, and 115), a VH CDR2 (SEQ ID NOS: 12, 15, 18, 21, 44, 47, 50, 53, 116, and 117), a VH CDR3 (SEQ ID NOS: 13, 16, 19, 22, 45, 48, 51, and 54), a VL CDR2 (SEQ ID NOS: 24, 27, 30, and 33) and a VL CDR3 (SEQ ID NOS: 25, 31, and 34); a VH CDR1 (SEQ ID NOS: 11, 14, 17, 20, 52, and 115), a VH CDR2 (SEQ ID NOS: 12, 15, 18, 21, 44, 47, 50, 53, 116, and 117), a VL CDR1 (SEQ ID NOS: 23, 26, 29, and 32), a VL CDR2 (SEQ ID NOS: 24, 27, 30, and 33), and a VL CDR3 (SEQ ID NOS: 25, 31, and 34); a VH CDR1 (SEQ ID NOS: 11, 14, 17, 20, 52, and 115), a VH CDR3 (SEQ ID NOS: 13, 16, 19, 22, 45, 48, 51, and 54), a VL CDR1 (SEQ ID NOS: 23, 26, 29, and 32), a VL CDR2 (SEQ ID NOS: 24, 27, 30, and 33), and a VL CDR3 (SEQ ID NOS: 25, 31, and 34); a VH CDR2 (SEQ ID NOS: 12, 15, 18, 21, 44, 47, 50, 53, 116, and 117), a VH CDR3 (SEQ ID NOS: 13, 16, 19, 22, 45, 48, 51, and 54), a VL CDR1 (SEQ ID NOS: 23, 26, 29, and 32), a VL CDR2 (SEQ ID NOS: 24, 27, 30, and 33), and a VL CDR3 (SEQ ID NOS: 25, 31, and 34); or any combination thereof of the VH CDRs (SEQ ID NOS: 11, 12, 13, 14, 15, 16, 17, 18, 19, 20, 21, 22, 44, 45, 47, 48, 50, 51, 52, 53, 54, 115, 116, 117) and VL CDRs (SEQ ID NOS: 23, 24, 25, 26, 27, 29, 30, 31, 32, 33) listed in Table 14 and Table 16.

In some embodiments, the isolated anti-SHBG antibody or antigen-binding comprises a heavy chain, wherein the heavy chain comprises a heavy chain complementarity determining region 1 (HCDR1), HCDR2, and HCDR3 comprising an amino acid sequence selected from the group consisting of SEQ ID NOs: 11-22, 44, 45, 47, 48, 50-54, 115, or 117. In some embodiments, the isolated anti-SHBG antibody or antigen-binding comprises and a light chain, wherein the light chain comprises a light chain complementarity determining region 1 (LCDR1), LCDR2, and LCDR3 comprising an amino acid sequence selected from the group consisting of SEQ ID NOs: 23-34. In some embodiments, the heavy chain is associated with the light chain to form a binding site for SHBG.

The anti-SHBG antibody or antigen-binding fragment thereof can comprise a heavy chain that comprises an amino acid sequence with at least 85%, preferably 90%, more preferably 95% or more, such as 95%, 96%, 97%, 98%, or 99% identity to an amino acid sequence selected from the group consisting of SEQ ID NOs: 4, 36, 124, or 126. In some embodiments, the anti-SHBG antibody or antigen-binding fragment thereof can comprise a heavy chain variable region that comprises an amino acid sequence with at least 85%, preferably 90%, more preferably 95% or more, such as 95%, 96%, 97%, 98%, or 99% identity to an amino acid sequence selected from the group consisting of SEQ ID NOs: 8 and 40. In some embodiments, anti-SHBG antibody or antigen-binding fragment thereof can comprises a light chain that comprises an amino acid sequence with at least 85%, preferably 90%, more preferably 95% or more, such as 95%, 96%, 97%, 98%, or 99% identity to an amino acid sequence selected from the group consisting of SEQ ID Nos: 6, 38, 125, or 127. In some embodiments, the anti-SHBG antibody or antigen-binding fragment thereof comprises a light chain variable region that comprises an amino acid sequence with at least 85%, preferably 90%, more preferably 95% or more, such as 95%, 96%, 97%, 98%, or 99% identity to an amino acid sequence selected from the group consisting of SEQ ID NOs: 10 and 42. In some embodiments, the heavy chain is associated with the light chain to form a binding site for SHBG. In some embodiments, the anti-SHBG antibody or antigen fragment thereof is humanized.

In some embodiments, the anti-SHBG antibody or antigen-binding fragment is monoclonal. In some embodiments, the anti-SHBG antibody or antigen-binding fragment is a FAB, Fab′, F(ab′)₂, Fv, scFv, (scFv)₂, single chain antibody molecule, dual variable region antibody, single variable region antibody, linear antibody, or a V region formed from antibody fragments. In some embodiments, the anti-SHBG antibody or antigen-binding fragment is humanized, human or chimeric.

In some embodiments, the anti-SHBG antibody is conjugated or recombinantly fused to a diagnostic or detectable agent. In some embodiments, the conjugated or recombinantly linked antibodies, include masked or activatable conjugates, and can be useful, for example, for preventing a disease or disorder such as preterm birth, preeclampsia or any other pregnancy related disorder. The conjugated or recombinantly fused anti-SHBG antibody or antigen-binding fragments thereof can be useful, for example, for monitoring or prognosing the onset, development, progression, and/or severity of preterm birth, preeclampsia or any other pregnancy related disorder.

In some embodiments, the diagnostic or detectable agent includes, for example: enzymes, including, but not limited to, horseradish peroxidase, alkaline phosphatase, beta-galactosidase, or acetylcholinesterase; prosthetic groups, including, but not limited to, streptavidin/biotin or avidin/biotin; fluorescent materials, including, but not limited to, umbelliferone, fluorescein, fluorescein isothiocynate, rhodamine, dichlorotriazinylamine fluorescein, dansyl chloride, or phycoerythrin; luminescent materials, including, but not limited to, luminol; bioluminescent materials, including, but not limited to, luciferase, luciferin, or aequorin; chemiluminescent material, including, but not limited to, an acridinium based compound or a HALOTAG; radioactive materials, including, but not limited to, iodine (131I, 125I, 123I, and 121I), carbon (14C), sulfur (35S), tritium (3H), indium (111In, 113In, 112In, and 111In), technetium (99Tc), thallium (201Ti), gallium (68Ga and 67Ga), palladium (103Pd), molybdenum (99Mo), xenon (133Xe), fluorine (18F), 153Sm, 177Lu, 159Gd, 149Pm, 140La, 175Yb, 166Ho, 90Y, 47Sc, 186Re, 188Re, 142Pr, 105Rh, 97Ru, 68Ge, 57Co, 65Zn, 85Sr, 32P, 153Gd, 169Yb, 51Cr, 54Mn, 75Se, 113Sn, or 117Sn; positron emitting metals using various positron emission tomographies; and non-radioactive paramagnetic metal ions.

In another aspect, this disclosure relates to a binding agent that binds to the same epitope as the anti-SHBG antibody or antigen-binding fragment disclosed herein. In some embodiments, a SHBG binding agent includes a polypeptide or a fragment thereof, that is not an antibody.

In another aspect, this disclosure relates to isolated nucleic acids encoding the anti-SHBG antibody or antigen-binding fragments thereof disclosed herein. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding anti-SHBG antibodies disclosed herein can be altered without changing the amino acid sequences of the proteins.

In another aspect, this disclosure relates to a nucleic acid molecule encoding a heavy chain and/or a light chain of an antibody or fragment thereof disclosed herein. The nucleic acid molecule is preferable an isolated nucleic acid molecule. The isolated nucleic acid molecule can be a nuclic acid molecule that is substantially pure. In some embodiments, the nucleic acid molecule encodes a heavy chain of an anti-SHBG antibody or fragment thereof. In some embodiments, the nucleic acid molecule encoding the heavy chain has at least 85%, 90%, 95%, 96%, 97%, 98%, or 99% identity to at least one of SEQ ID NOS: 3, 35, 200, or 202. In some embodiments, the nucleic acid molecule encodes a light chain of an anti-SHBG antibody or fragment thereof. In some embodiments, the nucleic acid molecule encoding the light chain has at least 85%, 90%, 95%, 96%, 97%, 98%, or 99% identity to SEQ ID NO: 5, 37, 201, or 203.

In another aspect, this disclosure relates to vectors comprising the isolated nucleic acids disclosed herein. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to embodiments disclosed herein. Such techniques are well known to those skilled in the art in view of the present disclosure.

In another aspect, this disclosure relates to host cells comprising the isolated nucleic acids encoding the anti-SHBG antibodies or antigen-binding fragments thereof disclosed herein. Any host cell known to those skilled in the art in view of the present disclosure can be used for recombinant expression of antibodies or antigen-binding fragments thereof disclosed herein. In some embodiments, the host cells are E. coli TG1 or BL21 cells (for expression of, e.g., an scFv or Fab antibody), CHO-DG44 or CHO-K1 cells or HEK293 cells (for expression of, e.g., a full-length IgG antibody). According to particular embodiments, the recombinant expression vector is transformed into host cells by conventional methods such as chemical transfection, heat shock, or electroporation, where it is stably integrated into the host cell genome such that the recombinant nucleic acid is effectively expressed.

In another aspect, this disclosure relates to a method of producing an anti-SHBG antibody or antigen-binding fragment thereof disclosed herein. The methods comprise culturing a cell comprising a nucleic acid encoding the anti-SHBG antibody or antigen-binding fragment thereof disclosed herein under conditions to produce the anti-SHBG antibody or antigen-binding fragment thereof, and recovering the antibody or antigen-binding fragment thereof from the cell or cell culture (e.g., from the supernatant). Expressed antibodies or antigen-binding fragments thereof can be harvested from the cells and purified according to conventional techniques known in the art and as described herein.

In another aspect, this disclosure relates to a buffered composition comprising the isolated anti-SHBG antibody or antigen-binding fragment thereof as provided herein. In some embodiments, the buffered composition can have at least 0.5%, 1%, 2%, 3%, 4%, 5%, 6%, 7%, 8%, 9%0, 10%, 15%, 20%, 25%, 30%, 35%, 40%, 45%, 50%, 55%, 60%, 65%, 70%, 75%, 80%, 85%, 90%, or more by weight of the anti-SHBG antibodies or other binding agents having an antigen binding fragment that specifically binds SHBG. In other embodiments, for example, the anti-SHBG antibodies or other binding agents having an antigen binding fragment that specifically binds to SHBG can constitute between about 2% to about 75% of the weight of the composition, between about 25% to about 60%, between about 50% to about 50% or any range therein.

In some embodiments, the anti-SHBG antibody provided herein binds to SHBG (e.g., human SHBG) with a dissociation constant (K_{D}) of ≤1 μM, ≤0.5 μM, ≤0.1 μM, ≤0.01 μM, ≤0.001 μM, ≤100 nM, ≤10 nM, ≤2 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). In some embodiments, the anti-SHBG antibody provided herein binds to SHBG (e.g., human SHBG) with a K_{D} of about 1×10⁻⁷ M or less, 1×10⁻⁸ M or less, 1×10⁻⁹ M or less, 1×10⁻¹⁰ M or less, 2×10⁻⁷ M or less, 2×10⁻⁸ M or less, 2×10⁻⁹ M or less, 2×10⁻¹⁰ M or less, 5×10⁻⁷ M or less, 5×10⁻⁸ M or less, 5×10⁻⁹ M or less, or 5×10⁻¹⁰ M or less. For example, an anti-SHBG antibody described herein may have a K_{D} of about 1.0×10⁻⁹M to about 2.5×10⁻⁹ M. In some embodiments, the anti-SHBG antibody has a K_{D} of about 1.5, 1.6, 1.7, 1.8, 1.9, 2.0, 2.1, 2.2, 2.3, 2.4, 2.5, 2.6, 2.7, 2.8 2.9, or 3.0 nM. In some embodiments, the anti-SHBG antibody has a K_{D} of about 5.0, 6.0, 7.0, 8.0, 9.0 or 10 nM. In some embodiments, the anti-SHBG antibody has a K_{D} of about 15, 20, or 25 nM. In particular embodiments, the anti-SHBG antibody has a K_{D} of about 22 nM. A variety of methods of measuring binding affinity are known in the art, any of which can be used for purposes of the present disclosure, including by RIA, for example, performed with the Fab version of an antibody of interest and its antigen (Chen et al., 1999, J. Mol Biol 293:865-81); by biolayer interferometry (BLI) or surface plasmon resonance (SPR) assays by Octet®, using, for example, an Octet®Red96 system, or by Biacore®, using, for example, a Biacore®TM-2000 or a Biacore®TM-3000. An “on-rate” or “rate of association” or “association rate” or “kon” may also be determined with the same biolayer interferometry (BLI) or surface plasmon resonance (SPR) techniques described above using, for example, the Octet®Red96, the Biacore®TM-2000, or the Biacore®TM-3000 system.

In some embodiments, the anti-SHBG antibodies provide herein are those described in Examples 1 and 2 below. Thus, in some embodiments, the antibody provided herein comprises one or more CDR sequences of any one of SEQ ID NOs: 11-34, 44, 45, 47, 48, 50-54, 115, or 117. CDR sequences can be determined according to well-known numbering systems. In some embodiments, the CDRs are according to ImMunoGeneTics (IMGT) numbering. IMGT is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues and are readily identified. Accordingly, in some embodiments an isolated anti-SHBG antibody or a fragment thereof comprises HCDR1, HCDR2, and HCDR3 as set forth in SEQ ID NOs: 11, 12 and 13, respectively. In some emboidments, the isolated anti-SHBG antibody or the fragment thereof comprises HCDR1, HCDR2, and HCDR3 as set forth in SEQ ID NOs: 11, 44, and 45, respectively. In some embodiments, the isolated anti-SHBG antibody or the fragment thereof comprises LCDR1, LCDR2, and LCDR3 as set forth in SEQ ID NOs: 23, 24, and 25, respectively. In some emobiments, the HCDRs are associated with the LCDRs to form a SHBG binding region.

In some embodiments, the CDRs are determined according to Kabat numbering (see, e.g., Kabat et al., 5^{th} Ed. Public Health Service, National Intitutes of Health, Bethesda, Md. (1991)). The Kabat numbering scheme is a scheme for the numbering of amino acid residues in antibodies based upon variable regions. The Kabat numbering of residues may be determined for a given antibody by alignment at regions of homology of the sequence of the antibody with a “standard Kabat numbered sequence. The Kabat numbering system is generally used when referring to a residue in the variable domain. Accordingly, in some embodiments an isolated anti-SHBG antibody or a fragment thereof comprises HCDR1, HCDR2, and HCDR3 as set forth in SEQ ID NOs: 115, 116, and 19, respectively. In some emboidments, the isolated anti-SHBG antibody or the fragment thereof comprises HCDR1, HCDR2, and HCDR3 as set forth in SEQ ID NOs: 115, 117, and 51, respectively. In some embodiments, the isolated anti-SHBG antibody or the fragment thereof comprises LCDR1, LCDR2, and LCDR3 as set forth in SEQ ID NOs: 29, 30, and 31, respectively. In some emobiments, the HCDRs are associated with the LCDRs to form a SHBG binding region.

In some embodiments, the CDRs are determined according to Chothia numbering. Chothia refers to the location of structural loops (see, e.g., Chothia and Lesk, J. Mol. Biol. 196:901-917 (1987)). Accordingly, in some embodiments an isolated anti-SHBG antibody or a fragment thereof comprises HCDR1, HCDR2, and HCDR3 as set forth in SEQ ID NOs: 14, 15, and 16, respectively. In some emboidments, the isolated anti-SHBG antibody or the fragment thereof comprises HCDR1, HCDR2, and HCDR3 as set forth in SEQ ID NOs: 14, 47, and 48, respectively. In some embodiments, the isolated anti-SHBG antibody or the fragment thereof comprises LCDR1, LCDR2, and LCDR3 as set forth in SEQ ID NOs: 26, 27, and 28, respectively. In some embodiments, the isolated anti-SHBG antibody or the fragment thereof comprises LCDR1, LCDR2, and LCDR3 as set forth in SEQ ID NOs: 26, 27, and 25, respectively. In some emobiments, the HCDRs are associated with the LCDRs to form a SHBG binding region.

In some embodiments, the CDRs are determined according to AbM numbering. The AbM hypervariable regions represent a compromise between the Kabat CDRs and Chothia structural loops, and are used by Oxford Moleculars AbMantibody modeling Software (see, e.g., Martin, in Anti body Engineering, Vol. 2, Chapter 3, Springer Verlag). Accordingly, in some embodiments an isolated anti-SHBG antibody or a fragment thereof comprises HCDR1, HCDR2, and HCDR3 as set forth in SEQ ID NOs: 17, 18, and 19, respectively. In some emboidments, the isolated anti-SHBG antibody or the fragment thereof comprises HCDR1, HCDR2, and HCDR3 as set forth in SEQ ID NOs: 17, 50, and 51, respectively. In some embodiments, the isolated anti-SHBG antibody or the fragment thereof comprises LCDR1, LCDR2, and LCDR3 as set forth in SEQ ID NOS: 29, 30, and 31, respectively. In some emobiments, the HCDRs are associated with the LCDRs to form a SHBG binding region.

In some embodiments, the CDRs are determined according to Contact numbering. Accordingly, in some embodiments an isolated anti-SHBG antibody or a fragment thereof comprises HCDR1, HCDR2, and HCDR3 as set forth in SEQ ID NOs: 20, 21, and 22, respectively. In some emboidments, the isolated anti-SHBG antibody or the fragment thereof comprises HCDR1, HCDR2, and HCDR3 as set forth in SEQ ID NOs: 52, 53, and 54, respectively. In some embodiments, the isolated anti-SHBG antibody or the fragment thereof comprises LCDR1, LCDR2, and LCDR3 as set forth in SEQ ID NOs: 32, 33, and 34, respectively. In some emobiments, the HCDRs are associated with the LCDRs to form a SHBG binding region.

In other embodiments, the CDRs are according to Contact numbering. In some embodiments, the anti-SHBG antibody is humanized. In some embodiments, the anti-SHBG antibody is partially humanized. In some embodiments, the anti-SHBG antibody is chimeric. In some embodiments, the anti-SHBG antibody is chimeric humanized. In some embodiments, the anti-SHBG is a mouse antibody. In some embodiments, the anti-SHBG antibody comprises an acceptor human framework, e.g., a human immunoglobulin framework or a human consensus framework. In some embodiments, the anti-SHBG antibody comprises an acceptor mouse framework, e.g., a mouse immunoglobulin framework or a mouse consensus framework.

In some embodiments, the anti-SHBG antibody provided herein comprises HCDR1, HCDR2, and HCDR3 as set forth in SEQ ID NOs: 11-13. In some embodiments, the anti-SHBG antibody provided herein comprises HCDR1, HCDR2, and HCDR3 as set forth in SEQ ID NOs: 14-16. In some embodiments, the anti-SHBG antibody provided herein comprises HCDR1, HCDR2, and HCDR3 as set forth in SEQ ID NOs: 17-19. In some embodiments, the anti-SHBG antibody provided herein comprises HCDR1, HCDR2, and HCDR3 as set forth in SEQ ID NOs: 20-22. In some embodiments, the anti-SHBG antibody provided herein comprises HCDR1, HCDR2, and HCDR3 as set forth in SEQ ID NOs: 115, 116, and 19. In some embodiments, the anti-SHBG antibody provided herein comprises HCDR1, HCDR2, and HCDR3 as set forth in SEQ ID NOs: 11, 44, and 45. In some embodiments, the anti-SHBG antibody provided herein comprises HCDR1, HCDR2, and HCDR3 as set forth in SEQ ID NOs: 14, 47, and 48. In some embodiments, the anti-SHBG antibody provided herein comprises HCDR1, HCDR2, and HCDR3 as set forth in SEQ ID NOs: 17, 50, and 51. In some embodiments, the anti-SHBG antibody provided herein comprises HCDR1, HCDR2, and HCDR3 as set forth in SEQ ID NOs: 52, 53, and 54. In some embodiments, the anti-SHBG antibody provided herein comprises HCDR1, HCDR2, and HCDR3 as set forth in SEQ ID NOs: 115, 117, and 51. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In some embodiments, the anti-SHBG antibody provided herein comprises LCDR1, LCDR2, and LCDR3 as set forth in SEQ ID NOs: 23, 24, and 25. In some embodiments, the anti-SHBG antibody provided herein comprises LCDR1, LCDR2, and LCDR3 as set forth in SEQ ID NOs: 26, 27, and 28. In some embodiments, the anti-SHBG antibody provided herein comprises LCDR1, LCDR2, and LCDR3 as set forth in SEQ ID NOs: 29, 30, and 31. In some embodiments, the anti-SHBG antibody provided herein comprises LCDR1, LCDR2, and LCDR3 as set forth in SEQ ID NOs: 32, 33, and 34. In some embodiments, the anti-SHBG antibody provided herein comprises LCDR1, LCDR2, and LCDR3 as set forth in SEQ ID NOs: 29, 30, and 31. In some embodiments, the anti-SHBG antibody provided herein comprises LCDR1, LCDR2, and LCDR3 as set forth in SEQ ID NOs: 26, 27, and 25. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In some embodiments, the antibody or the fragment thereof as provided herein comprises an HCDR1, an HCDR2, and an HCDR3 as set forth in SEQ ID NOs: 11, 12, and 13, and a LCDR1, a LCDR2, and a LCDR3 as set forth in SEQ ID NOs: 23, 24, and 25. In some embodiments, the antibody or antigen binding fragment provided herein comprises an HCDR1, an HCDR2, and an HCDR3 as set forth in SEQ ID NOs: 14, 15, and 16, and a LCDR1, a LCDR2, and a LCDR3 as set forth in SEQ ID NOs: 26, 27, and 28. In some embodiments, the antibody or antigen binding fragment provided herein comprises an HCDR1, an HCDR2, and an HCDR3 as set forth in SEQ ID NOs: 17, 18, and 19, and a LCDR1, a LCDR2, and a LCDR3 as set forth in SEQ ID NOs: 29, 30, and 31. In some embodiments, the antibody or antigen binding fragment provided herein comprises an HCDR1, an HCDR2, and an HCDR3 as set forth in SEQ ID NOs: 20, 21, and 22, and a LCDR1, a LCDR2, and a LCDR3 as set forth in SEQ ID NOs: 32, 33, and 34. In some embodiments, the antibody or antigen binding fragment provided herein comprises an HCDR1, an HCDR2, and an HCDR3 as set forth in SEQ ID NOs: 115, 116, and 19, and a LCDR1, a LCDR2, and a LCDR3 as set forth in SEQ ID NOs: 29, 30, and 31. According, CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In some embodiments, the antibody or the fragment thereof as provided herein comprises an HCDR1, an HCDR2, and an HCDR3 as set forth in SEQ ID NOs: 11, 44, and 45, and a LCDR1, a LCDR2, and a LCDR3 as set forth in SEQ ID NOs: 23, 24, and 25. In some embodiments, the antibody or antigen binding fragment provided herein comprises an HCDR1, an HCDR2, and an HCDR3 as set forth in SEQ ID NOs: 14, 47, and 48, and a LCDR1, a LCDR2, and a LCDR3 as set forth in SEQ ID NOs: 26, 27, and 25. In some embodiments, the antibody or antigen binding fragment provided herein comprises an HCDR1, an HCDR2, and an HCDR3 as set forth in SEQ ID NOs: 17, 50, and 51, and a LCDR1, a LCDR2, and a LCDR3 as set forth in SEQ ID NOs: 29, 30, and 31. In some embodiments, the antibody or antigen binding fragment provided herein comprises an HCDR1, an HCDR2, and an HCDR3 as set forth in SEQ ID NOs: 52, 53, and 54, and a LCDR1, a LCDR2, and a LCDR3 as set forth in SEQ ID NOs: 32, 33, and 34. In some embodiments, the antibody or antigen binding fragment provided herein comprises an HCDR1, an HCDR2, and an HCDR3 as set forth in SEQ ID NOs: 115, 117, and 51, and a LCDR1, a LCDR2, and a LCDR3 as set forth in SEQ ID NOs: 29, 30, and 31. According, CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering

In some embodiments, provided herein is an antibody or a fragment thereof that binds to SHBG comprising an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 11, 14, 17, 20, and 115; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 12, 15, 18, 21, and 116, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 13, 16, 19, and 22; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 23, 26, 29, and 32; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 24, 27, 30, and 33; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to any of SEQ ID NOs: 25, 28, 31, and 34. In some embodiments, the anti-SHBG antibody is humanized. In some embodiments, the anti-SHBG antibody comprises an acceptor human framework, e.g., a human immunoglobulin framework or a human consensus framework.

In some embodiments, provided herein is an antibody or a fragment thereof that binds to SHBG comprising an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 11, 14, 17, 52, and 115; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 44, 47, 50, and 53, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 45, 48, 51, and 54; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 23, 26, 29, and 32; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 24, 27, 30, and 33; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to any of SEQ ID NOs: 25, 31, and 34. In some embodiments, the anti-SHBG antibody is humanized. In some embodiments, the anti-SHBG antibody comprises an acceptor human framework, e.g., a human immunoglobulin framework or a human consensus framework.

In one aspect, the anti-SHBG antibody or fragment thereof disclosed herein can comprise a degree of variability with respect to the HCDR2 and/or the HCDR3. Accordingly, provided herein is an isolated anti-SHBG antibody or a fragment thereof, comprising a heavy chain comprising a HCDR1 comprising amino acid sequence GYTFTGYW (SEQ ID NO: 11), a HCDR2 comprising amino acid sequence IDPSX₁X₂YT (SEQ ID NO: 112). The residue identified as X₁ can be independently any naturally occurring amino acid. Likewise, the residue identified as X₂ can be independently any naturally occurring amino acid residue. The anti-SHBG antibody or the fragment thereof can further include a HCDR3 comprising amino acid sequence TRX₃PLVTADFX₃Y (SEQ ID NO: 113). The residue identified as X₃ can be independently any naturally occurring amino acid residue. In some embodiments, X₁ comprises threonine or aspartic acid. In some embodiments, X₂ comprises glycine or threonine. In some embodiments, X₃ comprises glutamic acid or aspartic acid. In some embodiments, the antibody or the fragment thereof the X₁ is a threonine, X₂ is a glycine, and X₃ is glutamic acid. In some embodiments, the anti-SHBG antibody or the fragment thereof comprises a light chain comprising a LCDR1 comprising amino acid sequence QNVDTN (SEQ ID NO: 23), a LCDR2 comprising amino acid sequence SAS (SEQ ID NO: 24), and a LCDR3 comprising amino acid sequence QQYDSYPY (SEQ ID NO: 25).

In some embodiments, the anti-SHBG antibody or the fragment thereof has a higher affinity for a biologically active form of a SHBG protein than for a biologically inactive form of SHBG. In some embodiments, biologically active SHBG is a version of SHBG that is able to enter a cell or bind to a target receptor. In some embodiments, biologically active SHBG comprises a version of SHBG that is glycosylated. Accordingly, in some embodiments, the anti-SHBG antibody or the fragrment thereof disclosed herein comprises a higher affinity for glycosylated SHBG than for non-glycosylated SHBG. In some embodiments, the anti-SHBG antibody or fragment thereof is able to differentially bind a SHBG protein that comprises an epitope comprising a glycosylated amino acid, wherein the glycosylated amino acid comprises a threonine at position 36. In some embodiments, the glycosylated amino acid comprises an 0-linked glycan.

In some embodiments, the antibody provided herein is a humanized antibody. Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FR1 is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system.

In some embodiments, the antibody or antigen binding fragment provided herein comprises a VH sequence having at least 75%, 80%, 85%, 86%, 87%, 88%, 89%, 90%, 91%, 92%, 93%, 94%, 95%, 96%, 97%, 98%, 99%, 100% sequence identity to sequence of SEQ ID NO: 8, and a VL sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 10. In some embodiments, the antibody or antigen binding fragment provided herein comprises a VH sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 40, and a VL sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 42.

In certain embodiments, an antibody described herein or an antigen binding fragment thereof comprises amino acid sequences with certain percent identity relative to any antibody provided herein, for example, those described in the Sequence Listing below.

The determination of percent identity between two sequences (e.g., amino acid sequences or nucleic acid sequences) can be accomplished using a mathematical algorithm. A non-limiting example of a mathematical algorithm utilized for the comparison of two sequences is the algorithm of Karlin and Altschul, Proc. Natl. Acad. Sci. U.S.A. 87:2264 2268 (1990), modified as in Karlin and Altschul, Proc. Natl. Acad. Sci. U.S.A. 90:5873 5877 (1993). Such an algorithm is incorporated into the NBLAST and XBLAST programs of Altschul et al., J. Mol. Biol. 215:403 (1990). BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res. 25:3389 3402 (1997). Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, CABIOS 4:11-17 (1998). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In some embodiments, the antibody provide herein contains substitutions (e.g., conservative substitutions), insertions, or deletions relative to the reference sequence, but the anti-SHBG antibody comprising that sequence retains the ability to bind to SHBG. In some embodiments, a total of 1 to 10 amino acids have been substituted, inserted and/or deleted in a reference amino acid sequence. In some embodiments, substitutions, insertions, or deletions occur in regions outside the CDRs (i.e., in the FRs). Optionally, the anti-SHBG antibody provided herein includes post-translational modifications of a reference sequence.

In some embodiments, functional epitopes can be mapped, e.g., by combinatorial alanine scanning, to identify amino acids in the SHBG protein that are necessary for interaction with anti-SHBG antibodies provided herein. In some embodiments, conformational and crystal structure of anti-SHBG antibody bound to SHBG may be employed to identify the epitopes. In some embodiments, the present disclosure provides an antibody that specifically binds to the same epitope as any of the anti-SHBG antibodies provided herein. For example, in some embodiments, the antibody or antigen binding fragment provided herein binds to the same epitope as an anti-SHBG antibody comprising a VH comprising the amino acid sequence of SEQ ID NO: 8, and a VL comprising the amino acid sequence of SEQ ID NO: 10. In some embodiments, the antibody or antigen binding fragment provided herein binds to the same epitope as an anti-SHBG antibody comprising a VH comprising the amino acid sequence of SEQ ID NO: 40, and a VL comprising the amino acid sequence of SEQ ID NO: 42.

In some embodiments, provided herein is an anti-SHBG antibody, or antigen binding fragment thereof, that specifically binds to SHBG competitively with any one of the anti-SHBG antibodies described herein. For example, in some embodiments, the antibody or antigen binding fragment provided herein binds to the same epitope as an anti-SHBG antibody comprises a VH comprising the amino acid sequence of SEQ ID NO: 8, and a VL comprising the amino acid sequence of SEQ ID NO: 10. In some embodiments, the antibody or antigen binding fragment provided herein binds to the same epitope as an anti-SHBG antibody comprising a VH comprising the amino acid sequence of SEQ ID NO: 40, and a VL comprising the amino acid sequence of SEQ ID NO: 42.

In some embodiments, provided herein is a SHBG binding agent comprising any one of the anti-SHBG antibodies described above. In some embodiments, the SHBG binding agent is a monoclonal antibody, including a mouse, chimeric, humanized or human antibody. In some embodiments, the anti-SHBG antibody is an antibody fragment, e.g., a scFv. In some embodiments, the SHBG binding agent is a fusion protein comprising the anti-SHBG antibody provided herein. In other embodiments, the SHBG binding agent is a multispecific antibody comprising the anti-SHBG antibody provided herein. Other exemplary SHBG binding agents are described in more detail in the following sections.

In some embodiments, the anti-SHBG antibody or antigen binding protein according to any of the above embodiments may incorporate any of the features, singly or in combination, as described below.

Antibodies that Bind to IBP4

Described herein are isolated anti-IBP4 antibodies or antigen-binding fragments thereof, polypeptides and fragments thereof, nucleic acids and expression vectors encoding the antibodies, recombinant cells containing the vectors, and compositions comprising the antibodies. The antibodies disclosed herein possess one or more desirable functional properties, including, but not limited, to high-affinity binding to IBP4 and specifically bind to IBP4, which as described herein provides the ability to detect, diagnosis, or treat certain conditions. The antibodies disclosed herein are likewise useful to assess a health status, or predict an adverse pregnancy event.

In some embodiments, the anti-IBP4 antibody provided herein binds to IBP4 (e.g., human IBP4) with a dissociation constant (K_{D}) of ≤1 μM, ≤0.5 μM, ≤0.1 μM, ≤0.01 μM, ≤0.001 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). In some embodiments, the anti-IBP4 antibody provided herein binds to IBP4 (e.g., human IBP4) with a K_{D} of about 1×10⁻⁷ M or less, 1×10⁻⁸ M or less, 1×10⁻⁹ M or less, 1×10⁻¹⁰ M or less, 2×10⁻⁷ M or less, 2×10⁻⁸ M or less, 2×10⁻⁹ M or less, 2×10⁻¹⁰ M or less, 5×10⁻⁷ M or less, 5×10⁻⁸ M or less, 5×10⁻⁹ M or less, or 5×10⁻¹⁰ M or less. For example, an anti-IBP4 antibody described herein may have a K_{D} of about 3×10⁻⁸ M to about 8×10⁻⁸ M. In some embodiments, the anti-IBP4 antibody has a K_{D} of about 1.5, 1.6, 1.7, 1.8, 1.9, 2.0, 2.1, 2.2, 2.3, 2.4, 2.5, 2.6, 2.7, 2.8 2.9, or 3.0 nM. In some embodiments, the anti-IBP4 antibody has a K_{D} of about 5.0, 6.0, 7.0, 8.0, 9.0 or 10 nM. In some embodiments, the anti-IBP4 antibody has a K_{D} of about 15, 20, or 25 nM. In particular embodiments, the anti-IBP4 antibody has a K_{D} of about 22 nM. A variety of methods of measuring binding affinity are known in the art, any of which can be used for purposes of the present disclosure, including by RIA, for example, performed with the Fab version of an antibody of interest and its antigen (Chen et al., 1999, J. Mol Biol 293:865-81); by biolayer interferometry (BLI) or surface plasmon resonance (SPR) assays by Octet®, using, for example, an Octet®Red96 system, or by Biacore®, using, for example, a Biacore®TM-2000 or a Biacore®TM-3000. An “on-rate” or “rate of association” or “association rate” or “kon” may also be determined with the same biolayer interferometry (BLI) or surface plasmon resonance (SPR) techniques described above using, for example, the Octet®Red96, the Biacore®TM-2000, or the Biacore®TM-3000 system.

In some embodiments, the anti-IBP4 antibodies provide herein are those described in Examples 1, 3, and 4 below. In one aspect, this disclosure relates to an isolated anti-IBP4 antibody or antigen-binding fragment thereof. In some embodiments, the isolated anti-IBP4 antibody or the fragment thereof binds to a IBP4 epitope. The IBP4 epitope can comprise a sequence that has at least 50%, 55%, 60%, 65%, 70%, 75%, 80%, 85%, 90%, 95%, 99%, or 100% identity to SEQ ID NO: 111 or 131.

In some embodiments, the antibodies provided herein comprise a heavy chain variable region. In other embodiments, the antibodies provided herein comprise a light chain variable region. In some embodiments, the antibodies provided herein have a combination of (i) a heavy chain variable region; and/or (ii) a light chain variable region.

In some embodiments, an antibody provided herein comprises or consists of six CDRs, for example, HCDR1, HCDR2, HCDR3, LCDR1, LCDR2, and/or LCDR3 as identified in Tables 18, 20, 24, 26, 28, 30, and 32. [00143]. In some embodiments, an antibody provided herein can comprise less than six CDRs.

In some embodiments, the antibody comprises or consists of one, two, three, four, or five CDRs selected from the group consisting of HCDR1, HCDR2, HCDR3, LCDR1, LCDR2, and/or LCDR3 identified in Tables 18, 20, 24, 26, 28, 30, and 32. In some embodiments, the antibody comprises or consists of one, two, three, four, or five CDRs selected from the group consisting of HCDR1, HCDR2, HCDR3, LCDR1, LCDR2, and/or LCDR3 of the monoclonal antibody selected from the group consisting of: (a) the antibody designated 5B3.G9; (b) the antibody designated variant 5B3.G9; (c) the antibody designated 7D6.G4; (d) the antibody designated variant 7D6.G4; (e) the antibody designated 1H9.B6; (f) the antibody designated variant 1H9.B6; (g) the antibody designated 5C11.A7; (h) the antibody designated variant 5C11.A7; (i) the antibody designated 7G11.H4; (j) the antibody designated variant 7G11.H4; (k) the antibody designated 3D4.E9; (1) the antibody designated variant 3D4.E9; (m) the antibody designated 1G3.A3; or (n) the antibody designated variant 1G3.A3. Accordingly, in some embodiments, the antibody comprises or consists of one, two, three four or five CDRs of anyone of the HCDR1, HCDR2, HCDR3, LCDR1, LCDR2, and/or LCDR3 identified in Tables 18, 20, 24, 26, 28, 30, and 32.

In some embodiments, the antibodies provided herein comprise one or more (e.g., one, two or three) VH CDRs listed in Table 18, Table 20, Table 24, Table 26, Table 28, Table 30, and Table 32. In other embodiments, the antibodies provided herein comprise one or more (e.g., one, two or three) VL CDRs listed in Table 18, Table 20, Table 24, Table 26, Table 28, Table 30, and Table 32. In yet other embodiments, the antibodies provided herein comprise one or more (e.g., one, two or three) VH CDRs listed in Table 18, Table 20, Table 24, Table 26, Table 28, Table 30, and Table 32 and one or more VL CDRs listed in Table 18, Table 20, Table 24, Table 26, Table 28, Table 30, and Table 32. Accordingly, in certain embodiments, the antibodies comprise a VH CDR1 having the amino acid sequence of any one of SEQ ID NOS: 75, 78, 81, 84, 118, 244, 247, 250, 253, 256, 288, 291, 294, and 314. In another embodiment, the antibodies comprise a VH CDR2 having the amino acid sequence of any one of SEQ ID NOS: 76, 79, 82, 85, 119, 216, 218, 220, 222, 224, 245, 248, 251, 254, 257, 284, 286, 289, 292, 295, 312, 315, 317, 319, and 321. In another embodiment, the antibodies comprise a VH CDR3 having the amino acid sequence of any one of SEQ ID NOS: 77, 80, 83, 86, 217, 219, 221, 223, 225, 246, 249, 252, 255, 258, 285, 287, 290, 293, 296, 313, 316, 318, 320, 322. In certain embodiments, the antibodies comprise a VH CDR1 and/or a VH CDR2 and/or a VH CDR3 independently selected from a VH CDR1, VH CDR2, VH CDR3 as depicted in any one of the amino acid sequences depicted in Table 18, Table 20, Table 24, Table 26, Table 28, Table 30, and Table 32. In certain embodiments, the antibodies comprise a VL CDR1 having the amino acid sequence of any one of SEQ ID NOS: 23, 87, 90, 93, 96, 226, 228, 230, 233, 272, 274, 275, 323, 325, 327, and 330. In another embodiment, the antibodies comprise a VL CDR2 having the amino acid sequence of any one of SEQ ID NOS: 24, 91, 94, 97, 229, 231, 234, 260, 261, 263, 273, 297, 299, 300, 302, 326, 328, and 331. In another embodiment, the antibodies comprise a VL CDR3 having the amino acid sequence of any one of SEQ ID NOS: 89, 95, 98, 99, 100, 101, 227, 232, 235, 259, 262, 264, 298, 301, 303, 324, 329, and 332. In certain embodiments, the antibodies comprise a VL CDR1 and/or a VL CDR2 and/or a VL CDR3 independently selected from a VL CDR1, VL CDR2, VL CDR3 as depicted in any one of the amino acid sequences depicted in Table 18, Table 20, Table 24, Table 26, Table 28, Table 30, and Table 32.

Also provided herein are antibodies comprising one or more (e.g., one, two or three) VH CDRs and one or more (e.g., one, two or three) VL CDRs listed in Table 18, Table 20, Table 24, Table 26, Table 28, Table 30, and Table 32. In particular, provided herein is an antibody comprising: a VH CDR1 (SEQ ID NOS: 75, 78, 81, 84, 118, 244, 247, 250, 253, 256, 288, 291, 294, and 314) and a VL CDR1 (SEQ ID NOS: 23, 87, 90, 93, 96, 226, 228, 230, 233, 272, 274, 275, 323, 325, 327, and 330); a VH CDR1 (SEQ ID NOS: 75, 78, 81, 84, 118, 244, 247, 250, 253, 256, 288, 291, 294, and 314) and a VL CDR2 (SEQ ID NOS: 24, 91, 94, 97, 229, 231, 234, 260, 261, 263, 273, 297, 299, 300, 302, 326, 328, and 331); a VH CDR1 (SEQ ID NOS: 75, 78, 81, 84, 118, 244, 247, 250, 253, 256, 288, 291, 294, and 314) and a VL CDR3 (SEQ ID NOS: 89, 95, 98, 99, 100, 101, 227, 232, 235, 259, 262, 264, 298, 301, 303, 324, 329, and 332); a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321) and a VL CDR1 (SEQ ID NOS: 23, 87, 90, 93, 96, 226, 228, 230, 233, 272, 274, 275, 323, 325, 327, and 330); a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321) and a VL CDR2 (SEQ ID NOS: 24, 91, 94, 97, 229, 231, 234, 260, 261, 263, 273, 297, 299, 300, 302, 326, 328, and 331); a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321) and a VL CDR3 (SEQ ID NOS: 89, 95, 98, 99, 100, 101, 227, 232, 235, 259, 262, 264, 298, 301, 303, 324, 329, and 332); a VH CDR3 (SEQ ID NOS: 77, 80, 83, 86, 217, 219, 221, 223, 225, 246, 249, 252, 255, 258, 285, 287, 290, 293, 296, 313, 316, 318, 320, 322) and a VL CDR1 (SEQ ID NOS: 23, 87, 90, 93, 96, 226, 228, 230, 233, 272, 274, 275, 323, 325, 327, and 330); a VH CDR3 (SEQ ID NOS: 77, 80, 83, 86, 217, 219, 221, 223, 225, 246, 249, 252, 255, 258, 285, 287, 290, 293, 296, 313, 316, 318, 320, 322) and a VL CDR2 (SEQ ID NOS: 24, 91, 94, 97, 229, 231, 234, 260, 261, 263, 273, 297, 299, 300, 302, 326, 328, and 331); a VH CDR3 (SEQ ID NOS: 77, 80, 83, 86, 217, 219, 221, 223, 225, 246, 249, 252, 255, 258, 285, 287, 290, 293, 296, 313, 316, 318, 320, 322) and a VL CDR3 (SEQ ID NOS: 89, 95, 98, 99, 100, 101, 227, 232, 235, 259, 262, 264, 298, 301, 303, 324, 329, and 332); a VH CDR1 (SEQ ID NOS: 75, 78, 81, 84, 118, 244, 247, 250, 253, 256, 288, 291, 294, and 314), a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321) and a VL CDR1 (SEQ ID NOS: 23, 87, 90, 93, 96, 226, 228, 230, 233, 272, 274, 275, 323, 325, 327, and 330); a VH CDR1 (SEQ ID NOS: 75, 78, 81, 84, 118, 244, 247, 250, 253, 256, 288, 291, 294, and 314), a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321) and a VL CDR2 (SEQ ID NOS: 24, 91, 94, 97, 229, 231, 234, 260, 261, 263, 273, 297, 299, 300, 302, 326, 328, and 331); a VH CDR1 (SEQ ID NOS: 75, 78, 81, 84, 118, 244, 247, 250, 253, 256, 288, 291, 294, and 314), a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321) and a VL CDR3 (SEQ ID NOS: 89, 95, 98, 99, 100, 101, 227, 232, 235, 259, 262, 264, 298, 301, 303, 324, 329, and 332); a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321), a VH CDR3 (SEQ ID NOS: 77, 80, 83, 86, 217, 219, 221, 223, 225, 246, 249, 252, 255, 258, 285, 287, 290, 293, 296, 313, 316, 318, 320, 322) and a VL CDR1 (SEQ ID NOS: 23, 87, 90, 93, 96, 226, 228, 230, 233, 272, 274, 275, 323, 325, 327, and 330), a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321), a VH CDR3 (SEQ ID NOS: 77, 80, 83, 86, 217, 219, 221, 223, 225, 246, 249, 252, 255, 258, 285, 287, 290, 293, 296, 313, 316, 318, 320, 322) and a VL CDR2 (SEQ ID NOS: 24, 91, 94, 97, 229, 231, 234, 260, 261, 263, 273, 297, 299, 300, 302, 326, 328, and 331); a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321), a VH CDR3 (SEQ ID NOS: 77, 80, 83, 86, 217, 219, 221, 223, 225, 246, 249, 252, 255, 258, 285, 287, 290, 293, 296, 313, 316, 318, 320, 322) and a VL CDR3 (SEQ ID NOS: 89, 95, 98, 99, 100, 101, 227, 232, 235, 259, 262, 264, 298, 301, 303, 324, 329, and 332); a VH CDR1 (SEQ ID NOS: 75, 78, 81, 84, 118, 244, 247, 250, 253, 256, 288, 291, 294, and 314), a VL CDR1 (SEQ ID NOS: 23, 87, 90, 93, 96, 226, 228, 230, 233, 272, 274, 275, 323, 325, 327, and 330) and a VL CDR2 (SEQ ID NOS: 24, 91, 94, 97, 229, 231, 234, 260, 261, 263, 273, 297, 299, 300, 302, 326, 328, and 331); a VH CDR1 (SEQ ID NOS: 75, 78, 81, 84, 118, 244, 247, 250, 253, 256, 288, 291, 294, and 314), a VL CDR1 (SEQ ID NOS: 23, 87, 90, 93, 96, 226, 228, 230, 233, 272, 274, 275, 323, 325, 327, and 330) and a VL CDR3 (SEQ ID NOS: 89, 95, 98, 99, 100, 101, 227, 232, 235, 259, 262, 264, 298, 301, 303, 324, 329, and 332); a VH CDR1 (SEQ ID NOS: 75, 78, 81, 84, 118, 244, 247, 250, 253, 256, 288, 291, 294, and 314), a VL CDR2 (SEQ ID NOS: 24, 91, 94, 97, 229, 231, 234, 260, 261, 263, 273, 297, 299, 300, 302, 326, 328, and 331) and a VL CDR3 (SEQ ID NOS: 89, 95, 98, 99, 100, 101, 227, 232, 235, 259, 262, 264, 298, 301, 303, 324, 329, and 332); a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321), a VL CDR1 (SEQ ID NOS: 23, 87, 90, 93, 96, 226, 228, 230, 233, 272, 274, 275, 323, 325, 327, and 330) and a VL CDR2 (SEQ ID NOS: 24, 91, 94, 97, 229, 231, 234, 260, 261, 263, 273, 297, 299, 300, 302, 326, 328, and 331); a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321), a VL CDR1 (SEQ ID NOS: 23, 87, 90, 93, 96, 226, 228, 230, 233, 272, 274, 275, 323, 325, 327, and 330) and a VL CDR3 (SEQ ID NOS: 89, 95, 98, 99, 100, 101, 227, 232, 235, 259, 262, 264, 298, 301, 303, 324, 329, and 332); a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321), a VL CDR2 (SEQ ID NOS: 24, 91, 94, 97, 229, 231, 234, 260, 261, 263, 273, 297, 299, 300, 302, 326, 328, and 331) and a VL CDR3 (SEQ ID NOS: 89, 95, 98, 99, 100, 101, 227, 232, 235, 259, 262, 264, 298, 301, 303, 324, 329, and 332); a VH CDR3 (SEQ ID NOS: 77, 80, 83, 86, 217, 219, 221, 223, 225, 246, 249, 252, 255, 258, 285, 287, 290, 293, 296, 313, 316, 318, 320, 322), a VL CDR1 (SEQ ID NOS: 23, 87, 90, 93, 96, 226, 228, 230, 233, 272, 274, 275, 323, 325, 327, and 330) and a VL CDR2 (SEQ ID NOS: 24, 91, 94, 97, 229, 231, 234, 260, 261, 263, 273, 297, 299, 300, 302, 326, 328, and 331); a VH CDR3 (SEQ ID NOS: 77, 80, 83, 86, 217, 219, 221, 223, 225, 246, 249, 252, 255, 258, 285, 287, 290, 293, 296, 313, 316, 318, 320, 322), a VL CDR1 (SEQ ID NOS: 23, 87, 90, 93, 96, 226, 228, 230, 233, 272, 274, 275, 323, 325, 327, and 330) and a VL CDR3 (SEQ ID NOS: 89, 95, 98, 99, 100, 101, 227, 232, 235, 259, 262, 264, 298, 301, 303, 324, 329, and 332); a VH CDR3 (SEQ ID NOS: 77, 80, 83, 86, 217, 219, 221, 223, 225, 246, 249, 252, 255, 258, 285, 287, 290, 293, 296, 313, 316, 318, 320, 322), a VL CDR2 (SEQ ID NOS: 24, 91, 94, 97, 229, 231, 234, 260, 261, 263, 273, 297, 299, 300, 302, 326, 328, and 331) and a VL CDR3 (SEQ ID NOS: 89, 95, 98, 99, 100, 101, 227, 232, 235, 259, 262, 264, 298, 301, 303, 324, 329, and 332); a VH CDR1 (SEQ ID NOS: 75, 78, 81, 84, 118, 244, 247, 250, 253, 256, 288, 291, 294, and 314), a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321), a VH CDR3 (SEQ ID NOS: 77, 80, 83, 86, 217, 219, 221, 223, 225, 246, 249, 252, 255, 258, 285, 287, 290, 293, 296, 313, 316, 318, 320, 322) and a VL CDR1 (SEQ ID NOS: 23, 87, 90, 93, 96, 226, 228, 230, 233, 272, 274, 275, 323, 325, 327, and 330); a VH CDR1 (SEQ ID NOS: 75, 78, 81, 84, 118, 244, 247, 250, 253, 256, 288, 291, 294, and 314), a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321), a VH CDR3 (SEQ ID NOS: 77, 80, 83, 86, 217, 219, 221, 223, 225, 246, 249, 252, 255, 258, 285, 287, 290, 293, 296, 313, 316, 318, 320, 322) and a VL CDR2 (SEQ ID NOS: 24, 91, 94, 97, 229, 231, 234, 260, 261, 263, 273, 297, 299, 300, 302, 326, 328, and 331); a VH CDR1 (SEQ ID NOS: 75, 78, 81, 84, 118, 244, 247, 250, 253, 256, 288, 291, 294, and 314), a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321), a VH CDR3 (SEQ ID NOS: 77, 80, 83, 86, 217, 219, 221, 223, 225, 246, 249, 252, 255, 258, 285, 287, 290, 293, 296, 313, 316, 318, 320, 322) and a VL CDR3 (SEQ ID NOS: 89, 95, 98, 99, 100, 101, 227, 232, 235, 259, 262, 264, 298, 301, 303, 324, 329, and 332); a VH CDR1 (SEQ ID NOS: 75, 78, 81, 84, 118, 244, 247, 250, 253, 256, 288, 291, 294, and 314), a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321), a VL CDR1 (SEQ ID NOS: 23, 87, 90, 93, 96, 226, 228, 230, 233, 272, 274, 275, 323, 325, 327, and 330) and a VL CDR2 (SEQ ID NOS: 24, 91, 94, 97, 229, 231, 234, 260, 261, 263, 273, 297, 299, 300, 302, 326, 328, and 331); a VH CDR1 (SEQ ID NOS: 75, 78, 81, 84, 118, 244, 247, 250, 253, 256, 288, 291, 294, and 314), a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321), a VL CDR1 (SEQ ID NOS: 23, 87, 90, 93, 96, 226, 228, 230, 233, 272, 274, 275, 323, 325, 327, and 330) and a VL CDR3 (SEQ ID NOS: 89, 95, 98, 99, 100, 101, 227, 232, 235, 259, 262, 264, 298, 301, 303, 324, 329, and 332); a VH CDR1 (SEQ ID NOS: 75, 78, 81, 84, 118, 244, 247, 250, 253, 256, 288, 291, 294, and 314), a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321), a VL CDR2 (SEQ ID NOS: 24, 91, 94, 97, 229, 231, 234, 260, 261, 263, 273, 297, 299, 300, 302, 326, 328, and 331) and a VL CDR3 (SEQ ID NOS: 89, 95, 98, 99, 100, 101, 227, 232, 235, 259, 262, 264, 298, 301, 303, 324, 329, and 332); a VH CDR1 (SEQ ID NOS: 75, 78, 81, 84, 118, 244, 247, 250, 253, 256, 288, 291, 294, and 314), a VH CDR3 (SEQ ID NOS: 77, 80, 83, 86, 217, 219, 221, 223, 225, 246, 249, 252, 255, 258, 285, 287, 290, 293, 296, 313, 316, 318, 320, 322), a VL CDR1 (SEQ ID NOS: 23, 87, 90, 93, 96, 226, 228, 230, 233, 272, 274, 275, 323, 325, 327, and 330) and a VL CDR2 (SEQ ID NOS: 24, 91, 94, 97, 229, 231, 234, 260, 261, 263, 273, 297, 299, 300, 302, 326, 328, and 331); a VH CDR1 (SEQ ID NOS: 75, 78, 81, 84, 118, 244, 247, 250, 253, 256, 288, 291, 294, and 314), a VH CDR3 (SEQ ID NOS: 77, 80, 83, 86, 217, 219, 221, 223, 225, 246, 249, 252, 255, 258, 285, 287, 290, 293, 296, 313, 316, 318, 320, 322), a VL CDR1 (SEQ ID NOS: 23, 87, 90, 93, 96, 226, 228, 230, 233, 272, 274, 275, 323, 325, 327, and 330) and a VL CDR3 (SEQ ID NOS: 89, 95, 98, 99, 100, 101, 227, 232, 235, 259, 262, 264, 298, 301, 303, 324, 329, and 332); a VH CDR1 (SEQ ID NOS: 75, 78, 81, 84, 118, 244, 247, 250, 253, 256, 288, 291, 294, and 314), a VH CDR3 (SEQ ID NOS: 77, 80, 83, 86, 217, 219, 221, 223, 225, 246, 249, 252, 255, 258, 285, 287, 290, 293, 296, 313, 316, 318, 320, 322), a VL CDR2 (SEQ ID NOS: 24, 91, 94, 97, 229, 231, 234, 260, 261, 263, 273, 297, 299, 300, 302, 326, 328, and 331) and a VL CDR3 (SEQ ID NOS: 89, 95, 98, 99, 100, 101, 227, 232, 235, 259, 262, 264, 298, 301, 303, 324, 329, and 332); a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321), a VH CDR3 (SEQ ID NOS: 77, 80, 83, 86, 217, 219, 221, 223, 225, 246, 249, 252, 255, 258, 285, 287, 290, 293, 296, 313, 316, 318, 320, 322), a VL CDR1 (SEQ ID NOS: 23, 87, 90, 93, 96, 226, 228, 230, 233, 272, 274, 275, 323, 325, 327, and 330) and a VL CDR2 (SEQ ID NOS: 24, 91, 94, 97, 229, 231, 234, 260, 261, 263, 273, 297, 299, 300, 302, 326, 328, and 331); a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321), a VH CDR3 (SEQ ID NOS: 77, 80, 83, 86, 217, 219, 221, 223, 225, 246, 249, 252, 255, 258, 285, 287, 290, 293, 296, 313, 316, 318, 320, 322), a VL CDR1 (SEQ ID NOS: 23, 87, 90, 93, 96, 226, 228, 230, 233, 272, 274, 275, 323, 325, 327, and 330) and a VL CDR3 (SEQ ID NOS: 89, 95, 98, 99, 100, 101, 227, 232, 235, 259, 262, 264, 298, 301, 303, 324, 329, and 332); a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321), a VH CDR3 (SEQ ID NOS: 77, 80, 83, 86, 217, 219, 221, 223, 225, 246, 249, 252, 255, 258, 285, 287, 290, 293, 296, 313, 316, 318, 320, 322), a VL CDR2 (SEQ ID NOS: 24, 91, 94, 97, 229, 231, 234, 260, 261, 263, 273, 297, 299, 300, 302, 326, 328, and 331) and a VL CDR3 (SEQ ID NOS: 89, 95, 98, 99, 100, 101, 227, 232, 235, 259, 262, 264, 298, 301, 303, 324, 329, and 332); a VH CDR1 (SEQ ID NOS: 75, 78, 81, 84, 118, 244, 247, 250, 253, 256, 288, 291, 294, and 314), a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321), a VH CDR3 (SEQ ID NOS: 77, 80, 83, 86, 217, 219, 221, 223, 225, 246, 249, 252, 255, 258, 285, 287, 290, 293, 296, 313, 316, 318, 320, 322), a VL CDR1 (SEQ ID NOS: 23, 87, 90, 93, 96, 226, 228, 230, 233, 272, 274, 275, 323, 325, 327, and 330) and a VL CDR2 (SEQ ID NOS: 24, 91, 94, 97, 229, 231, 234, 260, 261, 263, 273, 297, 299, 300, 302, 326, 328, and 331); a VH CDR1 (SEQ ID NOS: 75, 78, 81, 84, 118, 244, 247, 250, 253, 256, 288, 291, 294, and 314), a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321), a VH CDR3 (SEQ ID NOS: 77, 80, 83, 86, 217, 219, 221, 223, 225, 246, 249, 252, 255, 258, 285, 287, 290, 293, 296, 313, 316, 318, 320, 322), a VL CDR1 (SEQ ID NOS: 23, 87, 90, 93, 96, 226, 228, 230, 233, 272, 274, 275, 323, 325, 327, and 330) and a VL CDR3 (SEQ ID NOS: 89, 95, 98, 99, 100, 101, 227, 232, 235, 259, 262, 264, 298, 301, 303, 324, 329, and 332); a VH CDR1 (SEQ ID NOS: 75, 78, 81, 84, 118, 244, 247, 250, 253, 256, 288, 291, 294, and 314), a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321), a VH CDR3 (SEQ ID NOS: 77, 80, 83, 86, 217, 219, 221, 223, 225, 246, 249, 252, 255, 258, 285, 287, 290, 293, 296, 313, 316, 318, 320, 322), a VL CDR2 (SEQ ID NOS: 24, 91, 94, 97, 229, 231, 234, 260, 261, 263, 273, 297, 299, 300, 302, 326, 328, and 331) and a VL CDR3 (SEQ ID NOS: 89, 95, 98, 99, 100, 101, 227, 232, 235, 259, 262, 264, 298, 301, 303, 324, 329, and 332); a VH CDR1 (SEQ ID NOS: 75, 78, 81, 84, 118, 244, 247, 250, 253, 256, 288, 291, 294, and 314), a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321), a VL CDR1 (SEQ ID NOS: 23, 87, 90, 93, 96, 226, 228, 230, 233, 272, 274, 275, 323, 325, 327, and 330), a VL CDR2 (SEQ ID NOS: 24, 91, 94, 97, 229, 231, 234, 260, 261, 263, 273, 297, 299, 300, 302, 326, 328, and 331), and a VL CDR3 (SEQ ID NOS: 89, 95, 98, 99, 100, 101, 227, 232, 235, 259, 262, 264, 298, 301, 303, 324, 329, and 332); a VH CDR1 (SEQ ID NOS: 75, 78, 81, 84, 118, 244, 247, 250, 253, 256, 288, 291, 294, and 314), a VH CDR3 (SEQ ID NOS: 77, 80, 83, 86, 217, 219, 221, 223, 225, 246, 249, 252, 255, 258, 285, 287, 290, 293, 296, 313, 316, 318, 320, 322), a VL CDR1 (SEQ ID NOS: 23, 87, 90, 93, 96, 226, 228, 230, 233, 272, 274, 275, 323, 325, 327, and 330), a VL CDR2 (SEQ ID NOS: 24, 91, 94, 97, 229, 231, 234, 260, 261, 263, 273, 297, 299, 300, 302, 326, 328, and 331), and a VL CDR3 (SEQ ID NOS: 89, 95, 98, 99, 100, 101, 227, 232, 235, 259, 262, 264, 298, 301, 303, 324, 329, and 332); a VH CDR2 (SEQ ID NOS: 76, 79, 82, 85, 119, 216, 218, 220, 222, 224, 245, 248, 251, 254, 257, 284, 286, 289, 292, 295, 312, 315, 317, 319, and 321), a VH CDR3 (SEQ ID NOS: 77, 80, 83, 86, 217, 219, 221, 223, 225, 246, 249, 252, 255, 258, 285, 287, 290, 293, 296, 313, 316, 318, 320, 322), a VL CDR1 (SEQ ID NOS: 23, 87, 90, 93, 96, 226, 228, 230, 233, 272, 274, 275, 323, 325, 327, and 330), a VL CDR2 (SEQ ID NOS: 24, 91, 94, 97, 229, 231, 234, 260, 261, 263, 273, 297, 299, 300, 302, 326, 328, and 331), and a VL CDR3 (SEQ ID NOS: 89, 95, 98, 99, 100, 101, 227, 232, 235, 259, 262, 264, 298, 301, 303, 324, 329, and 332); or any combination thereof of the VH CDRs (SEQ ID NOS: 75, 76, 77, 78, 79, 80, 81, 82, 83, 84, 85, 86, 118, 119, 244, 247, 250, 253, 256, 288, 291, 294, 314, 216, 218, 220, 222, 224, 245, 248, 251, 254, 257, 284, 286, 289, 292, 295, 312, 315, 317, 319, 321, 217, 219, 221, 223, 225, 246, 249, 252, 255, 258, 285, 287, 290, 293, 296, 313, 316, 318, 320, and 322) and VL CDRs (SEQ ID NOS: 23, 24, 89, 91, 94, 95, 97, 98, 99, 100, 101, 226, 228, 230, 233, 272, 274, 275, 323, 325, 327, 330, 229, 231, 234, 260, 261, 263, 273, 297, 299, 300, 302, 326, 328, 331, 227, 232, 235, 259, 262, 264, 298, 301, 303, 324, 329, and 332) listed in Table 18, Table 20, Table 24, Table 26, Table 28, Table 30, and Table 32.

In some embodiments, the antibodies provided herein comprise one or more (e.g., one, two or three) HCDRs listed in Tables 18, 20, 24, 26, 28, 30, and 32. In other embodiments, the antibodies provided herein comprise one or more (e.g., one, two or three) LCDRs listed in Tables 14 and 16. In yet other embodiments, the antibodies provided herein comprise one or more (e.g., one, two or three) HCDRs listed in Tables 14 and 16 and one or more LCDRs listed in Tables 18, 20, 24, 26, 28, 30, and 32.

Thus, in some embodiments, the antibody provided herein comprises one or more CDR sequences of any one of SEQ ID NOs: 24, 75-99, 118-119, 216-240, 244-264, 272-275, 284-303, or 312-332. CDR sequences can be determined according to well-known numbering systems. In some embodiments, the CDRs are determined according to ImMunoGeneTics (IMGT) numbering. IMGT is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues and are readily identified. Accordingly, in some embodiments an isolated anti-IBP4 antibody or a fragment thereof comprises HCDR1, HCDR2, and HCDR3 as set forth in SEQ ID NOs: 75, 76, and 77, respectively. In some embodiments, the isolated anti-IBP4 antibody or the fragment thereof comprises HCDR1, HCDR2, and HCDR3 as set forth in SEQ ID NOs: 75, 216, and 217, respectively. In some embodiments, the isolated anti-IBP4 antibody or the fragment thereof comprises HCDR1, HCDR2, and HCDR3 as set forth in SEQ ID NOs: 244, 245, and 246, respectively. In some embodiments, the isolated anti-IBP4 antibody or the fragment thereof comprises HCDR1, HCDR2, and HCDR3 as set forth in SEQ ID NOs: 244, 245, and 217, respectively. In some embodiments, the isolated anti-IBP4 antibody or the fragment thereof comprises HCDR1, HCDR2, and HCDR3 as set forth in SEQ ID NOs: 244, 284, and 285, respectively. In some embodiments, the isolated anti-IBP4 antibody or the fragment thereof comprises HCDR1, HCDR2, and HCDR3 as set forth in SEQ ID NOs: 244, 312, and 313, respectively. In some embodiments, the isolated anti-IBP4 antibody or the fragment thereof comprises LCDR1, LCDR2, and LCDR3 as set forth in SEQ ID NOs: 87, 24, and 89, respectively. In some embodiments, the isolated anti-IBP4 antibody or the fragment thereof comprises LCDR1, LCDR2, and LCDR3 as set forth in SEQ ID NOs: 87, 24, and 99, respectively. In some embodiments, the isolated anti-IBP4 antibody or the fragment thereof comprises LCDR1, LCDR2, and LCDR3 as set forth in SEQ ID NOs: 23, 24, and 259, respectively. In some embodiments, the isolated anti-IBP4 antibody or the fragment thereof comprises LCDR1, LCDR2, and LCDR3 as set forth in SEQ ID NOs: 87, 24, and 259, respectively. In some embodiments, the isolated anti-IBP4 antibody or the fragment thereof comprises LCDR1, LCDR2, and LCDR3 as set forth in SEQ ID NOs: 226, 24, and 227, respectively. In some embodiments, the isolated anti-IBP4 antibody or the fragment thereof comprises LCDR1, LCDR2, and LCDR3 as set forth in SEQ ID NOs: 87, 297, and 298, respectively. In some embodiments, the isolated anti-IBP4 antibody or the fragment thereof comprises LCDR1, LCDR2, and LCDR3 as set forth in SEQ ID NOs: 323, 24, and 324, respectively. In some embodiments, the HCDRs are associated with the LCDRs to form a IBP4 binding region. For example, in some embodiments the CDR sequences can be any one or more of the CDR sequences as shown in Tables 18, 20, 24, 26, 28, 30, and 32.

In some embodiments, the CDRs are determined according to Kabat numbering (see, e.g., Kabat et al., 5th Ed. Public Health Service, National Institutes of Health, Bethesda, Md. (1991)). The Kabat numbering scheme is a scheme for the numbering of amino acid residues in antibodies based upon variable regions. The Kabat numbering of residues may be determined for a given antibody by alignment at regions of homology of the sequence of the antibody with a “standard Kabat numbered sequence. The Kabat numbering system is generally used when referring to a residue in the variable domain. Accordingly, in some embodiments an isolated anti-IBP4 antibody or a fragment thereof comprises HCDR1, HCDR2, and HCDR3 as set forth in SEQ ID NOs: 118, 119, and 83, respectively. In some embodiments, the isolated anti-IBP4 antibody or the fragment thereof comprises HCDR1, HCDR2, and HCDR3 as set forth in SEQ ID NOs: 118, 224, and 25, respectively. In some embodiments, the isolated anti-IBP4 antibody or the fragment thereof comprises HCDR1, HCDR2, and HCDR3 as set forth in SEQ ID NOs: 256, 257, and 258, respectively. In some embodiments, the isolated anti-IBP4 antibody or the fragment thereof comprises HCDR1, HCDR2, and HCDR3 as set forth in SEQ ID NOs: 294, 295, and 296, respectively. In some embodiments, the isolated anti-IBP4 antibody or the fragment thereof comprises HCDR1, HCDR2, and HCDR3 as set forth in SEQ ID NOs: 294, 326, and 324, respectively. In some embodiments, the isolated anti-IBP4 antibody or the fragment thereof comprises LCDR1, LCDR2, and LCDR3 as set forth in SEQ ID NOs: 93, 94, and 89, respectively. In some embodiments, the isolated anti-IBP4 antibody or the fragment thereof comprises LCDR1, LCDR2, and LCDR3 as set forth in SEQ ID NOs: 93, 94, and 99, respectively. In some embodiments, the isolated anti-IBP4 antibody or the fragment thereof comprises LCDR1, LCDR2, and LCDR3 as set forth in SEQ ID NOs: 230, 231, and 227, respectively. In some embodiments, the isolated anti-IBP4 antibody or the fragment thereof comprises LCDR1, LCDR2, and LCDR3 as set forth in SEQ ID NOs: 93, 261, and 259, respectively. In some embodiments, the isolated anti-IBP4 antibody or the fragment thereof comprises LCDR1, LCDR2, and LCDR3 as set forth in SEQ ID NOs: 274, 94, and 259, respectively. In some embodiments, the isolated anti-IBP4 antibody or the fragment thereof comprises LCDR1, LCDR2, and LCDR3 as set forth in SEQ ID NOs: 93, 300, and 298, respectively. In some embodiments, the isolated anti-IBP4 antibody or the fragment thereof comprises LCDR1, LCDR2, and LCDR3 as set forth in SEQ ID NOs: 327, 328, and 324, respectively. In some embodiments, the HCDRs are associated with the LCDRs to form a IBP4 binding region.

In some embodiments, the CDRs are determined according to Chothia numbering. Chothia refers to the location of structural loops (see, e.g., Chothia and Lesk, J. Mol. Biol. 196:901-917 (1987)). Accordingly, in some embodiments an isolated anti-IBP4 antibody or a fragment thereof comprises HCDR1, HCDR2, and HCDR3 as set forth in SEQ ID NOs: 78, 79, and 80, respectively. In some embodiments, the isolated anti-IBP4 antibody or the fragment thereof comprises HCDR1, HCDR2, and HCDR3 as set forth in SEQ ID NOs: 78, 218, and 219, respectively. In some embodiments, the isolated anti-IBP4 antibody or the fragment thereof comprises HCDR1, HCDR2, and HCDR3 as set forth in SEQ ID NOs: 247, 248, and 249, respectively. In some embodiments, the isolated anti-IBP4 antibody or the fragment thereof comprises HCDR1, HCDR2, and HCDR3 as set forth in SEQ ID NOs: 247, 286, and 287, respectively. In some embodiments, the isolated anti-IBP4 antibody or the fragment thereof comprises HCDR1, HCDR2, and HCDR3 as set forth in SEQ ID NOs: 314, 315, and 316, respectively. In some embodiments, the isolated anti-IBP4 antibody or the fragment thereof comprises LCDR1, LCDR2, and LCDR3 as set forth in SEQ ID NOs: 90, 91, and 89, respectively. In some embodiments, the isolated anti-IBP4 antibody or the fragment thereof comprises LCDR1, LCDR2, and LCDR3 as set forth in SEQ ID NOs: 78, 79, and 80, respectively. In some embodiments, the isolated anti-IBP4 antibody or the fragment thereof comprises LCDR1, LCDR2, and LCDR3 as set forth in SEQ ID NOs: 228, 229, and 227, respectively. In some embodiments, the isolated anti-IBP4 antibody or the fragment thereof comprises LCDR1, LCDR2, and LCDR3 as set forth in SEQ ID NOs: 90, 260, and 259, respectively. In some embodiments, the isolated anti-IBP4 antibody or the fragment thereof comprises LCDR1, LCDR2, and LCDR3 as set forth in SEQ ID NOs: 272, 273, and 259, respectively. In some embodiments, the isolated anti-IBP4 antibody or the fragment thereof comprises LCDR1, LCDR2, and LCDR3 as set forth in SEQ ID NOs: 90, 299, and 298, respectively. In some embodiments, the isolated anti-IBP4 antibody or the fragment thereof comprises LCDR1, LCDR2, and LCDR3 as set forth in SEQ ID NOs: 325, 326, and 324, respectively. In some embodiments, the HCDRs are associated with the LCDRs to form a IBP4 binding region.

In some embodiments, the CDRs are determined according to AbM numbering. The AbM hypervariable regions represent a compromise between the Kabat CDRs and Chothia structural loops, and are used by Oxford Moleculars AbMantibody modeling Software (see, e.g., Martin, in Anti body Engineering, Vol. 2, Chapter 3, Springer Verlag). Accordingly, in some embodiments an isolated anti-IBP4 antibody or a fragment thereof comprises HCDR1, HCDR2, and HCDR3 as set forth in SEQ ID NOs: 81, 82, and 83, respectively. In some embodiments, the anti-IBP4 antibody or a fragment thereof comprises HCDR1, HCDR2, and HCDR3 as set forth in SEQ ID NOs: 81, 220, and 221, respectively. In some embodiments, the anti-IBP4 antibody or a fragment thereof comprises HCDR1, HCDR2, and HCDR3 as set forth in SEQ ID NOs: 250, 251, and 252, respectively. In some embodiments, the anti-IBP4 antibody or a fragment thereof comprises HCDR1, HCDR2, and HCDR3 as set forth in SEQ ID NOs: 288, 289, and 290, respectively. In some embodiments, the anti-IBP4 antibody or a fragment thereof comprises HCDR1, HCDR2, and HCDR3 as set forth in SEQ ID NOs: 288, 317, and 318, respectively. In some embodiments, the isolated anti-IBP4 antibody or the fragment thereof comprises LCDR1, LCDR2, and LCDR3 as set forth in SEQ ID NOS: 93, 94, and 95, respectively. In some embodiments, the isolated anti-IBP4 antibody or the fragment thereof comprises LCDR1, LCDR2, and LCDR3 as set forth in SEQ ID NOS: 93, 94, and 100, respectively. In some embodiments, the isolated anti-IBP4 antibody or the fragment thereof comprises LCDR1, LCDR2, and LCDR3 as set forth in SEQ ID NOS: 230, 231, and 232, respectively. In some embodiments, the isolated anti-IBP4 antibody or the fragment thereof comprises LCDR1, LCDR2, and LCDR3 as set forth in SEQ ID NOS: 93, 261, and 262, respectively. In some embodiments, the isolated anti-IBP4 antibody or the fragment thereof comprises LCDR1, LCDR2, and LCDR3 as set forth in SEQ ID NOS: 274, 94, and 262, respectively. In some embodiments, the isolated anti-IBP4 antibody or the fragment thereof comprises LCDR1, LCDR2, and LCDR3 as set forth in SEQ ID NOS: 93, 300, and 301, respectively. In some embodiments, the isolated anti-IBP4 antibody or the fragment thereof comprises LCDR1, LCDR2, and LCDR3 as set forth in SEQ ID NOS: 327, 328, and 329, respectively. In some embodiments, the HCDRs are associated with the LCDRs to form a IBP4 binding region.

In some embodiments, the CDRs are determined according to Contact numbering. Accordingly, in some embodiments an isolated anti-IBP4 antibody or a fragment thereof comprises HCDR1, HCDR2, and HCDR3 as set forth in SEQ ID NOs: 84, 85, and 86, respectively. In some embodiments, the isolated anti-IBP4 antibody or the fragment thereof comprises HCDR1, HCDR2, and HCDR3 as set forth in SEQ ID NOs: 84, 222, and 223, respectively. In some embodiments, the isolated anti-IBP4 antibody or the fragment thereof comprises HCDR1, HCDR2, and HCDR3 as set forth in SEQ ID NOs: 253, 254, and 255, respectively. In some embodiments, the isolated anti-IBP4 antibody or the fragment thereof comprises HCDR1, HCDR2, and HCDR3 as set forth in SEQ ID NOs: 291, 292, and 293, respectively. In some embodiments, the isolated anti-IBP4 antibody or the fragment thereof comprises HCDR1, HCDR2, and HCDR3 as set forth in SEQ ID NOs: 291, 319, and 320, respectively. In some embodiments, the isolated anti-IBP4 antibody or the fragment thereof comprises LCDR1, LCDR2, and LCDR3 as set forth in SEQ ID NOs: 96, 97, and 98, respectively. In some embodiments, the isolated anti-IBP4 antibody or the fragment thereof comprises LCDR1, LCDR2, and LCDR3 as set forth in SEQ ID NOs: 96, 97, 101, respectively. In some embodiments, the isolated anti-IBP4 antibody or the fragment thereof comprises LCDR1, LCDR2, and LCDR3 as set forth in SEQ ID NOs: 233, 234, 235, respectively. In some embodiments, the isolated anti-IBP4 antibody or the fragment thereof comprises LCDR1, LCDR2, and LCDR3 as set forth in SEQ ID NOs: 233, 234, 235, respectively. In some embodiments, the isolated anti-IBP4 antibody or the fragment thereof comprises LCDR1, LCDR2, and LCDR3 as set forth in SEQ ID NOs: 96, 263, 264, respectively. In some embodiments, the isolated anti-IBP4 antibody or the fragment thereof comprises LCDR1, LCDR2, and LCDR3 as set forth in SEQ ID NOs: 275, 97, 264, respectively. In some embodiments, the isolated anti-IBP4 antibody or the fragment thereof comprises LCDR1, LCDR2, and LCDR3 as set forth in SEQ ID NOs: 96, 302, 298, respectively. In some embodiments, the isolated anti-IBP4 antibody or the fragment thereof comprises LCDR1, LCDR2, and LCDR3 as set forth in SEQ ID NOs: 330, 331, 332, respectively. In some embodiments, the HCDRs are associated with the LCDRs to form a IBP4 binding region.

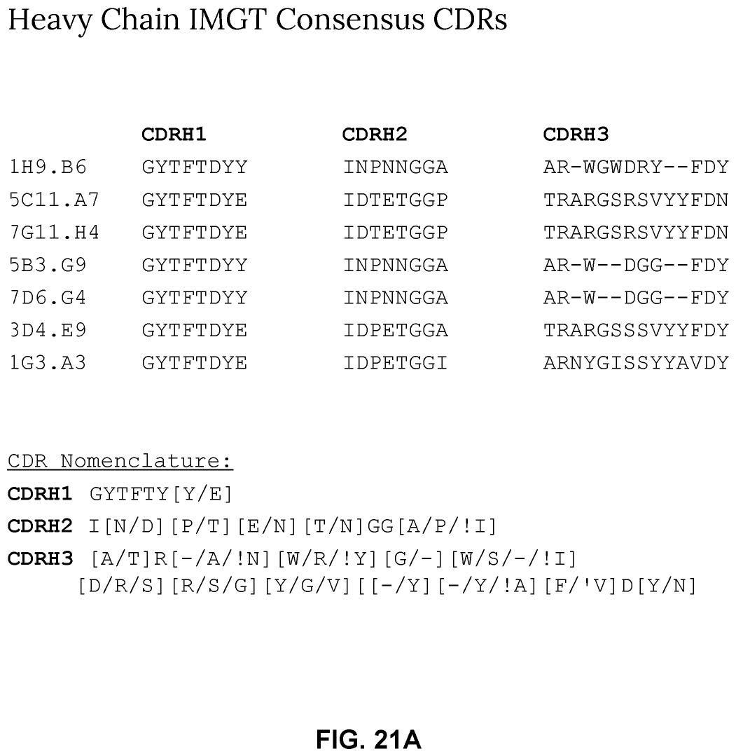

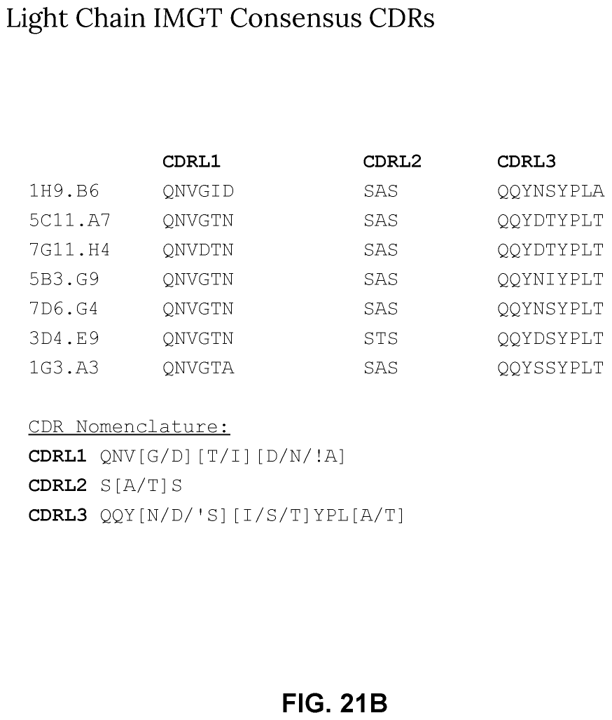

In some embodiments, the anti-IBP4 antibody is humanized. In some embodiments, the anti-IBP4 antibody is partially humanized. In some embodiments, the anti-IBP4 antibody is chimeric. In some embodiments, the anti-IBP4 antibody is chimeric humanized. In some embodiments, the anti-IBP4 is a mouse antibody. In some embodiments, the anti-IBP4 antibody comprises an acceptor human framework, e.g., a human immunoglobulin framework or a human consensus framework. In some embodiments, the anti-IBP4 antibody comprises an acceptor mouse framework, e.g., a mouse immunoglobulin framework or a mouse consensus framework. FIGS. 21A-21B show exemplary heavy chain consensus CDRs and light chain consensus CDRs for the anti-IBP4 antibodies, respectively.

In some embodiments, the anti-IBP4 antibody provided herein comprises HCDR1, HCDR2, and HCDR3 as set forth in SEQ ID NOs: 75-77, respectively. In some embodiments, the anti-IBP4 antibody provided herein comprises HCDR1, HCDR2, and HCDR3 as set forth in SEQ ID NOs: 78-80, respectively. In some embodiments, the anti-IBP4 antibody provided herein comprises HCDR1, HCDR2, and HCDR3 as set forth in SEQ ID NOs: 81-83, respectively. In some embodiments, the anti-IBP4 antibody provided herein comprises HCDR1, HCDR2, and HCDR3 as set forth in SEQ ID NOs: 84-86, respectively. In some embodiments, the anti-IBP4 antibody provided herein comprises HCDR1, HCDR2, and HCDR3 as set forth in SEQ ID NOs: 118, 119, and 83, respectively. In some embodiments, the anti-IBP4 antibody provided herein comprises HCDR1, HCDR2, and HCDR3 as set forth in SEQ ID NOs: 75, 216, and 217, respectively. In some embodiments, the anti-IBP4 antibody provided herein comprises HCDR1, HCDR2, and HCDR3 as set forth in SEQ ID NOs: 78, 218, and 219, respectively. In some embodiments, the anti-IBP4 antibody provided herein comprises HCDR1, HCDR2, and HCDR3 as set forth in SEQ ID NOs: 81, 220, and 221, respectively. In some embodiments, the anti-IBP4 antibody provided herein comprises HCDR1, HCDR2, and HCDR3 as set forth in SEQ ID NOs: 84, 222, and 223, respectively. In some embodiments, the anti-IBP4 antibody provided herein comprises HCDR1, HCDR2, and HCDR3 as set forth in SEQ ID NOs: 118, 224, and 225, respectively. In some embodiments, the anti-IBP4 antibody provided herein comprises HCDR1, HCDR2, and HCDR3 as set forth in SEQ ID NOs: 244, 245, and 246, respectively. In some embodiments, the anti-IBP4 antibody provided herein comprises HCDR1, HCDR2, and HCDR3 as set forth in SEQ ID NOs: 247, 248, and 249, respectively. In some embodiments, the anti-IBP4 antibody provided herein comprises HCDR1, HCDR2, and HCDR3 as set forth in SEQ ID NOs: 250, 251, and 252, respectively. In some embodiments, the anti-IBP4 antibody provided herein comprises HCDR1, HCDR2, and HCDR3 as set forth in SEQ ID NOs: 253, 254, and 255, respectively. In some embodiments, the anti-IBP4 antibody provided herein comprises HCDR1, HCDR2, and HCDR3 as set forth in SEQ ID NOs: 256, 257, and 258, respectively. In some embodiments, the anti-IBP4 antibody provided herein comprises HCDR1, HCDR2, and HCDR3 as set forth in SEQ ID NOs: 244, 284, and 285, respectively. In some embodiments, the anti-IBP4 antibody provided herein comprises HCDR1, HCDR2, and HCDR3 as set forth in SEQ ID NOs: 247, 286, and 287, respectively. In some embodiments, the anti-IBP4 antibody provided herein comprises HCDR1, HCDR2, and HCDR3 as set forth in SEQ ID NOs: 288, 289, and 290, respectively. In some embodiments, the anti-IBP4 antibody provided herein comprises HCDR1, HCDR2, and HCDR3 as set forth in SEQ ID NOs: 291, 292, and 293, respectively. In some embodiments, the anti-IBP4 antibody provided herein comprises HCDR1, HCDR2, and HCDR3 as set forth in SEQ ID NOs: 294, 295, and 296, respectively. In some embodiments, the anti-IBP4 antibody provided herein comprises HCDR1, HCDR2, and HCDR3 as set forth in SEQ ID NOs: 244, 312, and 313, respectively. In some embodiments, the anti-IBP4 antibody provided herein comprises HCDR1, HCDR2, and HCDR3 as set forth in SEQ ID NOs: 314, 315, and 316, respectively. In some embodiments, the anti-IBP4 antibody provided herein comprises HCDR1, HCDR2, and HCDR3 as set forth in SEQ ID NOs: 288, 317, and 318, respectively. In some embodiments, the anti-IBP4 antibody provided herein comprises HCDR1, HCDR2, and HCDR3 as set forth in SEQ ID NOs: 291, 319, and 320, respectively. In some embodiments, the anti-IBP4 antibody provided herein comprises HCDR1, HCDR2, and HCDR3 as set forth in SEQ ID NOs: 294, 321, and 322, respectively. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In some embodiments, the anti-IBP4 antibody provided herein comprises LCDR1, LCDR2, and LCDR3 as set forth in SEQ ID NOs: 87, 24, and 89, respectively. In some embodiments, the anti-IBP4 antibody provided herein comprises LCDR1, LCDR2, and LCDR3 as set forth in SEQ ID NOs: 90, 91, and 89, respectively. In some embodiments, the anti-IBP4 antibody provided herein comprises LCDR1, LCDR2, and LCDR3 as set forth in SEQ ID NOs: 93, 94, and 95, respectively. In some embodiments, the anti-IBP4 antibody provided herein comprises LCDR1, LCDR2, and LCDR3 as set forth in SEQ ID NOs: 96, 97, and 98, respectively. In some embodiments, the anti-IBP4 antibody provided herein comprises LCDR1, LCDR2, and LCDR3 as set forth in SEQ ID NOs: 93, 94, and 89, respectively. In some embodiments, the anti-IBP4 antibody provided herein comprises LCDR1, LCDR2, and LCDR3 as set forth in SEQ ID NOs: 87, 24, and 99, respectively. In some embodiments, the anti-IBP4 antibody provided herein comprises LCDR1, LCDR2, and LCDR3 as set forth in SEQ ID NOs: 90, 91, and 99, respectively. In some embodiments, the anti-IBP4 antibody provided herein comprises LCDR1, LCDR2, and LCDR3 as set forth in SEQ ID NOs: 93, 94, and 100, respectively. In some embodiments, the anti-IBP4 antibody provided herein comprises LCDR1, LCDR2, and LCDR3 as set forth in SEQ ID NOs: 96, 97, and 101, respectively. In some embodiments, the anti-IBP4 antibody provided herein comprises LCDR1, LCDR2, and LCDR3 as set forth in SEQ ID NOs: 93, 94, and 99, respectively. In some embodiments, the anti-IBP4 antibody provided herein comprises LCDR1, LCDR2, and LCDR3 as set forth in SEQ ID NOs: 226, 24, and 227, respectively. In some embodiments, the anti-IBP4 antibody provided herein comprises LCDR1, LCDR2, and LCDR3 as set forth in SEQ ID NOs: 228, 229, and 227, respectively. In some embodiments, the anti-IBP4 antibody provided herein comprises LCDR1, LCDR2, and LCDR3 as set forth in SEQ ID NOs: 230, 231, and 232, respectively. In some embodiments, the anti-IBP4 antibody provided herein comprises LCDR1, LCDR2, and LCDR3 as set forth in SEQ ID NOs: 233, 234, and 235, respectively. In some embodiments, the anti-IBP4 antibody provided herein comprises LCDR1, LCDR2, and LCDR3 as set forth in SEQ ID NOs: 230, 231, and 227, respectively. In some embodiments, the anti-IBP4 antibody provided herein comprises LCDR1, LCDR2, and LCDR3 as set forth in SEQ ID NOs: 87, 24, and 259, respectively. In some embodiments, the anti-IBP4 antibody provided herein comprises LCDR1, LCDR2, and LCDR3 as set forth in SEQ ID NOs: 90, 260, and 259, respectively. In some embodiments, the anti-IBP4 antibody provided herein comprises LCDR1, LCDR2, and LCDR3 as set forth in SEQ ID NOs: 93, 261, and 262, respectively. In some embodiments, the anti-IBP4 antibody provided herein comprises LCDR1, LCDR2, and LCDR3 as set forth in SEQ ID NOs: 96, 263, and 264, respectively. In some embodiments, the anti-IBP4 antibody provided herein comprises LCDR1, LCDR2, and LCDR3 as set forth in SEQ ID NOs: 93, 261 and 259, respectively. In some embodiments, the anti-IBP4 antibody provided herein comprises LCDR1, LCDR2, and LCDR3 as set forth in SEQ ID NOs: 23, 24, and 259, respectively. In some embodiments, the anti-IBP4 antibody provided herein comprises LCDR1, LCDR2, and LCDR3 as set forth in SEQ ID NOs: 272, 273, and 259, respectively. In some embodiments, the anti-IBP4 antibody provided herein comprises LCDR1, LCDR2, and LCDR3 as set forth in SEQ ID NOs: 275, 94, and 262, respectively. In some embodiments, the anti-IBP4 antibody provided herein comprises LCDR1, LCDR2, and LCDR3 as set forth in SEQ ID NOs: 275, 97, and 264, respectively. In some embodiments, the anti-IBP4 antibody provided herein comprises LCDR1, LCDR2, and LCDR3 as set forth in SEQ ID NOs: 274, 94, and 259, respectively. In some embodiments, the anti-IBP4 antibody provided herein comprises LCDR1, LCDR2, and LCDR3 as set forth in SEQ ID NOs: 87, 297, and 298, respectively. In some embodiments, the anti-IBP4 antibody provided herein comprises LCDR1, LCDR2, and LCDR3 as set forth in SEQ ID NOs: 90, 299, and 298, respectively. In some embodiments, the anti-IBP4 antibody provided herein comprises LCDR1, LCDR2, and LCDR3 as set forth in SEQ ID NOs: 93, 300, and 301, respectively. In some embodiments, the anti-IBP4 antibody provided herein comprises LCDR1, LCDR2, and LCDR3 as set forth in SEQ ID NOs: 96, 302, and 303, respectively. In some embodiments, the anti-IBP4 antibody provided herein comprises LCDR1, LCDR2, and LCDR3 as set forth in SEQ ID NOs: 93, 300, and 298, respectively. In some embodiments, the anti-IBP4 antibody provided herein comprises LCDR1, LCDR2, and LCDR3 as set forth in SEQ ID NOs: 323, 24, and 324, respectively. In some embodiments, the anti-IBP4 antibody provided herein comprises LCDR1, LCDR2, and LCDR3 as set forth in SEQ ID NOs: 325, 326, and 324, respectively. In some embodiments, the anti-IBP4 antibody provided herein comprises LCDR1, LCDR2, and LCDR3 as set forth in SEQ ID NOs: 327, 328, and 329, respectively. In some embodiments, the anti-IBP4 antibody provided herein comprises LCDR1, LCDR2, and LCDR3 as set forth in SEQ ID NOs: 330, 331, and 332, respectively. In some embodiments, the anti-IBP4 antibody provided herein comprises LCDR1, LCDR2, and LCDR3 as set forth in SEQ ID NOs: 327, 328, and 324, respectively. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In some embodiments, the antibody or the fragment thereof as provided herein comprises an HCDR1, an HCDR2, and an HCDR3 as set forth in SEQ ID NOs: 75, 76, and 77, and a LCDR1, a LCDR2, and a LCDR3 as set forth in SEQ ID NOs: 87, 24, and 89. In some embodiments, the antibody or antigen binding fragment provided herein comprises an HCDR1, an HCDR2, and an HCDR3 as set forth in SEQ ID NOs: 78, 79, and 80, and a LCDR1, a LCDR2, and a LCDR3 as set forth in SEQ ID NOs: 90, 91, and 89. In some embodiments, the antibody or antigen binding fragment provided herein comprises an HCDR1, an HCDR2, and an HCDR3 as set forth in SEQ ID NOs: 81, 82, and 83, and a LCDR1, a LCDR2, and a LCDR3 as set forth in SEQ ID NOs: 93, 94, and 95. In some embodiments, the antibody or antigen binding fragment provided herein comprises an HCDR1, an HCDR2, and an HCDR3 as set forth in SEQ ID NOs: 84, 85, and 86, and a LCDR1, a LCDR2, and a LCDR3 as set forth in SEQ ID NOs: 96, 97, and 98. In some embodiments, the antibody or antigen binding fragment provided herein comprises an HCDR1, an HCDR2, and an HCDR3 as set forth in SEQ ID NOs: 118, 119, and 83, and a LCDR1, a LCDR2, and a LCDR3 as set forth in SEQ ID NOs: 93, 94, and 89. In some embodiments, the antibody or antigen binding fragment provided herein comprises an HCDR1, an HCDR2, and an HCDR3 as set forth in SEQ ID NOs: 75, 216, and 217, and a LCDR1, a LCDR2, and a LCDR3 as set forth in SEQ ID NOs: 226, 24, and 227. In some embodiments, the antibody or antigen binding fragment provided herein comprises an HCDR1, an HCDR2, and an HCDR3 as set forth in SEQ ID NOs: 78, 218, and 219, and a LCDR1, a LCDR2, and a LCDR3 as set forth in SEQ ID NOs: 228, 229, and 227. In some embodiments, the antibody or antigen binding fragment provided herein comprises an HCDR1, an HCDR2, and an HCDR3 as set forth in SEQ ID NOs: 81, 220, and 221, and a LCDR1, a LCDR2, and a LCDR3 as set forth in SEQ ID NOs: 230, 231, and 232. In some embodiments, the antibody or antigen binding fragment provided herein comprises an HCDR1, an HCDR2, and an HCDR3 as set forth in SEQ ID NOs: 84, 222, and 223, and a LCDR1, a LCDR2, and a LCDR3 as set forth in SEQ ID NOs: 233, 234, and 235. In some embodiments, the antibody or antigen binding fragment provided herein comprises an HCDR1, an HCDR2, and an HCDR3 as set forth in SEQ ID NOs: 118, 224, and 225, and a LCDR1, a LCDR2, and a LCDR3 as set forth in SEQ ID NOs: 230, 231, and 227. In some embodiments, the antibody or antigen binding fragment provided herein comprises an HCDR1, an HCDR2, and an HCDR3 as set forth in SEQ ID NOs: 244, 245, and 246, and a LCDR1, a LCDR2, and a LCDR3 as set forth in SEQ ID NOs: 87, 24, and 259. In some embodiments, the antibody or antigen binding fragment provided herein comprises an HCDR1, an HCDR2, and an HCDR3 as set forth in SEQ ID NOs: 247, 248, and 249, and a LCDR1, a LCDR2, and a LCDR3 as set forth in SEQ ID NOs: 90, 260, and 259. In some embodiments, the antibody or antigen binding fragment provided herein comprises an HCDR1, an HCDR2, and an HCDR3 as set forth in SEQ ID NOs: 250, 251, and 252, and a LCDR1, a LCDR2, and a LCDR3 as set forth in SEQ ID NOs: 93, 261, and 262. In some embodiments, the antibody or antigen binding fragment provided herein comprises an HCDR1, an HCDR2, and an HCDR3 as set forth in SEQ ID NOs: 253, 254, and 255, and a LCDR1, a LCDR2, and a LCDR3 as set forth in SEQ ID NOs: 96, 263, and 264. In some embodiments, the antibody or antigen binding fragment provided herein comprises an HCDR1, an HCDR2, and an HCDR3 as set forth in SEQ ID NOs: 256, 257, and 258, and a LCDR1, a LCDR2, and a LCDR3 as set forth in SEQ ID NOs: 93, 261, and 259. In some embodiments, the antibody or antigen binding fragment provided herein comprises an HCDR1, an HCDR2, and an HCDR3 as set forth in SEQ ID NOs: 244, 245, and 246, and a LCDR1, a LCDR2, and a LCDR3 as set forth in SEQ ID NOs: 23, 24, and 259. In some embodiments, the antibody or antigen binding fragment provided herein comprises an HCDR1, an HCDR2, and an HCDR3 as set forth in SEQ ID NOs: 247, 248, and 249, and a LCDR1, a LCDR2, and a LCDR3 as set forth in SEQ ID NOs: 272, 273, and 259. In some embodiments, the antibody or antigen binding fragment provided herein comprises an HCDR1, an HCDR2, and an HCDR3 as set forth in SEQ ID NOs: 250, 252, and 252, and a LCDR1, a LCDR2, and a LCDR3 as set forth in SEQ ID NOs: 274, 94, and 262. In some embodiments, the antibody or antigen binding fragment provided herein comprises an HCDR1, an HCDR2, and an HCDR3 as set forth in SEQ ID NOs: 253, 254, and 255, and a LCDR1, a LCDR2, and a LCDR3 as set forth in SEQ ID NOs: 275, 97, and 264. In some embodiments, the antibody or antigen binding fragment provided herein comprises an HCDR1, an HCDR2, and an HCDR3 as set forth in SEQ ID NOs: 256, 257, and 258, and a LCDR1, a LCDR2, and a LCDR3 as set forth in SEQ ID NOs: 274, 94, and 259. In some embodiments, the antibody or antigen binding fragment provided herein comprises an HCDR1, an HCDR2, and an HCDR3 as set forth in SEQ ID NOs: 244, 284, and 285, and a LCDR1, a LCDR2, and a LCDR3 as set forth in SEQ ID NOs: 87, 297, and 298. In some embodiments, the antibody or antigen binding fragment provided herein comprises an HCDR1, an HCDR2, and an HCDR3 as set forth in SEQ ID NOs: 247, 286, and 287, and a LCDR1, a LCDR2, and a LCDR3 as set forth in SEQ ID NOs: 90, 299, and 298. In some embodiments, the antibody or antigen binding fragment provided herein comprises an HCDR1, an HCDR2, and an HCDR3 as set forth in SEQ ID NOs: 288, 289, and 290, and a LCDR1, a LCDR2, and a LCDR3 as set forth in SEQ ID NOs: 93, 300, and 301. In some embodiments, the antibody or antigen binding fragment provided herein comprises an HCDR1, an HCDR2, and an HCDR3 as set forth in SEQ ID NOs: 291, 292, and 293, and a LCDR1, a LCDR2, and a LCDR3 as set forth in SEQ ID NOs: 96, 302, and 303. In some embodiments, the antibody or antigen binding fragment provided herein comprises an HCDR1, an HCDR2, and an HCDR3 as set forth in SEQ ID NOs: 294, 295, and 296, and a LCDR1, a LCDR2, and a LCDR3 as set forth in SEQ ID NOs: 93, 300, and 298. In some embodiments, the antibody or antigen binding fragment provided herein comprises an HCDR1, an HCDR2, and an HCDR3 as set forth in SEQ ID NOs: 244, 312, and 313, and a LCDR1, a LCDR2, and a LCDR3 as set forth in SEQ ID NOs: 323, 24, and 324. In some embodiments, the antibody or antigen binding fragment provided herein comprises an HCDR1, an HCDR2, and an HCDR3 as set forth in SEQ ID NOs: 314, 315, and 316, and a LCDR1, a LCDR2, and a LCDR3 as set forth in SEQ ID NOs: 325, 326, and 324. In some embodiments, the antibody or antigen binding fragment provided herein comprises an HCDR1, an HCDR2, and an HCDR3 as set forth in SEQ ID NOs: 288, 317, and 318, and a LCDR1, a LCDR2, and a LCDR3 as set forth in SEQ ID NOs: 327, 328, and 329. In some embodiments, the antibody or antigen binding fragment provided herein comprises an HCDR1, an HCDR2, and an HCDR3 as set forth in SEQ ID NOs: 291, 319, and 320, and a LCDR1, a LCDR2, and a LCDR3 as set forth in SEQ ID NOs: 330, 331, and 332. In some embodiments, the antibody or antigen binding fragment provided herein comprises an HCDR1, an HCDR2, and an HCDR3 as set forth in SEQ ID NOs: 294, 321, and 322, and a LCDR1, a LCDR2, and a LCDR3 as set forth in SEQ ID NOs: 327, 328, and 324. Accordingly, CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In some embodiments, the antibody or the fragment thereof as provided herein comprises an HCDR1, an HCDR2, and an HCDR3 as set forth in SEQ ID NOs: 75, 76, and 77, and a LCDR1, a LCDR2, and a LCDR3 as set forth in SEQ ID NOs: 87, 24, and 99. In some embodiments, the antibody or antigen binding fragment provided herein comprises an HCDR1, an HCDR2, and an HCDR3 as set forth in SEQ ID NOs: 78, 79, 80, and a LCDR1, a LCDR2, and a LCDR3 as set forth in SEQ ID NOs: 90, 91, and 99. In some embodiments, the antibody or antigen binding fragment provided herein comprises an HCDR1, an HCDR2, and an HCDR3 as set forth in SEQ ID NOs: 81, 82, and 83, and a LCDR1, a LCDR2, and a LCDR3 as set forth in SEQ ID NOs: 93, 94, and 100. In some embodiments, the antibody or antigen binding fragment provided herein comprises an HCDR1, an HCDR2, and an HCDR3 as set forth in SEQ ID NOs: 84, 85, and 86, and a LCDR1, a LCDR2, and a LCDR3 as set forth in SEQ ID NOs: 96, 97, and 101. In some embodiments, the antibody or antigen binding fragment provided herein comprises an HCDR1, an HCDR2, and an HCDR3 as set forth in SEQ ID NOs: 118, 119, and 83, and a LCDR1, a LCDR2, and a LCDR3 as set forth in SEQ ID NOs: 93, 94, and 99. Accordingly, CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In some embodiments, provided herein is an antibody or a fragment thereof that binds to IBP4 comprising an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 75, 78, 81, 84, 118, 244, 247, 250, 253, 256, 288, 291, 284, and 314; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 76, 79, 82, 85, 119, 216, 218, 220, 222, 224, 245, 248, 251, 254, 257, 284, 286, 289, 292, 295, 312, 315, 317, 319, and 321, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 77, 80, 83, 86, 217, 219, 221, 223, 225, 246, 249, 252, 255, 258, 285, 287, 290, 293, 296, 313, 316, 318, 320, and 322; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 23, 87, 90, 93, 96, 226, 228, 230, 233, 272, 274, 275, 323, 325, 327, and 330; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 24, 91, 94, 97, 229, 231, 234, 260, 261, 263, 273, 297, 299, 300, 302, 326, 328, and 331; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to any of SEQ ID NOs: 89, 95, 98, 99, 100, 101, 227, 232, 235, 259, 262, 264, 298, 301, 303, 324, 329, and 332. In some embodiments, the anti-IBP4 antibody is humanized. In some embodiments, the anti-IBP4 antibody comprises an acceptor human framework, e.g., a human immunoglobulin framework or a human consensus framework.

In some embodiments, provided herein is an antibody or a fragment thereof that binds to IBP4 comprising an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 75, 78, 81, 84, 118, 244, 247, 250, 253, 256, 288, 291, 294, and 314; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 76, 79, 82, 85, 119, 216, 218, 220, 222, 224, 245, 248, 251, 254, 257, 284, 286, 289, 292, 295, 312, 315, 317, 319, and 321, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 77, 80, 83, 86, 217, 219, 221, 223, 225, 246, 249, 252, 255, 258, 285, 287, 290, 293, 296, 313, 316, 318, 320, 322; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 23, 87, 90, 93, 96, 226, 228, 230, 233, 272, 274, 275, 323, 325, 327, and 330; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 24, 91, 94, 97, 229, 231, 234, 260, 261, 263, 273, 297, 299, 300, 302, 326, 328, and 331; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to any of SEQ ID NOs: 89, 95, 98, 99, 100, 101, 227, 232, 235, 259, 262, 264, 298, 301, 303, 324, 329, and 332. In some embodiments, the anti-IBP4 antibody is humanized. In some embodiments, the anti-IBP4 antibody comprises an acceptor human framework, e.g., a human immunoglobulin framework or a human consensus framework.

In some embodiments, the antibody provided herein is a humanized antibody. Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FR1 is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system.

In some embodiments, the antibody or antigen binding fragment provided herein comprises a VH sequence having at least 75%, 80%, 85%, 86%, 87%, 88%, 89%, 90%, 91%, 92%, 93%, 94%, 95%, 96%, 97%, 98%, 99%, 100% sequence identity to sequence of SEQ ID NO: 107, and a VL sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 109. In some embodiments, the antibody or antigen binding fragment provided herein comprises a VH sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 72, and a VL sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 74. In some embodiments, the antibody or antigen binding fragment provided herein comprises a VH sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 213, and a VL sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 215. In some embodiments, the antibody or antigen binding fragment provided herein comprises a VH sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 241, and a VL sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 243. In some embodiments, the antibody or antigen binding fragment provided herein comprises a VH sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 241, and a VL sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 271. In some embodiments, the antibody or antigen binding fragment provided herein comprises a VH sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 281, and a VL sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 283. In some embodiments, the antibody or antigen binding fragment provided herein comprises a VH sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 309, and a VL sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 311. In further embodiments, one or more the amino acid residues “I”, “R”, “S” or “V” corresponding to the 58^{th}, 65^{th}, 116^{th}, and 117^{th} positions in the linear sequence of SEQ ID NO: 309, respectively, are not present (e.g., replaced with a different amino acid residue) in the VH sequence of the antibody or antigen binding fragment provided herein. In yet further embodiments, one or more the amino acid residues “T”, “A”, “L”, “N”, “T”, “M”, “V”, “S”, or “S” corresponding to the 14^{th}, 32^{nd}, 46^{th}, 53^{rd}, 56^{th}, 78^{th}, 87^{th}, 88^{th}, and 92^{nd} positions in the linear sequence of SEQ ID NO: 311, respectively, are not present (e.g., replaced with a different amino acid residue) in the VL sequence of the antibody or antigen binding fragment provided herein. In another embodiment, one or more the amino acid residues “R”, “T”, “L”, “E”, “H”, “T”, “P”, “V”, “H”, “G”, “A”, “D”, “E”, “T”, “I”, “R”, “I” “A”, “N”, “Y”, “I”, “S”, “Y”, “A”, “V”, “S”, or “V” corresponding to the 13^{th}, 19^{th}, 20^{th}, 33^{rd}, 35^{th}, 40^{th}, 41^{st}, 42^{nd}, 43^{rd}, 44^{th}, 50^{th}, 52^{nd}, 54^{th}, 55^{th}, 58^{th}, 65^{th}, 69^{th}, 72^{nd}, 99^{th}, 100^{th}, 102^{nd}, 104^{th}, 106^{th}, 107^{th}, 108^{th}, 116^{th}, and 117^{th} positions in the linear sequence of SEQ ID NO: 309, respectively, are not present (e.g., replaced with a different amino acid residue) in the VH sequence of the antibody or antigen binding fragment provided herein. In further embodiments, one or more the amino acid residues “T”, “E”, “I”, “A”, “L”, “N”, “T”, “M”, “D”, “V”, “S”, or “S” corresponding to the 14^{th}, 17^{th}, 21^{st}, 32^{nd}, 46^{th}, 53^{rd}, 56^{th}, 78^{th}, 85^{th}, 87^{th}, 88^{th}, and 92^{nd} positions in the linear sequence of SEQ ID NO: 311, respectively, are not present (e.g., replaced with a different amino acid residue) in the VL sequence of the antibody or antigen binding fragment provided herein. In another embodiment, one or more the amino acid residues “I”, “R”, “A”, “N”, “Y”, “I”, “Y”, “A”, “V”, “S”, or “V” corresponding to the 58^{th}, 65^{th}, 97^{th}, 99^{th}, 100^{th}, 102^{nd}, 105^{th}, 107^{th}, 108^{th}, 116^{th}, and 117^{th} positions in the linear sequence of SEQ ID NO: 309, respectively, are not present (e.g., replaced with a different amino acid residue) in the VH sequence of the antibody or antigen binding fragment provided herein. In further embodiments, one or more the amino acid residues “T”, “E”, “I”, “A”, “L”, “N”, “T”, “M”, “V”, “S”, “S”, “A”, or “L” corresponding to the 14^{th}, 17^{th}, 21^{st}, 32^{nd}, 46^{th}, 53^{rd}, 56^{th}, 78^{th}, 87^{th}, 88^{th}, 92^{nd}, 100^{th}, and 106^{th} positions in the linear sequence of SEQ ID NO: 311, respectively, are not present (e.g., replaced with a different amino acid residue) in the VL sequence of the antibody or antgen binding fragment provided herein.

In some embodiments, the antibody or antigen binding fragment provided herein comprises a heavy chain sequence having at least 75%, 80%, 85%, 86%, 87%, 88%, 89%, 90%, 91%, 92%, 93%, 94%, 95%, 96%, 97%, 98%, 99%, 100% sequence identity to any one of SEQ ID NOS: 68, 103, 123, 128, 209, 237, 266, 277, 305, 333, 335, 337, 339, or 341. In some embodiments, the heavy chain or light comprises a C-terminal Lysine residue (shown, for example, in underline in SEQ ID NOS: 68, 103, 209, 237, 266, 277, and 305). In some embodiments, the C-terminal Lysine residue is removed from the heavy chain. In some embodiments, the antibody or antigen binding fragment provided herein comprises a light chain sequence having at least 75%, 80%, 85%, 86%, 87%, 88%, 89%, 90%, 91%, 92%, 93%, 94%, 95%, 96%, 97%, 98%, 99%, 100% sequence identity to any one of SEQ ID NOS: 70, 105, 122, 129, 211, 239, 268, 279, or 307. In some embodiments, the heavy chain or light comprises a C-terminal Lysine residue (shown, for example, in underline in SEQ ID NOS: 70, and 105). In some embodiments, the C-terminal Lysine residue is removed from the light chain. In some embodiments the heavy chain sequence is associated with the light chain sequence to form an IBP4 binding region.

In certain embodiments, an antibody described herein or an antigen binding fragment thereof comprises amino acid sequences with certain percent identity relative to any antibody provided herein, for example, those described in the Sequence Listing below.

The determination of percent identity between two sequences (e.g., amino acid sequences or nucleic acid sequences) can be accomplished using a mathematical algorithm. A non-limiting example of a mathematical algorithm utilized for the comparison of two sequences is the algorithm of Karlin and Altschul, Proc. Natl. Acad. Sci. U.S.A. 87:2264 2268 (1990), modified as in Karlin and Altschul, Proc. Natl. Acad. Sci. U.S.A. 90:5873 5877 (1993). Such an algorithm is incorporated into the NBLAST and XBLAST programs of Altschul et al., J. Mol. Biol. 215:403 (1990). BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res. 25:3389 3402 (1997). Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, CABIOS 4:11-17 (1998). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In one aspect, the anti-IBP4 antibody or fragment thereof disclosed herein can comprise a degree of variability with respect to the LCDR3. Accordingly, provided herein is an isolated anti-IBP4 antibody or a fragment thereof comprising a heavy chain comprising a HCDR1 comprising amino acid sequence GYTFTDYY (SEQ ID NO: 75), a HCDR2 comprising amino acid sequence INPNNGGS (SEQ ID NO: 76), and a HCDR3 comprising amino acid sequence ARWDGGFDY (SEQ ID NO: 77); and a light chain comprises a LCDR1 comprising amino acid sequence QNVGTN (SEQ ID NO: 87), a LCDR2 comprising amino acid sequence SAS (SEQ ID NO: 24), and a LCDR3 comprising amino acid sequence QQYNX₁YPLT (SEQ ID NO: 114). The residue identied as X₁ can be independently any naturally occurring amino acid residue. For example, the residue identified as X₁ can be a isoleucine or serine. In some embodiments, X₁ is a isoleucine. In other embodiments, X₁ is a serine.

In another aspect, the anti-IBP4 antibody or fragment thereof disclosed herein can comprise a degree of variability with respect to one or more the HCDR1-HDCDR3 or LCDR1-LCDR3 disclosed herein. Accordingly, provided herein is an isolated anti-IBP4 antibody or a fragment thereof comprising (i) a heavy chain comprising a HCDR1 comprising amino acid sequence GYTFTYX₁ (SEQ ID NO: 354), a HCDR2 comprising amino acid sequence IX₂X₃X₄X₅GGX₆ (SEQ ID NO: 355), and a HCDR3 comprising amino acid sequence X₇RX₈X₉X₁₀X₁₁X₁₂X₁₃X₁₄X₁₅X₁₆X₁₇DX₁₈ (SEQ ID NO: 356); and (ii) a light chain comprising a LCDR1 comprising amino acid sequence QNVX₁₉X₂₀X₂₁ (SEQ ID NO: 357), a LCDR2 comprising amino acid sequence SX₂₂S (SEQ ID NO: 358), and a LCDR3 comprising amino acid sequence QQYX₂₃X₂₄YPLX₂₅ (SEQ ID NO: 359). In some embodiments, each of X₁-X₅, X₇, X₁₂-X₁₄, X₁₅-X₂₀, X₂₂, and X₂₄—X₂₅ are independently any naturally occurring amino acid residue, X₆ is independently any naturally occurring amino acid residue except isoleucine, Xₛ is independently a deletion or any naturally occurring amino acid residue except asparagine, X₉ is independently any naturally occurring amino acid residue except tyrosine, X₁₀ is independently a deletion or any naturally occurring amino acid residue, Xii is independently a deletion or any naturally occurring amino acid residue except isoleucine, X₁₅ is independently a deletion or any naturally occurring amino acid residue, X₁₆ is independently a deletion or any naturally occurring amino acid residue except alanine, X₁₇ is independently any naturally occurring amino acid residue except valine, X₂₁ is independently any naturally occurring amino acid residue except alanine, and X₂₃ is independently any naturally occurring amino acid residue except serine. In some embodiments, X₁ is a tyrosine or glutamic acid. In some embodiments, X₂ is an asparagine or aspartic acid. In some embodiments, X₃ is a proline or threonine. In some embodiments, X₄ is a glutamic acid or asparagine. In some embodiments, X₅ is a threonine or asparagine. In some embodiments, X₆ is an alanine or proline; X₇ is an alanine or threonine. In some embodiments, Xₛ is an alanine. In some embodiments, X₉ is a tryptophan or arginine. In some embodiments, X₁₀ is a glycine. In some embodiments, Xii is a tryptophan or serine. In some embodiments, X₁₂ is an aspartic acid or arginine or serine. In some embodiments, X₁₃ is an arginine or serine or glycine. In some embodiments, X₁₄ is a tyrosine or glycine or valine. In some embodiments, X₁₅ is a tyrosine. In some embodiments, X₁₆ is a tyrosine. In some embodiments, X₁₇ is a phenylalanine. In some embodiments, X₁ₛ is a tyrosine or asparagine. In some embodiments, X₁₉ is a glycine or aspartic acid. In some embodiments, X₂₀ is a threonine or isoleucine. In some embodiments, X₂₁ is an aspartic acid or asparagine. In some embodiments, X₂₂ is an alanine or threonine. In some embodiments, X₂₃ is an asparagine or aspartic acid. In some embodiments, X₂₄ is a isoleucine or serine or threonine. In some embodiments, X₂₅ is an alanine or threonine.

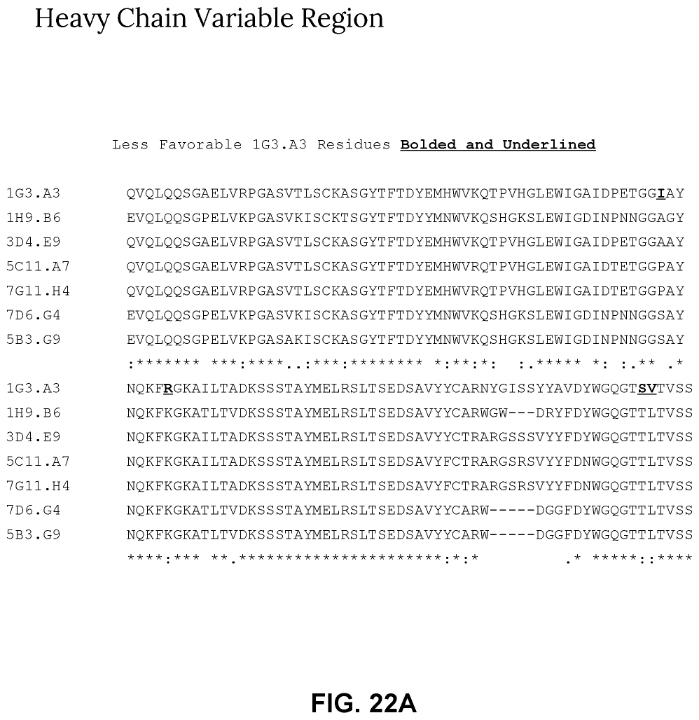

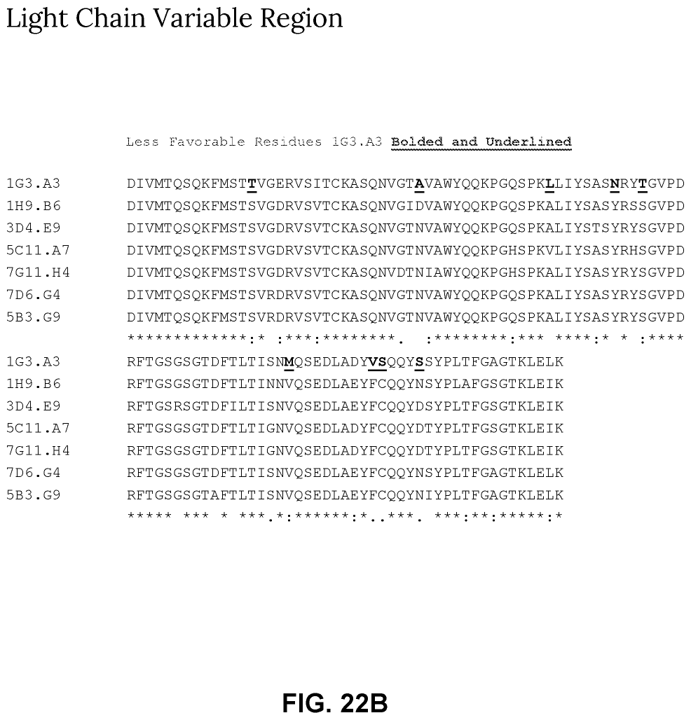

In some embodiments, the anti-IBP4 antibody or fragment thereof disclosed herein contains a HCDR1 comprising amino acid sequence GYTFTYX_{I} (SEQ ID NO: 354), a HCDR2 comprising amino acid sequence IX₂X₃X₄X₅GGX₆ (SEQ ID NO: 355), and a HCDR3 comprising amino acid sequence X₇RX₈X₉X₁₀X₁₁X₁₂X₁₃X₁₄X₁₅X₁₆X₁₇DX₁₈ (SEQ ID NO: 356); and (ii) a light chain comprising a LCDR1 comprising amino acid sequence QNVX₁₉X₂₀X₂₁ (SEQ ID NO: 357), a LCDR2 comprising amino acid sequence SX₂₂S (SEQ ID NO: 358), and a LCDR3 comprising amino acid sequence QQYX₂₃X₂₄YPLX₂₅ (SEQ ID NO: 359), wherein any one of the X₁ to X₂₅ positions have a combination of amino acid residues described herein. For example, for HCDR2, in some embodiments, X₂ is an asparagine or aspartic acid, X₃ is a proline or threonine, X₄ to X₅ are any naturally occurring amino acid residue, and X₆ is any naturally occurring amino acid residue except isoleucine. As another example, for LCDR1, in some embodiments, X₁₉ is any naturally occurring amino acid residue, X₂₀ is a threonine or isoleucine, and X₂₁ is any naturally occurring amino acid residue except alanine. In another example, for LCDR2, in some embodiments, X₂₂ is an alanine or threonine. In yet another example, for LCDR3, in some embodiments, X₂₃ is any naturally occurring amino acid residue except serine, X₂₄ is an isoleucine, serine, or threonine, and X₂₅ is an alanine or threonine. FIGS. 21A-21B, for instance, show exemplary heavy chain and light chain IMGT consensus CDRs (complementarity determining regions) for the anti-IBP4 antibodies 1H9.B6, 5C11.A7, 7G11.H4, 5B3.G9, 7D6.G4, 3D4.E9, and 1G3.A3. The CDR nomenclature is shown below the sequences. Exemplary HCDRs and LCDRs and their respective SEQ ID NOs. can be identified, for instance, in any of Tables 18, 20, 24, 26, 28, 30, and 32. Exemplary consensus CDR sequences for anti-SHBG and anti-IBP4 antibodies, and their respective SEQ ID NOs., can be identified, for instance, in Table 22. FIG. 21A shows exemplary heavy chain IMGT consensus CDRs. Sequences for each CDRH1, CDRH2, and CDRH3 are shown in the left, center, and right columns, respectively (SEQ ID NOs: 275, 216 and 217 for 1H9.B6; SEQ ID NOs: 244, 245, and 246 for 5C11.A7 and 7G11.H4; SEQ ID NOs: 75, 216 and 77 for 5B3.G9 and 7D6.G4; SEQ ID NOs: 244, 284 and 285 for 3D4.E9; and SEQ ID NOs. 244, 312 and 313 for 1G3.A3). FIG. 21B shows exemplary light chain IMGT consensus CDRs. Sequences for each CDRL1, CDRL2, and CDRL3 are shown in the left, center, and right columns, respectively (SEQ ID NOs. 226, 24 and 227 for 1H9.B6; SEQ ID NOs. 87, 24 and 259 for 5C11.A7; SEQ ID NOs. 23, 24 and 259 for 7G11.H4; SEQ ID NOs. 87, 24 and 89 for 5B3.G9; SEQ ID NOs. 87, 24 and 99 for 7D6.G4; SEQ ID NOs. 87, 297 and 298 for 3D4.E9; and SEQ ID NOs. 323, 24 and 324 for 1G3.A3). FIGS. 22A-22B show exemplary heavy chain variable regions and light chain variable regions for the anti-IBP4 antibodies 1G3.A3, 1H9.B6, 3D4.E9, 5C11.A7, 7G11.H4, 7D6.G4, and 5B3.G9. Bold and underlined residues indicate less favorable residues from the 1G3.A3 clone according to the alignment of these antibodies. Exemplary heavy chain variable regions and light chain variable regions and their respective SEQ ID NOs. can be identified, for instance, in any of Tables 17, 19, 23, 25, 27, 29, and 31. FIG. 22A shows the heavy chain variable regions for 1G3.A3 (SEQ ID NO: 309), 1H9.B6 (SEQ ID NO: 213), 3D4.E9 (SEQ ID NO: 281), 5C11.A7 (SEQ ID NO: 241), 7G11.H4 (SEQ ID NO: 241), 7D6.G4 (SEQ ID NO: 107), and 5B3.G9 (SEQ ID NO: 72). FIG. 22B shows the light chain variable regions for 1G3.A3 (SEQ ID NO: 311), 1H9.B6 (SEQ ID NO: 215), 3D4.E9 (SEQ ID NO: 283), 5C11.A7 (SEQ ID NO: 243), 7G11.H4 (SEQ ID NO: 271), 7D6.G4 (SEQ ID NO: 109), and 5B3.G9 (SEQ ID NO: 74).

In another aspect, provided herein is an isolated anti-IBP4 antibody or a fragment thereof, comprising: (i) a heavy chain comprising a HCDR1 comprising amino acid sequence GYTFTDYY (SEQ ID NO: 75), a HCDR2 comprising amino acid sequence INPNNGGX₁(SEQ ID NO: 360), and a HCDR3 comprising amino acid sequence ARWX₂X₃DX₄X₅FDY (SEQ ID NO: 361); and (ii) a light chain comprising a LCDR1 comprising amino acid sequence QNVGX₆X₇(SEQ ID NO: 362), a LCDR2 comprising amino acid sequence SAS (SEQ ID NO: 24), and a LCDR3 comprising amino acid sequence QQYNX₈YPLX₉ (SEQ ID NO: 363). In some embodiments, X₁ is independently any naturally occurring amino acid residue except isoleucine, each of X₂ and X₃ are independently a deletion or any naturally occurring amino acid residue, X₄ is independently any naturally occurring amino acid residue except serine, each of X₅, X₆, Xₛ, and X₉ are independently any naturally occurring amino acid residue, and X₇ is independently any naturally occurring amino acid residue except alanine. In some embodiments, X₁ is an alanine or serine. In some embodiments, X₂ is a glycine. In some embodiments, X₃ is a tryptophan. In some embodiments, X₄ is an arginine or glycine. In some embodiments, X₅ is a tyrosine or glycine. In some embodiments, X₆ is an isoleucine or threonine. In some embodiments, X₇ is an aspartic acid or asparagine. In some embodiments, X₈ is a serine or isoleucine. In some embodiments, X₉ is an alanine or threonine. In some embodiments, the anti-IBP4 antibody or fragment thereof disclosed herein contains a HCDR1 comprising amino acid sequence GYTFTDYY (SEQ ID NO: 75), a HCDR2 comprising amino acid sequence INPNNGGX₁(SEQ ID NO: 360), and a HCDR3 comprising amino acid sequence ARWX₂X₃DX₄X₅FDY (SEQ ID NO: 361); and (ii) a light chain comprising a LCDR1 comprising amino acid sequence QNVGX₆X₇(SEQ ID NO: 362), a LCDR2 comprising amino acid sequence SAS (SEQ ID NO: 24), and a LCDR3 comprising amino acid sequence QQYNX₈YPLX₉ (SEQ ID NO: 363), wherein any one of the X₁ to X₉ positions have a combination of amino acid residues described herein. For example, for HCDR3, in some embodiments, X₂ is a deletion, X₃ is a tryptophan, X₄ is an arginine or glycine, and X₅ is any naturally occurring amino acid residue. In another example, for LCDR1, in some embodiments, X₆ is any naturally occurring amino acid residue and X₇ is an aspartic acid or asparagine. In another example, for LCDR3, in some embodiments, X₈ is a serine or isoleucine and X₉ is any naturally occurring amino acid residue.

In another aspect, provided herein is an isolated anti-IBP4 antibody or a fragment thereof, comprising: (i) a heavy chain comprising a HCDR1 comprising amino acid sequence GYTFTDYE (SEQ ID NO: 244), a HCDR2 comprising amino acid sequence IDX₁ETGGX₂ (SEQ ID NO: 364), and a HCDR3 comprising amino acid sequence TRARGSX₃SVYYFDX₄ (SEQ ID NO: 365); and (ii) a light chain comprising a LCDR1 comprising amino acid sequence QNVX₅TN (SEQ ID NO: 366), a LCDR2 comprising amino acid sequence SX₆S (SEQ ID NO: 367), and a LCDR3 comprising amino acid sequence QQYDX₇YPLT (SEQ ID NO: 368). In some embodiments, each of X₁, X₃, X₄, X₅, X₆, and X₇ are independently any naturally occurring amino acid residue and X₂ is independently any naturally occurring amino acid residue except isoleucine. In some embodiments, X₁ is a proline or threonine. In some embodiments, X₂ is a proline or alanine. In some embodiments, X₃ is a serine or arginine. In some embodiments, X₄ is a tyrosine or asparagine. In some embodiments, X₅ is a glycine or aspartic acid. In some embodiments, X₆ is an alanine or threonine. In some embodiments, X₇ is a serine or threonine. In some embodiments, the anti-IBP4 antibody or fragment thereof disclosed herein contains a HCDR1 comprising amino acid sequence GYTFTDYE (SEQ ID NO: 244), a HCDR2 comprising amino acid sequence IDX₁ETGGX₂ (SEQ ID NO: 364), and a HCDR3 comprising amino acid sequence TRARGSX₃SVYYFDX₄ (SEQ ID NO: 365); and (ii) a light chain comprising a LCDR1 comprising amino acid sequence QNVX₅TN (SEQ ID NO: 366), a LCDR2 comprising amino acid sequence SX₆S (SEQ ID NO: 367), and a LCDR3 comprising amino acid sequence QQYDX₇YPLT (SEQ ID NO: 368), wherein any one of the X₁ to X₇ positions have a combination of amino acid residues described herein. For example, for HCDR2, in some embodiments, X₁ is a proline or threonine and X₂ is any naturally occurring amino acid residue. In another example, for HCDR3, in some embodiments, X₃ is any naturally occurring amino acid residue and X₄ is a tyrosine or asparagine. In another example, for LCDR3, in some embodiments, X₇ is a serine.

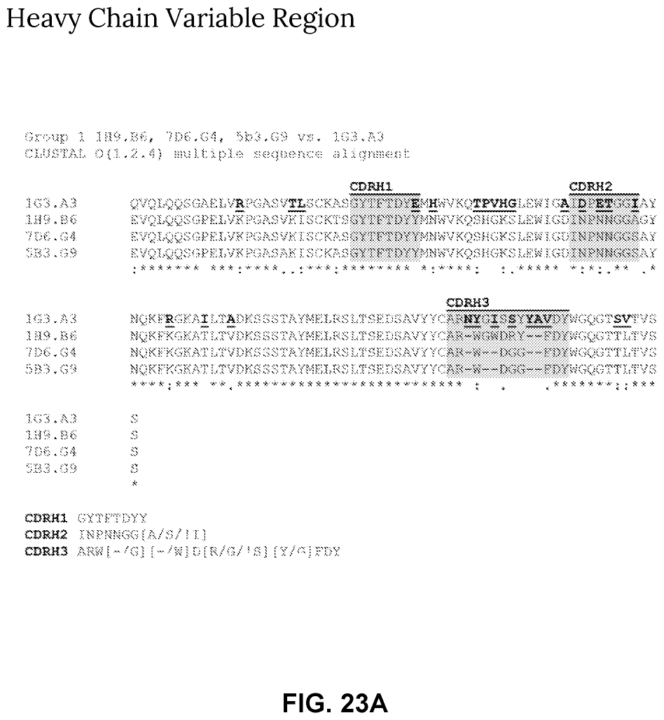

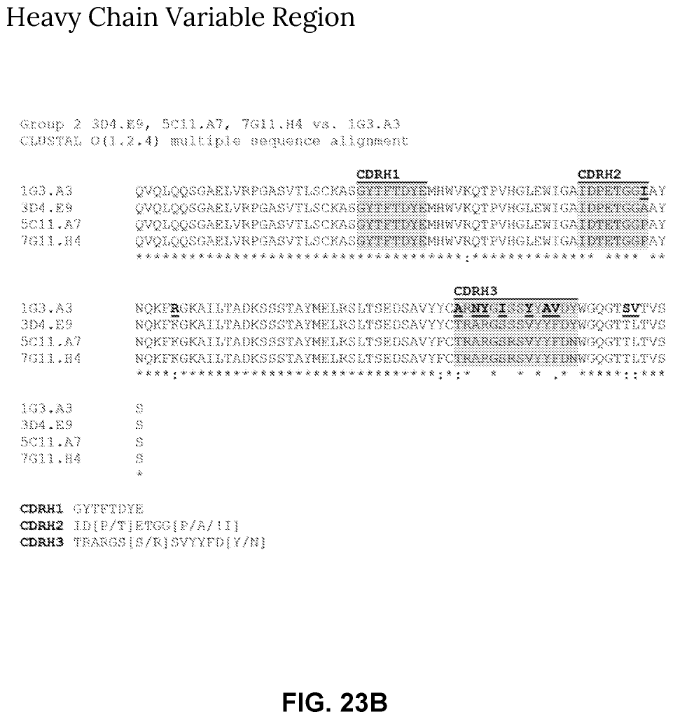

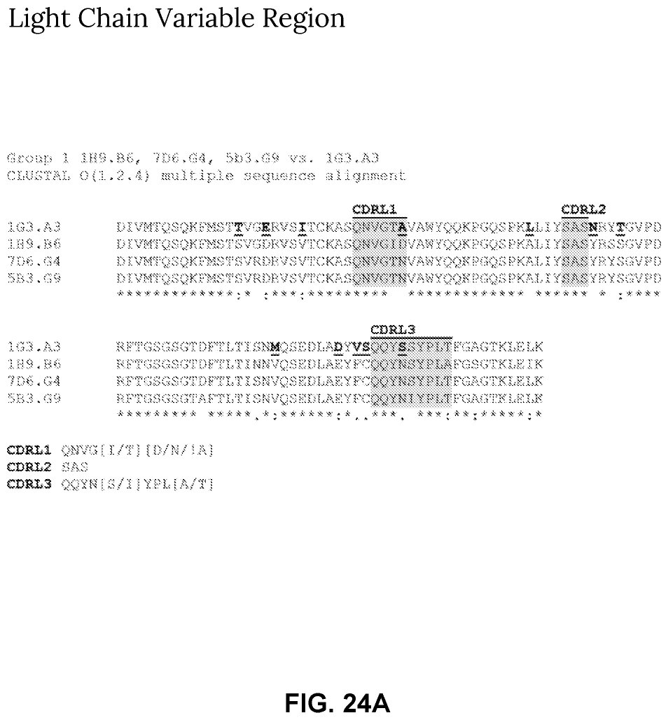

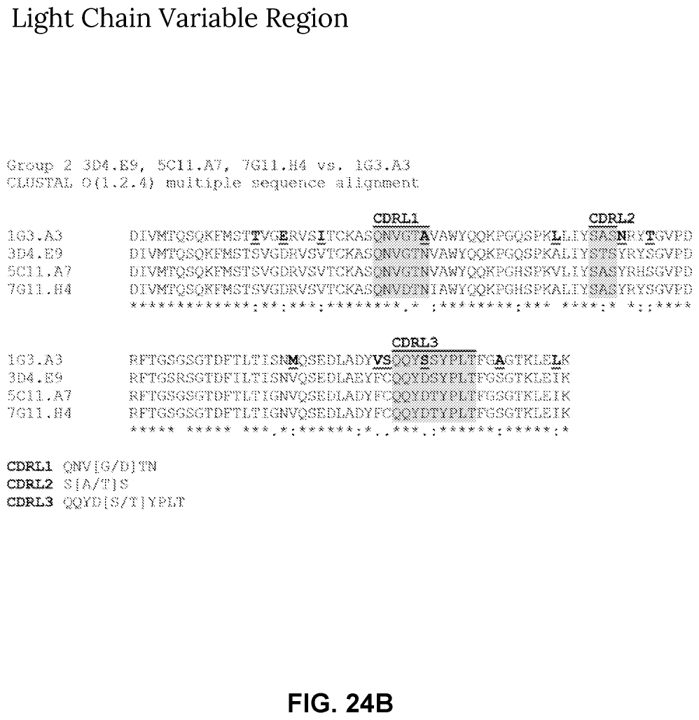

FIGS. 23A-23B, for instance, show exemplary CLUSTAL O (1.2.4) multiple sequence alignments for the heavy chain variable regions for the 2 groups of anti-IBP4 antibodies: 1H9.B6, 7D6.G4, 5B3.G9 and 1G3.A3 in one group (Group 1), and 3D4.E9, 5C1.A7, 7G11.H4, and 1G3.A3 in another group (Group 2). HCDRs for each alignment and group are highlighted. Bold and underlined residues indicate less favorable residues from the 1G3.A3 clone according to the alignment of these antibodies. CDR nomenclature is shown below the sequence alignment for each group. Exemplary consensus CDR sequences for anti-SHBG and anti-IBP4 antibodies, and their respective SEQ ID NOs., can be identified, for instance, in Table 22. FIG. 23A shows an exemplary sequence alignment for the heavy chain variable regions for Group 1: 1G3.A3 (SEQ ID NO: 309), 1H9.B6 (SEQ ID NO: 213), 7D6.G4 (SEQ ID NO: 107), and 5B3.G9 (SEQ ID NO: 72). FIG. 23B shows an exemplary sequence alignment for the heavy chain variable regions for Group 2: 1G3.A3 (SEQ ID NO: 309), 3D4.E9 (SEQ ID NO: 281), 5C11.A7 (SEQ ID NO: 241), and 7G11.H4 (SEQ ID NO: 241). FIGS. 24A-24B also show exemplary CLUSTAL O (1.2.4) multiple sequence alignments for the light chain variable regions for the 2 groups of anti-IBP4 antibodies: 1H9.B6, 7D6.G4, 5B3.G9 and 1G3.A3 (Group 1), and 3D4.E9, 5C11.A7, 7G11.H4, and 1G3.A3 (Group 2). LCDRs for each alignment and group are highlighted. Bold and underlined residues indicate less favorable residues from the 1G3.A3 clone according to the alignment of these antibodies. CDR nomenclature is shown below the sequence alignment for each group. FIG. 24A shows an exemplary sequence alignment for the light chain variable regions for Group 1: 1G3.A3 (SEQ ID NO: 311), 1H9.B6 (SEQ ID NO: 215), 7D6.G4 (SEQ ID NO: 109), and 5B3.G9 (SEQ ID NO: 74). FIG. 24B shows an exemplary sequence alignment for the light chain variable regions for Group 2: 1G3.A3 (SEQ ID NO: 311), 3D4.E9 (SEQ ID NO: 283), 5C11.A7 (SEQ ID NO: 243), and 7G11.H4 (SEQ ID NO: 271).

In some embodiments, the antibody provide herein contains substitutions (e.g., conservative substitutions), insertions, or deletions relative to the reference sequence, but the anti-IBP4 antibody comprising that sequence retains the ability to bind to IBP4. In some embodiments, a total of 1 to 10 amino acids have been substituted, inserted and/or deleted in a reference amino acid sequence. In some embodiments, substitutions, insertions, or deletions occur in regions outside the CDRs (i.e., in the FRs). Optionally, the anti-IBP4 antibody provided herein includes post-translational modifications of a reference sequence.

In some embodiments, functional epitopes can be mapped, e.g., by combinatorial alanine scanning, to identify amino acids in the IBP4 protein that are necessary for interaction with anti-IBP4 antibodies provided herein. In some embodiments, conformational and crystal structure of anti-IBP4 antibody bound to IBP4 may be employed to identify the epitopes. In some embodiments, the present disclosure provides an antibody that specifically binds to the same epitope as any of the anti-IBP4 antibodies provided herein. For example, in some embodiments, the antibody or antigen binding fragment provided herein binds to the same epitope as an anti-IBP4 antibody comprising a VH comprising the amino acid sequence of SEQ ID NO: 72, and a VL comprising the amino acid sequence of SEQ ID NO: 74. In some embodiments, the antibody or antigen binding fragment provided herein binds to the same epitope as an anti-IBP4 antibody comprising a VH comprising the amino acid sequence of SEQ ID NO: 107, and a VL comprising the amino acid sequence of SEQ ID NO: 109. In some embodiments, the antibody or antigen binding fragment provided herein binds to the same epitope as an anti-IBP4 antibody comprising a VH comprising the amino acid sequence of SEQ ID NO: 213, and a VL comprising the amino acid sequence of SEQ ID NO: 215. In some embodiments, the antibody or antigen binding fragment provided herein binds to the same epitope as an anti-IBP4 antibody comprising a VH comprising the amino acid sequence of SEQ ID NO: 241, and a VL comprising the amino acid sequence of SEQ ID NO: 243. In some embodiments, the antibody or antigen binding fragment provided herein binds to the same epitope as an anti-IBP4 antibody comprising a VH comprising the amino acid sequence of SEQ ID NO: 241, and a VL comprising the amino acid sequence of SEQ ID NO: 271. In some embodiments, the antibody or antigen binding fragment provided herein binds to the same epitope as an anti-IBP4 antibody comprising a VH comprising the amino acid sequence of SEQ ID NO: 281, and a VL comprising the amino acid sequence of SEQ ID NO: 283. In some embodiments, the antibody or antigen binding fragment provided herein binds to the same epitope as an anti-IBP4 antibody comprising a VH comprising the amino acid sequence of SEQ ID NO: 309, and a VL comprising the amino acid sequence of SEQ ID NO: 311.

In some embodiments, provided herein is an anti-IBP4 antibody, or antigen binding fragment thereof, that specifically binds to IBP4 competitively with any one of the anti-IBP4 antibodies described herein. For example, in some embodiments, the antibody or antigen binding fragment provided herein binds to the same epitope as an anti-IBP4 antibody comprises a VH comprising the amino acid sequence of SEQ ID NO: 72, and a VL comprising the amino acid sequence of SEQ ID NO: 74. In some embodiments, the antibody or antigen binding fragment provided herein binds to the same epitope as an anti-IBP4 antibody comprising a VH comprising the amino acid sequence of SEQ ID NO: 107, and a VL comprising the amino acid sequence of SEQ ID NO: 109. In some embodiments, the antibody or antigen binding fragment provided herein binds to the same epitope as an anti-IBP4 antibody comprising a VH comprising the amino acid sequence of SEQ ID NO: 213, and a VL comprising the amino acid sequence of SEQ ID NO: 215. In some embodiments, the antibody or antigen binding fragment provided herein binds to the same epitope as an anti-IBP4 antibody comprising a VH comprising the amino acid sequence of SEQ ID NO: 241, and a VL comprising the amino acid sequence of SEQ ID NO: 243. In some embodiments, the antibody or antigen binding fragment provided herein binds to the same epitope as an anti-IBP4 antibody comprising a VH comprising the amino acid sequence of SEQ ID NO: 241, and a VL comprising the amino acid sequence of SEQ ID NO: 271. In some embodiments, the antibody or antigen binding fragment provided herein binds to the same epitope as an anti-IBP4 antibody comprising a VH comprising the amino acid sequence of SEQ ID NO: 281, and a VL comprising the amino acid sequence of SEQ ID NO: 283. In some embodiments, the antibody or antigen binding fragment provided herein binds to the same epitope as an anti-IBP4 antibody comprising a VH comprising the amino acid sequence of SEQ ID NO: 309, and a VL comprising the amino acid sequence of SEQ ID NO: 311.

In some embodiments, provided herein is a IBP4 binding agent comprising any one of the anti-IBP4 antibodies described above. In some embodiments, the IBP4 binding agent is a monoclonal antibody, including a mouse, chimeric, humanized or human antibody. In some embodiments, the anti-IBP4 antibody is an antibody fragment, e.g., a scFv. In some embodiments, the IBP4 binding agent is a fusion protein comprising the anti-IBP4 antibody provided herein. In other embodiments, the IBP4 binding agent is a multispecific antibody comprising the anti-IBP4 antibody provided herein. Other exemplary IBP4 binding agents are described in more detail in the following sections.

In some embodiments, the anti-IBP4 antibody or antigen binding protein according to any of the above embodiments may incorporate any of the features, singly or in combination, as described below.

In another aspect, this disclosure relates to a nucleic acid molecule encoding a heavy chain and/or a light chain of an antibody or fragment thereof disclosed herein. The nucleic acid molecule is preferable an isolated nucleic acid molecule. The isolated nucleic acid molecule can be a nucleic acid molecule that is substantially pure. In some embodiments, the nucleic acid molecule encodes a heavy chain of an anti-IBP4 antibody or fragment thereof. In some embodiments, the nucleic acid molecule encoding the heavy chain has at least 85%, 90%, 95%, 96%, 97%, 98%, or 99% identity to at least one of SEQ ID NOS: 67, 102, 204, 206, 208, 236, 265, 276, 304, 334, 336, 338, 340, or 342. In some embodiments, the nucleic acid molecule encodes a light chain of an anti-IBP4 antibody or fragment thereof. In some embodiments, the nucleic acid molecule encoding the light chain has at least 85%, 90%, 95%, 96%, 97%, 98%, or 99% identity to SEQ ID NO: 69, 104, 205, 207, 210, 238, 267, 278, or 306.

### Methods and Compostions for Making Antibodies

Antibodies or fragments thereof that are useful in the present methods, compositions, kits, etc. can be produced by, for instance, recombinant expression techniques, from hybridomas, from myelomas or from other antibody expressing mammalian cells. As well recognized in the art, recombinant expression of an antibody or fragment thereof that binds to a target antigen typically involves construction of an expression vector containing a nucleic acid that encodes the polypeptides (e.g., heavy chain and light chain) that form the antibody or fragment thereof. Once a nucleic acid encoding such polypeptides has been obtained, the vector for the production of the antibody may be produced by recombinant DNA technology using techniques well known in the art. For example, for recombinant expression of an antibody, an expression vector may encode a heavy or light chain thereof, or a heavy or light chain variable domain, operably linked to a promoter. An expression vector may include, e.g., the nucleotide sequence encoding the constant region of the antibody molecule, and the variable domain of the antibody may be cloned into such a vector for the expression of the entire heavy or light chain. The expression vector may be transferred to a host cell by techniques known in the art.

In particular, techniques for the production of single chain antibodies may also be used in accordance with the methods disclosed herein. Single chain antibodies (e.g., scFvs or single chain variable fragments) comprise a heavy chain variable region and a light chain variable region connected with a linker peptide. In the scFv, the variable regions of the heavy chain and the light chain may be derived from the same antibody or different antibodies. ScFvs may be synthesized using recombinant techniques, for example by expression of a vector encoding the scFv in a host organism, such as, for instance, E. coli. DNA encoding scFv can be obtained by performing amplification using a partial DNA encoding the entire or a desired amino acid sequence of a DNA selected from a DNA encoding the heavy chain or the variable region of the heavy chain of the above-mentioned antibody, and a DNA encoding the light chain or the variable region of the light chain thereof as a template, by PCR using a primer pair that defines both ends thereof, and further performing amplification combining a DNA encoding a polypeptide linker portion and a primer pair that defines both ends thereof, so as to ligate both ends of the linker to the heavy chain and the light chain, respectively. An expression vector containing the DNA encoding scFv and a host transformed by the expression vector can be obtained according to conventional methods known in the art.

In one aspect, the present disclosure provides an antibody or fragment thereof produced by any of the methods disclosed herein. In some embodiments, the antibody or fragment thereof produced by any of the methods disclosed herein is an isolated anti-IBP4 antibody or fragment thereof as described herein. For example, the methods disclosed herein can be used to produce an isolated anti-IBP4 antibody or fragment thereof comprising (i) a heavy chain, wherein the heavy chain comprises: a HCDR1 comprising an amino acid sequence selected from the group consisting of SEQ ID NOS: 75, 78, 81, 84, 118, 244, 247, 250, 253, 256, 288, 291, 294, and 314; and a HCDR2 comprising an amino acid sequence selected from the group consisting of SEQ ID NOS: 76, 79, 82, 85, 119, 216, 218, 220, 222, 224, 245, 248, 251, 254, 257, 284, 286, 289, 292, 295, 312, 315, 317, 319, and 321; and a HCDR3 comprising an amino acid sequence selected from the group consisting of SEQ ID NOS: 77, 80, 83, 86, 217, 219, 221, 223, 225, 246, 249, 252, 255, 258, 285, 287, 290, 293, 296, 313, 316, 318, 320, 322; or (ii) a light chain, wherein the light chain comprises: a LCDR1 comprising an amino acid sequence selected from the group consisting of SEQ ID NOS: 23, 87, 90, 93, 96, 226, 228, 230, 233, 272, 274, 275, 323, 325, 327, and 330; and a LCDR2 comprising an amino acid sequence selected from the group consisting of SEQ ID NOS: 24, 91, 94, 97, 229, 231, 234, 260, 261, 263, 273, 297, 299, 300, 302, 326, 328, and 331; and a LCDR3 comprising an amino acid sequence selected from the group consisting of SEQ ID NOS: 89, 95, 98, 99, 100, 101, 227, 232, 235, 259, 262, 264, 298, 301, 303, 324, 329, and 332. In some embodiments, the present disclosure provides a nucleic acid molecule encoding the heavy chain of the anti-IBP4 antibody or the fragment thereof. In some embodiments, the nucleic acid molecule encoding the heavy chain has at least 85%, 90%, 95%, 96%, 97%, 98%, or 99% identity to at least one of SEQ ID NOS: 67, 102, 204,206, 208, 236, 265, 276, 304, 334, 336, 338, 340, or 342. In some embodiments, the present disclosure provides a nucleic acid molecule encoding the light chain of the anti-IBP4 antibody or fragment thereof. In some embodiments, the nucleic acid molecule encoding the light chain has at least 85%, 90%, 95%, 96%, 97%, 98%, or 99% identity to SEQ ID NO: 69, 104, 205, 207, 210, 238, 267, 278 or 306. In further embodiments, the present disclosure provides a nucleic acid molecule encoding the heavy chain and the light chain of the anti-IBP4 antibody or fragment thereof. In another aspect, the present disclosure provides a vector comprising the nucleic acid molecules encoding the heavy chain or the light chain of the anti-IBP4 antibody or the fragment thereof. In another aspect, the present disclosure provides an expression vector comprising the nucleic acid molecule encoding the heavy chain or the light chain of the anti-IBP4 antibody or the fragment thereof. In another aspect, the present disclosure provides a cell comprising any of the disclosed nucleic acid molecules, vectors, or expression vectors for the anti-IBP4 antibodies herein.

In some embodiments, the antibody or fragment thereof produced by any of the methods disclosed herein is an isolated SHBG antibody or a fragment thereof as described herein. For example, the methods disclosed herein can be used to produce an isolated anti-SHBG antibody or fragment thereof comprising: (i) a heavy chain, wherein the heavy chain comprises: a HCDR1 comprising an amino acid sequence selected from the group consisting of SEQ ID NOS: 11, 14, 17, 20, 52, and 115; and a HCDR2 comprising an amino acid sequence selected from the group consisting of SEQ ID NOS: 12, 15, 18, 21, 44, 47, 50, 53, 116, and 117; and a HCDR3 comprising an amino acid sequence selected from the group consisting of SEQ ID NOS: 13, 16, 19, 22, 45, 48, 51, and 54; or (ii) a light chain, wherein the light chain comprises: a LCDR1 comprising an amino acid sequence selected from the group consisting of SEQ ID NOS: 23, 26, 29, and 32; and a LCDR2 comprising an amino acid sequence selected from the group consisting of SEQ ID NOS: 24, 27, 30, and 33; and a LCDR3 comprising an amino acid sequence selected from the group consisting of SEQ ID NOS: 25, 31, and 34. In some embodiments, the present disclosure provides a nucleic acid molecule encoding the heavy chain of the anti-SHBG antibody or the fragment thereof. In some embodiments, the nucleic acid molecule encoding the heavy chain has at least 85%, 90%, 95%, 96%, 97%, 98%, or 99% identity to at least one of SEQ ID NOS: 3, 35, 200, or 202. In some embodiments, the present disclosure provides a nucleic acid molecule encoding the light chain of the anti-SHBG antibody or fragment thereof. In some embodiments, the nucleic acid molecule encoding the light chain has at least 85%, 90%, 95%, 96%, 97%, 98%, or 99% identity to SEQ ID NO: 5, 37, 201, or 203.

In further embodiments, the present disclosure provides a nucleic acid molecule encoding the heavy chain and the light chain of the anti-SHBG antibody or fragment thereof. In another aspect, the present disclosure provides a vector comprising the nucleic acid molecules encoding the heavy chain or the light chain of the anti-SHBG antibody or the fragment thereof. In another aspect, the present disclosure provides an expression vector comprising the nucleic acid molecule encoding the heavy chain or the light chain of the anti-SHBG antibody or the fragment thereof. In another aspect, the present disclosure provides a cell comprising any of the disclosed nucleic acid molecules, vectors, or expression vectors for the anti-SHBG antibodies herein.

In some embodiments, the present disclosure provides a vector or expression vector comprising a nucleic acid molecule encoding the heavy chain of the antibody or the fragment thereof of any of the anti-SHBG and anti-IBP4 antibodies disclosed herein. In some embodiments, the vector or expression vector comprises a nucleic acid molecule encoding the light chain of the antibody or the fragment thereof of any of the anti-SHBG and anti-IBP4 antibodies disclosed herein. In some embodiments, the present disclosure provides a cell comprising any of the nucleic acid molecules, vectors, or expression vectors disclosed herein.

In some embodiments, the anti-SHBG or anti-IBP4 antibody or fragment thereof can be a humanized antibody. Humanized antibodies are antibody molecules that bind the desired antigen and have one or more complementarity determining regions (CDRs) from a non-human species, and framework and constant regions from a human immunoglobulin molecule. Often, framework residues in the human framework regions will be substituted with the corresponding residue from the CDR donor antibody to alter, or preferably improve, antigen binding. As well recognized in the art, a variety of methods known in the art can be used to generate a humanized antibody having features of an anti-SHBG or anti-IBP4 antibody or fragment thereof described herein.

In some embodiments, the anti-SHBG or anti-IBP4 antibody is a human antibody. As well recognized in the art, human antibodies can be made by a variety of methods known in the art such as phage display methods using antibody libraries derived from human immunoglobulin sequences. Accordingly, a variety of methods known in the art can be used to generate a human antibody having features of an anti-SHBG or anti-IBP4 antibody or fragment thereof described herein.

### Compositions

In another aspect, this disclosure relates to a composition comprising an isolated antibody or antigen binding fragment thereof as described herein and a carrier (e.g., a pharmaceutically acceptable carrier). In some embodiments, the antibody or the fragment thereof comprises an anti-SHBG antibody or fragment thereof. In some embodiments, the anti-SHBG antibody or fragment thereof comprises a heavy chain and/or a light chain, wherein the heavy chain comprises an amino acid sequence selected from the group consisting of SEQ ID NOs:11-22, 44, 45, 47, 48, 50-54, 115, and 117 and wherein the light chain comprises an amino acid sequence selected from the group consisting of SEQ ID NOs: 23-34. In some embodiments, the heavy chain is associated with the light chain to form a binding site for SHBG. In another aspect, this disclosure relates to a composition comprising an isolated anti-IBP4 antibody or antigen binding fragment thereof as described herein and a carrier (e.g., pharmaceutically acceptable carrier). In some embodiments, the anti-IBP4 antibody or antigen-binding fragment thereof comprises heavy chain and/or a light chain, wherein the heavy chain comprises an amino acid sequence selected from the group consisting of SEQ ID NOs: 75-86, 118, 119, 216-225, 244-258, 284-296, and 312-322. and wherein the light chain comprises of an amino acid sequence selected from the group consisting of SEQ ID NOs: 23, 24, 87-101, 226-235, 259-264, 272-275, 297-303, and 323-332. In some embodiments, the heavy chain is associated with the light chain to form a binding site for IBP4.

In some embodiments, compositions of this disclosure comprise an anti-SHBG antibody or a fragment thereof that comprises a heavy chain, wherein the heavy chain comprises a heavy chain complementarity determining region 1 (HCDR1), HCDR2, and HCDR3 comprising an amino acid sequence selected from the group consisting of SEQ ID NOs: 11-22, 115 or 116, and a light chain, wherein the light chain comprises a light chain complementarity determining region 1 (LCDR1), LCDR2, and LCDR3 comprising an amino acid sequence selected from the group consisting of SEQ ID NOs:23-34. In some embodiments, the heavy chain is associated with the light chain to form a binding site for SHBG. In other embodiments, the anti-SHBG antibody or the fragment thereof comprises a heavy chain, wherein the heavy chain comprises a heavy chain complementarity determining region 1 (HCDR1), HCDR2, and HCDR3 comprising an amino acid sequence selected from the group consisting of SEQ ID NOs: 11, 14, 17, 44, 45, 47, 48, and 50-54, and/or a light chain, wherein the light chain comprises a light chain complementarity determining region 1 (LCDR1), LCDR2, and LCDR3 comprising an amino acid sequence selected from the group consisting of SEQ ID NOs: 23-34. In some embodiments, the heavy chain is associated with the light chain to form a binding site for SHBG.

In some embodiments, compositions of this disclosure include an anti-IBP4 antibody or a fragment thereof that comprises a heavy chain, wherein the heavy chain comprises a heavy chain complementarity determining region 1 (HCDR1), HCDR2, and HCDR3 comprising an amino acid sequence selected from the group consisting of SEQ ID NOs: 75-86, 118, 119, 216-225, 244-258, 284-296, and 312-322, and a light chain, wherein the light chain comprises a light chain complementarity determining region 1 (LCDR1), LCDR2, and LCDR3 comprising an amino acid sequence selected from the group consisting of SEQ ID NOs: 23, 87, 24, 89, 90, 91, 93, 94, 95-98, 226-235, 259-264, 272-275, 297-303, and 323-332. In some embodiments, the heavy chain is associated with the light chain to form a binding site for IBP4. In other embodiments, the anti-IBP4 antibody or the fragment thereof comprises a heavy chain, wherein the heavy chain comprises a heavy chain complementarity determining region 1 (HCDR1), HCDR2, and HCDR3 comprising an amino acid sequence selected from the group consisting of SEQ ID NOs: 75-86, 118, 119, 216-225, 244-258, 284-296, and 312-322, and/or a light chain, wherein the light chain comprises a light chain complementarity determining region 1 (LCDR1), LCDR2, and LCDR3 comprising an amino acid sequence selected from the group consisting of SEQ ID NOs: 87, 24, 90, 91, 93, 94, 96, 97, 99, 100, 101, 226-235, 259-264, 272-275, 297-303, and 323-332. In some embodiments, the heavy chain is associated with the light chain to form a binding site for IBP4.

In some embodiments, the composition can have at least 0.1% by weight the anti-SHBG antibody or antigen-binding fragment thereof. In some embodiments, the composition can have at least 0.5%, 1%, 2%, 3%, 4%, 5%, 6%, 7% 8%, 9%, 10%, 15%, 20%, 25%, 30%, 35%, 40%, 45%, 50%, 55%, 60%, 65%, 70%, 75%, 80%, 85%, 90%, or more by weight of the anti-SHBG antibody or antigen-binding fragment thereof. In some embodiments, the composition can have at least 0.1% by weight the anti-IBP4 antibody or antigen-binding fragment thereof. In some embodiments, the composition can have at least 0.5%, 1%, 2%, 3%, 4%, 5%, 6%, 7% 8%, 9%, 10%, 15%, 20%, 25%, 30%, 35%, 40%, 45%, 50%, 55%, 60%, 65%, 70%, 75%, 80%, 85%, 90%, or more by weight of the anti-IBP4 antibody or antigen-binding fragment thereof. In some embodiments, the composition can have at least 0.1% by weight the anti-SHBG antibody or antigen-binding fragment thereof, in combination with the anti-IBP4 antibody or antigen binding fragment thereof. In some embodiments, the composition can have at least 0.5%, 1%, 2%, 3%, 4%, 5%, 6%, 7% 8%, 9%, 10, 1%, 20%, 25%, 30%, 35%, 40%, 45%, 50%, 55%, 60%, 65%, 70%, 75%, 80%, 85%, 90%, or more by weight of the anti-SHBG antibody or antigen-binding fragment thereof and the anti-IBP4 antibody or the fragment thereof.

In some embodiments, the composition is designed for use in assay to detect SHBG and/or IBP4 from a sample. Suitable samples include, for example, blood, plasma, serum, amniotic fluid, vaginal secretions, saliva, and urine. In some embodiments, the biological sample is selected from the group consisting of whole blood, plasma, and serum. In a particular embodiment, the biological sample is serum. In some embodiments, the assay can be used for detecting SHBG and/or IBP4 in a sample from a pregnant female. As discussed herein, detection of SHBG and/or IBP4 can inform on a health status of the female, such as a probililty or preterm birth or preeclampsia.

In some embodiments, the composition is adapted for use in an immoprecipation assay, e.g., a co-immunoprecipitation assay. The assay can be used to detect the presence or absence of SHBG and/or anti-IBP4 antibodies in a sample from a pregnant female. In some embodiments, compositions of this disclosure comprise antibody capture agents that specifically bind to a SHBG or a IBP4 protein. The capture agents can comprise any one or more of the anti-SHBG and/or anti-IBP4 antibodies, or fragments thereof, described herein. Such capture agents can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986). The antibody capture agents can comprise any antibody or fragment thereof, as described herein, whether natural or wholly or partially synthetically produced. In some embodiments, the capture agent is linked to a solid support, such as a bead, e.g., a magnetic bead.

For example, in some embodiments, compositions of this disclosure comprise one or more anti-SHBG antibodies or fragments thereof, and/or one or more anti-IBP4 antibodies or fragments thereof, linked to a solid support which can be used to capture, isolate, and/or detect one or more SHBG proteins or fragments thereof and/or IBP4 proteins or fragments thereof from a sample. The solid support can comprise any substrate. Exemplary substrates include antibody-coated 96-well plates or nitrocellulose membranes that are subsequently probed for the presence of the SHBG and/or IBP4 proteins. Alternatively, substrates can include microspheres, microparticles, microbeads, beads, or other particles can be used for capture and detection of SHBG and/or IBP4. In some embodiments, compositions of this disclosure comprise one or more anti-IBP4 antibodies or fragments thereof linked to a sold support which can be used to capture and isolate one or more IBP4 proteins or fragments thereof from a sample.

For example, in some embodiments the substrate is a bead. The bead can be covalently linked with polypeptides that bind to SHBG and/or IBP4 proteins. The polypeptides can comprise a sequence as disclosed in the Sequence Listing below. The bead can be used in an assay to detect or measure the presence or absence of SHBG and/or IBP4 in a biological sample. For example, the bead can be deposited into the sample under conditions sufficient for the polypeptides to bind with SHBG and/or IBP4. After binding, the bead can be recovered from the sample with, for example, a magnet. Any SHBG and/or IBP4 present in the sample can be detected or measured by identifying SHBG and/or IBP4 bound to the recovered bead. SHBG and/or IBP4 can be identified by, for example, mass spectrometry.

Accordingly, compositions described herein can be useful for any existing, available, or conventional separation, detection and quantification methods that can be used to measure the presence or absence (e.g., a readout being present vs. absent; or detectable amount vs. undetectable amount) and/or quantity (e.g., readout being an absolute or relative quantity, such as, for example, absolute or relative concentration) of SHBG or IBP4 proteins, peptides, polypeptides, proteins and/or fragments thereof and optionally of the one or more other biomarkers or fragments thereof in samples.

In some embodiments, detection and/or quantification of SHBG or IBP4 comprises an assay comprises an enzyme immunoassay (EIA), enzyme-linked immunosorbent assay (ELISA), and radioimmunoassay (RIA). In some embodiments, detection and/or quantification of one or of SHBG and IBP4 further comprises mass spectrometry (MS). In yet further embodiments, the mass spectrometry is co-immunoprecitipation-mass spectrometry (co-IP MS), where coimmunoprecipitation, a technique suitable for the isolation of whole protein complexes is followed by mass spectrometric analysis.

In some embodiments, the anti-SHBG and anti-IBP4 antibodies described herein include a carrier. A carrier can be any type of material, including soluble and insoluble materials, as long as it is useful to carrier the anti-SHBG antibody and/or the anti-IBP4 antibody in an assay or during a therapeutic treatment. For example, examples include beads, such as magnetic beads, and particles. The material and the size of the carrier may be selected as appropriate according to the type of the assay or therapeutic use. In some embodiments, the carrier is a pharmaceutically acceptable carrier, which includes a liquid, semi-solid, i.e., pastes, or solid carriers. Examples of carriers or diluents include fats, oils, water, saline solutions, lipids, liposomes, resins, binders, fillers, and the like, or combinations thereof. The pharmaceutically acceptable carrier can include aqueous solvents (e.g., water, alcoholic/aqueous solutions, ethanol, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings (e.g., lecithin), surfactants, antioxidants, preservatives (e.g., antibacterial or antifungal agents, anti-oxidants, chelating agents, inert gases, parabens (e.g., methylparabens, propylparabens), chlorobutanol, phenol, sorbic acid, thimerosal), isotonic agents (e.g., sugars, sodium chloride), absorption delaying agents (e.g., aluminum monostearate, gelatin), salts, drugs, drug stabilizers (e.g., buffers, amino acids, such as glycine and lysine, carbohydrates, such as dextrose, mannose, galactose, fructose, lactose, sucrose, maltose, sorbitol, mannitol, etc), gels, binders, excipients, disintegration agents, lubricants, sweetening agents, flavoring agents, dyes, fluid and nutrient replenishers, such like materials and combinations thereof, as would be known to one of ordinary skill in the art.

Except insofar as any conventional media, agent, diluent, or carrier is detrimental to the recipient or to the therapeutic effectiveness of the composition contained therein, its use in administrable composition for use in practicing the methods is appropriate. The pH and exact concentration of the various components in a pharmaceutical composition are adjusted according to well-known parameters. In accordance with certain aspects of the present disclosure, the composition can be combined with the carrier in any convenient and practical manner, i.e., by solution, suspension, emulsification, admixture, encapsulation, absorption, grinding, and the like. Such procedures are routine for those skilled in the art.

In some embodiments, a pharmaceutically acceptable carrier can be an aqueous pH buffered solution. Examples include buffers such as phosphate, citrate, and other organic acids; antioxidants including ascorbic acid; low molecular weight (e.g., less than about 10 amino acid residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™

In some embodiments, pharmaceutically acceptable carriers can be sterile liquids, such as water and oils, including those of petroleum, animal, vegetable or synthetic origin, such as peanut oil, soybean oil, mineral oil, sesame oil and the like. Water can be a carrier, particularly when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, polysorbate-80 and the like. The composition can also contain minor amounts of wetting or emulsifying agents, or pH buffering agents. These compositions can take the form of solutions, suspensions, emulsion, tablets, pills, capsules, powders, sustained-release formulations and the like.

Certain embodiments of the present disclosure can have different types of carriers depending on whether it is to be administered in solid, liquid, or aerosol form, and whether it needs to be sterile for the route of administration, such as injection. The compositions can be formulated for administration intravenously, intradermally, transdermally, intrathecally, intraarterially, intraperitoneally, intranasally, intravaginally, intrarectally, intramuscularly, subcutaneously, mucosally, orally, topically, locally, by inhalation (e.g., aerosol inhalation), by injection, by infusion, by continuous infusion, by localized perfusion bathing target cells directly, via a catheter, via a lavage, in lipid compositions (e.g., liposomes), or by other methods or any combination of the forgoing as would be known to one of ordinary skill in the art (see, for example, Remington's Pharmaceutical Sciences, 18th Ed., 1990, incorporated herein by reference). Typically, such compositions can be prepared as either liquid solutions or suspensions; solid forms suitable for use to prepare solutions or suspensions upon the addition of a liquid prior to injection can also be prepared; and, the preparations can also be emulsified.

The anti-SHBG antibodies or other molecules having an antigen binding fragment that specifically binds to SHBG can be formulated into a composition in a free base, neutral, or salt form. Likewise, the anti-IBP4 antibodies or other molecules having an antigen binding fragment that specifically binds to IBP4 can be formulated into a composition in a free base, neutral, or salt form. Pharmaceutically acceptable salts include the acid addition salts, e.g., those formed with the free amino groups of a proteinaceous composition, or which are formed with inorganic acids, such as, for example, hydrochloric or phosphoric acids, or such organic acids as acetic, oxalic, tartaric, or mandelic acid. Salts formed with the free carboxyl groups can also be derived from inorganic bases, such as, for example, sodium, potassium, ammonium, calcium, or ferric hydroxides; or such organic bases as isopropylamine, trimethylamine, 2-ethylamino ethanol, histidine, or procaine.

In further embodiments, provided herein are pharmaceutical compositions having a lipid. A lipid can broadly include a class of substances that are characteristically insoluble in water and extractable with an organic solvent. Examples include compounds that contain long-chain aliphatic hydrocarbons and their derivatives. A lipid can be naturally occurring or synthetic (i.e., designed or produced by man). A lipid can be a biological substance. Biological lipids are well known in the art, and include for example, neutral fats, phospholipids, phosphoglycerides, steroids, terpenes, lysolipids, glycosphingolipids, glycolipids, sulphatides, lipids with ether- and ester-linked fatty acids, polymerizable lipids, and combinations thereof. Compounds other than those specifically described herein that are understood by one of skill in the art as lipids can also be used.

One of ordinary skill in the art would be familiar with the range of techniques that can be employed for dispersing a composition in a lipid vehicle. For example, antibodies can be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

As a person of ordinary skill in the art would understand, the compositions described herein are not limited by the particular nature of the therapeutic preparation. For example, such compositions can be provided in formulations together with physiologically tolerable liquid, gel, or solid carriers, diluents, and excipients. These therapeutic preparations can be administered to mammals for veterinary use, such as with domestic animals, and clinical use in humans in a manner similar to other therapeutic agents. In general, the dosage required for therapeutic efficacy varies according to the type of use and mode of administration, as well as the particularized requirements of individual subjects. The actual dosage amount of a composition administered to an animal patient, including a human patient, can be determined by physical and physiological factors, such as body weight, severity of condition, the type of disease being treated, previous or concurrent therapeutic interventions, idiopathy of the patient, and on the route of administration. Depending upon the dosage and the route of administration, the number of administrations of a preferred dosage and/or an effective amount can vary according to the response of the subject. The practitioner responsible for administration will, in any event, determine the concentration of active ingredient(s) in a composition and appropriate dose(s) for the individual subject.

### Kits

In another aspect, this disclosure relates to a kit. In some embodiments, the kit includes an isolated anti-SHBG antibody or a fragment thereof described herein, one or more ancillary reagents, a solid support and packaging for same. In some embodiments, the kit includes an isolated anti-IBP4 antibody or a fragment thereof, one or more ancillary reagents, a solid support and packaging for same. In some embodiments, the kit includes an isolated anti-SHBG antibody or a fragment thereof and an isolated anti-IBP4 antibody or a fragment thereof, one or more ancillary reagents, a solid support and packaging for same.

In some embodiments, the anti-SHBG antibody or the fragment thereof comprises a heavy chain, wherein the heavy chain comprises a heavy chain complementarity determining region 1 (HCDR1), HCDR2, and HCDR3 comprising an amino acid sequence selected from the group consisting of SEQ ID NOs: 11-22, 115 or 116, and a light chain, wherein the light chain comprises a light chain complementarity determining region 1 (LCDR1), LCDR2, and LCDR3 comprising an amino acid sequence selected from the group consisting of SEQ ID NOs:23-34. In some embodiments, the heavy chain is associated with the light chain to form a binding site for SHBG. In other embodiments, the anti-SHBG antibody or the fragment thereof comprises a heavy chain, wherein the heavy chain comprises a heavy chain complementarity determining region 1 (HCDR1), HCDR2, and HCDR3 comprising an amino acid sequence selected from the group consisting of SEQ ID NOs: 11, 14, 17, 44, 45, 47, 48, and 50-54, and/or a light chain, wherein the light chain comprises a light chain complementarity determining region 1 (LCDR1), LCDR2, and LCDR3 comprising an amino acid sequence selected from the group consisting of SEQ ID NOs:23-34. In some embodiments, the heavy chain is associated with the light chain to form a binding site for SHBG.

In some embodiments, the anti-IBP4 antibody or the fragment thereof comprises a heavy chain, wherein the heavy chain comprises a heavy chain complementarity determining region 1 (HCDR1), HCDR2, and HCDR3 comprising an amino acid sequence selected from the group consisting of SEQ ID NOs: 75-86, 118, 119, 216-225, 244-258, 284-296, and 312-322, and a light chain, wherein the light chain comprises a light chain complementarity determining region 1 (LCDR1), LCDR2, and LCDR3 comprising an amino acid sequence selected from the group consisting of SEQ ID NOs: 87, 24, 89, 90, 91, 93, 94, 95-98, 226-235, 259-264, 272-275, 297-303, and 323-332. In some embodiments, the heavy chain is associated with the light chain to form a binding site for IBP4. In other embodiments, the anti-IBP4 antibody or the fragment thereof comprises a heavy chain, wherein the heavy chain comprises a heavy chain complementarity determining region 1 (HCDR1), HCDR2, and HCDR3 comprising an amino acid sequence selected from the group consisting of SEQ ID NOs: 75-86, 118, 119, 216-225, 244-258, 284-296, and 312-322, and/or a light chain, wherein the light chain comprises a light chain complementarity determining region 1 (LCDR1), LCDR2, and LCDR3 comprising an amino acid sequence selected from the group consisting of SEQ ID NOs: 24, 87, 90, 91, 93, 94, 96, 97, 99, 100, 101, 226-235, 259-264, 272-275, 297-303, and 323-332. In some embodiments, the heavy chain is associated with the light chain to form a binding site for IBP4.

In some embodiments, the one or more ancillary reagents is selected from the group consisting of a secondary antibody, an incubation buffer, an immobilization buffer, a wash buffer, a blocking buffer, a detection buffer, a detection reagent, and a detection instrument, or any combination thereof. In some embodiments, the detection probe includes a label. In some embodiments, the label is selected from the group consisting of a fluorophore, enzyme, chemiluminescent moiety, radioactive moiety, organic dye and small molecule.

Secondary antibodies can include, for example, an anti-human IgA antibody, an anti-human IgD antibody, an anti-human IgE antibody, an anti-human IgG antibody, or an anti-human IgM antibody. In some embodiments, the secondary antibodies are anti-bovine antibodies. Secondary detection antibodies can be monoclonal or polyclonal antibodies. Secondary antibodies can be derived from any mammalian organism, including mice, rats, hamsters, goats, camels, chicken, rabbit, and others. Secondary antibodies can be conjugated to enzymes (e.g., horseradish peroxidase (HRP), alkaline phosphatase (AP), luciferase, and the like) or dyes (e.g., colorimetric dyes, fluorescent dyes, fluorescence resonance energy transfer (FRET)-dyes, time-resolved (TR)-FRET dyes, and the like). In some embodiments, the secondary antibody is a polyclonal rabbit-anti-human IgG antibody, which is HRP-conjugated.

In some embodiments, the detection buffer can include a fluorescent detection reagent or a luminescent detection reagent. In some other aspects, the luminescent detection reagent contains luminol or luciferin. Any detection buffer known in the art can be included in a kit of this disclosure. In some embodiments the detection buffer is a citrate-phosphate buffer (e.g., about pH 4.2).

In some embodiments, the detection reagent is a colorimetric detection reagent, a fluorescent detection reagent, or a chemiluminescent detection reagent. In some embodiments, the colorimetric detection reagent includes PNPP (p-nitrophenyl phosphate), ABTS (2,2′-azino-bis(3-ethylbenzothiazoline-6-sulphonic acid)) or OPD (0-phenylenediamine). In some embodiments, the fluorescent detection reagent includes QuantaBlu™ or QuantaRed™ (Thermo Scientific, Waltham, MA). In some embodiments, the luminescent detection reagent includes luminol or luciferin. In some embodiments, the detection reagent includes a trigger (e.g., H₂O₂) and a tracer (e.g., isoluminol-conjugate).

In some embodiments, the wash buffer can include those known in the art, such as tris(hydroxymethyl)aminomethane (Tris)-based buffers (e.g., Tris-buffered saline, TBS) or phosphate buffers (e.g., phosphate-buffered saline, PBS). Washing buffers can include detergents, such as ionic or non-ionic detergents. In some embodiments, the washing buffer is a PBS buffer (e.g., about pH 7.4) including Tween®20 (e.g., about 0.05% Tween®20).

In some embodiments, the solid support can include any support known in the art on which a protein of this disclosure can be immobilized. In some embodiments, the solid support is selected from the group consisting of microtiter well plates, slides (e.g., glass slides), chips (e.g., protein chips, biosensor chips, such as Biacore chips), microfluidic cartridges, cuvettes, beads (e.g., magnetic beads), resins, beads, spheres, particles, membranes, plates, and test tubes. In some embodiments, the bead, sphere or particle includes micrometer or nanometer dimensions. In some embodiments, the membrane is selected from the group consisting of nitrocellulose, nylon, polyvinylidene fluoride (PVDF) and polyvinylidene difluoride. In some embodiments, the anti-SHBG antibody or antigen-binding fragment is conjugated to the solid support.

In some embodiments, the packaging in the kit described herein can include a label indicating the kit is used for diagnosis, prognosis or monitoring of preterm birth or preeclampsia. In some embodiments, the packaging of the kit includes an FDA-approved label. FDA approved labels can include notification of an FDA-approved use and instructions. In some embodiments, the kit packaging can include a suitable container means, which is a container that does not react with components of the kit. In some embodiments, the container means includes one or more sealed vials, such as an Eppendorf tube, containing the anti-SHBG antibody or antigen-binding fragments described herein. The container can be made from sterilizable materials, such as plastic or glass.

The kits provided herein can be tailored to specific assay technologies. In some embodiments, the kit is an ELISA kit, Dot Blot kit, chemiluminescence immunoassay (CIA) kit or multiplex kit. In some embodiments, the ELSA kit can include a washing buffer, a sample diluents, a secondary antibody-enzyme conjugate, a detection reagent and a stop solution. In some embodiments, the Dot Blot kit includes a washing buffer, a sample diluents, a secondary antibody-enzyme conjugate, a detection reagent, and a stop solution. In some embodiments, the CIA kit includes a washing buffer, a sample diluent, a tracer (e.g., isoluminol-conjugate) and a trigger (e.g., H₂O₂). In some embodiments, the multiplex kit includes a washing buffer, a sample diluents and a secondary antibody-enzyme conjugate

In some embodiments, the kit includes a positive control. In some embodiments, the positive control is a sample containing SHBG. In some embodiments, the positive control is a sample containing IBP4. In some other embodiments, the kits provided herein include instructions for using the subunits of the kit for detecting SHBG or glycosylated SHBG or IBP4 in the sample from the subject.

Provided herein are also kits that can be used as a companion diagnostic for probability of preterm birth and preeclampsia. In some embodiments, the kits can be used to select patients for a particular intervention therapy designed to reduce the probability of preterm birth and preeclampsia. The intervention therapies include those known in the art including but not limited to the administration of progesterone.

### Methods of Use

According to one aspect, this disclosure provides a method for detecting a presence or absence of one or more biomarkers from a sample. In some embodiments, the one or more biomarkers comprise SHBG. In some embodiments, the one or more biomarkers comprise IBP4. In some embodiments, the one or more biomarkers comprise SHBG and IBP4. For example, in some embodiments, the disclosure provides a method that includes contacting a biological sample comprising one or more biomarkers to an anti-SHBG antibody, or fragment thereof, and/or an anti-IBP4 antibody, or fragment thereof, as described herein. The method further includes detecting binding of the antibody or the fragment thereof to the one or more biomarkers. Detecting the binding can involve, for example, a proteomics workflow that comprises mass spectrometry quantification. In some embodiments, the biomarker that is detected is a SHBG protein. In some embodiments, the biomarker that is detected is a glycosylated SHBG protein. In some embodiments, the biomarker that is detected is an unglycosylated SHBG protein. In some embodiments, the biomarker that is detected is a IBP4 protein. In some embodiments, the biological sample is blood or serum. In some embodiments, detecting comprises separating the one or more biomarkers bound to the antibody or the antibody fragment thereof from the biological sample. In some embodiments, detecting comprises an immunoassay selected from the group consisting of sandwich immunoassay, immunohistochemistry, enzyme-linked immunosorbent assay (ELISA), Western blotting, nephelometry, turbidimetry, immunoradiometric assay, lateral flow, and immunehisto/cyto-chemistry.

However, a person skilled in the art will appreciate that a number of methods can be used to detect or determine the presence or absence of one or more biomarkers, including mass spectrometry approaches, such as MS/MS, LC-MS/MS, multiple reaction monitoring (MMR) or SRM and product-ion monitoring (PIM) and also including antibody based methods such as immunoassays such as Western blots, enzyme-linked immunosorbant assay (ELISA), immunoprecipitation, immunohistochemistry, immunofluorescence, radioimmunoassay, dot blotting, and FACS. Accordingly, in some embodiments, determining the level of the at least one biomarker comprises using an immunoassay and/or mass spectrometric methods. In additional embodiments, the mass spectrometric methods are selected from MS, MS/MS, LC-MS/MS, SRM, PIM, and other such methods that are known in the art. In other embodiments, LC-MS/MS further comprises 1D LC-MS/MS, 2D LC-MS/MS or 3D LC-MS/MS. Immunoassay techniques and protocols are generally known to those skilled in the art (Price and Newman, Principles and Practice of Immunoassay, 2nd Edition, Grove's Dictionaries, 1997; and Gosling, Immunoassays: A Practical Approach, Oxford University Press, 2000). A variety of immunoassay techniques, including competitive and non-competitive immunoassays, can be used (Self et al., Curr. Opin. Biotechnol., 7:60-65 (1996).

In one aspect, this disclosure relates to a method of determining probability of preterm birth in a pregnant female, the method comprising measuring in a biological sample from said pregnant female one or more biomarkers. In some embodiments, the one or more biomarkers comprise SHBG. In some embodiments, the one or more biomarkers comprise IBP4. In some embodiments, the one or more biomarkers comprise SHBG and IBP4. According to this disclosure the method can be used to determine the probability of preterm birth.

In some embodiments, the method involves an immunoassay in which the one or more biomarkers are labeled and/or detected by the binding an anti-SHBG or anti-IBP4 antibody described herein. In some embodiments, a detection anti-SHBG and/or anti-IBP4 antibody can be labeled by different types of labels able to generate different types of signals that could be visualized or detected using a variety of standard procedures, such as detection of luminescence, chemiluminescence, fluorescence, absorbance, radioactivity, or by microscopy, imaging, etc. Immunoassays may include immunohistochemistry, enzyme-linked immunosorbent assay (ELISA), Western blotting, nephelometry, turbidimetry, immunoradiometric assay, lateral flow, immunohisto/cyto-chemistry and other methods known to those of skill in the art.

In some embodiments, the method can be used to determine the presence or absence of a biomarker in a sample as well as the amount of a biomarker in a sample. The presence or absence or amount of biomarker in the sample can be determined by comparison to (or as a ratio to) a reference or standard, such as a sample taken from a healthy pregnant female. The amount of biomarker in the sample can also be determined by comparison to a reference or standard, such as the amount of the endogenous or recombinant or synthetic biomarker fragments in a reference or control sample. Accordingly, the amount of a biomarker in a sample need not be quantified in absolute terms, but may be measured in relative terms with respect to a reference or control.

In another aspect, this disclosure relates to a method of determining probability of preeclampsia in a pregnant female, the method comprising measuring in a biological sample from said pregnant female one or more biomarkers. In some embodiments, the one or more biomarkers comprise SHBG. In some embodiments, the one or more biomarkers comprise IBP4. In some embodiments, the one or more biomarkers comprise SHBG and IBP4. According to this disclosure, the method can be used, to determine the probability of preeclampsia.

In another aspect, this disclosure relates to a method of detecting one or more biomarkers in a biological sample. The method can include obtaining a biological sample, such as a biological sample (e.g., serum or urine) from a pregnant female. The method for detecting whether the one or more biomarkers are present in the biological sample includes contacting the biological sample comprising one or more biomarkers with an antibody or a fragment thereof as disclosed herein and detecting binding of the antibody or the fragment thereof to one or more biomarkers. Binding can be detected using a label. Binding can also be detected by mass spectrometry. In some embodiments, the one or more biomarkers comprise SHBG. In some embodiments, the one or more biomarkers comprise IBP4. In some embodiments, the one or more biomarkers comprise SHBG and IBP4.

In another aspect, this disclosure provides a method for assessing or predicting a health status of a subject, the method comprising: (i) obtaining a biological sample comprising one or more biomarkers from a subject; (ii) contacting an anti-SHBG or anti-IBP4 antibody or the fragment thereof as described herein to the biological sample under conditions suitable for the antibody or the fragment thereof to bind to the one or more biomarkers; and (iii) detecting the binding of the antibody or the fragment thereof to the one or more biomarker, wherein detecting the binding of the antibody to the one or more biomarkers indicates the presence of the one or more biomarkers in the biological sample and not detecting the binding of the antibody to the one or more biomarkers indicates the absence of the one or more biomarkers in the biological sample, and where the presence or absence of the one or more biomarker in the biological sample is used to assess or predict the health status of the subject. In some embodiments, the method further includes quantitating an amount of the one or more biomarkers bound to the antibody or the fragment thereof in the biological sample, wherein the amount of the one or more biomarker is used to assess or predict the health status of the subject. In some embodiments, methods include detecting SHBG. In some embodiments, methods include detecting IBP4. In some embodiments, methods include detecting SHBG and IBP4. In some embodiments, the health status comprises any one of abnormal glucola, gestational diabetes, hypertension, preeclampsia, intrauterine growth restriction, stillbirth, fetal growth restriction, HELLP syndrome, oligohyramnios, chorioamnionitis, chorioamnionitis, placental previa, placental acreta, abruption, abruptio placenta, placental hemorrhage, preterm premature rupture of membranes, preterm labor, unfavorable cervix, postterm pregnancy, cholelithiasis, or uterine over distention.

Accordingly, in some embodiments, methods of this disclosure make use of an anti-SHBG antibody or a fragment thereof that comprises a heavy chain, wherein the heavy chain comprises a heavy chain complementarity determining region 1 (HCDR1), HCDR2, and HCDR3 comprising an amino acid sequence selected from the group consisting of SEQ ID NOs: 11-22, 115 or 116, and a light chain, wherein the light chain comprises a light chain complementarity determining region 1 (LCDR1), LCDR2, and LCDR3 comprising an amino acid sequence selected from the group consisting of SEQ ID NOs:23-34. In some embodiments, the heavy chain is associated with the light chain to form a binding site for SHBG. In other embodiments, the anti-SHBG antibody or the fragment thereof comprises a heavy chain, wherein the heavy chain comprises a heavy chain complementarity determining region 1 (HCDR1), HCDR2, and HCDR3 comprising an amino acid sequence selected from the group consisting of SEQ ID NOs: 11, 14, 17, 44, 45, 47, 48, and 50-54, and/or a light chain, wherein the light chain comprises a light chain complementarity determining region 1 (LCDR1), LCDR2, and LCDR3 comprising an amino acid sequence selected from the group consisting of SEQ ID NOs:23-34. In some embodiments, the heavy chain is associated with the light chain to form a binding site for SHBG. In some embodiments, methods of this disclosure make use of an anti-IBP4 antibody or fragment thereof that comprises a heavy chain complementarity determining region 1 (HCDR1), HCDR2, and HCDR3 comprising an amino acid sequence selected from the group consisting of SEQ ID NOs: 75-86, 118, 119, 216-225, 244-258, 284-296, and 312-322, and a light chain, wherein the light chain comprises a light chain complementarity determining region 1 (LCDR1), LCDR2, and LCDR3 comprising an amino acid sequence selected from the group consisting of SEQ ID NOs: 87, 24, 89, 90, 91, 93, 94, 95-98, 226-235, 259-264, 272-275, 297-303, and 323-332. In some embodiments, the heavy chain is associated with the light chain to form a binding site for IBP4. In other embodiments, the anti-IBP4 antibody or the fragment thereof comprises a heavy chain, wherein the heavy chain comprises a heavy chain complementarity determining region 1 (HCDR1), HCDR2, and HCDR3 comprising an amino acid sequence selected from the group consisting of SEQ ID NOs: 75-86, 118, 119, 216-225, 244-258, 284-296, and 312-322, and/or a light chain, wherein the light chain comprises a light chain complementarity determining region 1 (LCDR1), LCDR2, and LCDR3 comprising an amino acid sequence selected from the group consisting of SEQ ID NOs: 87, 24, 90, 91, 93, 94, 96, 97, 99, 100, 101, 226-235, 259-264, 272-275, 297-303, and 323-332. In some embodiments, the heavy chain is associated with the light chain to form a binding site for IBP4.

In further embodiments, although described and exemplified with reference to methods of determining probability for preterm birth or preeclampsia in a pregnant female, the present disclosure is similarly applicable to methods of predicting an abnormal glucose test, gestational diabetes, hypertension, intrauterine growth restriction, stillbirth, fetal growth restriction, HELLP syndrome, oligohyramnios, chorioamnionitis, chorioamnionitis, placental previa, placental acreta, abruption, abruptio placenta, placental hemorrhage, preterm premature rupture of membranes, preterm labor, unfavorable cervix, postterm pregnancy, cholelithiasis, uterine over distention, stress.

In some embodiments, a suitable biological sample includes, for example, whole blood, plasma, serum, amniotic fluid, vaginal secretions, saliva, and urine. In a particular embodiment, the biological sample is serum.

In some embodiments, the biological sample is obtained between between 19 and 21 weeks of gestational age. In some embodiments, the pregnant female is between 17 and 28 weeks of gestation at the time the biological sample is collected, also referred to as GABD (Gestational Age at Blood Draw). In other embodiments, the pregnant female is between 16 and 29 weeks, between 17 and 28 weeks, between 18 and 27 weeks, between 19 and 26 weeks, between 20 and 25 weeks, between 21 and 24 weeks, or between 22 and 23 weeks of gestation at the time the biological sample is collected. In further embodiments, the pregnant female is between about 17 and 22 weeks, between about 16 and 22 weeks between about 22 and 25 weeks, between about 13 and 25 weeks, between about 26 and 28, or between about 26 and 29 weeks of gestation at the time the biological sample is collected. Accordingly, the gestational age of a pregnant female at the time the biological sample is collected can be 3, 4, 5, 6, 7, 8, 9, 10, 11, 12, 13, 14, 15, 16, 17, 18, 19, 20, 21, 22, 23, 24, 25, 26, 27, 28, 29 or 30 weeks. In particular embodiments, the biological sample is collected between 19 and 21 weeks of gestational age. In particular embodiments, the biological sample is collected between 19 and 22 weeks of gestational age. In particular embodiments, the biological sample is collected between 19 and 21 weeks of gestational age. In particular embodiments, the biological sample is collected between 19 and 22 weeks of gestational age. In particular embodiments, the biological sample is collected at 18 weeks of gestational age. In further embodiments, the highest performing reversals for consecutive or overlapping time windows can be combined in a single classifier to predict the probability of sPTB over a wider window of gestational age at blood draw.

In some embodiments, the disclosed methods for determining the probability of preterm birth or preeclampsia encompass detecting and/or quantifying SHBG using a proteomics work-flow comprised of mass spectrometry, a capture agent or a combination thereof. In yet further embodiments, the mass spectrometry can be liquid chromatography-mass spectrometry (LC-MS), multiple reaction monitoring (MRM), selected reaction monitoring (SRM), or co-immunoprecipitation-mass spectrometry (co-IP MS), where coimmunoprecipitation, a technique suitable for the isolation of whole protein complexes is followed by mass spectrometric analysis. In additional embodiments, the MRM or SRM can further encompass scheduled MRM or scheduled SRM.

For mass-spectrometry based analysis, differential tagging with isotopic reagents, e.g., isotope-coded affinity tags (ICAT) or the more recent variation that uses isobaric tagging reagents, iTRAQ (Applied Biosystems, Foster City, Calif), or tandem mass tags, TMT, (Thermo Scientific, Rockford, IL), followed by multidimensional liquid chromatography (LC) and tandem mass spectrometry (MS/MS) analysis can provide a further methodology in practicing the methods of this disclosure.

In some embodiments, the capture agent includes an antibody or a fragment thereof. In some embodiments, the capture agent comprises an anti-SHBG antibody or fragment thereof. In some embodiments, the capture agent comprises an anti-IBP4 antibody or fragment thereof. In some embodiments, the capture agent includes an anti-SHBG antibody or fragment thereof and an anti-IBP4 antibody or fragment thereof.

In additional embodiments, the disclosed methods for determining the probability of preterm birth or preeclampsia includes encompass detecting and/or quantifying SHBG using an assay including enzyme immunoassay (EIA), enzyme-linked immunosorbent assay (ELISA), and radioimmunoassay (RIA). In additional embodiments, the assay is an enzyme immunoassay (EIA), enzyme-linked immunosorbent assay (ELISA), and radioimmunoassay (RIA).

In further embodiments, the immunoassay is selected from Western blot, ELISA, immunoprecipitation, immunohistochemistry, immunofluorescence, radioimmunoassay (RIA), dot blotting, and FACS. In certain embodiments, the immunoassay is an ELISA. In yet a further embodiment, the ELISA is direct ELISA (enzyme-linked immunosorbent assay), indirect ELISA, sandwich ELISA, competitive ELISA, multiplex ELISA, ELISPOT technologies, and other similar techniques known in the art. Principles of these immunoassay methods are known in the art, for example John R. Crowther, The ELISA Guidebook, 1st ed., Humana Press 2000, ISBN 0896037282. Typically ELISAs are performed with antibodies but they can be performed with any capture agents that bind specifically to one or more biomarkers of this disclosure and that can be detected. Multiplex ELISA allows simultaneous detection of two or more analytes within a single compartment (e.g., microplate well) usually at a plurality of array addresses (Nielsen and Geierstanger 2004. J Immunol Methods 290: 107-20 (2004) and Ling et al. 2007. Expert Rev Mol Diagn 7: 87-98 (2007)).

In some embodiments, Radioimmunoassay (RIA) can be used to detect one or more biomarkers in the methods of this disclosure. RIA is a competition-based assay that is well known in the art and involves mixing known quantities of radioactively-labelled (e.g., ¹⁵²I or ¹³¹I-labelled) target analyte with antibody specific for the analyte, then adding non-labeled analyte from a sample and measuring the amount of labeled analyte that is displaced (see, e.g., An Introduction to Radioimmunoassay and Related Techniques, by Chard T, ed., Elsevier Science 1995, ISBN 0444821198 for guidance).

In further embodiments, a chemiluminescence assay using a chemiluminescent antibody can be used for sensitive, non-radioactive detection of protein levels. An antibody labeled with fluorochrome also can be suitable. Examples of fluorochromes include, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), beta-galactosidase, urease, and the like. Detection systems using suitable substrates for horseradish-peroxidase, alkaline phosphatase, and beta-galactosidase are well known in the art.

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif) in accordance with the manufacturer's instructions. If desired, assays used to practice aspects of this disclosure can be automated or performed robotically, and the signal from multiple samples can be detected simultaneously.

A detectable label can be used in the assays described herein for direct or indirect detection of the biomarkers in the methods of this disclosure. A wide variety of detectable labels can be used, with the choice of label depending on the sensitivity required, ease of conjugation with the antibody, stability requirements, and available instrumentation and disposal provisions. Those skilled in the art are familiar with selection of a suitable detectable label based on the assay detection of the biomarkers in the methods of this disclosure. Suitable detectable labels include, but are not limited to, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), enzymes (e.g., luciferase, horseradish peroxidase, alkaline phosphatase, etc.), nanoparticles, biotin, digoxigenin, metals, and the like.

As described above, chromatography can also be used in practicing the methods of this disclosure. Chromatography encompasses methods for separating chemical substances and generally involves a process in which a mixture of analytes is carried by a moving stream of liquid or gas (“mobile phase”) and separated into components as a result of differential distribution of the analytes as they flow around or over a stationary liquid or solid phase (“stationary phase”), between the mobile phase and said stationary phase. The stationary phase can be usually a finely divided solid, a sheet of filter material, or a thin film of a liquid on the surface of a solid, or the like. Chromatography is well understood by those skilled in the art as a technique applicable for the separation of chemical compounds of biological origin, such as, e.g., amino acids, proteins, fragments of proteins or peptides, etc.

Chromatography can be columnar (i.e., wherein the stationary phase is deposited or packed in a column), preferably liquid chromatography, and yet more preferably high-performance liquid chromatography (HPLC), or ultra high performance/pressure liquid chromatography (UHPLC). Particulars of chromatography are well known in the art (Bidlingmeyer, Practical HPLC Methodology and Applications, John Wiley & Sons Inc., 1993). Exemplary types of chromatography include, without limitation, high-performance liquid chromatography (HPLC), UHPLC, normal phase HPLC (NP-HPLC), reversed phase HPLC (RP-HPLC), ion exchange chromatography (IEC), such as cation or anion exchange chromatography, hydrophilic interaction chromatography (HILIC), hydrophobic interaction chromatography (HIC), size exclusion chromatography (SEC) including gel filtration chromatography or gel permeation chromatography, chromatofocusing, affinity chromatography such as immuno-affinity, immobilized metal affinity chromatography, and the like. Chromatography, including single-, two- or more-dimensional chromatography, can be used as a peptide fractionation method in conjunction with a further peptide analysis method, such as for example, with a downstream mass spectrometry analysis as described elsewhere in this specification.

Further peptide or polypeptide separation, identification or quantification methods can be used, optionally in conjunction with any of the above described analysis methods, for measuring biomarkers in the present disclosure. Such methods include, without limitation, chemical extraction partitioning, isoelectric focusing (IEF) including capillary isoelectric focusing (CIEF), capillary isotachophoresis (CITP), capillary electrochromatography (CEC), and the like, one-dimensional polyacrylamide gel electrophoresis (PAGE), two-dimensional polyacrylamide gel electrophoresis (2D-PAGE), capillary gel electrophoresis (CGE), capillary zone electrophoresis (CZE), micellar electrokinetic chromatography (MEKC), free flow electrophoresis (FFE), etc.

In additional embodiments, the methods of determining probability for preterm birth or preeclampsia in a pregnant female further encompasses detecting a measurable feature for one or more risk indicia associated with preterm birth or preeclampsia. The one or more risk indicia includes previous low birth weight or preterm delivery, multiple 2nd trimester spontaneous abortions, prior first trimester induced abortion, familial and intergenerational factors, history of infertility, nulliparity, gravidity, primigravida, multigravida, placental abnormalities, cervical and uterine anomalies (i.e. short cervical length measurements), gestational bleeding, intrauterine growth restriction, in utero diethylstilbestrol exposure, multiple gestations, infant sex, short stature, low prepregnancy weight/low or high body mass index, diabetes, hypertension, urogenital infections (i.e. urinary tract infection), maternal characteristics, education, age, race, ethnicity, medical history, past pregnancy history, obstetrical history, hypothyroidism, asthma, low educational attainment, cigarette smoking, drug use and alcohol consumption, and anxiety and depression. Demographic risk indicia for preterm birth or preeclampsia can include, for example, maternal age, race/ethnicity, single marital status, low socioeconomic status, maternal education, employment-related physical activity, occupational exposures and environment exposures and stress. Further risk indicia can include, inadequate prenatal care, cigarette smoking, use of marijuana and other illicit drugs, cocaine use, alcohol consumption, caffeine intake, maternal weight gain, dietary intake, sexual activity during late pregnancy and leisure-time physical activities. (Preterm Birth: Causes, Consequences, and Prevention, Institute of Medicine (US) Committee on Understanding Premature Birth and Assuring Healthy Outcomes; Behrman R E, Butler A S, editors. Washington (DC): National Academies Press (US); 2007). Additional risk indicia can be identified using learning algorithms known in the art, such as linear discriminant analysis, support vector machine classification, recursive feature elimination, prediction analysis of microarray, logistic regression, CART, FlexTree, LART, random forest, MART, and/or survival analysis regression, which are known to those of skill in the art and are further described herein

In another aspect, this disclosure provides a method for treating a subject having a SHBG-associated disease or condition. The SHBG-associated disease or condition can comprise abnormal glucola, gestational diabetes, hypertension, preeclampsia, intrauterine growth restriction, stillbirth, fetal growth restriction, HELLP syndrome, oligohyramnios, chorioamnionitis, chorioamnionitis, placental previa, placental acreta, abruption, abruptio placenta, placental hemorrhage, preterm premature rupture of membranes, preterm labor, unfavorable cervix, postterm pregnancy, cholelithiasis, placental dysfunction, premature cervical shortening, microbial dysbiosis, or uterine over distention. In some embodiments, the method includes administering an effective amount of an anti-SHBG antibody or fragment thereof as disclosed herein. The anti-SHBG antibody or fragment thereof can be administered intravenously or orally in combination with a pharmaceutically acceptable carrier.

In another aspect, this disclosure provides a method for treating a subject having an IBP4-associated disease or condition. The IBP4-associated disease or condition can comprise abnormal glucola, gestational diabetes, hypertension, preeclampsia, intrauterine growth restriction, stillbirth, fetal growth restriction, HELLP syndrome, oligohyramnios, chorioamnionitis, chorioamnionitis, placental previa, placental acreta, abruption, abruptio placenta, placental hemorrhage, preterm premature rupture of membranes, preterm labor, unfavorable cervix, postterm pregnancy, cholelithiasis, placental dysfunction, premature cervical shortening, microbial dysbiosis, or uterine over distention. In some embodiments, the method includes administering an effective amount of an anti-IBP4 antibody or fragment thereof as disclosed herein. The anti-IBP4 antibody or fragment thereof can be administered intravenously or orally in combination with a pharmaceutically acceptable carrier.

The composition, kit or method comprising any of the disclosed anti-SHBG or anti-IBP4 antibodies or fragments thereof may be administered before, during, or after the onset of a disease or an injury. The present disclosure also provides treatment of ongoing disease, where the treatment stabilizes or reduces the undesirable clinical symptoms of the patient or subject. In some embodiments, the patient or subject in need of a treatment has a disease, a condition, and/or an injury that can be contained, ameliorated, and/or improved with treatment of any of the disclosed compositions, methods, kits, and antibodies herein.

As used herein, the administration of any of the compositions, kits, or antibodies herein includes any route of introducing or delivering to a patient or subject the composition, kit, antibodies, etc. to perform its intended function. Suitable dosage formulations and methods of administering such agents are known in the art. The method of determining the most effective route of administration are known to those of skill in the art and will vary with the composition, kit, or antibody used for treatment, the purpose of the treatment, the health condition or disease stage of the patient or subject being treated. Non-limiting examples of route of administration include oral administration, nasal administration, injection, topical application, and by suppository. Administration includes self-administration and administration by another. It also also appreciated that the various modes of treatment or prevention of medical conditions as described herein includes total, but also less than total, treatment or prevention, and wherein some biologically or medically relevant result is achieved.

Administration can be effected in one dose, continuously or intermittently throughout the course of treatment. Methods of determining the most effective means and dosage of administration are known to those of skill in the art and will vary with the composition, kit, and/or antibody used for therapy, the purpose of the therapy, the target cell or antigen being treated, and the subject being treated. Single or multiple administrations can be carried out with the dose level and pattern being selected by the treating physician, practitioner, or healthcare professional.

It is understood that modifications which do not substantially affect the activity of the various embodiments of this disclosure are also ecompassed by what is described herein. Accordingly, the following is a description of various methods and materials used in the studies, and are put forth so as to provide those of ordinary skill in the art with a complete disclosure and description of how to make and use the present disclosure, and are intended to illustrate and not intended to limit the scope of what the inventors regard as their disclosure nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present disclosure. Efforts have been made to ensure accuracy with respect to numbers used (e.g., amounts, percentages, etc.), but some experimental errors and deviations should be accounted for.

### EXAMPLES

### Example 1: Production of Anti-SHBG and Anti-IBP4 Antibodies

Immunization: three Balb/c mice between 8-12 weeks old were immunized with 50-100 μg of SHBG or IBP4 immunogen (peptide or protein) via intraperitoneal injection using Freund's adjuvant. The amino acid sequence for the SHBG immunogen is referenced as SEQ ID NO: 110. The amino acid sequence for the IBP4 immunogen is referenced as SEQ ID NO: 111.

At about 49 days post immunization, tail bleeds from the mice immunized with SHBG and tail bleeds from the mice immunized with IBP4 were screened by an enzyme-linked immunosorbent assay (ELISA) with peptides conjugated to EMCS-activated bovine serum albumin (BSA) or protein. Screening material was bound to standard ELISA plates, washed, and blocked with BSA. The tail bleeds were serially diluted 3-fold (starting at 1:500) to generate a logarithmic curve, and H1RP-conjugated goat anti-mouse IgG was used as the secondary antibody.

Fusion of spleen cells: a mouse was identified and selected as positive for an anti-SHBG antibody and for an anti-IBP4 antibody. Both mice were euthanized. The spleens were excised from the mice. The spleen cells were disassociated and fused with a NS1 (murine myeloma) cell line via standard PEG protocols and plated into 16 plates.

Screening for reactive clones: all plates were screened by ELISA with peptides conjugated to EMCS-activated BSA or protein. Up to 94 positive wells from each cell line were selected for expansion and subsequent re-confirmation assays. Up to 20 positive wells were selected from the re-confirmation assays for each cell line. A portion of the cells were frozen down. Supernatants containing anti-SHBG or anti-IBP4 antibodies were used for additional testing.

Sub-cloning: positive parental cell lines were chosen for further expansion and were screened again by ELISA. The two best daughter clones from each cell line were further expanded and isotyped, with a final deliverable of 10 mL supernatant from each cell line being sent to a third party for further evaluation.

Production: supernatants from selected clones were scaled up with roller-bottle production. The resultant material was purified by Protein G (Cytiva Life Sciences #P-00067) chromatography. no doubt aware, our antibodies were developed using a standard mouse inoculation to hybridoma development process.

After selecting the antibodies and their associated hybridomas, light and heavy chain genes were sequenced for integration into expression vectors used by a commercial research and development organization. The variable regions from the sequenced hybridomas were integrated into a standard backbone consistent with the isotype of the original antibody. The backbone had been codon optimized to work well in a proprietary Chinese Hamster Ovary (CHO) cell line via transient expression. The antibody backbone was based on a particular strain of mice which is slightly different from the mice that were initially inoculated and used to produce the hybridomas. As a result, a sequence difference appeared in the light chain of the anti-IBP4 antibody clone 5B3.G9, giving rise to variant 5B3.G9. In particular, the light chain expressed in the CHO cell platform, a lysine was observed in place of an arginine (compare SEQ ID NO: 68 to SEQ ID NO: 122).

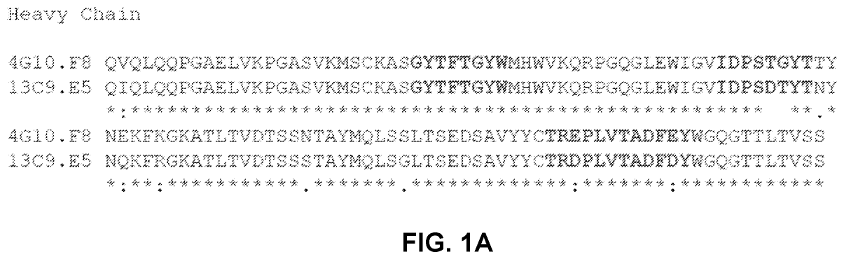

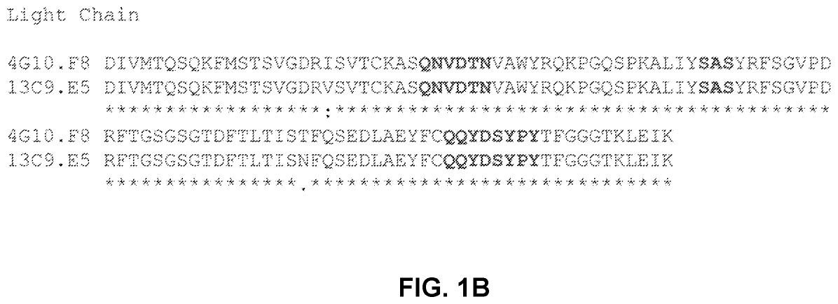

FIGS. 1A-1B show sequence alignments of heavy chain variable regions and light chain variable regions of anti-SHBG antibodies designated as 4G10.F8 and 13C9.E5. Boundaries of exemplary complementarity-determining regions (CDRs) are in bold. FIG. 1A shows the sequence alignment of heavy chain variable regions of anti-SHBG antibodies 4G10.F8 (SEQ ID NO: 8) and 13C9.E5 (SEQ ID NO: 40). FIG. 1B shows the sequence alignments of light chain variable regions of anti-SHBG antibodies 4G10.F8 (SEQ ID NO: 10) and 13C9.E5 (SEQ ID NO: 42).

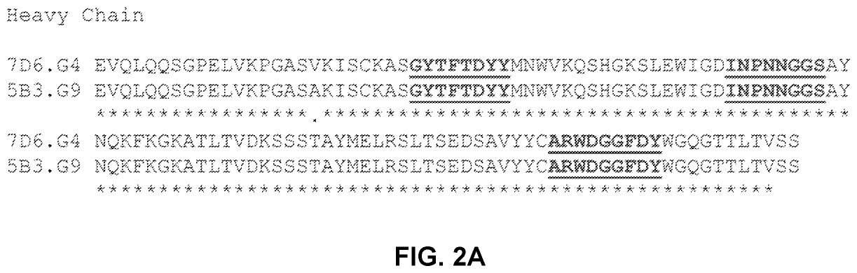

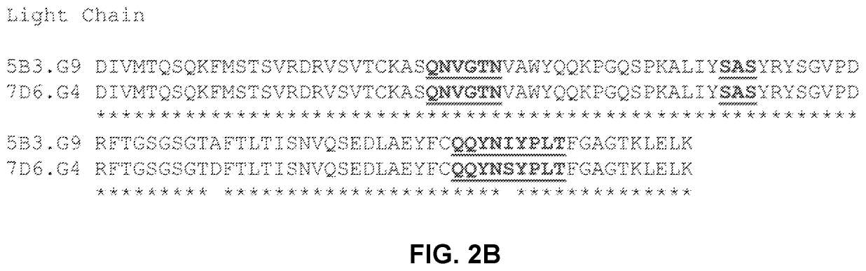

FIGS. 2A-2B show sequence alignments of heavy chain variable regions and light chain variable regions of anti-IBP4 antibodies designated as 5B3.G9 and 7D6.G4. Boundaries of exemplary CDRs are in bold. FIG. 2A shows the sequence alignment of heavy chain variable regions of anti-IBP4 antibodies 7D6.G4 (SEQ ID NO: 107) and 5B3.G9 (SEQ ID NO: 72). FIG. 2B shows the sequence alignment of the light chain variable regions of anti-IBP4 antibodies 5B3.G9 (SEQ ID NO: 74) and 7D6.G4 (SEQ ID NO: 109).

Additional production of anti-IBP4 antibodies: Additional exemplary antibodies that bind to the same or similar epitope of anti-IBP4 antibodies 5B3.G9 and 7D6.G4 (i.e., additional anti-IBP4 antibodies) were identified through further rounds of antibody production. These additional antibodies include anti-IBP4 antibody clone designations 1H9.B6, 5C11.A7, 7G11.H4, 3D4.E9, 1G3.A3, 8G4.G11, 5F10.B2, and 3D8.A11. These additional anti-IBP4 antibodies were created using the method as described above, except for the selection of the immunogen. The immunogen used was either a recombinant version of the full length IBP4 protein (“FL IBP4”) or a synthetic peptide, as shown in Table 1 below. These additional anti-IBP4 antibodies were then screened against full length IBP4 protein as well as the C-terminal IBP4 fragment (“C-term IBP4”) (amino acids 157-258) using the same identification/isolation methods and assays described above for the anti-IBP4 antibody 5B3.G9. A summary of the additional anti-IBP4 antibodies and their respective immunogens is shown in Table 1 below.

TABLE 1
Summary of Immunogens for Additional
Production of Anti-IBP4 Antibodies

| M-PVA- | |
|---|---|
| antibody | Immunogen |
| IBP4-1H9.B6 | FL IBP4 |
| IBP4-5C11.A7 | FL IBP4 |
| IBP4-7G11.H4 | FL IBP4 |
| *IBP4-5B3.G9 | C-term IBP4 |
| IBP4-3D4.E9 | FL IBP4 |
| IBP4-1G3.A3 | FL IBP4 |
| IBP4-8G4.G11 | FL IBP4 |
| IBP4-5F10.B2 | [Cyc(1,22)]Aoa- |
| | CVDRKTGVKLPGGLEPKGELDCHQ- |
| | amide |
| | (SEQ ID NO: 343) |
| IBP4-3D8.A11 | [Cyc(1,22)]Aoa- |
| | CVDRKTGVKLPGGLEPKGELDCHQ- |
| | amide |
| | (SEQ ID NO: 343) |

*anti-IBP4 antibody 5B3.G9 served as a reference/comparison for production of the additional anti-IBP4 antibodies.
“FL” immunogen indicates “full length IBP4 immunogen”
“C-term IBP4” immunogen indicates a C-terminus fragment of IBP4 comprising amino acids 157-258.
[Cyc(1,22)] indicates cyclic peptide formed by disulfide bonds between the 1^{st} and 22^{nd} amino acids of the peptide (both Cys)

### Example 2: Validation and Characterization of SHBG Antibodies

Antibodies were generated from hybridoma supernatants as described in Example 1. The antibodies were then screened for SHBG binding by western blot analyses and surface plasmon resonance assays. Western blotting was used to assess two properties of the antibodies: specificity—the ability of the antibody to recognize and bind to its target SHBG antigen; and selectivity—the preference of the antibody to bind its target SHBG antigen in the presence of a heterogeneous mixture of competing sample proteins. For western blotting, tail bleed anti-sera, hybridoma supernatants were screened by immunoblotting for reactivity toward purified human SHBG protein in the presence of competing proteins after denaturing SDS PAGE and electro-transfer to an immunoblotting membrane.

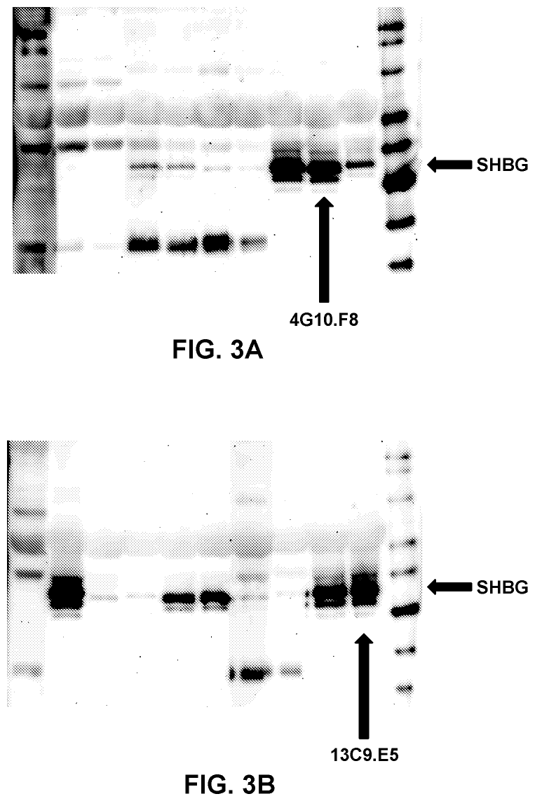

FIGS. 3A and 3B show image data from the western blots confirming anti-SHBG antibody clones variant 4G10.F8 and variant 13C9.E5 bind human SHBG protein. The images demonstrate that the anti-SHBG antibody variant 4G10.F8 and anti-SHBG antibody variant 13C9.E5 (FIGS. 3A and 3B, respectively) produce a strong signal against human serum containing SHB4 protein. Signals corresponding to the anti-SHBG antibodies are indicated by the black arrows. The signals are indicative of binding of the anti-SHBG antibodies with SHBG protein.

To assess binding affinities of anti-SHBG antibodies to target epitopes, Biacore-based assays were performed. The Biocore assay provides a measure of biomolecular interactions such as protein-protein interactions or small molecule/fragment-protein interactions.

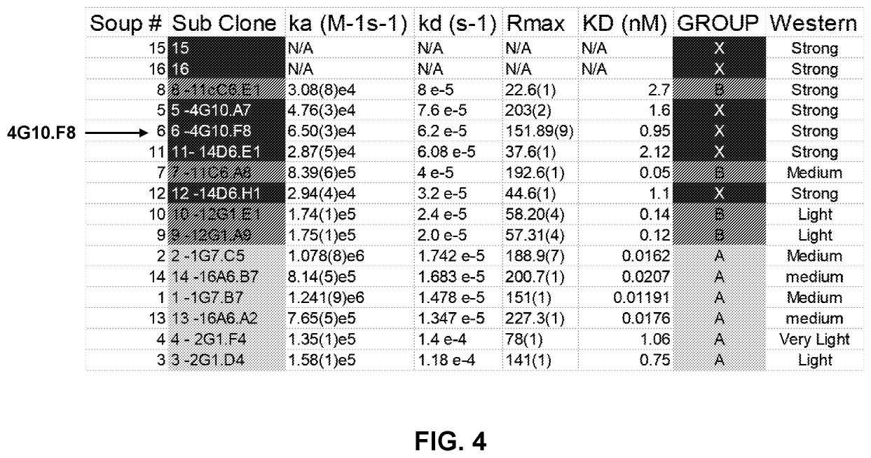

FIG. 4 shows Biocore data of exemplary anti-SHBG antibody clones. One exemplary clone, variant 4G10.F8, is identified by the black arrow. These data indicate a strong binding affinity of the variant 4G10.F8 clone toward SHBG protein.

To determine target epitopes of the anti-SHBG antibodies, electrochemiluminescence immunoassay (ECLIA) assays were performed to assess competitive binding properties of certain peptides. In particular, a first competition assay was performed with peptide pools having known epitopes to approximate the epitope of clones variant 4G10.F8 or variant 13C9.E5. A second competition assay was performed with single ones of the peptides from the pools to map the target epitope of the SHBG clones variant 4G10.F8 and variant 13C9.E5 based on peptides with known epitopes.

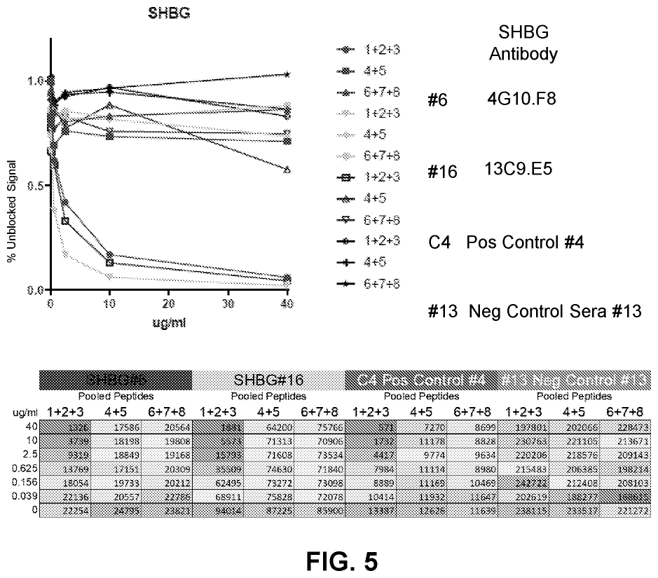

FIG. 5 shows data from a pooled peptide competition assay. The competition assay was performed to evaluate competitive binding of certain peptide pools (i.e., 1+2+3; 4+5; or 6+7+8) having known target epitopes with anti-SHBG antibody clones variant 4G10.F8 and variant 13C9.E5. The top panel provides a line graph showing the pool of peptides 1+2+3 competes with variant 4G10.F8 and variant 13C9.E5 for binding with a target epitope, which is evidenced by a reduction in unblocked signal (y axis) from data corresponding to the competition assays of 1+2+3 peptides with variant 4G10.F8 and variant 13C9.E5 (see lines with a solid upside-down triangle and a solid circle, respectively). The lower panel provides a heatmap of the data presented in the upper panel. As illustrated, peptides 1+2+3 competes with variant 4G10.F8 and variant 13C9.E5 for binding with a target epitope, suggesting peptides 1+2+3 share a similar target epitope as antibody clones variant 4G10.F8 and variant 13C9.E5.

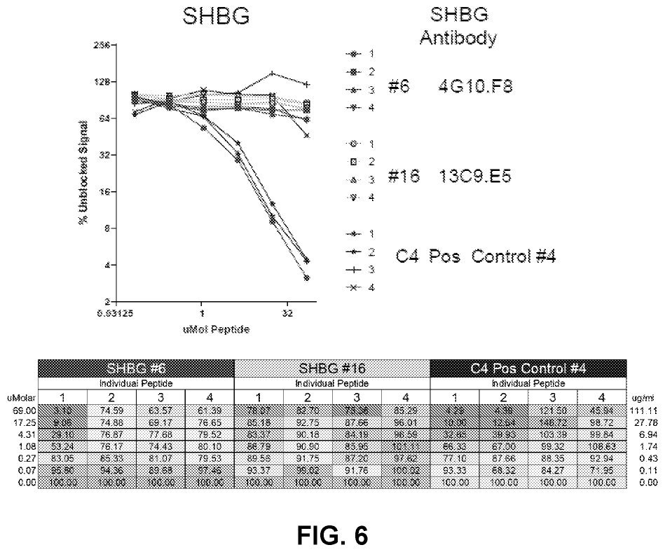

FIG. 6 shows data from single peptide competition assays. The single peptides are from the peptide pool 1+2+3 described above. These data suggest variant 4G10.F8 (SHBG #6) and variant 13C9.E5 (SHBG #16) are at least partially blocked by binding of peptide 1. These data further suggest variant 13C9.E5 is at least partially blocked by peptides 1 and 3, whereas variant 4G10.F8 is primarily blocked by peptide 1. Accordingly, these data suggest variant 4G10.F8 and variant 13C9.E5 at least partially bind to the same epitope of SHBG as peptide 1.

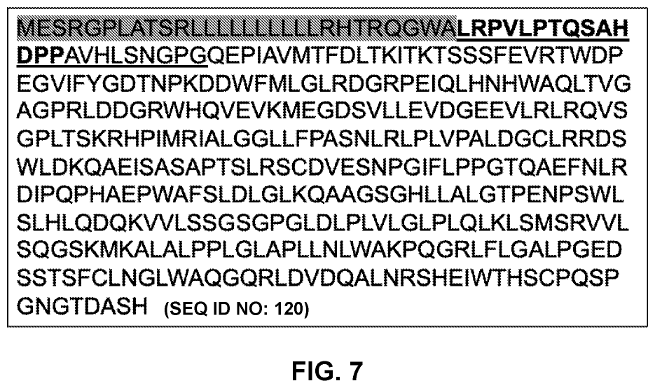

FIG. 7 illustrates an SHBG target epitope sequence (SEQ ID NO: 120) mapped against blocking peptides 1+2+3. The shaded amino acid represents a signal sequence, which is not present in mature SHBG protein. Peptides of the pool 1+2+3, of the competition assay described above, map against the underlined portion of the sequence. In particular, peptide 1 maps against the bolded section of SEQ ID NO: 120. Thus, variant 4G10.F8 and variant 13C9.E5 at least partially bind the bolded section of SEQ ID NO: 120.

Protein glycosylation is a common posttranslational modification (PTM) found on many proteins. Protein glycosylation involves the covalent attachment of a glycan (e.g., an O-linked glycan) to a protein and can have a significant influence on the activity of the protein, for example, by altering its structure. Glycosylation can also impact the ability of an antibody to bind with a target epitope. To assess the impact of glycosylation on anti-SHBG antibody binding, ECLIA assays were performed with SHBG proteins treated, or not treated, with de-glycosylation enzymes.

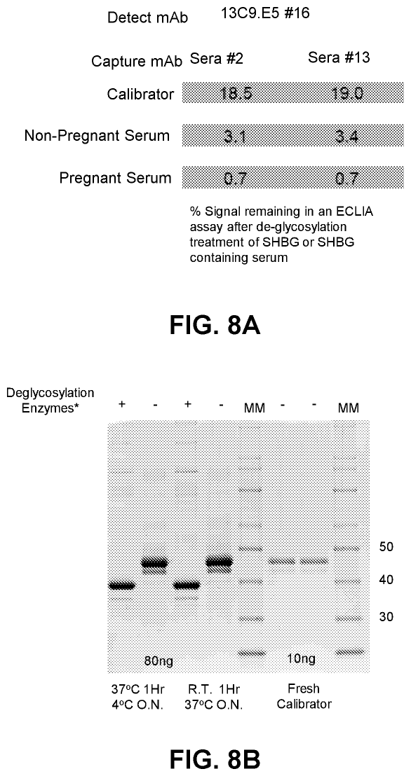

FIGS. 8A and 8B shows data from ECLIA assays characterizing the impact of SHBG glycosylation on antibody binding. These data suggest that the anti-SHBG antibody preferentially binds with glycosylated SHBG protein. In particular, FIG. 8A shows the percent signal remaining in an ECLIA assay in which variant 13C9.E5 antibody is used to bind SHBG serum from pregnant and non-pregnant females. The signal from the non-pregnant female, i.e., percent signal remaining, is substantially reduced in the serum from the pregnant female. As illustrated, in FIG. 8B, samples treated with a de-glycosylation enzyme (wells labeled with “+”) produce a lower signal than cells not treated with de-glycosylation enzyme (e.g., compare wells 1 and 2), suggesting that the antibody preferentially binds with SHBG protein that is glycosylated.

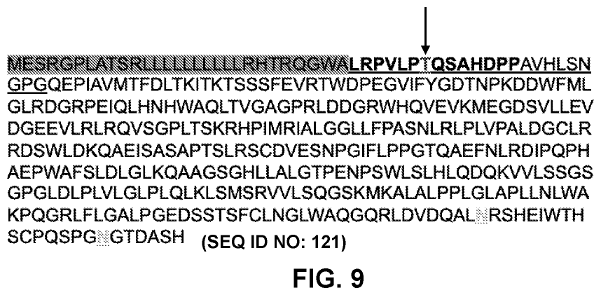

FIG. 9 illustrates a glycosylation site of the SHBG epitope. In particular, FIG. 9 shows SHBG includes an O-linked glycosylation site at T36 is in the epitope mapped region of variant 4G10.F8, and variant 13C9.E5. The epitope mapped region is underlined and in bold. The T36 site having the O-linked glycosylation is identified by the black arrow. As evidenced by the data above, the presence of the O-linked glycosylation appears to enhance the ability of anti-SHBG antibodies to bind with the target epitope.

### Example 3: Validation and Characterization of Anti-IBP4 Antibodies

Anti-IBP4 antibodies were generated as described above in Example 1, including the additional anti-IBP4 antibodies 1H9.B6, 5C11.A7, 7G11.H4, 3D4.E9 and 1G3.A3. The anti-IBP4 antibodies were initially screened by ELISA testing. Two anti-IBP4 antibody clones (variant 5B3.G9 and variant 7D6.G4) were identified as being of particular interest based on the ELISA testing and chosen for further characterization as discussed below. The antibodies were screened for IBP4 binding by western blotting. Briefly, tail bleed anti-sera, hybridoma supernatants were screened by immunoblotting for reactivity toward purified human IBP4 protein in the presence of competing proteins after denaturing SDS PAGE and electro-transfer to an immunoblotting membrane.

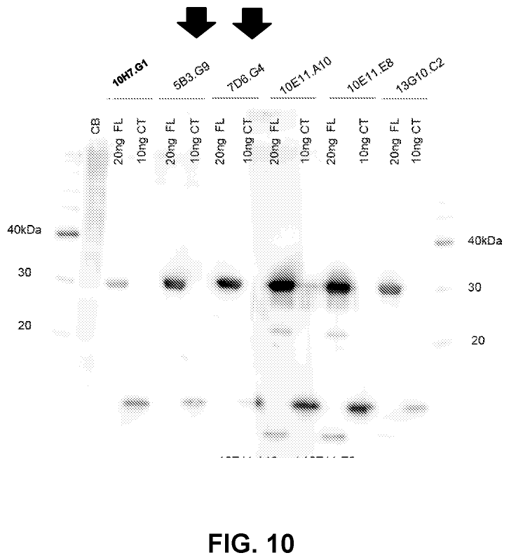

FIG. 10 shows an image from a western blot analysis confirming anti-IBP4 antibody variant clones 5B3.G9 and 7D6.G4 bind human IBP4 protein. The images demonstrate that anti-IBP4 variant antibodies 5B3.G9 and 7D6.G4 produce a strong signal against human serum containing IBP4 protein. Signals corresponding to the anti-IBP4 variant antibodies 5B3.G9 and 7D6.G4 are indicated by the black arrows. The signals are indicative of binding of the anti-IBP4 antibodies with IBP4 protein.

To determine epitopes of the anti-IBP4 antibodies, competitive binding assays were performed. In particular, three strategies were used to identify the epitopes of each of the antibody. First, competitive assays were performed to infer the epitope based on the peptide sequence used as an immunogen. The competitive assays were performed to group antibodies into bins based on whether or not they competed with for binding. This allowed for quick grouping of antibodies to possible shared epitopes. The third approach was to map epitopes using a peptide versus antibody competition strategy as discussed above, i.e., by initially using pools of peptides and then separating out individual peptides from a competing pool that block the binding of an antibody to IBP4.

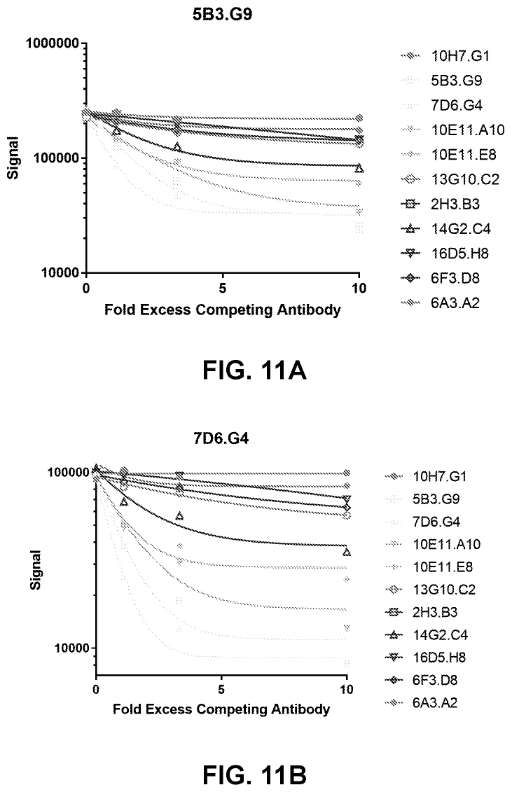

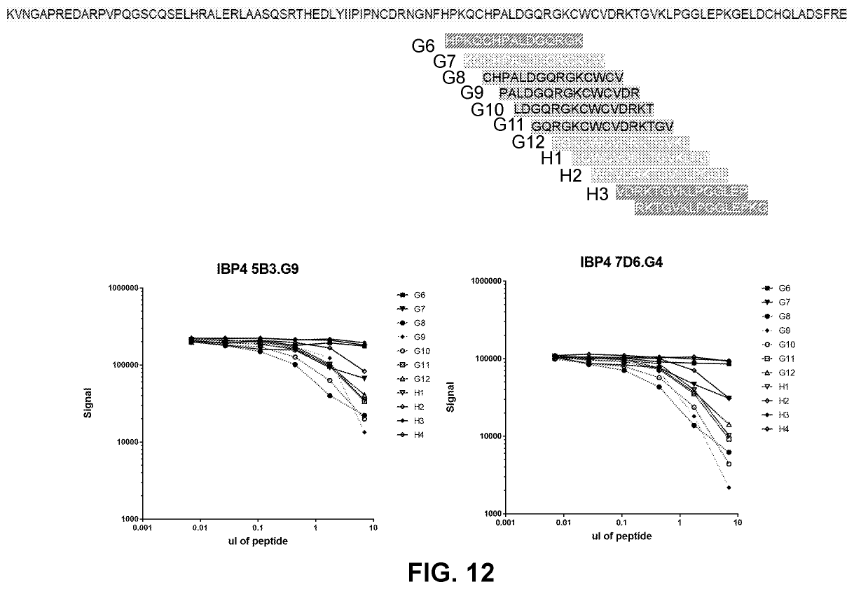

FIGS. 11A and 11B shows data from direct ECLIA competitive binding assays performed using anti-IBP4 antibody clones variant 5B3.G9 and variant 7D6.G4. FIG. 11A illustrates that the signal of clone variant 5B3.G9 decreases with increased concentration of variant 7D6.G4. FIG. 11B illustrates that the signal of variant 7D6.G4 decreases with increased concentration of antibody clone variant 5B3.G9. Together, these data suggest that variant 5B3.G9 and variant 7D6.G4 at least partially share the same epitope.

FIG. 12 shows data from ECLIA competitive binding assays performed to map the epitope of variant 5B3.G9 and variant 7D6.G4 antibody clones (SEQ ID NOs. 344-353, 369). These data suggest variant 5B3.G9 and variant 7D6.G4 at least partially compete for binding with peptides G8, G9, and G10. Further binding assays were performed to determine that that these clones bind with IBP4 epitope having sequence CHPALDGQRGKCWCVDRKTGVK (SEQ ID NO: 131).

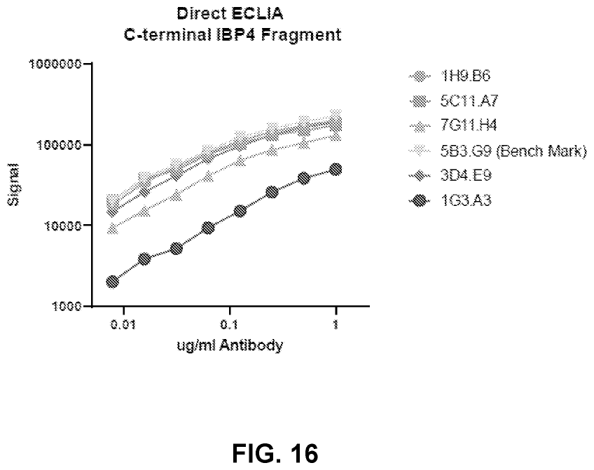

FIG. 16 shows data from a direct ECLIA binding assay generated from the additional anti-IBP4 antibodies (1H9.B6, 5C11.A7, 7G11.H4, 3D4.E9, and 1G3.A3) being compared with 5B3.G9, demonstrating their similar affinity to a C-terminal IBP4 fragment like that of 5B3.G9. 1G3.A3 was a notable outlier from the other antibodies, showing a notiablely lower binding affinity.

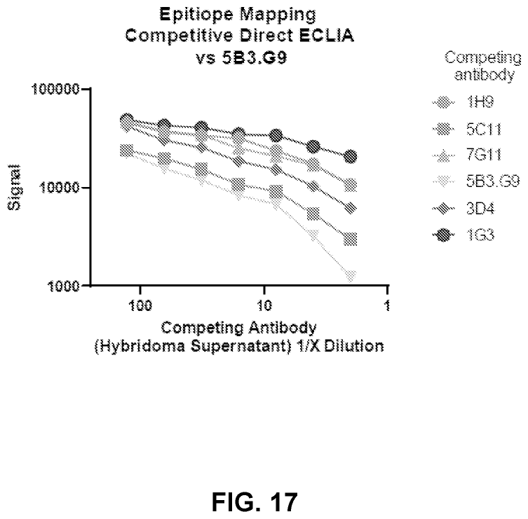

FIG. 17 shows data from ECLIA direct competitive binding assays of the antibodies 1H9.B6, 5C11.A7, 7G11.H4, 3D4.E9, and 1G3.A3, demonstrating each antibody's ability to block 5B3.G9 from binding to its respective epitope, therefore suggesting they bind to the same or similar epitopes. Again, 1G3.A3 was a notable outlier from the other antibodies, showing the lowest about of competitive inhibition of 5B3.G9 binding.

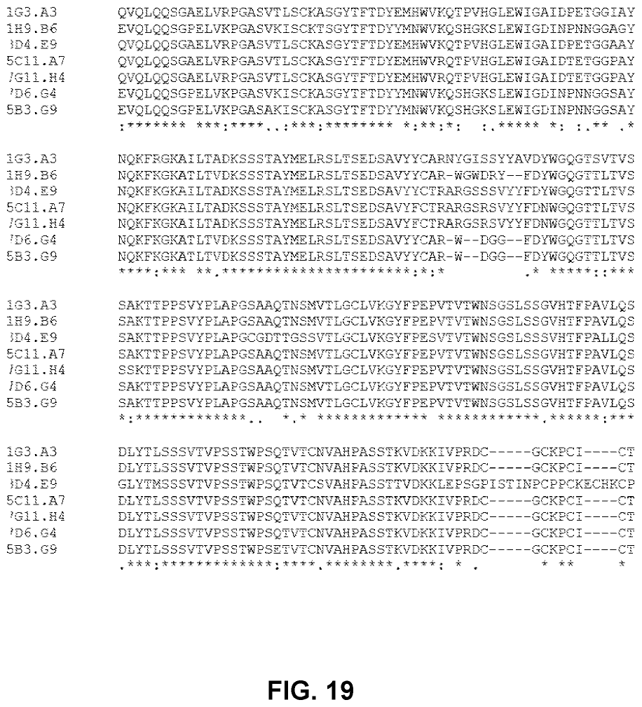

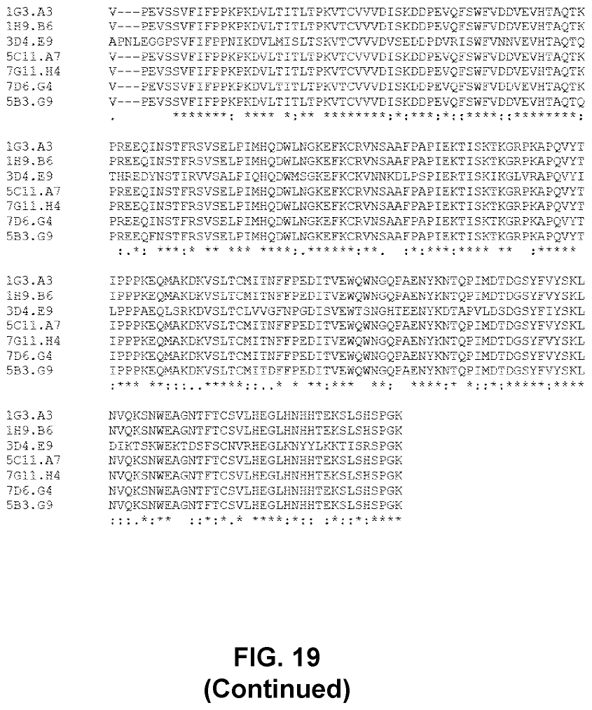

FIG. 19 shows the heavy chain CLUSTAL 0 (1.2.4) full-length multiple sequence alignment for the 1G3.A3 (SEQ ID NO: 305), 1H9.B6 (SEQ ID NO: 209), 3D4.E9 (SEQ ID NO: 277), 5C11.A7 (SEQ ID NO: 237), 7G11.H4 (SEQ ID NO: 266), 7D6.G4 (SEQ ID NO: 103), and 5B3.G9 (SEQ ID NO: 68) anti-IBP4 antibodies.

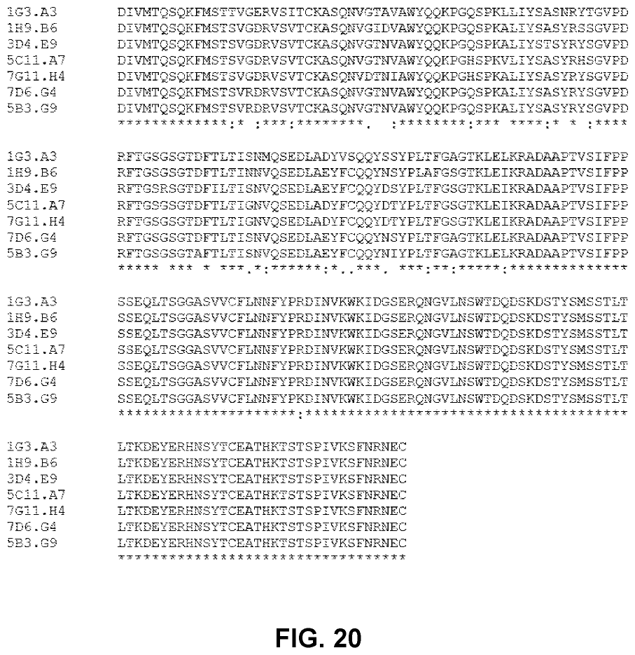

FIG. 20 shows the light chain CLUSTAL 0 (1.2.4) full-length multiple sequence alignment for the 1G3.A3 (SEQ ID NO: 307), 1H9.B6 (SEQ ID NO: 70), 3D4.E9 (SEQ ID NO: 279), 5C11.A7 (SEQ ID NO: 239), 7G11.H4 (SEQ ID NO: 268), 7D6.G4 (SEQ ID NO: 105), and 5B3.G9 (SEQ ID NO: 70) anti-IBP4 antibodies.

Summary. The above assays indicate that antibodies 5B3.G9 and 7D6.G4 demonstrated binding to IBP4. The additional 1H9.B6, 5C11.A7, 7G11.H4, 3D4.E9, and 1G3.A3 antibodies were also identified as binding to IBP4 at the same or similar epitope as 5B3.G9 by competitive inhibition.

### Example 4. Assay Optimization and Development

Selection of PerkinElmer M-PVA epoxy magnetic beads: three commercially available bead types were explored for affinity capture. These included Thermo Ultralink polyacrylamide beads, Thermo Dynabeads and PerkinElmer M-PVA beads. PerkinElmer M-PVA epoxy magnetic beads were selected for development because of reasonable cost, efficient and consistent coupling to antibodies, excellent capture and elution of target proteins from serum and low non-specific background.

Coupling of antibody to M-PVA epoxy beads: the protocol for coupling antibody to the M-PVA epoxy beads as provided by the manufacturer (0.1M Na PO₄ pH 8 at 37° C.) was found to result in <20% incorporation of antibody under the recommended conditions of 1 mg/ml of antibody per 10 mg of beads. Further optimization determined that a coupling reaction containing 600 μg/ml antibody per 10 mg of beads in 0.1M sodium borate pH 9.0, 1M (NH₄)₂SO₄ improved coupling efficiency of antibody to the M-PVA beads to 80-95% incorporation after 4 hrs at 37° C.

Affinity capture buffer and bead wash buffers: Serum samples were diluted in TBS buffer (50 mM Tris pH 7.5, 150 mM NaCl) containing either 0.05% of the mass spectrometry compatible detergent NG-310 (Anatrace) or 0.1% Tween-20. To evaluate capture and wash buffers a mix of M-PVA beads coupled to control anti-IBP4 and anti-SHBG antibodies was used to affinity capture IBP4 and SHBG proteins from pooled pregnant serum (QC2) in TBS buffer containing detergents identified in Table 2. Beads were washed once or twice in 50 mM Tris pH 7.5, 0.5M NaCl (THS) then eluted and digested with trypsin and analyzed by an MRM-MS assay as described (Saade, et al). The use of Tween-20 required multiple washing steps to remove traces of this detergent prior to performing mass spectrometry (MS) analysis. A single wash step with THS buffer was found to be sufficient to remove the non-bound serum proteins and excess detergent when using 0.05% NG-310 and give equivalent results.

TABLE 2
AC-MS capture and wash buffer test.

| AC-MS μl QC2 serum: | | | | | |
|---|---|---|---|---|---|
| 4 reps each | | IBP4-QCHPALDGQR | | SHBG-IALGGLLFPASNLR | |
| | # of THS | peptide (SEQ ID NO: 370) | | peptide (SEQ ID NO: 371) | |
| AC buffer | washes | Ave RR* | % CV | Ave RR* | % CV |
| TBS 0.1% Tween-20 | 2 | 0.545 | 1.3 | 2.839 | 1.7 |
| TBS 0.05% NG-310 | 2 | 0.554 | 1.1 | 2.856 | 4.1 |
| TBS 0.05% NG-310 | 1 | 0.539 | 2.2 | 3.025 | 4.0 |

*Response Ratio (RR): peptide peak area divided by control SIS peptide peak area.

Evaluation of elution buffers. Initially target elution from the washed capture beads was performed using 0.5M glycine pH 2.25, 2M urea. However, several anti-IBP4 and anti-SHBG antibodies coupled to M-PVA beads lost binding activity after each round of elution, which limited reuse of the coupled beads. Evaluation of alternative elution buffers determined that 0.25M glycine pH 2.7 allowed for complete elution of the target proteins yet did not compromise reuse of the capture beads for at least 3 rounds of capture and elution. See Table 3.

To optimize the elution conditions, a mix of M-PVA beads coupled to IBP4-5B3.G9 and SHBG-13C9.E5 variant antibodies was used to capture IBP4 and SHBG from serum samples (4 replicates each), washed once with THS buffer and eluted with two sequential aliquots of the buffers identified in Table 3. The combined eluted aliquots were adjusted to pH 8.0, processed for trypsin digestion and analyzed by MRM-MS.

TABLE 3
AC-MS elution buffer performance comparison.

| AC-MS: IBP4-5B3.G9/SHBG-13C9.E5 | IBP4 results | | SHBG Results | |
|---|---|---|---|---|
| Serum/elution buffer | ave RR | CV | ave RR | CV |
| GW 11-1365/ 0.5M glycine pH 2.25, | 0.276 | 5.5 | 0.948 | 6.1 |
| 2M urea | | | | |
| GW 11-1365/ 0.25M glycine pH 2.7 | 0.271 | 1.5 | 1.015 | 11.4 |
| GW 11-1366/ 0.5M glycine pH 2.25, | 0.365 | 7.8 | 1.968 | 8.7 |
| 2M urea | | | | |
| GW 11-1366/ 0.25M glycine pH 2.7 | 0.325 | 3.2 | 2.056 | 7.7 |
| GW 11-1436/ 0.5M glycine pH 2.25, | 0.493 | 6.1 | 1.103 | 7.7 |
| 2M urea | | | | |
| GW 11-1436/ 0.25M glycine pH 2.7 | 0.506 | 4.1 | 1.298 | 4.7 |
| GW 15-9689/ 0.5M glycine pH 2.25, | 0.401 | 2.2 | 1.070 | 4.7 |
| 2M urea | | | | |
| GW 15-9689/ 0.25M glycine pH 2.7 | 0.415 | 3.9 | 1.288 | 7.4 |
| GW 15-9803/ 0.5M glycine pH 2.25, | 0.346 | 6.5 | 1.753 | 7.9 |
| 2M urea | | | | |
| GW 15-9803/ 0.25M glycine pH 2.7 | 0.369 | 1.2 | 1.847 | 12.5 |
| QC1/ 0.5M glycine pH 2.25, 2M urea | 0.368 | 3.0 | 0.156 | 11.0 |
| QC1/ 0.25M glycine pH 2.7 | 0.378 | 2.9 | 0.184 | 10.1 |

Sample volume and mass of coupled beads for AC-MS. Serum volume and the mass of coupled beads required for affinity capture to achieve suitable MS response ratios for the analytes IBP4 and SHBG were determined empirically. M-PVA beads coupled to the anti-IBP4 antibody 10H7.G1 (“BP4-10H7.G1”) and the ani-SHBG antibody 4G10.F8 (“SHBG-4G10.F8”) were separately assayed for AC-MS of IBP4 and SHBG respectively. The volume of QC2 serum and mass of coupled beads analyzed in replicate AC-MS samples are indicated in Table 4. The optimal mass of coupled beads was chosen to be double the lowest mass that gave MS response ratios that were proportional to serum volumes up to 20 ul. By this criterion the optimal mass of coupled M-PVA beads was 0.25 mg of anti-IBP4 antibody beads and 0.5 mg of anti-SHBG antibody beads.

TABLE 4
Serum and bead titration for AC-MS.

| | M-PVA:IBP4- | | M-PVA:SHBG- | |
|---|---|---|---|---|
| Coupled mAb | 10H7.G1 | | 4G10.F8 | |
| Serum volume/bead mass | ave RR | fold RR | ave RR | fold RR |
| 5 ul QC2_0.125 mg beads | 0.245 | 1 | 0.638 | 1.0 |
| 10 ul QC2_0.125 mg beads | 0.507 | 2.1 | 1.309 | 2.1 |
| 20 ul QC2_0.125 mg beads | 1.039 | 4.2 | 1.942 | 3.0 |
| 5 ul QC2_0.25 mg beads | 0.279 | 1 | 0.824 | 1.0 |
| 10 ul QC2_0.25 mg beads | 0.549 | 2.0 | 1.741 | 2.1 |
| 20 ul QC2_0.25 mg beads | 1.072 | 3.8 | 3.193 | 3.9 |
| 5 ul QC2_0.5 mg beads | 0.276 | 1 | 0.856 | 1.0 |
| 10 ul QC2_0.5 mg beads | 0.577 | 2.1 | 1.807 | 2.1 |
| 20 ul QC2_0.5 mg beads | 1.096 | 4.0 | 3.712 | 4.3 |
| 5 ul QC2_1.0 mg beads | ND | ND | 0.899 | 1.0 |
| 10 ul QC2_1.0 mg beads | ND | ND | 1.828 | 2.0 |
| 20 ul QC2_1.0 mg beads | ND | ND | 3.871 | 4.3 |

Elution, neutralization, reduction and alkylation. IBP4 and SHBG proteins were eluted from the M-PVA affinity capture beads by two sequential aliquots of 0.25M glycine pH 2.7. The combined elution steps were neutralized to pH 8 followed by reduction of disulfide bonds by addition of dithiothreitol to 10 mM and incubation at 60° C. After reduction iodoacetamide was added to 10 mM to alkylate cysteine thiols after which samples were digested with trypsin.

Trypsin digestion optimization. Trypsin digestion incubation time was varied from 1.5 to 3.5 hrs and the digestion temperature was tested at 37° C., 42° C. and 70° C. A standard amount of 2 μg of Promega Trypsin Gold enzyme per sample was used. A panel of 8 serum samples were used to evaluate the above conditions. The criteria included the maximum response ratios achieved for the IBP4 and SHBG analytes as well as the correlation of the response ratios (RR's) and derived biomarker scores to the data obtained by the commercial process with the same sample set. Although the data from the 1.5 hr and 2.5 hr trypsin digestions at 37° C. were similar, overall, the 2.5 hr digestion correlated best with the CLIA assay results and was selected. See Table 5.

TABLE 5
Trypsin digest temperature and time optimization.
Correlation plot R² values: AC-MS vs CLIA Data

| | | | IBP4/SHBG |
|---|---|---|---|
| Digestion condition | IBP4_RR | SHBG RR | Score |
| Trypsin 37° C. 1.5 hr | 0.732 | 0.987 | 0.946 |
| Trypsin 37° C. 2.5 hr | 0.841 | 0.980 | 0.939 |
| Trypsin 37° C. 3.5 hr | 0.676 | 0.948 | 0.932 |
| Trypsin 42° C. 1.5 hr | 0.554 | 0.974 | 0.927 |
| Trypsin 70° C. 1.25 hr | 0.829 | 0.989 | 0.935 |

Anti-IBP4 antibody evaluation and selection. Custom antibodies to IBP4 protein were generated to the C-terminal fragment of IBP4 (amino acids 157-258) and to peptides from within this fragment as discussed in Example 1. To access their suitability for AC-MS, antibodies were purified from primary hybridoma supernatants using protein-G spin columns, then desalted and coupled to Dynabead epoxy magnetic beads as recommended by the manufacturer. Initially only a single isolate 10H7.G1 was positive for efficient IBP4 capture from pooled pregnant serum (QC2) in the AC-MS assay when coupled to Dynabeads. See Table 6.

TABLE 6
Initial candidate anti-IBP4 antibodies screened by AC-MS.

| | IBP4-QCHPALDGQR |
|---|---|
| | (SEQ ID NO: 370) |
| Dynabead-antibody | Response Ratio |
| IBP4-163 control | 0.1735 |
| IBP4-182 control | 0.871 |
| IBP4-185 control | 0.9354 |
| IBP4-190 control | 0.0574 |
| IBP4-7G3.H3 | 0.0038 |
| IBP4-10H7.G1 | 0.0263 |
| IBP4-11B8.H3 | 0.0035 |
| IBP4-14H10.H3 | 0.0063 |
| IBP4-8C7.C2 | 0.0076 |
| IBP4-15B5.B5 | 0.0028 |
| IBP4-1E2.G1 | 0.0016 |

Further screening of candidate anti-IBP4 antibodies. Additional candidate anti-IBP4 antibodies generated by the methods of Example 1 were screened by AC-MS. To compare the relative IBP4 capture efficiency and the reuse ability of the antibody-coupled beads candidate anti-IBP4 antibody clones were repeatedly assayed by AC-MS 3 times. The results of this analysis showed that 3 anti-IBP4 antibody clones (10H7.G1, variant 5B3.G9 and variant 7D6.G4) were superior as determined by the magnitude of response ratios obtained and the relative consistent response ratios upon second and third reuse of the coupled beads. See Table 7.

Each purified anti-IBP4 antibody was coupled to M-PVA epoxy beads and assayed in duplicate. Samples of QC2 serum (10 μl) were processed by AC-MS for all samples in each round. Beads were stored at 4° C. in 50 mM Tris pH 7.5, 150 mM NaCl, 0.05% NG-310, 0.05% NaN₃, 1 mg/ml BSA between AC-MS rounds.

TABLE 7
Repeated AC-MS screening of candidate anti-IBP4 antibodies.

| AC-MS 10 μl QC2 | Round 1 | Round 2 | Round 3 |
|---|---|---|---|
| M-PVA-mAb | ave RR | ave RR | ave RR |
| IBP4-10H7.G1 | 0.4699 | 0.3924 | 0.5071 |
| IBP4-5B3.G9 | 0.7730 | 0.5634 | 0.5748 |
| IBP4-10E11.A10 | 0.0250 | 0.0060 | 0.0051 |
| IBP4-7D6.G4 | 0.5535 | 0.3519 | 0.3212 |
| IBP4-13G10.C2 | 0.1738 | 0.0965 | 0.1299 |
| IBP4-6A3.A2 | 0.0167 | 0.0063 | 0.0203 |
| IBP4-6F3.D8 | 0.2024 | 0.1283 | 0.1192 |
| IBP4-14G2.C4 | 0.0106 | 0.0032 | 0.0088 |
| IBP4-16D5.H8 | 0.0547 | 0.0200 | 0.0196 |

Further evaluation of the anti-IBP4 antibody 10H7.G1 (“IBP4-10H7.G1”) determined that this antibody was specific for the capture of the C-terminal fragment of IBP4 only and failed to capture full length IBP4 protein. For this reason, “IBP4-10H7.G1” was dropped for further consideration for the AC-MS assay.

### Example 5. Validation of Anti-IBP4 Antibody AC-MS Assay

AC-MS of IBP4 Correlation Pools. A set of ten serum sample pools that cover a range of IBP4 protein levels were generated to be used as a test set to compare the performance of the AC-MS process with a clinically validated and proprietary blood biomarker assay using MS without antibody enrichment assay (CLIA). AC-MS assays of the ten IBP4 correlation pools using M-PVA beads coupled to the anti-IBP4 5B3.G9 (“IBP4-5B3.G9”) variant antibody or the anti-IBP4 7D6.G4 (“IBP4-7D6.G4”) variant antibody were performed.

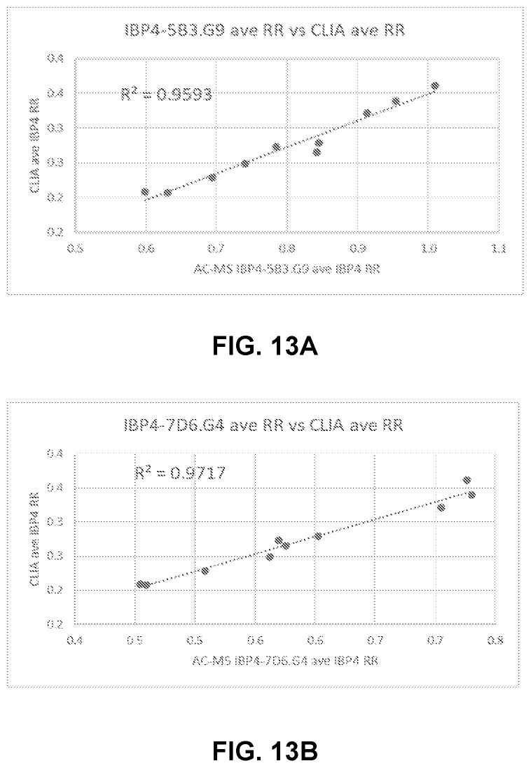

Four replicates of each of the ten sample pools were assayed by AC-MS and yielded an average CV for IBP4 RR of 5.5% for the IBP4-5B3.G9 assay and 5.6% for the “IBP4-7D6.G4” assay. Correlation plots comparing the average IBP4 RR from the AC-MS assay to the average IBP4 RR from the CLIA assay were generated. The correlation agreement was excellent resulting in an R² value of 0.959 with “IBP4-5B3.G9” and an R² of 0.972 with “IBP4-7D6.G4” (FIGS. 13A and 13B).

FIGS. 13A and 13B show correlation plots comparing AC-MS and CLIA IBP4 RR (response ratios). Ten serum pools were assayed by AC-MS and the CLIA assay sometimes referred to herein as CLIA. FIG. 13A shows M-PVA beads coupled to “IBP4-5B3.G9” antibody for AC-MS. FIG. 13B shows beads coupled to “IBP4-7D6.G4” antibody for AC-MS. IBP4 response ratios from each AC-MS assay were plotted against the IBP4 RR data from the CLIA assay.

### Example 6. Validation of Anti-SHBG Antibody AC-MS Assay

Custom antibodies to SHBG protein were generated by a commercial vendor as described above in Example 1. To access their suitability for AC-MS, antibodies were purified from primary hybridoma supernatants using protein-G spin columns, desalted, and coupled to Dynabead epoxy magnetic beads as recommended by the manufacturer. Multiple clones were positive in the AC-MS assay of QC2 serum and 6 anti-SHBG antibodies were carried forward for additional evaluation: 1G7.C5, 2G1.D4, 4G10.F8, 11C6.A8, 16A6.A2 and variant 13C9.E5. See Table 8.

TABLE 8
AC-MS screening of candidate anti-SHBG antibodies.

| AC-MS QC2 serum | SHBG-IALGGLLFPASNLR |
|---|---|
| Dynabead-antibody | RR |
| control Mouse IgG | 0.001 |
| SHBG-2G1.D4 | 1.008 |
| SHBG-1G7.C5 | 4.248 |
| SHBG-4G10.F8 | 1.994 |
| SHBG-11C6.A8 | 1.878 |
| SHBG-16A6.A2 | 4.808 |
| SHBG-13C9.E5 | 2.900 |
| SHBG-6001 control | 6.595 |

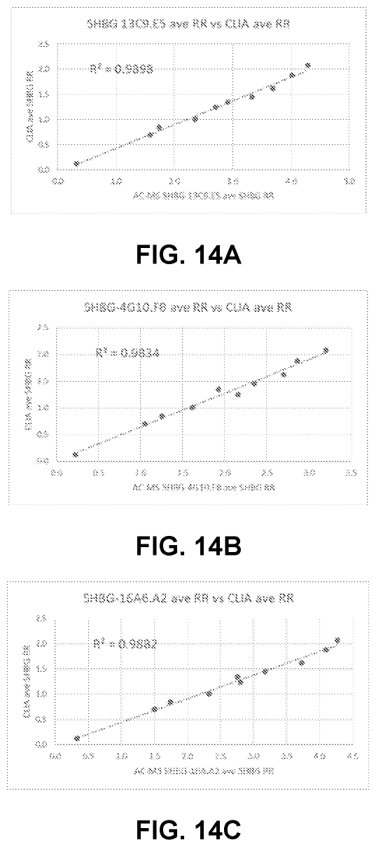

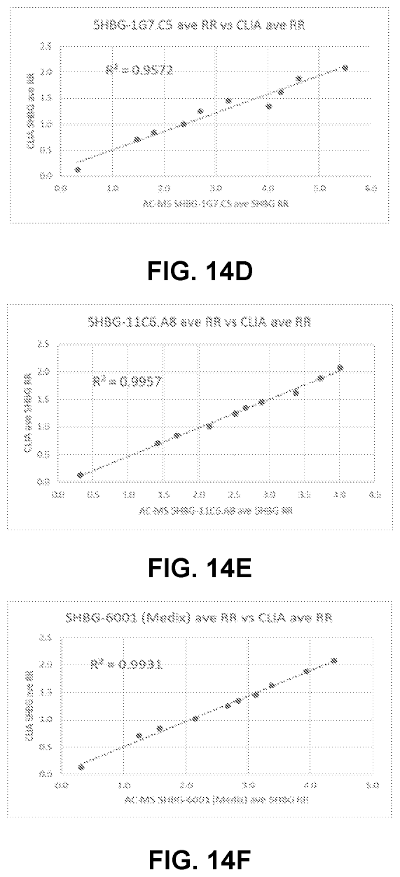

AC-MS of SHBG correlation pools. A set often serum sample pools that cover a range of SHBG protein levels was generated to be used as a test set to compare the performance of the AC-MS process with the CLIA assay. AC-MS assay of each anti-SHBG antibody was carried out with 4 replicates each of the ten sample pools. Average CVs for the SHBG response ratio were ≤6% for each AC-MS assay. Correlation plots comparing the average SHBG RR to the average RR from the CLIA assay were generated and the correlation agreement was excellent for all custom anti-SHBG antibodies with R² values ranging from 0.957 to 0.996. See FIGS. 14A-14F.

FIGS. 14A-14F show correlation plots comparing AC-MS and CLIA SHBG RRs. Ten serum pools were assayed by AC-MS and the CLIA assay. M-PVA beads coupled to various anti-SHBG antibodies are shown in: FIG. 14A: variant 13C9.E5; FIG. 14B: variant 4G10.F8; FIG. 14C: 16A6.A2; FIG. 14D: 1G7.C5; FIG. 14E: 11C6.A8; and FIG. 14F: SHBG-6001 control anti-SHBG antibody. SHBG response ratios from AC-MS assay were plotted against the response ratios from CLIA assay.

### Example 7. Validation of Combined IBP4/SHBG AC-MS Assay

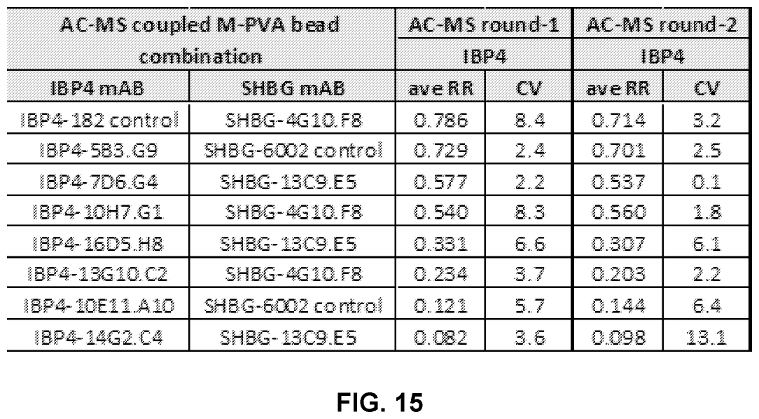

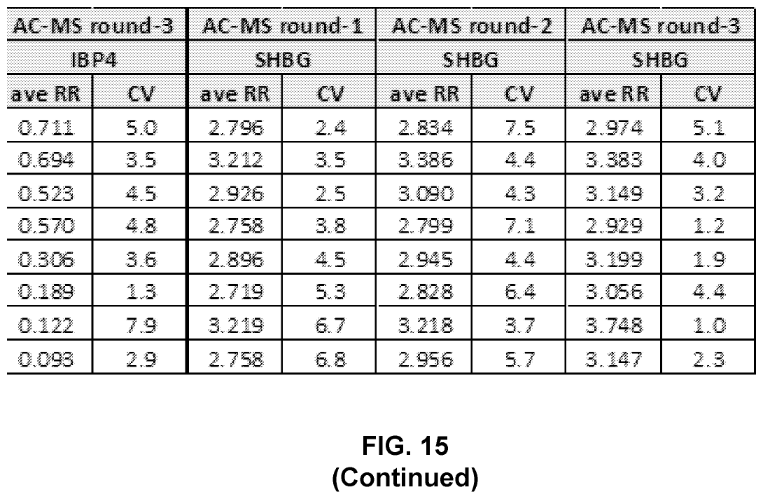

To access the performance of various anti-IBP4 and ant-SHBG antibody combinations in the AC-MS process, mixtures of coupled M-PVA beads were assayed 3 times over a 5-day period. Each combination of anti-IBP4 and anti-SHBG antibody beads were assayed in triplicate with QC2 serum in each round. (FIG. 15). The results indicate that all combinations showed a high level of precision with RR CVs of <8.5% for both IBP4 and SHBG analytes in each of the 3 repeats. Moreover, the average IBP4 and SHBG RRs from each of the 3 rounds showed excellent repeatability with CVs ranging from 2.1% to 9.0% for all bead mixtures. This indicates the performance the AC-MS process with M-PVA coupled beads for determining IBP4 and SHBG levels in serum is highly consistent through multiple uses and correlates well with the CLIA assay. The custom anti-IBP4 antibodies that produced the highest IBP4 RRs were variant 5B3.G9 and variant 7D6.G4 and the custom anti-SHBG antibodies with the greatest SHBG RRs were variant 13C9.E5 and variant 4G10.F8. Pairwise combinations of these antibodies in the AC-MS assay format will be further evaluated for their clinical performance. See, FIG. 15.

### Example 8. Anti-SHBG and Anti-IBP4 Antibodies Binding Characterization

Surface plasmon resonance experiments were performed on a BiacoreT100 to characterize the anti-SHBG and anti-IBP4 antibodies. Anti-mouse-IgG antibody was amine coupled to ˜500 RU on a C1 sensor chip using standard NHS/EDC activation. Test monoclonal antibodies were captured on the anti-mouse-IgG surfaces. The binding for the Sex Hormone Binding Globulin Antigen to anti-SHBG variant antibodies 4G10.F8 and 13C9.E5 were measured in a multi-cycle kinetic mode. Binding studies were performed in PBS buffer+0.05% P20+3 mM EDTA at 25 degrees C. SHBG was captured for 1 minute at 200 nM and regenerated with 1/100 H3PO4 after each cycle. SHBG was captured to similar densities on 2 different flow cells followed by an injection of the SHBG antigen in a 3-fold titration series up to 600 nM over the two surfaces. The responses were processed by subtracting the buffer injection to correct for drift. The data sets fit well to a simple 1:1 interaction model using SCRUBBER (Biologic Software Pty Ltd). The antigen concentration series was tested three times over each surface, giving rise to the kinetic an affinity parameters summarized below.

Provided herein are data showing binding kinetics of anti-SHBG antibody clone variants 4G10.F8 and 13C9.E5. The SHBG clone variant 4G10.F8 includes a heavy chain with SEQ ID NO: 124 with light chain with SEQ ID NO: 125. The SHBG clone variant 13C9.E5 includes a heavy chain with SEQ ID NO: 126 with light chain with SEQ ID NO: 127. The biocore data from are shown below.

SHBG (4G10.F8)

| Anti SHBG | | | |
|---|---|---|---|
| v1 | kₐ (M⁻¹s⁻¹) | k_{d} (s⁻¹) | K_{D} (nM) |
| assay 1 | 4.366(2)e4 | 4.57(7)e−5 | 1.05(2) |
| assay 2 | 4.391(2)e4 | 8.37(6)e−5 | 1.91(1) |
| assay 3 | 3.9638(9)e4 | 1.450(4)e−4 | 3.66(1) |
| average | 4.2[2]E4 | 9[5]E−5 | 2[1] |

SHBG (13C9.E5)

| Anti SHBG | | | |
|---|---|---|---|
| v2 | kₐ (M⁻¹s⁻¹) | k_{d} (s⁻¹) | K_{D} (nM) |
| assay 1 | 1.0942(4)e5 | 4.54(5)e−5 | 0.415(4) |
| assay 2 | 1.0385(4)e5 | 7.94(4)e−5 | 0.765(4) |
| assay 3 | 9.410(3)e4 | 1.191(3)e−4 | 1.266(4) |
| average | 1.03[8]E5 | 8[4]E-5 | 0.800[4] |

In particular, shown are the determined kinetic and affinity constants for the two anti-SHBG antibody variants. Numbers in parentheses represent the statistical standard deviation reported in the last significant figure from the non-linear least squares analysis. For example, 1.05(2) represents 1.05±0.02 nM.

Kinetic and affinity parameters for anti-IBP4 antibodies using the same anti-mouse IgG capture approach noted above resulted in complex kinetics. For this reason, anti-IBP4 antibodies were directly amine coupled using standard NHS/EDC coupling to a C1 sensor chip. Variant 5B3.G9 was coupled on two surfaces at 350 RU and the other at 150 RU. Variant 7D6.G4 was coupled at ˜125 RU. Antibodies were measured in a multi-cycle kinetic mode. Binding studies were performed in PBS buffer+0.05% P20+3 mM EDTA at 25 degrees C. CT-IBP4 was tested in a 3-fold concentration series up to 333 nM and injected at 100 uL/min for 3 minutes followed by a 10 minute dissociation phase. Surfaces were regenerated with 1/100 H3PO4 after each cycle. The antigen binding responses were processed by subtracting the signal from a reference surface with no antibody captured along with a buffer injection. The processed response data were globally fit to a simple 1:1 interaction using SCRUBBER (Biologic Software Pty Ltd). The antigen concentration series was tested three times over each surface, giving rise to the kinetic an affinity parameters summarized in the tables below.

In particular, shown below are the determined kinetic and affinity constants for the two anti-IBP4 antibody variants, 5B3.G9 and 7D6.G4. The IBP4 clone variant 5B3.G9 includes a heavy chain with SEQ ID NO: 123 with light chain with SEQ ID NO: 122. The IBP4 clone variant 7D6.G4 includes a heavy chain with SEQ ID NO: 128 with light chain with SEQ ID NO: 129. Numbers in parentheses represent the statistical standard deviation reported in the last significant figure from the non-linear least squares analysis. For example, 38.25 (9) represents 38.25±0.09 nM.

IBP4 (5B3.G9)

| | Rₘₐₓ | | | |
|---|---|---|---|---|
| V1 | (RU) | kₐ (M⁻¹s⁻¹) | k_{d} (s⁻¹) | K_{D} (nM) |
| Assay | 13.60(1) | 1.170(2)e5 | 0.004474(8) | 38.25(9) |
| 1 | | | | |
| Assay | 12.69(1) | 1.133(3)e5 | 0.00419(1) | 37.0(1) |
| 2 | | | | |
| Assay | 13.02(2) | 1.080(3)e5 | 0.00466(2) | 43.1(2) |
| 3 | | | | |
| Assay | 6.407(8) | 1.569(5)e5 | 0.00404(1) | 25.8(1) |
| 1 | | | | |
| Assay | 5.398(7) | 2.042(6)e5 | 0.00350(1) | 17.12(8) |
| 2 | | | | |
| Assay | 6.036(7) | 1.758(5)e5 | 0.00392(1) | 22.27(8) |
| 3 | | | | |
| | average | 1.5[4]E5 | 0.0041[4] | 31[10] |

IBP4 (7D6.G4)

| | Rₘₐₓ | | | |
|---|---|---|---|---|
| V2 | (RU) | kₐ (M⁻¹s⁻¹) | k_{d} (s⁻¹) | K_{D} (nM) |
| Assay | 5.71(1) | 1.520(7)e5 | 0.01462(4) | 96.2(5) |
| 1 | | | | |
| Assay | 4.41(1) | 2.11(1)e5 | 0.01186(3) | 56.1(3) |
| 2 | | | | |
| Assay | 5.39(1) | 1.683(8)e5 | 0.01277(3) | 75.9(4) |
| 3 | | | | |
| | average | 1.8[3]E5 | 0.013[1] | 76[20] |

Also provided herein is kinetic data for the additional anti-IBP4 antibodies 1H9.B6, 5C11. A7, 7G11.H4, 3D4.E9, and 1G3.A3, generated by surface plasmon resonance. Results are shown below in Table 9. The results demonstrate that these antibodies have similar or improved kinetic properties compared to 5B3.G9.

TABLE 9
Kinetic Data for Additional Antibodies Generated by
Surface Plasmon Resonance

| | | | | | ave IBP4 |
|---|---|---|---|---|---|
| mAb | kₐ (M⁻¹s⁻¹) | k_{d} (s⁻¹) | K_{D} (nM) | t_{1/2} sec | AC-MS RR |
| 1H9.B6 | 6.00E+05 | 9.00E−04 | 1.5 | 770 | 0.588 |
| 5C11.A7 | 4.50E+05 | 7.40E−04 | 1.6 | 936 | 0.548 |
| 7G11.H4 | 3.70E+05 | 1.02E−03 | 2.8 | 679 | 0.525 |
| *5B3.G9 | 2.00E+05 | 2.10E−03 | 10 | 330 | 0.482 |
| 3D4.E9 | 2.70E+05 | 2.21E−03 | 8 | 314 | 0.439 |
| 1G3.A3 | 1.60E+05 | 3.30E−03 | 22 | 210 | 0.272 |

*5B3.G9 served as a control for Kinetic Data.

Summary. These additional antibodies were selected due to the fact that they performed equally well or surpassed the performance of the anti-IBP4 antibodies 5B3.G9 and 7D6.G4. The kinetic performance correlates well with the final AC-MS performance.

Anti-IBP4 antibody evaluation and selection. A total of 8 hybridoma clones (1H9.B6, 5C11.A7, 7G11.H4, 3D4.E9, 1G3.A3, 8G4.G11, 5F10.B2, and 3D8.A11) were produced according to the “additional production of anti-IBP4 antibodies” methods disclosed in Example 1 and screened by AC-MS, shown in Table 10 below. Monoclonal antibodies from these clones purified by protein-G affinity chromatography were covalently coupled to M-PVA magnetic beads and used in AC-MS experiments using pooled pregnant serum. The 5B3.G9 anti-IBP4 antibody clone was included in this analysis for comparison to the performance of the additional IBP4 antibodies, as shown and italicized in Table 10 below. Table 10 includes a summary of the additional antibodies Response Ratios and shared epitope binding results as determined by the competitive binding assays of Example 3. The results of this analysis show that 3 of the anti-IBP4 antibody clones (1H9.B6, 5C11.A7 and 7G11.H4) were superior to clone 5B3.G9, as determined by the magnitude of response ratios obtained.

TABLE 10
Additional Candidate Anti-IBP4 Antibodies Screened by AC-MS

| | IBP4-QCHPALDGQR | Shares epitope |
|---|---|---|
| | (SEQ ID NO: 370) | with |
| M-PVA-antibody | Response Ratio | 5B3.G9 |
| IBP4-1H9.B6 | 0.588 | Y (competition) |
| IBP4-5C11.A7 | 0.548 | Y (competition) |
| IBP4-7G11.H4 | 0.525 | Y (competition) |
| *IBP4-5B3.G9 | 0.482 | — |
| IBP4-3D4.E9 | 0.439 | Y (competition) |
| IBP4-1G3.A3 | 0.272 | Y (competition) |
| IBP4-8G4.G11 | 0.011 | N (no competition) |
| IBP4-5F10.B2 | 0.030 | N (no competition) |
| IBP4-3D8.A11 | 0.005 | N (no competition) |

*Anti-IBP4 antibody clone 5B3.G9 used as control for AC-MS analysis.

Further screening of candidate anti-IBP4 antibodies. To compare the relative IBP4 capture efficiency and the reuse ability of the antibody-coupled beads, the additional anti-IBP4 antibody clones were repeatedly assayed by AC-MS three times. The results of this analysis, shown in Table 11 below, indicate that three of the additional anti-IBP4 antibody clones (1H9.B6, 5C11.A7 and 7G11.H4) were superior to antibody 5B3.G9 as determined by the magnitude of response ratios obtained and the relative consistent response ratios upon second the third reuse of the coupled beads.

TABLE 11
Repeated AC-MS screening of new candidate anti-IBP4 antibodies

| AC-MS 10 ml QC2 | Round 1 | Round 2 | Round 3 |
|---|---|---|---|
| M-PVA-mAb | ave RR | ave RR | ave RR |
| IBP4-1H9.B6 | 0.602 | 0.607 | 0.620 |
| IBP4-5C11.A7 | 0.651 | 0.609 | 0.619 |
| IBP4-7G11.H4 | 0.598 | 0.560 | 0.547 |
| *IBP4-5B3.G9 | 0.533 | 0.534 | 0.462 |
| IBP4-3D4.E9 | 0.491 | 0.468 | 0.454 |
| IBP4-1G3.A3 | 0.281 | 0.291 | 0.289 |
| IBP4-8G4.G11 | 0.158 | 0.167 | 0.173 |
| IBP4-5F10.B2 | 0.042 | 0.036 | 0.035 |
| IBP4-3D8.A11 | 0.043 | 0.017 | 0.016 |

*Anti-IBP4 antibody clone 5B3.G9 served as a control for repeated AC-MS analysis.

### Example 9. Validation of Additional Anti-IBP4 Antibodies in AC-MS Assay

AC-MS of IBP4 Correlation Pools. A set of eight serum sample pools that cover a range of IBP4 protein levels were generated to be used as a test set to compare the performance of the AC-MS process with a clinically validated and proprietary blood biomarker assay using MS without antibody enrichment assay (CLIA). AC-MS assays of the eight IBP4 correlation pools using M-PVA beads coupled to the anti-IBP4 antibodies 1H9.B6, 5C11.A7 or 7G11.H4 variant antibodies were performed.

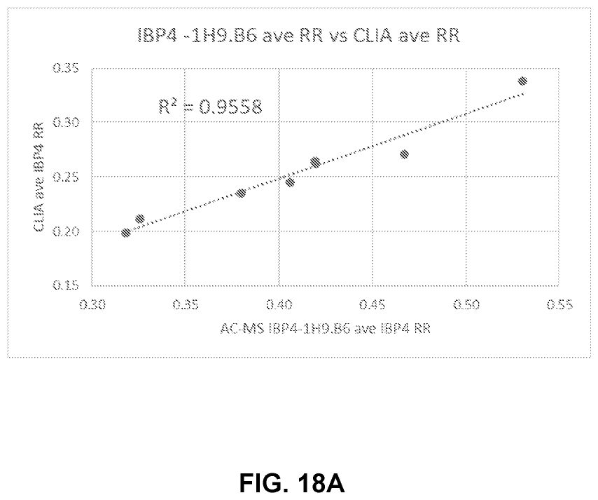

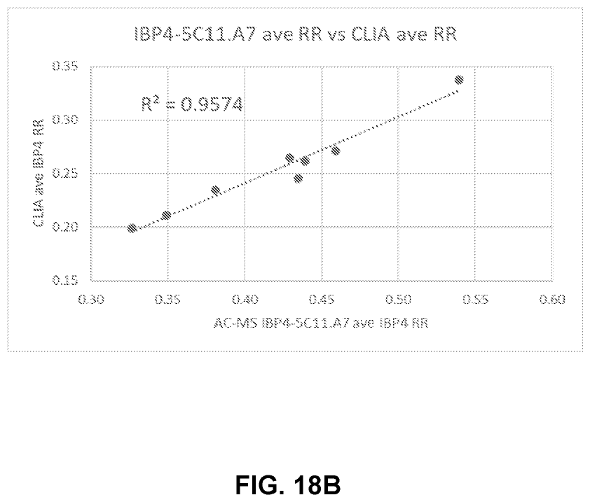

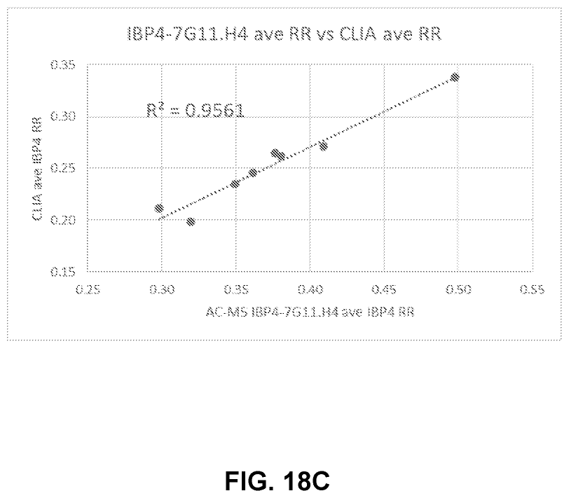

Four replicates of each of the eight sample pools were assayed by AC-MS and yielded an average CV for IBP4 RR (response ratio). of 3.7% for the 1H9.B6 assay, 6.1% for the 5C11.A7 assay and 4.1% for the 7G11.H4 assay. Correlation plots comparing the average IBP4 RR from the AC-MS assay to the average IBP4 RR from the CLIA assay were generated. The correlation agreement was excellent resulting in R² values of 0.956 with 1H9.B6, 0.957 for 5C11.A7 and 0.956 with 7G11.H4 (see, e.g., FIGS. 18A, 18B and 18C).

FIGS. 18A, 18B and 18C show correlation plots comparing AC-MS and CLIA IBP4 RRs (response ratios). Eight serum pools were assayed by AC-MS and the CLIA assay sometimes referred to herein as CLIA. FIG. 18A shows M-PVA beads coupled to the 1H9.B6 anti-IBP4 antibody for AC-MS. FIG. 18B shows beads coupled to the 5C11.A7 anti-IBP4 antibody for AC-MS. FIG. 18C shows beads coupled to the 7G11.H4 anti-IBP4 antibody for AC-MS. IBP4 response ratios from each AC-MS assay were plotted against the IBP4 RR (response ratio) data from the CLIA assay.

AC-MS performance at reduced IBP4 antibody coupling density. IBP4 antibodies were coupled to M-PVA beads at concentrations of 400, 100 and 25 g/ml in the coupling reaction to test the performance of M-PVA beads with reduced density of coupled antibody. Comparison of the IBP4 response ratios obtained in AC-MS experiments with M-PVA beads with a low density of coupled mAb should reveal differences in the apparent affinity of the antibodies to IBP4 protein.

M-PVA beads coupled to IBP4 mAbs 1H9.B6, 5C11.A7, 7G11.H4 or 5B3.G9 at each concentration were used in AC-MS experiments with 10 μl of QC2 serum. This experiment demonstrated that 5B3.G9 showed a greater reduction in AC-MS performance at 25 mg/ml coupling density than the other tested IBP4 mAbs. The percentage of the IBP4 RR (response ratio) at 25 mg/ml relative to the 400 mg/ml coupling density was reduced to 49% for the 5B3.G9 antibody. In contrast, the other mAbs (1H9.B6, 5C11.A7, and 7G11.H4) maintained greater relative performance at 25 mg/ml coupling density compared to 400 mg/ml (82%, 1H9.B6; 79%, 5C11.A7; 75%, 7G11.H4).

TABLE 12
AC-MS performance at reduced IBP4 antibody coupling density.

| mAb/M-PVA | | | |
|---|---|---|---|
| bead coupling | 25 | 100 | 400 |
| density | μg/ml | μg/ml | μg/ml |
| Antibody | ave RR | ave RR | ave RR |
| IBP4-1H9.B6 | 0.479 | 0.562 | 0.586 |
| IBP4-5C11.A7 | 0.470 | 0.567 | 0.595 |
| IBP4-7G11.H4 | 0.404 | 0.492 | 0.538 |
| IBP4-5B3.G9 | 0.231 | 0.449 | 0.470 |

Throughout this application various publications have been referenced. The disclosures of these publications in their entireties are hereby incorporated by reference in this application in order to more fully describe the state of the art to which this disclosure pertains. Although this disclosure has been described with reference to the examples provided above, it should be understood that various modifications can be made without departing from the spirit of what is described herein.

TABLE 13
Sequences of heavy chain variable (VH) region and
light chain variable (VL) region of
anti-SHBG antibody 4G10.F8 and variant 4G10.F8.

| | Nucleotide Sequence | Amino Acid Sequence |
|---|---|---|
| Heavy | CAGGTCCAACTGCAGCAGCCTGGGGCT | QVQLQQPGAELVKPGASVKMSCKASGYTFT |
| Chain | GAGCTGGTGAAGCCTGGGGCTTCAGTG | GYWMHWVKQRPGQGLEWIGVIDPSTGYTT |
| 4G10.F8 | AAGATGTCTTGTAAGGCTTCTGGCTAC | YNEKFKGKATLTVDTSSNTAYMQLSSLTSED |
| | ACCTTCACCGGCTACTGGATGCACTGG | SAVYYCTREPLVTADFEYWGQGTTLTVSSA |
| | GTGAAGCAGAGGCCTGGACAAGGCCT | KTTAPSVYPLAPVCGDTTGSSVTLGCLVKGY |
| | TGAGTGGATCGGAGTAATTGATCCTTC | FPEPVTLTWNSGSLSSGVHTFPAVLQSDLYT |
| | TACTGGCTATACTACCTACAATGAAAA | LSSSVTVTSSTWPSQSITCNVAHPASSTKVDK |
| | ATTCAAGGGCAAGGCCACATTGACTGT | KIEPRGPTIKPCPPCKCPAPNLLGGPSVFIFPP |
| | AGACACATCCTCCAACACAGCCTACAT | KIKDVLMISLSPIVTCVVVDVSEDDPDVQISW |
| | GCAGCTCAGCAGCCTGACCTCTGAGGA | FVNNVEVHTAQTQTHREDYNSTLRVVSALPI |
| | CTCTGCGGTCTATTACTGTACAAGAGA | QHQDWMSGKEFKCKVNNKDLPAPIERTISKP |
| | GCCGTTAGTCACTGCGGACTTTGAGTA | KGSVRAPQVYVLPPPEEEMTKKQVTLTCMV |
| | TTGGGGCCAAGGCACCACTCTCACAGT | TDFMPEDIYVEWTNNGKTELNYKNTEPVLD |
| | CTCCTCAGCCAAAACAACAGCCCCATC | SDGSYFMYSKLRVEKKNWVERNSYSCSVVH |
| | GGTCTATCCACTGGCCCCTGTGTGTGG | EGLHNHHTTKSFSRTPGK |
| | AGATACAACTGGCTCCTCGGTGACTCT | (SEQ ID NO: 4) |
| | GGGATGCCTGGTCAAGGGTTATTTCCC | |
| | TGAGCCAGTGACCTTGACCTGGAACTC | |
| | TGGATCCCTGTCCAGTGGTGTGCACAC | |
| | CTTCCCAGCTGTCCTGCAGTCTGACCTC | |
| | TACACCCTCAGCAGCTCAGTGACTGTA | |
| | ACCTCGAGCACCTGGCCCAGCCAGTCC | |
| | ATCACCTGCAATGTGGCCCACCCGGCA | |
| | AGCAGCACCAAGGTGGACAAGAAAAT | |
| | TGAGCCCAGAGGGCCCACAATCAAGC | |
| | CCTGTCCTCCATGCAAATGCCCAGCAC | |
| | CTAACCTCTTGGGTGGACCATCCGTCT | |
| | TCATCTTCCCTCCAAAGATCAAGGATG | |
| | TACTCATGATCTCCCTGAGCCCCATAG | |
| | TCACATGTGTGGTGGTGGATGTGAGCG | |
| | AGGATGACCCAGATGTCCAGATCAGCT | |
| | GGTTTGTGAACAACGTGGAAGTACACA | |
| | CAGCTCAGACACAAACCCATAGAGAG | |
| | GATTACAACAGTACTCTCCGGGTGGTC | |
| | AGTGCCCTCCCCATCCAGCACCAGGAC | |
| | TGGATGAGTGGCAAGGAGTTCAAATGC | |
| | AAGGTCAACAACAAAGACCTCCCAGC | |
| | GCCCATCGAGAGAACCATCTCAAAACC | |
| | CAAAGGGTCAGTAAGAGCTCCACAGG | |
| | TATATGTCTTGCCTCCACCAGAAGAAG | |
| | AGATGACTAAGAAACAGGTCACTCTGA | |
| | CCTGCATGGTCACAGACTTCATGCCTG | |
| | AAGACATTTACGTGGAGTGGACCAACA | |
| | ACGGGAAAACAGAGCTAAACTACAAG | |
| | AACACTGAACCAGTCCTGGACTCTGAT | |
| | GGTTCTTACTTCATGTACAGCAAGCTG | |
| | AGAGTGGAAAAGAAGAACTGGGTGGA | |
| | AAGAAATAGCTACTCCTGTTCAGTGGT | |
| | CCACGAGGGTCTGCACAATCACCACAC | |
| | GACTAAGAGCTTCTCCCGGACTCCGGG | |
| | TAAATGA (SEQ ID NO: 3) | |
| Heavy | CAGGTCCAACTGCAGCAGCCTGGGGCT | QVQLQQPGAELVKPGASVKMSCKASGYTFT |
| Chain of | GAGCTGGTGAAGCCTGGGGCTTCAGTG | GYWMHWVKQRPGQGLEWIGVIDPSTGYTT |
| variant | AAGATGTCTTGTAAGGCTTCTGGCTAC | YNEKFKGKATLTVDTSSNTAYMQLSSLTSED |
| 4G10.F8 | ACCTTCACCGGCTACTGGATGCACTGG | SAVYYCTREPLVTADFEYWGQGTTLTVSSA |
| | GTGAAGCAGAGGCCTGGACAAGGCCT | KTTAPSVYPLAPVCGDTTGSSVTLGCLVKGY |
| | TGAGTGGATCGGAGTAATTGATCCTTC | FPEPVTLTWNSGSLSSGVHTFPAVLQSDLYT |
| | TACTGGCTATACTACCTACAATGAAAA | LSSSVTVTSSTWPSQSITCNVAHPASSTKVDK |
| | ATTCAAGGGCAAGGCCACATTGACTGT | KIEPRGPTIKPCPPCKCPAPNLLGGPSVFIFPP |
| | AGACACATCCTCCAACACAGCCTACAT | KIKDVLMISLSPIVTCVVVDVSEDDPDVQISW |
| | GCAGCTCAGCAGCCTGACCTCTGAGGA | FVNNVEVHTAQTQTHREDYNSTLRVVSALPI |
| | CTCTGCGGTCTATTACTGTACAAGAGA | QHQDWMSGKEFKCKVNNKDLPAPIERTISKP |
| | GCCGTTAGTCACTGCGGACTTTGAGTA | KGSVRAPQVYVLPPPEEEMTKKQVTLTCMV |
| | TTGGGGCCAAGGCACCACTCTCACAGT | TDFMPEDIYVEWTNNGKTELNYKNTEPVLD |
| | CTCCTCAGCCAAAACAACAGCCCCATC | SDGSYFMYSKLRVEKKNWVERNSYSCSVVH |
| | GGTCTATCCACTGGCCCCTGTGTGTGG | EGLHNHHTTKSFSRTPG (SEQ ID NO: 124) |
| | AGATACAACTGGCTCCTCGGTGACTCT | |
| | GGGATGCCTGGTCAAGGGTTATTTCCC | |
| | TGAGCCAGTGACCTTGACCTGGAACTC | |
| | TGGATCCCTGTCCAGTGGTGTGCACAC | |
| | CTTCCCAGCTGTCCTGCAGTCTGACCTC | |
| | TACACCCTCAGCAGCTCAGTGACTGTA | |
| | ACCTCGAGCACCTGGCCCAGCCAGTCC | |
| | ATCACCTGCAATGTGGCCCACCCGGCA | |
| | AGCAGCACCAAGGTGGACAAGAAAAT | |
| | TGAGCCCAGAGGGCCCACAATCAAGC | |
| | CCTGTCCTCCATGCAAATGCCCAGCAC | |
| | CTAACCTCTTGGGTGGACCATCCGTCT | |
| | TCATCTTCCCTCCAAAGATCAAGGATG | |
| | TACTCATGATCTCCCTGAGCCCCATAG | |
| | TCACATGTGTGGTGGTGGATGTGAGCG | |
| | AGGATGACCCAGATGTCCAGATCAGCT | |
| | GGTTTGTGAACAACGTGGAAGTACACA | |
| | CAGCTCAGACACAAACCCATAGAGAG | |
| | GATTACAACAGTACTCTCCGGGTGGTC | |
| | AGTGCCCTCCCCATCCAGCACCAGGAC | |
| | TGGATGAGTGGCAAGGAGTTCAAATGC | |
| | AAGGTCAACAACAAAGACCTCCCAGC | |
| | GCCCATCGAGAGAACCATCTCAAAACC | |
| | CAAAGGGTCAGTAAGAGCTCCACAGG | |
| | TATATGTCTTGCCTCCACCAGAAGAAG | |
| | AGATGACTAAGAAACAGGTCACTCTGA | |
| | CCTGCATGGTCACAGACTTCATGCCTG | |
| | AAGACATTTACGTGGAGTGGACCAACA | |
| | ACGGGAAAACAGAGCTAAACTACAAG | |
| | AACACTGAACCAGTCCTGGACTCTGAT | |
| | GGTTCTTACTTCATGTACAGCAAGCTG | |
| | AGAGTGGAAAAGAAGAACTGGGTGGA | |
| | AAGAAATAGCTACTCCTGTTCAGTGGT | |
| | CCACGAGGGTCTGCACAATCACCACAC | |
| | GACTAAGAGCTTCTCCCGGACTCCGGG | |
| | TTGA (SEQ ID NO: 200) | |
| Light | GACATTGTGATGACCCAGTCTCAAAAA | DIVMTQSQKFMSTSVGDRISVTCKASQNVDT |
| Chain | TTCATGTCCACATCAGTTGGAGACAGG | NVAWYROKPGQSPKALIYSASYRFSGVPDRF |
| 4G10.F8 | ATCAGCGTCACCTGCAAGGCCAGTCAG | TGSGSGTDFTLTISTFQSEDLAEYFCQQYDSY |
| | AATGTGGATACTAATGTAGCCTGGTAT | PYTFGGGTKLEIKRADAAPTVSIFPPSSEQLTS |
| | CGACAGAAACCAGGGCAATCTCCTAA | GGASVVCFLNNFYPKDINVKWKIDGSERQN |
| | AGCACTGATTTACTCGGCATCCTACCG | GVLNSWTDQDSKDSTYSMSSTLTLTKDEYE |
| | CTTCAGTGGAGTCCCTGATCGCTTCAC | RHNSYTCEATHKTSTSPIVKSFNRNECK (SEQ |
| | AGGCAGTGGATCTGGGACAGATTTCAC | ID NO: 6) |
| | TCTCACCATCAGCACTTTCCAGTCTGA | |
| | AGACTTGGCAGAATATTTCTGTCAGCA | |
| | ATATGACAGCTATCCTTACACGTTCGG | |
| | AGGGGGGACCAAGCTGGAAATAAAAC | |
| | GGGCTGATGCTGCACCAACTGTATCCA | |
| | TCTTCCCACCATCCAGTGAGCAGTTAA | |
| | CATCTGGAGGTGCCTCAGTCGTGTGCT | |
| | TCTTGAACAACTTCTACCCCAAAGACA | |
| | TCAATGTCAAGTGGAAGATTGATGGCA | |
| | GTGAACGACAAAATGGCGTCCTGAAC | |
| | AGTTGGACTGATCAGGACAGCAAAGA | |
| | CAGCACCTACAGCATGAGCAGCACCCT | |
| | CACGTTGACCAAGGACGAGTATGAAC | |
| | GACATAACAGCTATACCTGTGAGGCCA | |
| | CTCACAAGACATCAACTTCACCCATTG | |
| | TCAAGAGCTTCAACAGGAATGAGTGTA | |
| | AATAG (SEQ ID NO: 5) | |
| Light | GACATTGTGATGACCCAGTCTCAAAAA | DIVMTQSQKFMSTSVGDRISVTCKASQNVDT |
| Chain of | TTCATGTCCACATCAGTTGGAGACAGG | NVAWYROKPGQSPKALIYSASYRFSGVPDRF |
| variant | ATCAGCGTCACCTGCAAGGCCAGTCAG | TGSGSGTDFTLTISTFQSEDLAEYFCQQYDSY |
| 4G10.F8 | AATGTGGATACTAATGTAGCCTGGTAT | PYTFGGGTKLEIKRADAAPTVSIFPPSSEQLTS |
| | CGACAGAAACCAGGGCAATCTCCTAA | GGASVVCFLNNFYPKDINVKWKIDGSERQN |
| | AGCACTGATTTACTCGGCATCCTACCG | GVLNSWTDQDSKDSTYSMSSTLTLTKDEYE |
| | CTTCAGTGGAGTCCCTGATCGCTTCAC | RHNSYTCEATHKTSTSPIVKSFNRNEC (SEQ |
| | AGGCAGTGGATCTGGGACAGATTTCAC | ID NO: 125) |
| | TCTCACCATCAGCACTTTCCAGTCTGA | |
| | AGACTTGGCAGAATATTTCTGTCAGCA | |
| | ATATGACAGCTATCCTTACACGTTCGG | |
| | AGGGGGGACCAAGCTGGAAATAAAAC | |
| | GGGCTGATGCTGCACCAACTGTATCCA | |
| | TCTTCCCACCATCCAGTGAGCAGTTAA | |
| | CATCTGGAGGTGCCTCAGTCGTGTGCT | |
| | TCTTGAACAACTTCTACCCCAAAGACA | |
| | TCAATGTCAAGTGGAAGATTGATGGCA | |
| | GTGAACGACAAAATGGCGTCCTGAAC | |
| | AGTTGGACTGATCAGGACAGCAAAGA | |
| | CAGCACCTACAGCATGAGCAGCACCCT | |
| | CACGTTGACCAAGGACGAGTATGAAC | |
| | GACATAACAGCTATACCTGTGAGGCCA | |
| | CTCACAAGACATCAACTTCACCCATTG | |
| | TCAAGAGCTTCAACAGGAATGAGTGTT | |
| | AG (SEQ ID NO: 201) | |
| Heavy | CAGGTCCAACTGCAGCAGCCTGGGGCT | QVQLQQPGAELVKPGASVKMSCKASGYTFT |
| Chain | GAGCTGGTGAAGCCTGGGGCTTCAGTG | GYWMHWVKQRPGQGLEWIGVIDPSTGYTT |
| Variable | AAGATGTCTTGTAAGGCTTCTGGCTAC | YNEKFKGKATLTVDTSSNTAYMQLSSLTSED |
| Region | ACCTTCACCGGCTACTGGATGCACTGG | SAVYYCTREPLVTADFEYWGQGTTLTVSS |
| 4G10.F8 | GTGAAGCAGAGGCCTGGACAAGGCCT | (SEQ ID NO: 8) |
| | TGAGTGGATCGGAGTAATTGATCCTTC | |
| | TACTGGCTATACTACCTACAATGAAAA | |
| | ATTCAAGGGCAAGGCCACATTGACTGT | |
| | AGACACATCCTCCAACACAGCCTACAT | |
| | GCAGCTCAGCAGCCTGACCTCTGAGGA | |
| | CTCTGCGGTCTATTACTGTACAAGAGA | |
| | GCCGTTAGTCACTGCGGACTTTGAGTA | |
| | TTGGGGCCAAGGCACCACTCTCACAGT | |
| | CTCCTCAG (SEQ ID NO: 7) | |
| Light | GACATTGTGATGACCCAGTCTCAAAAA | DIVMTQSQKFMSTSVGDRISVTCKASQNVDT |
| Chain | TTCATGTCCACATCAGTTGGAGACAGG | NVAWYRQKPGQSPKALIYSASYRFSGVPDRF |
| Variable | ATCAGCGTCACCTGCAAGGCCAGTCAG | TGSGSGTDFTLTISTFQSEDLAEYFCQQYDSY |
| Region | AATGTGGATACTAATGTAGCCTGGTAT | PYTFGGGTKLEIK (SEQ ID NO: 10) |
| 4G10.F8 | CGACAGAAACCAGGGCAATCTCCTAA | |
| | AGCACTGATTTACTCGGCATCCTACCG | |
| | CTTCAGTGGAGTCCCTGATCGCTTCAC | |
| | AGGCAGTGGATCTGGGACAGATTTCAC | |
| | TCTCACCATCAGCACTTTCCAGTCTGA | |
| | AGACTTGGCAGAATATTTCTGTCAGCA | |
| | ATATGACAGCTATCCTTACACGTTCGG | |
| | AGGGGGGACCAAGCTGGAAATAAAAC | |
| | (SEQ ID NO: 9) | |

Underlined residues indicate position(s) that were altered in the variant of clone 4G10.F8.

TABLE 14
CDR Sequences of anti-SHBG antibody 4G10.F8 and variant 4G10.F8.

| | Region | | | |
|---|---|---|---|---|
| | Definition | CDR1 | CDR2 | CDR3 |
| Heavy | IMGT | GYTFTGYW | IDPSTGYT | TREPLVTADFEY |
| Chain | | (SEQ ID NO: 11) | (SEQ ID NO: 12) | (SEQ ID NO: 13) |
| | Chothia | GYTFTGYWMH | VIDPSTGYTTYNEKFK | EPLVTADFEY |
| | | (SEQ ID NO: 14) | G | (SEQ ID NO: 16) |
| | | | (SEQ ID NO: 15) | |
| | AbM | GYTFTGYWMHWVK | IGVIDPSTGYTTYNEK | REPLVTADFEYW |
| | | (SEQ ID NO: 17) | FKGKA | (SEQ ID NO: 19) |
| | | | (SEQ ID NO: 18) | |
| | Contact | TGYWMHWVK | LEWIGVIDPSTGYTTY | CTREPLVTADFEY |
| | | (SEQ ID NO: 20) | NEKFKGKA | (SEQ ID NO: 22) |
| | | | (SEQ ID NO: 21) | |
| | Kabat | YWMHWVK | IGVIDPSTGYTTYNEK | REPLVTADFEYW |
| | | (SEQ ID NO: 115) | FKGKATLT | (SEQ ID NO: 19) |
| | | | (SEQ ID NO: 116) | |
| Light | IMGT | QNVDTN | SAS | QQYDSYPYT |
| Chain | | (SEQ ID NO: 23) | (SEQ ID NO: 24) | (SEQ ID NO: 25) |
| | Chothia | KASQNVDTNVA | SASYRFS | QQYDSYPYT |
| | | (SEQ ID NO: 26) | (SEQ ID NO: 27) | (SEQ ID NO: 25) |
| | AbM | TCKASQNVDTNVAWY | SASYRFSGVPD | FCQQYDSYPYTF |
| | | (SEQ ID NO: 29) | (SEQ ID NO: 30) | (SEQ ID NO: 31) |
| | Contact | NVDTNVAWYRQ | ALIYSASYRFSGVP | FCQQYDSYPYT |
| | | (SEQ ID NO: 32) | (SEQ ID NO: 33) | (SEQ ID NO: 34) |
| | Kabat | TCKASQNVDTNVAWY | SASYRFSGVPD | FCQQYDSYPYTF |
| | | (SEQ ID NO: 29) | (SEQ ID NO: 30) | (SEQ ID NO: 31) |

TABLE 15
Sequences of heavy chain variable (VH) region and
light chain variable (VL) region
of anti-SHBG antibody 13C9.E5 and variant 13C9.E5.

| | Nucleotide Sequence | Amino Acid Sequence |
|---|---|---|
| Heavy | CAGATCCAACTGCAGCAGCCTGGGGCT | QIQLQQPGAELVKPGASVKMSCKASGYTFT |
| Chain | GAGCTGGTGAAGCCTGGGGCTTCAGTG | GYWMHWVKQRPGQGLEWIGVIDPSDTYTN |
| 13C9.E5 | AAGATGTCCTGCAAGGCTTCTGGCTAC | YNQKFRGKATLTVDTSSSTAYMQLSGLTSE |
| | ACCTTCACCGGCTACTGGATGCACTGG | DSAVYYCTRDPLVTADFDYWGQGTTLTVSS |
| | GTGAAGCAGAGGCCTGGACAAGGCCT | AKTTPPSVYPLAPGSAAQTNSMVTLGCLVK |
| | TGAGTGGATCGGAGTGATTGATCCTTC | GYFPEPVTVTWNSGSLSSGVHTFPAVLQSDL |
| | TGATACTTATACTAACTACAATCAAAA | YTLSSSVTVPSSTWPSETVTCNVAHPASSTK |
| | ATTCAGGGGCAAGGCCACATTGACTGT | VDKKIVPRDCGCKPCICTVPEVSSVFIFPPKP |
| | AGACACATCCTCCAGCACAGCCTACAT | KDVLTITLTPKVTCVVVDISKDDPEVQFSWF |
| | GCAGCTCAGCGGCCTGACCTCTGAGGA | VDDVEVHTAQTQPREEQFNSTFRSVSELPIM |
| | CTCTGCGGTCTATTACTGTACAAGAGA | HQDWLNGKEFKCRVNSAAFPAPIEKTISKTK |
| | TCCGTTAGTAACTGCGGACTTTGACTA | GRPKAPQVYTIPPPKEQMAKDKVSLTCMITD |
| | CTGGGGCCAAGGCACCACTCTCACAGT | FFPEDITVEWQWNGQPAENYKNTQPIMDTD |
| | CTCCTCAGCCAAAACGACACCCCCATC | GSYFVYSKLNVQKSNWEAGNTFTCSVLHEG |
| | TGTCTATCCACTGGCCCCTGGATCTGCT | LHNHHTEKSLSHSPGK (SEQ ID NO: 36) |
| | GCCCAAACTAACTCCATGGTGACCCTG | |
| | GGATGCCTGGTCAAGGGCTATTTCCCT | |
| | GAGCCAGTGACAGTGACCTGGAACTCT | |
| | GGATCCCTGTCCAGCGGTGTGCACACC | |
| | TTCCCAGCTGTCCTGCAGTCTGACCTCT | |
| | ACACTCTGAGCAGCTCAGTGACTGTCC | |
| | CCTCCAGCACCTGGCCCAGCGAGACCG | |
| | TCACCTGCAACGTTGCCCACCCGGCCA | |
| | GCAGCACCAAGGTGGACAAGAAAATT | |
| | GTGCCCAGGGATTGTGGTTGTAAGCCT | |
| | TGCATATGTACAGTCCCAGAAGTATCA | |
| | TCTGTCTTCATCTTCCCCCCAAAGCCCA | |
| | AGGATGTGCTCACCATTACTCTGACTC | |
| | CTAAGGTCACGTGTGTTGTGGTAGACA | |
| | TCAGCAAGGATGATCCCGAGGTCCAGT | |
| | TCAGCTGGTTTGTAGATGATGTGGAGG | |
| | TGCACACAGCTCAGACGCAACCCCGGG | |
| | AGGAGCAGTTCAACAGCACTTTCCGCT | |
| | CAGTCAGTGAACTTCCCATCATGCACC | |
| | AGGACTGGCTCAATGGCAAGGAGTTCA | |
| | AATGCAGGGTCAACAGTGCAGCTTTCC | |
| | CTGCCCCCATCGAGAAAACCATCTCCA | |
| | AAACCAAAGGCAGACCGAAGGCTCCA | |
| | CAGGTGTACACCATTCCACCTCCCAAG | |
| | GAGCAGATGGCCAAGGATAAAGTCAG | |
| | TCTGACCTGCATGATAACAGACTTCTT | |
| | CCCTGAAGACATTACTGTGGAGTGGCA | |
| | GTGGAATGGGCAGCCAGCGGAGAACT | |
| | ACAAGAACACTCAGCCCATCATGGACA | |
| | CAGATGGCTCTTACTTCGTCTACAGCA | |
| | AGCTCAATGTGCAGAAGAGCAACTGG | |
| | GAGGCAGGAAATACTTTCACCTGCTCT | |
| | GTGTTACATGAGGGCCTGCACAACCAC | |
| | CATACTGAGAAGAGCCTCTCCCACTCT | |
| | CCTGGTAAATGA (SEQ ID NO: 35) | |
| Heavy | CAGATCCAACTGCAGCAGCCTGGGGCT | QIQLQQPGAELVKPGASVKMSCKASGYTFT |
| Chain of | GAGCTGGTGAAGCCTGGGGCTTCAGTG | GYWMHWVKQRPGQGLEWIGVIDPSDTYTN |
| variant | AAGATGTCCTGCAAGGCTTCTGGCTAC | YNQKFRGKATLTVDTSSSTAYMQLSGLTSE |
| 13C9.E5 | ACCTTCACCGGCTACTGGATGCACTGG | DSAVYYCTRDPLVTADFDYWGQGTTLTVSS |
| | GTGAAGCAGAGGCCTGGACAAGGCCT | AKTTPPSVYPLAPGSAAQTNSMVTLGCLVK |
| | TGAGTGGATCGGAGTGATTGATCCTTC | GYFPEPVTVTWNSGSLSSGVHTFPAVLQSDL |
| | TGATACTTATACTAACTACAATCAAAA | YTLSSSVTVPSSTWPSETVTCNVAHPASSTK |
| | ATTCAGGGGCAAGGCCACATTGACTGT | VDKKIVPRDCGCKPCICTVPEVSSVFIFPPKP |
| | AGACACATCCTCCAGCACAGCCTACAT | KDVLTITLTPKVTCVVVDISKDDPEVQFSWF |
| | GCAGCTCAGCGGCCTGACCTCTGAGGA | VDDVEVHTAQTQPREEQFNSTFRSVSELPIM |
| | CTCTGCGGTCTATTACTGTACAAGAGA | HQDWLNGKEFKCRVNSAAFPAPIEKTISKTK |
| | TCCGTTAGTAACTGCGGACTTTGACTA | GRPKAPQVYTIPPPKEQMAKDKVSLTCMITD |
| | CTGGGGCCAAGGCACCACTCTCACAGT | FFPEDITVEWQWNGQPAENYKNTQPIMDTD |
| | CTCCTCAGCCAAAACGACACCCCCATC | GSYFVYSKLNVQKSNWEAGNTFTCSVLHEG |
| | TGTCTATCCACTGGCCCCTGGATCTGCT | LHNHHTEKSLSHSPG (SEQ ID NO: 126) |
| | GCCCAAACTAACTCCATGGTGACCCTG | |
| | GGATGCCTGGTCAAGGGCTATTTCCCT | |
| | GAGCCAGTGACAGTGACCTGGAACTCT | |
| | GGATCCCTGTCCAGCGGTGTGCACACC | |
| | TTCCCAGCTGTCCTGCAGTCTGACCTCT | |
| | ACACTCTGAGCAGCTCAGTGACTGTCC | |
| | CCTCCAGCACCTGGCCCAGCGAGACCG | |
| | TCACCTGCAACGTTGCCCACCCGGCCA | |
| | GCAGCACCAAGGTGGACAAGAAAATT | |
| | GTGCCCAGGGATTGTGGTTGTAAGCCT | |
| | TGCATATGTACAGTCCCAGAAGTATCA | |
| | TCTGTCTTCATCTTCCCCCCAAAGCCCA | |
| | AGGATGTGCTCACCATTACTCTGACTC | |
| | CTAAGGTCACGTGTGTTGTGGTAGACA | |
| | TCAGCAAGGATGATCCCGAGGTCCAGT | |
| | TCAGCTGGTTTGTAGATGATGTGGAGG | |
| | TGCACACAGCTCAGACGCAACCCCGGG | |
| | AGGAGCAGTTCAACAGCACTTTCCGCT | |
| | CAGTCAGTGAACTTCCCATCATGCACC | |
| | AGGACTGGCTCAATGGCAAGGAGTTCA | |
| | AATGCAGGGTCAACAGTGCAGCTTTCC | |
| | CTGCCCCCATCGAGAAAACCATCTCCA | |
| | AAACCAAAGGCAGACCGAAGGCTCCA | |
| | CAGGTGTACACCATTCCACCTCCCAAG | |
| | GAGCAGATGGCCAAGGATAAAGTCAG | |
| | TCTGACCTGCATGATAACAGACTTCTT | |
| | CCCTGAAGACATTACTGTGGAGTGGCA | |
| | GTGGAATGGGCAGCCAGCGGAGAACT | |
| | ACAAGAACACTCAGCCCATCATGGACA | |
| | CAGATGGCTCTTACTTCGTCTACAGCA | |
| | AGCTCAATGTGCAGAAGAGCAACTGG | |
| | GAGGCAGGAAATACTTTCACCTGCTCT | |
| | GTGTTACATGAGGGCCTGCACAACCAC | |
| | CATACTGAGAAGAGCCTCTCCCACTCT | |
| | CCTGGTTGA (SEQ ID NO: 202) | |
| Light | GACATTGTGATGACCCAGTCTCAAAAA | DIVMTQSQKFMSTSVGDRVSVTCKASQNVD |
| Chain | TTCATGTCCACATCAGTTGGAGACAGG | TNVAWYRQKPGQSPKALIYSASYRFSGVPD |
| 13C9.E5 | GTCAGCGTCACCTGCAAGGCCAGTCAG | RFTGSGSGTDFTLTISNFQSEDLAEYFCQQYD |
| | AATGTGGATACTAATGTAGCCTGGTAT | SYPYTFGGGTKLEIKRADAAPTVSIFPPSSEQ |
| | CGACAGAAACCAGGGCAATCTCCTAA | LTSGGASVVCFLNNFYPKDINVKWKIDGSER |
| | AGCGCTGATTTACTCGGCATCCTACCG | QNGVLNSWTDQDSKDSTYSMSSTLTLTKDE |
| | ATTCAGTGGAGTCCCTGATCGCTTCAC | YERHNSYTCEATHKTSTSPIVKSFNRNECK |
| | AGGCAGTGGATCTGGGACAGATTTCAC | (SEQ ID NO: 38) |
| | TCTCACCATCAGCAATTTCCAGTCTGA | |
| | GGACTTGGCAGAATATTTCTGTCAGCA | |
| | ATATGACAGCTATCCTTACACGTTCGG | |
| | AGGGGGGACCAAGCTGGAAATAAAAC | |
| | GGGCTGATGCTGCACCAACTGTATCCA | |
| | TCTTCCCACCATCCAGTGAGCAGTTAA | |
| | CATCTGGAGGTGCCTCAGTCGTGTGCT | |
| | TCTTGAACAACTTCTACCCCAAAGACA | |
| | TCAATGTCAAGTGGAAGATTGATGGCA | |
| | GTGAACGACAAAATGGCGTCCTGAAC | |
| | AGTTGGACTGATCAGGACAGCAAAGA | |
| | CAGCACCTACAGCATGAGCAGCACCCT | |
| | CACGTTGACCAAGGACGAGTATGAAC | |
| | GACATAACAGCTATACCTGTGAGGCCA | |
| | CTCACAAGACATCAACTTCACCCATTG | |
| | TCAAGAGCTTCAACAGGAATGAGTGTA | |
| | AATAG (SEQ ID NO: 37) | |
| Light | GACATTGTGATGACCCAGTCTCAAAAA | DIVMTQSQKFMSTSVGDRVSVTCKASQNVD |
| Chain of | TTCATGTCCACATCAGTTGGAGACAGG | TNVAWYRQKPGQSPKALIYSASYRFSGVPD |
| variant | GTCAGCGTCACCTGCAAGGCCAGTCAG | RFTGSGSGTDFTLTISNFQSEDLAEYFCQQYD |
| 13C9.E5 | AATGTGGATACTAATGTAGCCTGGTAT | SYPYTFGGGTKLEIKRADAAPTVSIFPPSSEQ |
| | CGACAGAAACCAGGGCAATCTCCTAA | LTSGGASVVCFLNNFYPKDINVKWKIDGSER |
| | AGCGCTGATTTACTCGGCATCCTACCG | QNGVLNSWTDQDSKDSTYSMSSTLTLTKDE |
| | ATTCAGTGGAGTCCCTGATCGCTTCAC | YERHNSYTCEATHKTSTSPIVKSFNRNEC |
| | AGGCAGTGGATCTGGGACAGATTTCAC | (SEQ ID NO: 127) |
| | TCTCACCATCAGCAATTTCCAGTCTGA | |
| | GGACTTGGCAGAATATTTCTGTCAGCA | |
| | ATATGACAGCTATCCTTACACGTTCGG | |
| | AGGGGGGACCAAGCTGGAAATAAAAC | |
| | GGGCTGATGCTGCACCAACTGTATCCA | |
| | TCTTCCCACCATCCAGTGAGCAGTTAA | |
| | CATCTGGAGGTGCCTCAGTCGTGTGCT | |
| | TCTTGAACAACTTCTACCCCAAAGACA | |
| | TCAATGTCAAGTGGAAGATTGATGGCA | |
| | GTGAACGACAAAATGGCGTCCTGAAC | |
| | AGTTGGACTGATCAGGACAGCAAAGA | |
| | CAGCACCTACAGCATGAGCAGCACCCT | |
| | CACGTTGACCAAGGACGAGTATGAAC | |
| | GACATAACAGCTATACCTGTGAGGCCA | |
| | CTCACAAGACATCAACTTCACCCATTG | |
| | TCAAGAGCTTCAACAGGAATGAGTGTT | |
| | AG (SEQ ID NO: 203) | |
| Heavy | CAGATCCAACTGCAGCAGCCTGGGGCT | QIQLQQPGAELVKPGASVKMSCKASGYTFT |
| Chain | GAGCTGGTGAAGCCTGGGGCTTCAGTG | GYWMHWVKQRPGQGLEWIGVIDPSDTYTN |
| Variable | AAGATGTCCTGCAAGGCTTCTGGCTAC | YNQKFRGKATLTVDTSSSTAYMQLSGLTSE |
| Region | ACCTTCACCGGCTACTGGATGCACTGG | DSAVYYCTRDPLVTADFDYWGQGTTLTVSS |
| 13C9.E5 | GTGAAGCAGAGGCCTGGACAAGGCCT | (SEQ ID NO: 40) |
| | TGAGTGGATCGGAGTGATTGATCCTTC | |
| | TGATACTTATACTAACTACAATCAAAA | |
| | ATTCAGGGGCAAGGCCACATTGACTGT | |
| | AGACACATCCTCCAGCACAGCCTACAT | |
| | GCAGCTCAGCGGCCTGACCTCTGAGGA | |
| | CTCTGCGGTCTATTACTGTACAAGAGA | |
| | TCCGTTAGTAACTGCGGACTTTGACTA | |
| | CTGGGGCCAAGGCACCACTCTCACAGT | |
| | CTCCTCAG (SEQ ID NO: 39) | |
| Light | GACATTGTGATGACCCAGTCTCAAAAA | DIVMTQSQKFMSTSVGDRVSVTCKASQNVD |
| Chain | TTCATGTCCACATCAGTTGGAGACAGG | TNVAWYROKPGQSPKALIYSASYRFSGVPD |
| Variable | GTCAGCGTCACCTGCAAGGCCAGTCAG | RFTGSGSGTDFTLTISNFQSEDLAEYFCQQYD |
| Region | AATGTGGATACTAATGTAGCCTGGTAT | SYPYTFGGGTKLEIK (SEQ ID NO: 42) |
| 13C9.E5 | CGACAGAAACCAGGGCAATCTCCTAA | |
| | AGCGCTGATTTACTCGGCATCCTACCG | |
| | ATTCAGTGGAGTCCCTGATCGCTTCAC | |
| | AGGCAGTGGATCTGGGACAGATTTCAC | |
| | TCTCACCATCAGCAATTTCCAGTCTGA | |
| | GGACTTGGCAGAATATTTCTGTCAGCA | |
| | ATATGACAGCTATCCTTACACGTTCGG | |
| | AGGGGGGACCAAGCTGGAAATAAAAC | |
| | (SEQ ID NO: 41) | |

Underlined residues indicate position(s) that were altered in the variant of clone 13C9.E5.

TABLE 16
CDR Sequences of anti-SHBG antibody 13C9.E5 and variant 13C9.E5.

| | Region | | | |
|---|---|---|---|---|
| | Definition | CDR1 | CDR2 | CDR3 |
| Heavy | IMGT | GYTFTGYW | IDPSDTYT | TRDPLVTADFDY |
| Chain | | (SEQ ID NO: 11) | (SEQ ID NO: 44) | (SEQ ID NO: 45) |
| | Chothia | GYTFTGYWMH | VIDPSDTYTNYNQKFR | DPLVTADFDY |
| | | (SEQ ID NO: 14) | G | (SEQ ID NO: 48) |
| | | | (SEQ ID NO: 47) | |
| | AbM | GYTFTGYWMHWVK | IGVIDPSDTYTNYNQK | RDPLVTADFDYW |
| | | (SEQ ID NO: 17) | FRGKA | (SEQ ID NO: 51) |
| | | | (SEQ ID NO: 50) | |
| | Contact | TGYWMHVK | LEWIGVIDPSDTYTNY | CTRDPLVTADFDY |
| | | (SEQ ID NO: 52) | NQKFRGKA | (SEQ ID NO: 54) |
| | | | (SEQ ID NO: 53) | |
| | Kabat | YWMHWVK | IGVIDPSDTYTNYNQK | RDPLVTADFDYW |
| | | (SEQ ID NO: 115) | FRGKATLT | (SEQ ID NO: 51) |
| | | | (SEQ ID NO: 117) | |
| Light | IMGT | QNVDTN | SAS | QQYDSYPYT |
| Chain | | (SEQ ID NO: 23) | (SEQ ID NO: 24) | (SEQ ID NO: 25) |
| | Chothia | KASQNVDTNVA | SASYRFS | QQYDSYPYT |
| | | (SEQ ID NO: 26) | (SEQ ID NO: 27) | (SEQ ID NO: 25) |
| | AbM | TCKASQNVDTNVAWY | SASYRFSGVPD | FCQQYDSYPYTF |
| | | (SEQ ID NO: 29) | (SEQ ID NO: 30) | (SEQ ID NO: 31) |
| | Contact | NVDTNVAWYRQ | ALIYSASYRFSGVP | FCQQYDSYPYT |
| | | (SEQ ID NO: 32) | (SEQ ID NO: 33) | (SEQ ID NO: 34) |
| | Kabat | TCKASQNVDTNVAWY | SASYRFSGVPD | FCQQYDSYPYTF |
| | | (SEQ ID NO: 29) | (SEQ ID NO: 30) | (SEQ ID NO: 31) |

TABLE 17
Sequences of heavy chain variable (VH) region and
light chain variable (VL) region
of anti-IBP4 antibody 5B3.G9 and a variant 5B3.G9.

| | Nucleotide Sequence | Amino Acid Sequence |
|---|---|---|
| Heavy | GAGGTCCAGCTGCAACAATCTGGACCT | EVQLQQSGPELVKPGASAKISCKASGYTFTD |
| Chain | GAGCTGGTGAAGCCTGGGGCTTCAGCG | YYMNWVKQSHGKSLEWIGDINPNNGGSAY |
| 5B3.G9 | AAGATATCCTGTAAGGCTTCTGGATAC | NQKFKGKATLTVDKSSSTAYMELRSLTSEDS |
| | ACGTTCACTGACTACTACATGAACTGG | AVYYCARWDGGFDYWGQGTTLTVSSAKTT |
| | GTGAAGCAGAGCCATGGAAAGAGCCT | PPSVYPLAPGSAAQTNSMVTLGCLVKGYFPE |
| | TGAGTGGATTGGAGATATTAATCCTAA | PVTVTWNSGSLSSGVHTFPAVLQSDLYTLSS |
| | CAATGGTGGTTCTGCCTACAACCAGAA | SVTVPSSTWPSQTVTCNVAHPASSTKVDKKI |
| | GTTCAAGGGCAAGGCCACATTGACTGT | VPRDCGCKPCICTVPEVSSVFIFPPKPKDVLTI |
| | AGACAAGTCCTCCAGCACAGCCTACAT | TLTPKVTCVVVDISKDDPEVQFSWFVDDVE |
| | GGAGCTCCGCAGCCTGACATCTGAGGA | VHTAQTKPREEQINSTFRSVSELPIMHQDWL |
| | CTCTGCAGTCTATTACTGTGCAAGATG | NGKEFKCRVNSAAFPAPIEKTISKTKGRPKAP |
| | GGATGGAGGCTTTGACTACTGGGGCCA | QVYTIPPPKEQMAKDKVSLTCMITNFFPEDIT |
| | AGGCACCACTCTCACAGTCTCCTCAGC | VEWQWNGQPAENYKNTQPIMDTDGSYFVY |
| | CAAAACGACACCCCCATCTGTCTATCC | SKLNVQKSNWEAGNTFTCSVLHEGLHNHHT |
| | ACTGGCCCCTGGATCTGCTGCCCAAAC | EKSLSHSPGK (SEQ ID NO: 68) |
| | TAACTCCATGGTGACCCTGGGATGCCT | |
| | GGTCAAGGGCTATTTCCCTGAGCCAGT | |
| | GACAGTGACCTGGAACTCTGGATCCCT | |
| | GTCCAGCGGTGTGCACACCTTCCCAGC | |
| | TGTCCTGCAGTCTGACCTCTACACTCTG | |
| | AGCAGCTCAGTGACTGTCCCCTCCAGC | |
| | ACCTGGCCCAGCCAGACCGTCACCTGC | |
| | AACGTTGCCCACCCGGCCAGCAGCACC | |
| | AAGGTGGACAAGAAAATTGTGCCCAG | |
| | GGATTGTGGTTGTAAGCCTTGCATATG | |
| | TACAGTCCCAGAAGTATCATCTGTCTT | |
| | CATCTTCCCCCCAAAGCCCAAGGATGT | |
| | GCTCACCATTACTCTGACTCCTAAGGT | |
| | CACGTGTGTTGTGGTAGACATCAGCAA | |
| | GGATGATCCCGAGGTCCAGTTCAGCTG | |
| | GTTTGTAGATGATGTGGAGGTGCACAC | |
| | AGCTCAGACGAAACCCCGGGAGGAGC | |
| | AGATCAACAGCACTTTCCGTTCAGTCA | |
| | GTGAACTTCCCATCATGCACCAGGACT | |
| | GGCTCAATGGCAAGGAGTTCAAATGCA | |
| | GGGTCAACAGTGCAGCTTTCCCTGCCC | |
| | CCATCGAGAAAACCATCTCCAAAACCA | |
| | AAGGCAGACCGAAGGCTCCACAGGTG | |
| | TACACCATTCCACCTCCCAAGGAGCAG | |
| | ATGGCCAAGGATAAAGTCAGTCTGACC | |
| | TGCATGATAACAAACTTCTTCCCTGAA | |
| | GACATTACTGTGGAGTGGCAGTGGAAT | |
| | GGGCAGCCAGCGGAGAACTACAAGAA | |
| | CACTCAGCCCATCATGGACACAGATGG | |
| | CTCTTACTTCGTCTACAGCAAGCTCAA | |
| | TGTGCAGAAGAGCAACTGGGAGGCAG | |
| | GAAATACTTTCACCTGCTCTGTGTTAC | |
| | ATGAGGGCCTGCACAACCACCATACTG | |
| | AGAAGAGCCTCTCCCACTCTCCTGGTA | |
| | AATGA (SEQ ID NO: 67) | |
| Heavy | GTTCAAGGGCAAGGCCACATTGACTGT | EVQLQQSGPELVKPGASAKISCKASGYTFTD |
| Chain of | AGACAAGTCCTCCAGCACAGCCTACAT | YYMNWVKQSHGKSLEWIGDINPNNGGSAY |
| variant | GGAGCTCCGCAGCCTGACATCTGAGGA | NQKFKGKATLTVDKSSSTAYMELRSLTSEDS |
| 5B3.G9 | CTCTGCAGTCTATTACTGTGCAAGATG | AVYYCARWDGGFDYWGQGTTLTVSSAKTT |
| | GGATGGAGGCTTTGACTACTGGGGCCA | PPSVYPLAPGSAAQTNSMVTLGCLVKGYFPE |
| | AGGCACCACTCTCACAGTCTCCTCAGC | PVTVTWNSGSLSSGVHTFPAVLQSDLYTLSS |
| | CAAAACGACACCCCCATCTGTCTATCC | SVTVPSSTWPSQTVTCNVAHPASSTKVDKKI |
| | ACTGGCCCCTGGATCTGCTGCCCAAAC | VPRDCGCKPCICTVPEVSSVFIFPPKPKDVLTI |
| | TAACTCCATGGTGACCCTGGGATGCCT | TLTPKVTCVVVDISKDDPEVQFSWFVDDVE |
| | GGTCAAGGGCTATTTCCCTGAGCCAGT | VHTAQTKPREEQINSTFRSVSELPIMHQDWL |
| | GACAGTGACCTGGAACTCTGGATCCCT | NGKEFKCRVNSAAFPAPIEKTISKTKGRPKAP |
| | GTCCAGCGGTGTGCACACCTTCCCAGC | QVYTIPPPKEQMAKDKVSLTCMITNFFPEDIT |
| | TGTCCTGCAGTCTGACCTCTACACTCTG | VEWQWNGQPAENYKNTQPIMDTDGSYFVY |
| | AGCAGCTCAGTGACTGTCCCCTCCAGC | SKLNVQKSNWEAGNTFTCSVLHEGLHNHHT |
| | ACCTGGCCCAGCCAGACCGTCACCTGC | EKSLSHSPG (SEQ ID NO: 123) |
| | AACGTTGCCCACCCGGCCAGCAGCACC | |
| | AAGGTGGACAAGAAAATTGTGCCCAG | |
| | GGATTGTGGTTGTAAGCCTTGCATATG | |
| | TACAGTCCCAGAAGTATCATCTGTCTT | |
| | CATCTTCCCCCCAAAGCCCAAGGATGT | |
| | GCTCACCATTACTCTGACTCCTAAGGT | |
| | CACGTGTGTTGTGGTAGACATCAGCAA | |
| | GGATGATCCCGAGGTCCAGTTCAGCTG | |
| | GTTTGTAGATGATGTGGAGGTGCACAC | |
| | AGCTCAGACGAAACCCCGGGAGGAGC | |
| | AGATCAACAGCACTTTCCGTTCAGTCA | |
| | GTGAACTTCCCATCATGCACCAGGACT | |
| | GGCTCAATGGCAAGGAGTTCAAATGCA | |
| | GGGTCAACAGTGCAGCTTTCCCTGCCC | |
| | CCATCGAGAAAACCATCTCCAAAACCA | |
| | AAGGCAGACCGAAGGCTCCACAGGTG | |
| | TACACCATTCCACCTCCCAAGGAGCAG | |
| | ATGGCCAAGGATAAAGTCAGTCTGACC | |
| | TGCATGATAACAAACTTCTTCCCTGAA | |
| | GACATTACTGTGGAGTGGCAGTGGAAT | |
| | GGGCAGCCAGCGGAGAACTACAAGAA | |
| | CACTCAGCCCATCATGGACACAGATGG | |
| | CTCTTACTTCGTCTACAGCAAGCTCAA | |
| | TGTGCAGAAGAGCAACTGGGAGGCAG | |
| | GAAATACTTTCACCTGCTCTGTGTTAC | |
| | ATGAGGGCCTGCACAACCACCATACTG | |
| | AGAAGAGCCTCTCCCACTCTCCTGGTT | |
| | GA (SEQ ID NO: 204) | |
| Light | GACATTGTGATGACCCAGTCTCAAAAA | DIVMTQSQKFMSTSVRDRVSVTCKASQNVG |
| Chain | TTCATGTCCACATCAGTAAGAGACAGG | TNVAWYQQKPGQSPKALIYSASYRYSGVPD |
| 5B3.G9 | GTCAGCGTCACCTGCAAGGCCAGTCAG | RFTGSGSGTAFTLTISNVQSEDLAEYFCQQY |
| | AATGTGGGTACTAATGTAGCCTGGTAT | NIYPLTFGAGTKLELKRADAAPTVSIFPPSSE |
| | CAACAGAAACCAGGGCAATCTCCTAA | QLTSGGASVVCFLNNFYPRDINVKWKIDGSE |
| | AGCACTGATTTACTCGGCATCCTACCG | RQNGVLNSWTDQDSKDSTYSMSSTLTLTKD |
| | GTACAGTGGAGTCCCTGATCGCTTCAC | EYERHNSYTCEATHKTSTSPIVKSFNRNECK |
| | AGGCAGTGGATCTGGGACAGCTTTCAC | (SEQ ID NO: 70); or |
| | TCTCACCATCAGCAATGTGCAGTCTGA | |
| | AGACTTGGCAGAGTATTTCTGTCAGCA | |
| | ATATAACATCTATCCTCTCACGTTCGGT | |
| | GCTGGGACCAAGCTGGAGCTGAAACG | |
| | GGCTGATGCTGCACCAACTGTATCCAT | |
| | CTTCCCACCATCCAGTGAGCAGTTAAC | |
| | ATCTGGAGGTGCCTCAGTCGTGTGCTT | |
| | CTTGAACAACTTCTACCCCAGAGACAT | |
| | CAATGTCAAGTGGAAGATTGATGGCAG | |
| | TGAACGACAAAATGGTGTCCTGAACAG | |
| | TTGGACTGATCAGGACAGCAAAGACA | |
| | GCACCTACAGCATGAGCAGCACCCTCA | |
| | CATTGACCAAGGACGAGTATGAACGA | |
| | CATAACAGCTATACCTGTGAGGCCACT | |
| | CACAAGACATCAACTTCACCCATCGTC | |
| | AAGAGCTTCAACAGGAATGAGTGTAA | |
| | ATAG (SEQ ID NO: 69) | |
| Light | GACATTGTGATGACCCAGTCTCAAAAA | DIVMTQSQKFMSTSVRDRVSVTCKASQNVG |
| Chain of | TTCATGTCCACATCAGTAAGAGACAGG | TNVAWYQQKPGQSPKALIYSASYRYSGVPD |
| variant | GTCAGCGTCACCTGCAAGGCCAGTCAG | RFTGSGSGTAFTLTISNVQSEDLAEYFCQQY |
| 5B3.G9 | AATGTGGGTACTAATGTAGCCTGGTAT | NIYPLTFGAGTKLELKRADAAPTVSIFPPSSE |
| | CAACAGAAACCAGGGCAATCTCCTAA | QLTSGGASVVCFLNNFYPKDINVKWKIDGSE |
| | AGCACTGATTTACTCGGCATCCTACCG | RQNGVLNSWTDQDSKDSTYSMSSTLTLTKD |
| | GTACAGTGGAGTCCCTGATCGCTTCAC | EYERHNSYTCEATHKTSTSPIVKSFNRNEC |
| | AGGCAGTGGATCTGGGACAGCTTTCAC | (SEQ ID NO: 122) |
| | TCTCACCATCAGCAATGTGCAGTCTGA | |
| | AGACTTGGCAGAGTATTTCTGTCAGCA | |
| | ATATAACATCTATCCTCTCACGTTCGGT | |
| | GCTGGGACCAAGCTGGAGCTGAAACG | |
| | GGCTGATGCTGCACCAACTGTATCCAT | |
| | CTTCCCACCATCCAGTGAGCAGTTAAC | |
| | ATCTGGAGGTGCCTCAGTCGTGTGCTT | |
| | CTTGAACAACTTCTACCCCAGAGACAT | |
| | CAATGTCAAGTGGAAGATTGATGGCAG | |
| | TGAACGACAAAATGGTGTCCTGAACAG | |
| | TTGGACTGATCAGGACAGCAAAGACA | |
| | GCACCTACAGCATGAGCAGCACCCTCA | |
| | CATTGACCAAGGACGAGTATGAACGA | |
| | CATAACAGCTATACCTGTGAGGCCACT | |
| | CACAAGACATCAACTTCACCCATCGTC | |
| | AAGAGCTTCAACAGGAATGAGTGTTAG | |
| | (SEQ ID NO: 205) | |
| Heavy | GAGGTCCAGCTGCAACAATCTGGACCT | EVQLQQSGPELVKPGASAKISCKASGYTFTD |
| Chain | GAGCTGGTGAAGCCTGGGGCTTCAGCG | YYMNWVKQSHGKSLEWIGDINPNNGGSAY |
| Variable | AAGATATCCTGTAAGGCTTCTGGATAC | NQKFKGKATLTVDKSSSTAYMELRSLTSEDS |
| Region | ACGTTCACTGACTACTACATGAACTGG | AVYYCARWDGGFDYWGQGTTLTVSS (SEQ |
| 5B3.G9 | GTGAAGCAGAGCCATGGAAAGAGCCT | ID NO: 72) |
| | TGAGTGGATTGGAGATATTAATCCTAA | |
| | CAATGGTGGTTCTGCCTACAACCAGAA | |
| | GTTCAAGGGCAAGGCCACATTGACTGT | |
| | AGACAAGTCCTCCAGCACAGCCTACAT | |
| | GGAGCTCCGCAGCCTGACATCTGAGGA | |
| | CTCTGCAGTCTATTACTGTGCAAGATG | |
| | GGATGGAGGCTTTGACTACTGGGGCCA | |
| | AGGCACCACTCTCACAGTCTCCTCAG | |
| | (SEQ ID NO: 71) | |
| Light | GACATTGTGATGACCCAGTCTCAAAAA | DIVMTQSQKFMSTSVRDRVSVTCKASQNVG |
| Chain | TTCATGTCCACATCAGTAAGAGACAGG | TNVAWYQQKPGQSPKALIYSASYRYSGVPD |
| Variable | GTCAGCGTCACCTGCAAGGCCAGTCAG | RFTGSGSGTAFTLTISNVQSEDLAEYFCQQY |
| Region | AATGTGGGTACTAATGTAGCCTGGTAT | NIYPLTFGAGTKLELK (SEQ ID NO: 74) |
| 5B3.G9 | CAACAGAAACCAGGGCAATCTCCTAA | |
| | AGCACTGATTTACTCGGCATCCTACCG | |
| | GTACAGTGGAGTCCCTGATCGCTTCAC | |
| | AGGCAGTGGATCTGGGACAGCTTTCAC | |
| | TCTCACCATCAGCAATGTGCAGTCTGA | |
| | AGACTTGGCAGAGTATTTCTGTCAGCA | |
| | ATATAACATCTATCCTCTCACGTTCGGT | |
| | GCTGGGACCAAGCTGGAGCTGAAAC | |
| | (SEQ ID NO: 73) | |

Underlined residues indicate position(s) that were altered in the variant of clone 5B3.G9.

TABLE 18
CDR Sequences of anti-IBP4 antibody 5B3.G9 and variant 5B3.G9.

| | Region | | | |
|---|---|---|---|---|
| | Definition | CDR1 | CDR2 | CDR3 |
| Heavy | IMGT | GYTFTDYY | INPNNGGS | ARWDGGFDY |
| Chain | | (SEQ ID NO: 75) | (SEQ ID NO: 76) | (SEQ ID NO: 77) |
| | Chothia | GYTFTDYYMN | DINPNNGGSAYNQKFKG | WDGGFDY |
| | | (SEQ ID NO: 78) | (SEQ ID NO: 79) | (SEQ ID NO: 80) |
| | AbM | GYTFTDYYMNWVK | IGDINPNNGGSAYNQKFKGKA | RWDGGFDYW |
| | | (SEQ ID NO: 81) | (SEQ ID NO: 82) | (SEQ ID NO: 83) |
| | Contact | TDYYMNWVK | LEWIGDINPNNGGSAYNQKFKGKA | CARWDGGFDY |
| | | (SEQ ID NO: 84) | (SEQ ID NO: 85) | (SEQ ID NO: 86) |
| | Kabat | YYMNWVK | IGDINPNNGGSAYNQKFKGKATLT | RWDGGFDYW |
| | | (SEQ ID NO: 118) | (SEQ ID NO: 119) | (SEQ ID NO: 83) |
| Light | IMGT | QNVGTN | SAS | QQYNIYPLT |
| Chain | | (SEQ ID NO: 87) | (SEQ ID NO: 24) | (SEQ ID NO: 89) |
| | Chothia | KASQNVGTNVA | SASYRYS | QQYNIYPLT |
| | | (SEQ ID NO: 90) | (SEQ ID NO: 91) | (SEQ ID NO: 89) |
| | AbM | TCKASQNVGTNVAWY | SASYRYSGVPD | FCQQYNIYPLTF |
| | | (SEQ ID NO: 93) | (SEQ ID NO: 94) | (SEQ ID NO: 95) |
| | Contact | NVGTNVAWYQQ | ALIYSASYRYSGVP | FCQQYNIYPLT |
| | | (SEQ ID NO: 96) | (SEQ ID NO: 97) | (SEQ ID NO: 98) |
| | Kabat | TCKASQNVGTNVAWY | SASYRYSGVPD | QQYNIYPLT |
| | | (SEQ ID NO: 93) | (SEQ ID NO: 94) | (SEQ ID NO: 89) |

TABLE 19
Sequences of heavy chain variable (VH) region and light chain
variable (VL) region of anti-IBP4 antibody 7D6.G4 and variant 7D6.G4.

| | Nucleotide Sequence | Amino Acid Sequence |
|---|---|---|
| Heavy | GAGGTCCAGCTGCAACAATCTGGACCT | EVQLQQSGPELVKPGASVKISCKASGYTFTD |
| Chain | GAGCTGGTGAAGCCTGGGGCTTCAGTG | YYMNWVKQSHGKSLEWIGDINPNNGGSAY |
| 7D6.G4 | AAGATATCCTGTAAGGCTTCTGGATAC | NQKFKGKATLTVDKSSSTAYMELRSLTSEDS |
| | ACGTTCACTGACTACTACATGAACTGG | AVYYCARWDGGFDYWGQGTTLTVSSAKTT |
| | GTGAAGCAGAGCCATGGAAAGAGCCT | PPSVYPLAPGSAAQTNSMVTLGCLVKGYFPE |
| | TGAGTGGATTGGAGATATTAATCCTAA | PVTVTWNSGSLSSGVHTFPAVLQSDLYTLSS |
| | CAATGGTGGTTCTGCCTACAACCAGAA | SVTVPSSTWPSQTVTCNVAHPASSTKVDKKI |
| | GTTCAAGGGCAAGGCCACATTGACTGT | VPRDCGCKPCICTVPEVSSVFIFPPKPKDVLTI |
| | AGACAAGTCCTCCAGCACAGCCTACAT | TLTPKVTCVVVDISKDDPEVQFSWFVDDVE |
| | GGAGCTCCGCAGCCTGACATCTGAGGA | VHTAQTKPREEQINSTFRSVSELPIMHQDWL |
| | CTCTGCAGTCTATTACTGTGCAAGATG | NGKEFKCRVNSAAFPAPIEKTISKTKGRPKAP |
| | GGATGGAGGCTTTGACTACTGGGGCCA | QVYTIPPPKEQMAKDKVSLTCMITNFFPEDIT |
| | AGGCACCACTCTCACAGTCTCCTCAGC | VEWQWNGQPAENYKNTQPIMDTDGSYFVY |
| | CAAAACGACACCCCCATCTGTCTATCC | SKLNVQKSNWEAGNTFTCSVLHEGLHNHHT |
| | ACTGGCCCCTGGATCTGCTGCCCAAAC | EKSLSHSPGK (SEQ ID NO: 103) |
| | TAACTCCATGGTGACCCTGGGATGCCT | |
| | GGTCAAGGGCTATTTCCCTGAGCCAGT | |
| | GACAGTGACCTGGAACTCTGGATCCCT | |
| | GTCCAGCGGTGTGCACACCTTCCCAGC | |
| | TGTCCTGCAGTCTGACCTCTACACTCTG | |
| | AGCAGCTCAGTGACTGTCCCCTCCAGC | |
| | ACCTGGCCCAGCCAGACCGTCACCTGC | |
| | AACGTTGCCCACCCGGCCAGCAGCACC | |
| | AAGGTGGACAAGAAAATTGTGCCCAG | |
| | GGATTGTGGTTGTAAGCCTTGCATATG | |
| | TACAGTCCCAGAAGTATCATCTGTCTT | |
| | CATCTTCCCCCCAAAGCCCAAGGATGT | |
| | GCTCACCATTACTCTGACTCCTAAGGT | |
| | CACGTGTGTTGTGGTAGACATCAGCAA | |
| | GGATGATCCCGAGGTCCAGTTCAGCTG | |
| | GTTTGTAGATGATGTGGAGGTGCACAC | |
| | AGCTCAGACGAAACCCCGGGAGGAGC | |
| | AGATCAACAGCACTTTCCGTTCAGTCA | |
| | GTGAACTTCCCATCATGCACCAGGACT | |
| | GGCTCAATGGCAAGGAGTTCAAATGCA | |
| | GGGTCAACAGTGCAGCTTTCCCTGCCC | |
| | CCATCGAGAAAACCATCTCCAAAACCA | |
| | AAGGCAGACCGAAGGCTCCACAGGTG | |
| | TACACCATTCCACCTCCCAAGGAGCAG | |
| | ATGGCCAAGGATAAAGTCAGTCTGACC | |
| | TGCATGATAACAAACTTCTTCCCTGAA | |
| | GACATTACTGTGGAGTGGCAGTGGAAT | |
| | GGGCAGCCAGCGGAGAACTACAAGAA | |
| | CACTCAGCCCATCATGGACACAGATGG | |
| | CTCTTACTTCGTCTACAGCAAGCTCAA | |
| | TGTGCAGAAGAGCAACTGGGAGGCAG | |
| | GAAATACTTTCACCTGCTCTGTGTTAC | |
| | ATGAGGGCCTGCACAACCACCATACTG | |
| | AGAAGAGCCTCTCCCACTCTCCTGGTA | |
| | AATGA (SEQ ID NO: 102) | |
| Heavy | GAGGTCCAGCTGCAACAATCTGGACCT | EVQLQQSGPELVKPGASVKISCKASGYTFTD |
| Chain of | GAGCTGGTGAAGCCTGGGGCTTCAGTG | YYMNWVKQSHGKSLEWIGDINPNNGGSAY |
| variant | AAGATATCCTGTAAGGCTTCTGGATAC | NQKFKGKATLTVDKSSSTAYMELRSLTSEDS |
| 7D6.G4 | ACGTTCACTGACTACTACATGAACTGG | AVYYCARWDGGFDYWGQGTTLTVSSAKTT |
| | GTGAAGCAGAGCCATGGAAAGAGCCT | PPSVYPLAPGSAAQTNSMVTLGCLVKGYFPE |
| | TGAGTGGATTGGAGATATTAATCCTAA | PVTVTWNSGSLSSGVHTFPAVLQSDLYTLSS |
| | CAATGGTGGTTCTGCCTACAACCAGAA | SVTVPSSTWPSQTVTCNVAHPASSTKVDKKI |
| | GTTCAAGGGCAAGGCCACATTGACTGT | VPRDCGCKPCICTVPEVSSVFIFPPKPKDVLTI |
| | AGACAAGTCCTCCAGCACAGCCTACAT | TLTPKVTCVVVDISKDDPEVQFSWFVDDVE |
| | GGAGCTCCGCAGCCTGACATCTGAGGA | VHTAQTKPREEQINSTFRSVSELPIMHQDWL |
| | CTCTGCAGTCTATTACTGTGCAAGATG | NGKEFKCRVNSAAFPAPIEKTISKTKGRPKAP |
| | GGATGGAGGCTTTGACTACTGGGGCCA | QVYTIPPPKEQMAKDKVSLTCMITNFFPEDIT |
| | AGGCACCACTCTCACAGTCTCCTCAGC | VEWQWNGQPAENYKNTQPIMDTDGSYFVY |
| | CAAAACGACACCCCCATCTGTCTATCC | SKLNVQKSNWEAGNTFTCSVLHEGLHNHHT |
| | ACTGGCCCCTGGATCTGCTGCCCAAAC | EKSLSHSPG (SEQ ID NO: 128) |
| | TAACTCCATGGTGACCCTGGGATGCCT | |
| | GGTCAAGGGCTATTTCCCTGAGCCAGT | |
| | GACAGTGACCTGGAACTCTGGATCCCT | |
| | GTCCAGCGGTGTGCACACCTTCCCAGC | |
| | TGTCCTGCAGTCTGACCTCTACACTCTG | |
| | AGCAGCTCAGTGACTGTCCCCTCCAGC | |
| | ACCTGGCCCAGCCAGACCGTCACCTGC | |
| | AACGTTGCCCACCCGGCCAGCAGCACC | |
| | AAGGTGGACAAGAAAATTGTGCCCAG | |
| | GGATTGTGGTTGTAAGCCTTGCATATG | |
| | TACAGTCCCAGAAGTATCATCTGTCTT | |
| | CATCTTCCCCCCAAAGCCCAAGGATGT | |
| | GCTCACCATTACTCTGACTCCTAAGGT | |
| | CACGTGTGTTGTGGTAGACATCAGCAA | |
| | GGATGATCCCGAGGTCCAGTTCAGCTG | |
| | GTTTGTAGATGATGTGGAGGTGCACAC | |
| | AGCTCAGACGAAACCCCGGGAGGAGC | |
| | AGATCAACAGCACTTTCCGTTCAGTCA | |
| | GTGAACTTCCCATCATGCACCAGGACT | |
| | GGCTCAATGGCAAGGAGTTCAAATGCA | |
| | GGGTCAACAGTGCAGCTTTCCCTGCCC | |
| | CCATCGAGAAAACCATCTCCAAAACCA | |
| | AAGGCAGACCGAAGGCTCCACAGGTG | |
| | TACACCATTCCACCTCCCAAGGAGCAG | |
| | ATGGCCAAGGATAAAGTCAGTCTGACC | |
| | TGCATGATAACAAACTTCTTCCCTGAA | |
| | GACATTACTGTGGAGTGGCAGTGGAAT | |
| | GGGCAGCCAGCGGAGAACTACAAGAA | |
| | CACTCAGCCCATCATGGACACAGATGG | |
| | CTCTTACTTCGTCTACAGCAAGCTCAA | |
| | TGTGCAGAAGAGCAACTGGGAGGCAG | |
| | GAAATACTTTCACCTGCTCTGTGTTAC | |
| | ATGAGGGCCTGCACAACCACCATACTG | |
| | AGAAGAGCCTCTCCCACTCTCCTGGTT | |
| | GA (SEQ ID NO: 206) | |
| Light | GACATTGTGATGACCCAGTCTCAAAAA | DIVMTQSQKFMSTSVRDRVSVTCKASQNVG |
| Chain | TTCATGTCCACATCAGTAAGAGACAGG | TNVAWYQQKPGQSPKALIYSASYRYSGVPD |
| 7D6.G4 | GTCAGCGTCACCTGCAAGGCCAGTCAG | RFTGSGSGTDFTLTISNVQSEDLAEYFCQQY |
| | AATGTGGGTACTAATGTAGCCTGGTAT | NSYPLTFGAGTKLELKRADAAPTVSIFPPSSE |
| | CAACAGAAACCAGGGCAATCTCCTAA | QLTSGGASVVCFLNNFYPRDINVKWKIDGSE |
| | AGCACTGATTTACTCGGCATCCTACCG | RQNGVLNSWTDQDSKDSTYSMSSTLTLTKD |
| | GTACAGTGGAGTCCCTGATCGCTTCAC | EYERHNSYTCEATHKTSTSPIVKSFNRNECK |
| | AGGCAGTGGATCTGGGACAGATTTCAC | (SEQ ID NO: 105) |
| | TCTCACCATCAGCAATGTGCAGTCTGA | |
| | AGACTTGGCAGAGTATTTCTGTCAGCA | |
| | ATATAACAGCTATCCTCTCACGTTCGG | |
| | TGCTGGGACCAAGCTGGAGCTGAAAC | |
| | GGGCTGATGCTGCACCAACTGTATCCA | |
| | TCTTCCCACCATCCAGTGAGCAGTTAA | |
| | CATCTGGAGGTGCCTCAGTCGTGTGCT | |
| | TCTTGAACAACTTCTACCCCAGAGACA | |
| | TCAATGTCAAGTGGAAGATTGATGGCA | |
| | GTGAACGACAAAATGGTGTCCTGAACA | |
| | GTTGGACTGATCAGGACAGCAAAGAC | |
| | AGCACCTACAGCATGAGCAGCACCCTC | |
| | ACATTGACCAAGGACGAGTATGAACG | |
| | ACATAACAGCTATACCTGTGAGGCCAC | |
| | TCACAAGACATCAACTTCACCCATCGT | |
| | CAAGAGCTTCAACAGGAATGAGTGTA | |
| | AATAG (SEQ ID NO: 104) | |
| Light | GACATTGTGATGACCCAGTCTCAAAAA | DIVMTQSQKFMSTSVRDRVSVTCKASQNVG |
| Chain of | TTCATGTCCACATCAGTAAGAGACAGG | TNVAWYQQKPGQSPKALIYSASYRYSGVPD |
| variant | GTCAGCGTCACCTGCAAGGCCAGTCAG | RFTGSGSGTDFTLTISNVQSEDLAEYFCQQY |
| 7D6.G4 | AATGTGGGTACTAATGTAGCCTGGTAT | NSYPLTFGAGTKLELKRADAAPTVSIFPPSSE |
| | CAACAGAAACCAGGGCAATCTCCTAA | QLTSGGASVVCFLNNFYPKDINVKWKIDGSE |
| | AGCACTGATTTACTCGGCATCCTACCG | RQNGVLNSWTDQDSKDSTYSMSSTLTLTKD |
| | GTACAGTGGAGTCCCTGATCGCTTCAC | EYERHNSYTCEATHKTSTSPIVKSFNRNEC |
| | AGGCAGTGGATCTGGGACAGATTTCAC | (SEQ ID NO: 129) |
| | TCTCACCATCAGCAATGTGCAGTCTGA | |
| | AGACTTGGCAGAGTATTTCTGTCAGCA | |
| | ATATAACAGCTATCCTCTCACGTTCGG | |
| | TGCTGGGACCAAGCTGGAGCTGAAAC | |
| | GGGCTGATGCTGCACCAACTGTATCCA | |
| | TCTTCCCACCATCCAGTGAGCAGTTAA | |
| | CATCTGGAGGTGCCTCAGTCGTGTGCT | |
| | TCTTGAACAACTTCTACCCCAGAGACA | |
| | TCAATGTCAAGTGGAAGATTGATGGCA | |
| | GTGAACGACAAAATGGTGTCCTGAACA | |
| | GTTGGACTGATCAGGACAGCAAAGAC | |
| | AGCACCTACAGCATGAGCAGCACCCTC | |
| | ACATTGACCAAGGACGAGTATGAACG | |
| | ACATAACAGCTATACCTGTGAGGCCAC | |
| | TCACAAGACATCAACTTCACCCATCGT | |
| | CAAGAGCTTCAACAGGAATGAGTGTTA | |
| | G (SEQ ID NO: 207) | |
| Heavy | GAGGTCCAGCTGCAACAATCTGGACCT | EVQLQQSGPEVKPGASVKISCKASGYTFTD |
| Chain | GAGCTGGTGAAGCCTGGGGCTTCAGTG | YYMNWVKQSHGKSLEWIGDINPNNGGSAY |
| Variable | AAGATATCCTGTAAGGCTTCTGGATAC | NQKFKGKATLTVDKSSSTAYMELRSLTSEDS |
| Region | ACGTTCACTGACTACTACATGAACTGG | AVYYCARWDGGFDYWGQGTTLTVSS (SEQ |
| 7D6.G4 | GTGAAGCAGAGCCATGGAAAGAGCCT | ID NO: 107) |
| | TGAGTGGATTGGAGATATTAATCCTAA | |
| | CAATGGTGGTTCTGCCTACAACCAGAA | |
| | GTTCAAGGGCAAGGCCACATTGACTGT | |
| | AGACAAGTCCTCCAGCACAGCCTACAT | |
| | GGAGCTCCGCAGCCTGACATCTGAGGA | |
| | CTCTGCAGTCTATTACTGTGCAAGATG | |
| | GGATGGAGGCTTTGACTACTGGGGCCA | |
| | AGGCACCACTCTCACAGTCTCCTCAG | |
| | (SEQ ID NO: 106) | |
| Light | GACATTGTGATGACCCAGTCTCAAAAA | DIVMTQSQKFMSTSVRDRVSVTCKASQNVG |
| Chain | TTCATGTCCACATCAGTAAGAGACAGG | TNVAWYQQKPGQSPKALIYSASYRYSGVPD |
| Variable | GTCAGCGTCACCTGCAAGGCCAGTCAG | RFTGSGSGTDFTLTISNVQSEDLAEYFCQQY |
| Region | AATGTGGGTACTAATGTAGCCTGGTAT | NSYPLTFGAGTKLELK (SEQ ID NO: 109) |
| 7D6.G4 | CAACAGAAACCAGGGCAATCTCCTAA | |
| | AGCACTGATTTACTCGGCATCCTACCG | |
| | GTACAGTGGAGTCCCTGATCGCTTCAC | |
| | AGGCAGTGGATCTGGGACAGATTTCAC | |
| | TCTCACCATCAGCAATGTGCAGTCTGA | |
| | AGACTTGGCAGAGTATTTCTGTCAGCA | |
| | ATATAACAGCTATCCTCTCACGTTCGG | |
| | TGCTGGGACCAAGCTGGAGCTGAAAC | |
| | (SEQ ID NO: 108) | |

Underlined residues indicate position(s) that were altered in the variant of clone
7D6.G4.

TABLE 20
CDR Sequences of anti-IBP4 antibody 7D6.G4 and variant 7D6.G4.

| | Region | | | |
|---|---|---|---|---|
| | Definition | CDR1 | CDR2 | CDR3 |
| Heavy | IMGT | GYTFTDYY | INPNNGGS | ARWDGGFDY |
| Chain | | (SEQ ID NO: 75) | (SEQ ID NO: 76) | (SEQ ID NO: 77) |
| | Chothia | GYTFTDYYMN | DINPNNGGSAYNQKFKG | WDGGFDY |
| | | (SEQ ID NO: 78) | (SEQ ID NO: 79) | (SEQ ID NO: 80) |
| | AbM | GYTFTDYYMNWVK | IGDINPNNGGSAYNQKFKGKA | RWDGGFDYW |
| | | (SEQ ID NO: 81) | (SEQ ID NO: 82) | (SEQ ID NO: 83) |
| | Contact | TDYYMNWVK | LEWIGDINPNNGGSAYNQKFKGKA | CARWDGGFDY |
| | | (SEQ ID NO: 84) | (SEQ ID NO: 85) | (SEQ ID NO: 86) |
| | Kabat | YYMNWVK | IGDINPNNGGSAYNQKFKGKATLT | RWDGGFDYW |
| | | (SEQ ID NO: 118) | (SEQ ID NO: 119) | (SEQ ID NO: 83) |
| Light | IMGT | QNVGTN | SAS | QQYNSYPLT |
| Chain | | (SEQ ID NO: 87) | (SEQ ID NO: 24) | (SEQ ID NO: 99) |
| | Chothia | KASQNVGTNVA | SASYRYS | QQYNSYPLT |
| | | (SEQ ID NO: 90) | (SEQ ID NO: 91) | (SEQ ID NO: 99) |
| | AbM | TCKASQNVGTNVAWY | SASYRYSGVPD | FCQQYNSYPLTF |
| | | (SEQ ID NO: 93) | (SEQ ID NO: 94) | (SEQ ID NO: 100) |
| | Contact | NVGTNVAWYQQ | ALIYSASYRYSGVP | FCQQYNSYPLT |
| | | (SEQ ID NO: 96) | (SEQ ID NO: 97) | (SEQ ID NO: 101) |
| | Kabat | TCKASQNVGTNVAWY | SASYRYSGVPD | QQYNSYPLT |
| | | (SEQ ID NO: 93) | (SEQ ID NO: 94) | (SEQ ID NO: 99) |

TABLE 21
SHBG and IBP4 target epitopes.

| Antibody | Epitope Sequence | SEQ ID NO. |
|---|---|---|
| SHBG | LRPVLPTQSAHDPPAVHLSNGPG | SEQ ID NO: 110 |
| IBP4 | KQCHPALDGQRGKCWCV | SEQ ID NO: 111 |
| IBP4 | CHPALDGQRGKCWCVDRKTGVK | SEQ ID NO: 131 |

TABLE 22
Consensus CDR Sequences of anti-SHBG and anti-IBP4 antibody clones.

| Antibody | CDR | Sequence | SEQ ID NO. |
|---|---|---|---|
| SHBG | HCDR2 | IDPSX₁X₂YT | SEQ ID NO: 112 |
| SHBG | HCDR3 | TRX₃PLVTADFX₃Y | SEQ ID NO: 113 |
| IBP4 | HCDR3 | QQYNX1YPLT | SEQ ID NO: 114 |
| IBP4 | HCDR1 | GYTFTYX₁ | SEQ ID NO: 354 |
| IBP4 | HCDR2 | IX₂X₃X₄X₅GGX₆ | SEQ ID NO: 355 |
| IBP4 | HCDR3 | X₇RX₈X₉X₁₀X₁₁X₁₂X₁₃X₁₄X₁₅X₁₆X₁₇DX₁₈ | SEQ ID NO: 356 |
| IBP4 | LCDR1 | QNVX₁₉X₂₀X₂₁ | SEQ ID NO: 357 |
| IBP4 | LCDR2 | SX₂₂S | SEQ ID NO: 358 |
| IBP4 | LCDR3 | QQYX₂₃X₂₄YPLX₂₅ | SEQ ID NO: 359 |
| IBP4 | HCDR2 | INPNNGGX₁ | SEQ ID NO: 360 |
| IBP4 | HCDR3 | ARWX₂X₃DX₄X₅FDY | SEQ ID NO: 361 |
| IBP4 | LCDR1 | QNVGX₆X₇ | SEQ ID NO: 362 |
| IBP4 | LCDR3 | QQYNX₈YPLX₉ | SEQ ID NO: 363 |
| IBP4 | HCDR2 | IDX₁ETGGX₂ | SEQ ID NO: 364 |
| IBP4 | HCDR3 | TRARGSX₃SVYYFDX₄ | SEQ ID NO: 365 |
| IBP4 | LCDR1 | QNVX₅TN | SEQ ID NO: 366 |
| IBP4 | LCDR2 | SX₆S | SEQ ID NO: 367 |
| IBP4 | LCDR3 | QQYDX₇YPLT | SEQ ID NO: 368 |

TABLE 23
Sequences of heavy chain variable (VH) region and light chain
variable (VL) region of anti-IBP4 antibody 1H9.B6 and variant 1H9.B6.

| | Nucleotide Sequence | Amino Acid Sequence |
|---|---|---|
| Heavy | GAGGTCCAGCTGCAACAATCTGGACCT | EVQLQQSGPELVKPGASVKISCKTSGYTFTD |
| Chain | GAGCTGGTGAAGCCTGGGGCTTCAGTG | YYMNWVKQSHGKSLEWIGDINPNNGGAGY |
| 1H9.B6 | AAGATATCCTGTAAGACTTCTGGATAC | NQKFKGKATLTVDKSSSTAYMELRSLTSEDS |
| | ACGTTCACTGACTACTACATGAACTGG | AVYYCARWGWDRYFDYWGQGTTLTVSSA |
| | GTGAAGCAGAGCCATGGAAAGAGCCT | KTTPPSVYPLAPGSAAQTNSMVTLGCLVKG |
| | TGAGTGGATTGGAGATATTAATCCTAA | YFPEPVTVTWNSGSLSSGVHTFPAVLQSDLY |
| | CAATGGTGGTGCTGGCTACAACCAGAA | TLSSSVTVPSSTWPSQTVTCNVAHPASSTKV |
| | GTTCAAGGGCAAGGCCACATTGACTGT | DKKIVPRDCGCKPCICTVPEVSSVFIFPPKPK |
| | AGACAAGTCCTCCAGCACAGCCTACAT | DVLTITLTPKVTCVVVDISKDDPEVQFSWFV |
| | GGAGCTCCGCAGCCTGACATCTGAGGA | DDVEVHTAQTKPREEQINSTFRSVSELPIMH |
| | CTCTGCAGTCTATTACTGTGCAAGATG | QDWLNGKEFKCRVNSAAFPAPIEKTISKTKG |
| | GGGCTGGGACCGGTACTTTGACTACTG | RPKAPQVYTIPPPKEQMAKDKVSLTCMITNF |
| | GGGCCAAGGCACCACTCTCACAGTCTC | FPEDITVEWQWNGQPAENYKNTQPIMDTDG |
| | CTCAGCCAAAACGACACCCCCATCTGT | SYFVYSKLNVQKSNWEAGNTFTCSVLHEGL |
| | CTATCCACTGGCCCCTGGATCTGCTGC | HNHHTEKSLSHSPGK (SEQ ID NO: 209) |
| | CCAAACTAACTCCATGGTGACCCTGGG | |
| | ATGCCTGGTCAAGGGCTATTTCCCTGA | |
| | GCCAGTGACAGTGACCTGGAACTCTGG | |
| | ATCCCTGTCCAGCGGTGTGCACACCTT | |
| | CCCAGCTGTCCTGCAGTCTGACCTCTA | |
| | CACTCTGAGCAGCTCAGTGACTGTCCC | |
| | CTCCAGCACCTGGCCCAGCCAGACCGT | |
| | CACCTGCAACGTTGCCCACCCGGCCAG | |
| | CAGCACCAAGGTGGACAAGAAAATTG | |
| | TGCCCAGGGATTGTGGTTGTAAGCCTT | |
| | GCATATGTACAGTCCCAGAAGTATCAT | |
| | CTGTCTTCATCTTCCCCCCAAAGCCCA | |
| | AGGATGTGCTCACCATTACTCTGACTC | |
| | CTAAGGTCACGTGTGTTGTGGTAGACA | |
| | TCAGCAAGGATGATCCCGAGGTCCAGT | |
| | TCAGCTGGTTTGTAGATGATGTGGAGG | |
| | TGCACACAGCTCAGACGAAACCCCGG | |
| | GAGGAGCAGATCAACAGCACTTTCCGT | |
| | TCAGTCAGTGAACTTCCCATCATGCAC | |
| | CAGGACTGGCTCAATGGCAAGGAGTTC | |
| | AAATGCAGGGTCAACAGTGCAGCTTTC | |
| | CCTGCCCCCATCGAGAAAACCATCTCC | |
| | AAAACCAAAGGCAGACCGAAGGCTCC | |
| | ACAGGTGTACACCATTCCACCTCCCAA | |
| | GGAGCAGATGGCCAAGGATAAAGTCA | |
| | GTCTGACCTGCATGATAACAAACTTCT | |
| | TCCCTGAAGACATTACTGTGGAGTGGC | |
| | AGTGGAATGGGCAGCCAGCGGAGAAC | |
| | TACAAGAACACTCAGCCCATCATGGAC | |
| | ACAGATGGCTCTTACTTCGTCTACAGC | |
| | AAGCTCAATGTGCAGAAGAGCAACTG | |
| | GGAGGCAGGAAATACTTTCACCTGCTC | |
| | TGTGTTACATGAGGGCCTGCACAACCA | |
| | CCATACTGAGAAGAGCCTCTCCCACTC | |
| | TCCTGGTAAATGA (SEQ ID NO: 208) | |
| Heavy | GAGGTCCAGCTGCAACAATCTGGACCT | EVQLQQSGPELVKPGASVKISCKTSGYTFTD |
| Chain of | GAGCTGGTGAAGCCTGGGGCTTCAGTG | YYMNWVKQSHGKSLEWIGDINPNNGGAGY |
| variant | AAGATATCCTGTAAGACTTCTGGATAC | NQKFKGKATLTVDKSSSTAYMELRSLTSEDS |
| 1H9.B6 | ACGTTCACTGACTACTACATGAACTGG | AVYYCARWGWDRYFDYWGQGTTLTVSSA |
| | GTGAAGCAGAGCCATGGAAAGAGCCT | KTTPPSVYPLAPGSAAQTNSMVTLGCLVKG |
| | TGAGTGGATTGGAGATATTAATCCTAA | YFPEPVTVTWNSGSLSSGVHTFPAVLQSDLY |
| | CAATGGTGGTGCTGGCTACAACCAGAA | TLSSSVTVPSSTWPSQTVTCNVAHPASSTKV |
| | GTTCAAGGGCAAGGCCACATTGACTGT | DKKIVPRDCGCKPCICTVPEVSSVFIFPPKPK |
| | AGACAAGTCCTCCAGCACAGCCTACAT | DVLTITLTPKVTCVVVDISKDDPEVQFSWFV |
| | GGAGCTCCGCAGCCTGACATCTGAGGA | DDVEVHTAQTKPREEQINSTFRSVSELPIMH |
| | CTCTGCAGTCTATTACTGTGCAAGATG | QDWLNGKEFKCRVNSAAFPAPIEKTISKTKG |
| | GGGCTGGGACCGGTACTTTGACTACTG | RPKAPQVYTIPPPKEQMAKDKVSLTCMITNF |
| | GGGCCAAGGCACCACTCTCACAGTCTC | FPEDITVEWQWNGQPAENYKNTQPIMDTDG |
| | CTCAGCCAAAACGACACCCCCATCTGT | SYFVYSKLNVQKSNWEAGNTFTCSVLHEGL |
| | CTATCCACTGGCCCCTGGATCTGCTGC | HNHHTEKSLSHSPG (SEQ ID NO: 333) |
| | CCAAACTAACTCCATGGTGACCCTGGG | |
| | ATGCCTGGTCAAGGGCTATTTCCCTGA | |
| | GCCAGTGACAGTGACCTGGAACTCTGG | |
| | ATCCCTGTCCAGCGGTGTGCACACCTT | |
| | CCCAGCTGTCCTGCAGTCTGACCTCTA | |
| | CACTCTGAGCAGCTCAGTGACTGTCCC | |
| | CTCCAGCACCTGGCCCAGCCAGACCGT | |
| | CACCTGCAACGTTGCCCACCCGGCCAG | |
| | CAGCACCAAGGTGGACAAGAAAATTG | |
| | TGCCCAGGGATTGTGGTTGTAAGCCTT | |
| | GCATATGTACAGTCCCAGAAGTATCAT | |
| | CTGTCTTCATCTTCCCCCCAAAGCCCA | |
| | AGGATGTGCTCACCATTACTCTGACTC | |
| | CTAAGGTCACGTGTGTTGTGGTAGACA | |
| | TCAGCAAGGATGATCCCGAGGTCCAGT | |
| | TCAGCTGGTTTGTAGATGATGTGGAGG | |
| | TGCACACAGCTCAGACGAAACCCCGG | |
| | GAGGAGCAGATCAACAGCACTTTCCGT | |
| | TCAGTCAGTGAACTTCCCATCATGCAC | |
| | CAGGACTGGCTCAATGGCAAGGAGTTC | |
| | AAATGCAGGGTCAACAGTGCAGCTTTC | |
| | CCTGCCCCCATCGAGAAAACCATCTCC | |
| | AAAACCAAAGGCAGACCGAAGGCTCC | |
| | ACAGGTGTACACCATTCCACCTCCCAA | |
| | GGAGCAGATGGCCAAGGATAAAGTCA | |
| | GTCTGACCTGCATGATAACAAACTTCT | |
| | TCCCTGAAGACATTACTGTGGAGTGGC | |
| | AGTGGAATGGGCAGCCAGCGGAGAAC | |
| | TACAAGAACACTCAGCCCATCATGGAC | |
| | ACAGATGGCTCTTACTTCGTCTACAGC | |
| | AAGCTCAATGTGCAGAAGAGCAACTG | |
| | GGAGGCAGGAAATACTTTCACCTGCTC | |
| | TGTGTTACATGAGGGCCTGCACAACCA | |
| | CCATACTGAGAAGAGCCTCTCCCACTC | |
| | TCCTGGTTGA (SEQ ID NO: 334) | |
| Light | GACATTGTGATGACCCAGTCTCAAAAA | DIVMTQSQKFMSTSVGDRVSVTCKASQNVG |
| Chain | TTCATGTCCACTTCAGTAGGAGACAGG | IDVAWYQQKPGQSPKALIYSASYRSSGVPDR |
| 1H9.B6 | GTCAGCGTCACCTGCAAGGCCAGTCAG | FTGSGSGTDFTLTINNVQSEDLAEYFCQQYN |
| | AATGTGGGTATTGATGTAGCCTGGTAT | SYPLAFGSGTKLEIKRADAAPTVSIFPPSSEQL |
| | CAACAGAAACCAGGACAATCTCCTAA | TSGGASVVCFLNNFYPRDINVKWKIDGSERQ |
| | AGCACTGATTTACTCGGCATCCTACCG | NGVLNSWTDQDSKDSTYSMSSTLTLTKDEY |
| | GTCCAGTGGAGTCCCTGATCGCTTCAC | ERHNSYTCEATHKTSTSPIVKSFNRNEC |
| | AGGCAGTGGATCTGGGACAGATTTCAC | (SEQ ID NO: 211) |
| | TCTCACCATCAACAATGTGCAGTCTGA | |
| | AGACTTGGCAGAGTATTTCTGTCAGCA | |
| | ATATAACAGCTATCCTCTAGCGTTCGG | |
| | CTCGGGGACAAAGTTGGAAATAAAAC | |
| | GGGCTGATGCTGCACCAACTGTATCCA | |
| | TCTTCCCACCATCCAGTGAGCAGTTAA | |
| | CATCTGGAGGTGCCTCAGTCGTGTGCT | |
| | TCTTGAACAACTTCTACCCCAGAGACA | |
| | TCAATGTCAAGTGGAAGATTGATGGCA | |
| | GTGAACGACAAAATGGTGTCCTGAACA | |
| | GTTGGACTGATCAGGACAGCAAAGAC | |
| | AGCACCTACAGCATGAGCAGCACCCTC | |
| | ACATTGACCAAGGACGAGTATGAACG | |
| | ACATAACAGCTATACCTGTGAGGCCAC | |
| | TCACAAGACATCAACTTCACCCATCGT | |
| | CAAGAGCTTCAACAGGAATGAGTGTTA | |
| | G (SEQ ID NO: 210) | |
| Light | GACATTGTGATGACCCAGTCTCAAAAA | DIVMTQSQKFMSTSVGDRVSVTCKASQNVG |
| Chain of | TTCATGTCCACTTCAGTAGGAGACAGG | IDVAWYQQKPGQSPKALIYSASYRSSGVPDR |
| variant | GTCAGCGTCACCTGCAAGGCCAGTCAG | FTGSGSGTDFTLTINNVQSEDLAEYFCQQYN |
| 1H9.B6 | AATGTGGGTATTGATGTAGCCTGGTAT | SYPLAFGSGTKLEIKRADAAPTVSIFPPSSEQL |
| | CAACAGAAACCAGGACAATCTCCTAA | TSGGASVVCFLNNFYPRDINVKWKIDGSERQ |
| | AGCACTGATTTACTCGGCATCCTACCG | NGVLNSWTDQDSKDSTYSMSSTLTLTKDEY |
| | GTCCAGTGGAGTCCCTGATCGCTTCAC | ERHNSYTCEATHKTSTSPIVKSFNRNEC (SEQ |
| | AGGCAGTGGATCTGGGACAGATTTCAC | ID NO: 211) |
| | TCTCACCATCAACAATGTGCAGTCTGA | |
| | AGACTTGGCAGAGTATTTCTGTCAGCA | |
| | ATATAACAGCTATCCTCTAGCGTTCGG | |
| | CTCGGGGACAAAGTTGGAAATAAAAC | |
| | GGGCTGATGCTGCACCAACTGTATCCA | |
| | TCTTCCCACCATCCAGTGAGCAGTTAA | |
| | CATCTGGAGGTGCCTCAGTCGTGTGCT | |
| | TCTTGAACAACTTCTACCCCAGAGACA | |
| | TCAATGTCAAGTGGAAGATTGATGGCA | |
| | GTGAACGACAAAATGGTGTCCTGAACA | |
| | GTTGGACTGATCAGGACAGCAAAGAC | |
| | AGCACCTACAGCATGAGCAGCACCCTC | |
| | ACATTGACCAAGGACGAGTATGAACG | |
| | ACATAACAGCTATACCTGTGAGGCCAC | |
| | TCACAAGACATCAACTTCACCCATCGT | |
| | CAAGAGCTTCAACAGGAATGAGTGTTA | |
| | G (SEQ ID NO: 210) | |
| Heavy | GAGGTCCAGCTGCAACAATCTGGACCT | EVQLQQSGPELVKPGASVKISCKTSGYTFTD |
| Chain | GAGCTGGTGAAGCCTGGGGCTTCAGTG | YYMNWVKQSHGKSLEWIGDINPNNGGAGY |
| Variable | AAGATATCCTGTAAGACTTCTGGATAC | NQKFKGKATLTVDKSSSTAYMELRSLTSEDS |
| Region | ACGTTCACTGACTACTACATGAACTGG | AVYYCARWGWDRYFDYWGQGTTLTVSS |
| 1H9.B6 | GTGAAGCAGAGCCATGGAAAGAGCCT | (SEQ ID NO: 213) |
| | TGAGTGGATTGGAGATATTAATCCTAA | |
| | CAATGGTGGTGCTGGCTACAACCAGAA | |
| | GTTCAAGGGCAAGGCCACATTGACTGT | |
| | AGACAAGTCCTCCAGCACAGCCTACAT | |
| | GGAGCTCCGCAGCCTGACATCTGAGGA | |
| | CTCTGCAGTCTATTACTGTGCAAGATG | |
| | GGGCTGGGACCGGTACTTTGACTACTG | |
| | GGGCCAAGGCACCACTCTCACAGTCTC | |
| | CTCAG (SEQ ID NO: 212) | |
| Light | GACATTGTGATGACCCAGTCTCAAAAA | DIVMTQSQKFMSTSVGDRVSVTCKASQNVG |
| Chain | TTCATGTCCACTTCAGTAGGAGACAGG | IDVAWYQQKPGQSPKALIYSASYRSSGVPDR |
| Variable | GTCAGCGTCACCTGCAAGGCCAGTCAG | FTGSGSGTDFTLTINNVQSEDLAEYFCQQYN |
| Region | AATGTGGGTATTGATGTAGCCTGGTAT | SYPLAFGSGTKLEIK (SEQ ID NO: 215) |
| 1H9.B6 | CAACAGAAACCAGGACAATCTCCTAA | |
| | AGCACTGATTTACTCGGCATCCTACCG | |
| | GTCCAGTGGAGTCCCTGATCGCTTCAC | |
| | AGGCAGTGGATCTGGGACAGATTTCAC | |
| | TCTCACCATCAACAATGTGCAGTCTGA | |
| | AGACTTGGCAGAGTATTTCTGTCAGCA | |
| | ATATAACAGCTATCCTCTAGCGTTCGG | |
| | CTCGGGGACAAAGTTGGAAATAAAAC | |
| | (SEQ ID NO: 214) | |

Underlined residues indicate position(s) that were altered in the variant of clone
1H9.B6.

TABLE 24
CDR Sequences of anti-IBP4 antibody 1H9.B6 and variant 1H9.B6.

| | Region | | | |
|---|---|---|---|---|
| | Definition | CDR1 | CDR2 | CDR3 |
| Heavy | IMGT | GYTFTDYY | INPNNGGA | ARWGWDRYFDY |
| Chain | | (SEQ ID NO: 75) | (SEQ ID NO: 216) | (SEQ ID NO: 217) |
| | Chothia | GYTFTDYYMN | DINPNNGGAGYNQKFKG | WGWDRYFDY |
| | | (SEQ ID NO: 78) | (SEQ ID NO: 218) | (SEQ ID NO: 219) |
| | AbM | GYTFTDYYMNWVK | IGDINPNNGGAGYNQKFKGKA | RWGWDRYFDYW |
| | | (SEQ ID NO: 81) | (SEQ ID NO: 220) | (SEQ ID NO: 221) |
| | Contact | TDYYMNWVK | LEWIGDINPNNGGAGYNQKFKGKA | CARWGWDRYFDY |
| | | (SEQ ID NO: 84) | (SEQ ID NO: 222) | (SEQ ID NO: 223) |
| | Kabat | YYMNWVK | IGDINPNNGGAGYNQKFKGKATLT | WGWDRYFDYW |
| | | (SEQ ID NO: 118) | (SEQ ID NO: 224) | (SEQ ID NO: 225) |
| Light | IMGT | QNVGID | SAS | QQYNSYPLA |
| Chain | | (SEQ ID NO: 226) | (SEQ ID NO: 24) | (SEQ ID NO: 227) |
| | Chothia | KASQNVGIDVA | SASYRS | QQYNSYPLA |
| | | (SEQ ID NO: 228) | (SEQ ID NO: 229) | (SEQ ID NO: 227) |
| | AbM | TCKASQNVGIDVAWY | SASYRSSGVPD | FCQQYNSYPLAF |
| | | (SEQ ID NO: 230) | (SEQ ID NO: 231) | (SEQ ID NO: 232) |
| | Contact | NVGIDVAWYQQ | ALIYSASYRSSGVP | FCQQYNSYPLA |
| | | (SEQ ID NO: 233) | (SEQ ID NO: 234) | (SEQ ID NO: 235) |
| | Kabat | TCKASQNVGIDVAWY | SASYRSSGVPD | QQYNSYPLA |
| | | (SEQ ID NO: 230) | (SEQ ID NO: 231) | (SEQ ID NO: 227) |

TABLE 25
Sequences of heavy chain variable (VH) region and light chain
variable (VL) region of anti-IBP4 antibody 5C11.A7 and variant 5C11.A7.

| | Nucleotide Sequence | Amino Acid Sequence |
|---|---|---|
| Heavy | CAGGTTCAACTGCAGCAGTCTGGGGCT | QVQLQQSGAELVRPGASVTLSCKASGYTFT |
| Chain | GAACTGGTGAGGCCTGGGGCTTCAGTG | DYEMHWVRQTPVHGLEWIGAIDTETGGPAY |
| 5C11.A7 | ACGCTGTCCTGCAAGGCTTCGGGCTAC | NQKFKGKAILTADKSSSTAYMELRSLTSEDS |
| | ACATTTACTGACTATGAAATGCACTGG | AVYFCTRARGSRSVYYFDNWGQGTTLTVSS |
| | GTGAGGCAGACACCTGTGCATGGCCTG | AKTTPPSVYPLAPGSAAQTNSMVTLGCLVK |
| | GAATGGATTGGAGCTATTGATACTGAA | GYFPEPVTVTWNSGSLSSGVHTFPAVLQSDL |
| | ACTGGTGGTCCTGCCTACAATCAGAAG | YTLSSSVTVPSSTWPSQTVTCNVAHPASSTK |
| | TTCAAGGGCAAGGCCATACTGACTGCA | VDKKIVPRDCGCKPCICTVPEVSSVFIFPPKP |
| | GACAAATCCTCCAGCACAGCCTACATG | KDVLTITLTPKVTCVVVDISKDDPEVQFSWF |
| | GAGCTCCGCAGCCTGACATCTGAGGAC | VDDVEVHTAQTKPREEQINSTFRSVSELPIM |
| | TCTGCCGTCTATTTCTGTACAAGAGCTC | HQDWLNGKEFKCRVNSAAFPAPIEKTISKTK |
| | GCGGTAGTAGGTCCGTGTACTACTTTG | GRPKAPQVYTIPPPKEQMAKDKVSLTCMITN |
| | ACAACTGGGGCCAAGGCACCACTCTCA | FFPEDITVEWQWNGQPAENYKNTQPIMDTD |
| | CAGTCTCCTCAGCCAAAACGACACCCC | GSYFVYSKLNVQKSNWEAGNTFTCSVLHEG |
| | CATCTGTCTATCCACTGGCCCCTGGAT | LHNHHTEKSLSHSPGK |
| | CTGCTGCCCAAACTAACTCCATGGTGA | (SEQ ID NO: 237) |
| | CCCTGGGATGCCTGGTCAAGGGCTATT | |
| | TCCCTGAGCCAGTGACAGTGACCTGGA | |
| | ACTCTGGATCCCTGTCCAGCGGTGTGC | |
| | ACACCTTCCCAGCTGTCCTGCAGTCTG | |
| | ACCTCTACACTCTGAGCAGCTCAGTGA | |
| | CTGTCCCCTCCAGCACCTGGCCCAGCC | |
| | AGACCGTCACCTGCAACGTTGCCCACC | |
| | CGGCCAGCAGCACCAAGGTGGACAAG | |
| | AAAATTGTGCCCAGGGATTGTGGTTGT | |
| | AAGCCTTGCATATGTACAGTCCCAGAA | |
| | GTATCATCTGTCTTCATCTTCCCCCCAA | |
| | AGCCCAAGGATGTGCTCACCATTACTC | |
| | TGACTCCTAAGGTCACGTGTGTTGTGG | |
| | TAGACATCAGCAAGGATGATCCCGAG | |
| | GTCCAGTTCAGCTGGTTTGTAGATGAT | |
| | GTGGAGGTGCACACAGCTCAGACGAA | |
| | ACCCCGGGAGGAGCAGATCAACAGCA | |
| | CTTTCCGTTCAGTCAGTGAACTTCCCAT | |
| | CATGCACCAGGACTGGCTCAATGGCAA | |
| | GGAGTTCAAATGCAGGGTCAACAGTGC | |
| | AGCTTTCCCTGCCCCCATCGAGAAAAC | |
| | CATCTCCAAAACCAAAGGCAGACCGA | |
| | AGGCTCCACAGGTGTACACCATTCCAC | |
| | CTCCCAAGGAGCAGATGGCCAAGGAT | |
| | AAAGTCAGTCTGACCTGCATGATAACA | |
| | AACTTCTTCCCTGAAGACATTACTGTG | |
| | GAGTGGCAGTGGAATGGGCAGCCAGC | |
| | GGAGAACTACAAGAACACTCAGCCCA | |
| | TCATGGACACAGATGGCTCTTACTTCG | |
| | TCTACAGCAAGCTCAATGTGCAGAAGA | |
| | GCAACTGGGAGGCAGGAAATACTTTCA | |
| | CCTGCTCTGTGTTACATGAGGGCCTGC | |
| | ACAACCACCATACTGAGAAGAGCCTCT | |
| | CCCACTCTCCTGGTAAATGA (SEQ ID | |
| | NO: 236) | |
| Heavy | CAGGTTCAACTGCAGCAGTCTGGGGCT | QVQLQQSGAELVRPGASVTLSCKASGYTFT |
| Chain of | GAACTGGTGAGGCCTGGGGCTTCAGTG | DYEMHWVRQTPVHGLEWIGAIDTETGGPAY |
| variant | ACGCTGTCCTGCAAGGCTTCGGGCTAC | NQKFKGKAILTADKSSSTAYMELRSLTSEDS |
| 5C11.A7 | ACATTTACTGACTATGAAATGCACTGG | AVYFCTRARGSRSVYYFDNWGQGTTLTVSS |
| | GTGAGGCAGACACCTGTGCATGGCCTG | AKTTPPSVYPLAPGSAAQTNSMVTLGCLVK |
| | GAATGGATTGGAGCTATTGATACTGAA | GYFPEPVTVTWNSGSLSSGVHTFPAVLQSDL |
| | ACTGGTGGTCCTGCCTACAATCAGAAG | YTLSSSVTVPSSTWPSQTVTCNVAHPASSTK |
| | TTCAAGGGCAAGGCCATACTGACTGCA | VDKKIVPRDCGCKPCICTVPEVSSVFIFPPKP |
| | GACAAATCCTCCAGCACAGCCTACATG | KDVLTITLTPKVTCVVVDISKDDPEVQFSWF |
| | GAGCTCCGCAGCCTGACATCTGAGGAC | VDDVEVHTAQTKPREEQINSTFRSVSELPIM |
| | TCTGCCGTCTATTTCTGTACAAGAGCTC | HQDWLNGKEFKCRVNSAAFPAPIEKTISKTK |
| | GCGGTAGTAGGTCCGTGTACTACTTTG | GRPKAPQVYTIPPPKEQMAKDKVSLTCMITN |
| | ACAACTGGGGCCAAGGCACCACTCTCA | FFPEDITVEWQWNGQPAENYKNTQPIMDTD |
| | CAGTCTCCTCAGCCAAAACGACACCCC | GSYFVYSKLNVQKSNWEAGNTFTCSVLHEG |
| | CATCTGTCTATCCACTGGCCCCTGGAT | LHNHHTEKSLSHSPG |
| | CTGCTGCCCAAACTAACTCCATGGTGA | (SEQ ID NO: 335) |
| | CCCTGGGATGCCTGGTCAAGGGCTATT | |
| | TCCCTGAGCCAGTGACAGTGACCTGGA | |
| | ACTCTGGATCCCTGTCCAGCGGTGTGC | |
| | ACACCTTCCCAGCTGTCCTGCAGTCTG | |
| | ACCTCTACACTCTGAGCAGCTCAGTGA | |
| | CTGTCCCCTCCAGCACCTGGCCCAGCC | |
| | AGACCGTCACCTGCAACGTTGCCCACC | |
| | CGGCCAGCAGCACCAAGGTGGACAAG | |
| | AAAATTGTGCCCAGGGATTGTGGTTGT | |
| | AAGCCTTGCATATGTACAGTCCCAGAA | |
| | GTATCATCTGTCTTCATCTTCCCCCCAA | |
| | AGCCCAAGGATGTGCTCACCATTACTC | |
| | TGACTCCTAAGGTCACGTGTGTTGTGG | |
| | TAGACATCAGCAAGGATGATCCCGAG | |
| | GTCCAGTTCAGCTGGTTTGTAGATGAT | |
| | GTGGAGGTGCACACAGCTCAGACGAA | |
| | ACCCCGGGAGGAGCAGATCAACAGCA | |
| | CTTTCCGTTCAGTCAGTGAACTTCCCAT | |
| | CATGCACCAGGACTGGCTCAATGGCAA | |
| | GGAGTTCAAATGCAGGGTCAACAGTGC | |
| | AGCTTTCCCTGCCCCCATCGAGAAAAC | |
| | CATCTCCAAAACCAAAGGCAGACCGA | |
| | AGGCTCCACAGGTGTACACCATTCCAC | |
| | CTCCCAAGGAGCAGATGGCCAAGGAT | |
| | AAAGTCAGTCTGACCTGCATGATAACA | |
| | AACTTCTTCCCTGAAGACATTACTGTG | |
| | GAGTGGCAGTGGAATGGGCAGCCAGC | |
| | GGAGAACTACAAGAACACTCAGCCCA | |
| | TCATGGACACAGATGGCTCTTACTTCG | |
| | TCTACAGCAAGCTCAATGTGCAGAAGA | |
| | GCAACTGGGAGGCAGGAAATACTTTCA | |
| | CCTGCTCTGTGTTACATGAGGGCCTGC | |
| | ACAACCACCATACTGAGAAGAGCCTCT | |
| | CCCACTCTCCTGGTTGA | |
| | (SEQ ID NO: 336) | |
| Light | GACATTGTGATGACCCAGTCTCAAAAA | DIVMTQSQKFMSTSVGDRVSVTCKASQNVG |
| Chain | TTCATGTCCACATCAGTGGGAGACAGG | TNVAWYQQKPGHSPKVLIYSASYRHSGVPD |
| 5C11.A7 | GTCAGCGTCACCTGCAAGGCCAGTCAG | RFTGSGSGTDFTLTIGNVQSEDLADYFCQQY |
| | AATGTGGGTACTAATGTAGCCTGGTAT | DTYPLTFGSGTKLEIKRADAAPTVSIFPPSSEQ |
| | CAGCAGAAACCAGGGCACTCTCCTAAA | LTSGGASVVCFLNNFYPRDINVKWKIDGSER |
| | GTACTGATTTACTCGGCATCCTACCGG | QNGVLNSWTDQDSKDSTYSMSSTLTLTKDE |
| | CACAGTGGAGTCCCTGATCGCTTCACA | YERHNSYTCEATHKTSTSPIVKSFNRNEC |
| | GGCAGTGGATCTGGGACAGATTTCACT | (SEQ ID NO: 239) |
| | CTCACCATCGGCAATGTGCAGTCTGAG | |
| | GACTTGGCAGACTATTTCTGTCAGCAA | |
| | TATGACACCTATCCTCTCACGTTCGGCT | |
| | CGGGGACAAAGTTGGAAATAAAACGG | |
| | GCTGATGCTGCACCAACTGTATCCATC | |
| | TTCCCACCATCCAGTGAGCAGTTAACA | |
| | TCTGGAGGTGCCTCAGTCGTGTGCTTC | |
| | TTGAACAACTTCTACCCCAGAGACATC | |
| | AATGTCAAGTGGAAGATTGATGGCAGT | |
| | GAACGACAAAATGGTGTCCTGAACAGT | |
| | TGGACTGATCAGGACAGCAAAGACAG | |
| | CACCTACAGCATGAGCAGCACCCTCAC | |
| | ATTGACCAAGGACGAGTATGAACGAC | |
| | ATAACAGCTATACCTGTGAGGCCACTC | |
| | ACAAGACATCAACTTCACCCATCGTCA | |
| | AGAGCTTCAACAGGAATGAGTGTTAG | |
| | (SEQ ID NO: 238) | |
| Light | GACATTGTGATGACCCAGTCTCAAAAA | DIVMTQSQKFMSTSVGDRVSVTCKASQNVG |
| Chain of | TTCATGTCCACATCAGTGGGAGACAGG | TNVAWYQQKPGHSPKVLIYSASYRHSGVPD |
| variant | GTCAGCGTCACCTGCAAGGCCAGTCAG | RFTGSGSGTDFTLTIGNVQSEDLADYFCQQY |
| 5C11.A7 | AATGTGGGTACTAATGTAGCCTGGTAT | DTYPLTFGSGTKLEIKRADAAPTVSIFPPSSEQ |
| | CAGCAGAAACCAGGGCACTCTCCTAAA | LTSGGASVVCFLNNFYPRDINVKWKIDGSER |
| | GTACTGATTTACTCGGCATCCTACCGG | QNGVLNSWTDQDSKDSTYSMSSTLTLTKDE |
| | CACAGTGGAGTCCCTGATCGCTTCACA | YERHNSYTCEATHKTSTSPIVKSFNRNEC |
| | GGCAGTGGATCTGGGACAGATTTCACT | (SEQ ID NO: 239) |
| | CTCACCATCGGCAATGTGCAGTCTGAG | |
| | GACTTGGCAGACTATTTCTGTCAGCAA | |
| | TATGACACCTATCCTCTCACGTTCGGCT | |
| | CGGGGACAAAGTTGGAAATAAAACGG | |
| | GCTGATGCTGCACCAACTGTATCCATC | |
| | TTCCCACCATCCAGTGAGCAGTTAACA | |
| | TCTGGAGGTGCCTCAGTCGTGTGCTTC | |
| | TTGAACAACTTCTACCCCAGAGACATC | |
| | AATGTCAAGTGGAAGATTGATGGCAGT | |
| | GAACGACAAAATGGTGTCCTGAACAGT | |
| | TGGACTGATCAGGACAGCAAAGACAG | |
| | CACCTACAGCATGAGCAGCACCCTCAC | |
| | ATTGACCAAGGACGAGTATGAACGAC | |
| | ATAACAGCTATACCTGTGAGGCCACTC | |
| | ACAAGACATCAACTTCACCCATCGTCA | |
| | AGAGCTTCAACAGGAATGAGTGTTAG | |
| | (SEQ ID NO: 238) | |
| Heavy | CAGGTTCAACTGCAGCAGTCTGGGGCT | QVQLQQSGAEVRPGASVTLSCKASGYTFT |
| Chain | GAACTGGTGAGGCCTGGGGCTTCAGTG | DYEMHWVRQTPVHGLEWIGAIDTETGGPAY |
| Variable | ACGCTGTCCTGCAAGGCTTCGGGCTAC | NQKFKGKAILTADKSSSTAYMELRSLTSEDS |
| Region | ACATTTACTGACTATGAAATGCACTGG | AVYFCTRARGSRSVYYFDNWGQGTTLTVSS |
| 5C11.A7 | GTGAGGCAGACACCTGTGCATGGCCTG | (SEQ ID NO: 241) |
| | GAATGGATTGGAGCTATTGATACTGAA | |
| | ACTGGTGGTCCTGCCTACAATCAGAAG | |
| | TTCAAGGGCAAGGCCATACTGACTGCA | |
| | GACAAATCCTCCAGCACAGCCTACATG | |
| | GAGCTCCGCAGCCTGACATCTGAGGAC | |
| | TCTGCCGTCTATTTCTGTACAAGAGCTC | |
| | GCGGTAGTAGGTCCGTGTACTACTTTG | |
| | ACAACTGGGGCCAAGGCACCACTCTCA | |
| | CAGTCTCCTCAG (SEQ ID NO: 240) | |
| Light | GACATTGTGATGACCCAGTCTCAAAAA | DIVMTQSQKFMSTSVGDRVSVTCKASQNVG |
| Chain | TTCATGTCCACATCAGTGGGAGACAGG | TNVAWYQQKPGHSPKVLIYSASYRHSGVPD |
| Variable | GTCAGCGTCACCTGCAAGGCCAGTCAG | RFTGSGSGTDFTLTIGNVQSEDLADYFCQQY |
| Region | AATGTGGGTACTAATGTAGCCTGGTAT | DTYPLTFGSGTKLEIK (SEQ ID NO: 243) |
| 5C11.A7 | CAGCAGAAACCAGGGCACTCTCCTAAA | |
| | GTACTGATTTACTCGGCATCCTACCGG | |
| | CACAGTGGAGTCCCTGATCGCTTCACA | |
| | GGCAGTGGATCTGGGACAGATTTCACT | |
| | CTCACCATCGGCAATGTGCAGTCTGAG | |
| | GACTTGGCAGACTATTTCTGTCAGCAA | |
| | TATGACACCTATCCTCTCACGTTCGGCT | |
| | CGGGGACAAAGTTGGAAATAAAAC | |
| | (SEQ ID NO: 242) | |

Underlined residues indicate position(s) that were altered in the variant of clone
5C11.A7.

TABLE 26
CDR Sequences of anti-IBP4 antibody 5C11.A7 and variant 5C11.A7.

| | Region | | | |
|---|---|---|---|---|
| | Definition | CDR1 | CDR2 | CDR3 |
| Heavy | IMGT | GYTFTDYE | IDTETGGP | TRARGSRSVYYFDN |
| Chain | | (SEQ ID NO: 244) | (SEQ ID NO: 245) | (SEQ ID NO: 246) |
| | Chothia | GYTFTDYEMH | AIDTETGGPAYNQKFKG | ARGSRSVYYFDN |
| | | (SEQ ID NO: 247) | (SEQ ID NO: 248) | (SEQ ID NO: 249) |
| | AbM | GYTFTDYEMHWVR | IGAIDTETGGPAYNQKFKGKA | RARGSRSVYYFDNW |
| | | (SEQ ID NO: 250) | (SEQ ID NO: 251) | (SEQ ID NO: 252) |
| | Contact | TDYEMHWVR | LEWIGAIDTETGGPAYNQKFKGKA | CTRARGSRSVYYFDN |
| | | (SEQ ID NO: 253) | (SEQ ID NO: 254) | (SEQ ID NO: 255) |
| | Kabat | YEMHWVR | IGAIDTETGGPAYNQKFKGKAILT | ARGSRSVYYFDNW |
| | | (SEQ ID NO: 256) | (SEQ ID NO: 257) | (SEQ ID NO: 258) |
| Light | IMGT | QNVGTN | SAS | QQYDTYPLT |
| Chain | | (SEQ ID NO: 87) | (SEQ ID NO: 24) | (SEQ ID NO: 259) |
| | Chothia | KASQNVGTNVA | SASYRH | QQYDTYPLT |
| | | (SEQ ID NO: 90) | (SEQ ID NO: 260) | (SEQ ID NO: 259) |
| | AbM | TCKASQNVGTNVAWY | SASYRHSGVPD | FCQQYDTYPLTF |
| | | (SEQ ID NO: 93) | (SEQ ID NO: 261) | (SEQ ID NO: 262) |
| | Contact | NVGTNVAWYQQ | VLIYSASYRHSGVP | FCQQYDTYPLT |
| | | (SEQ ID NO: 96) | (SEQ ID NO: 263) | (SEQ ID NO: 264) |
| | Kabat | TCKASQNVGTNVAWY | SASYRHSGVPD | QQYDTYPLT |
| | | (SEQ ID NO: 93) | (SEQ ID NO: 261) | (SEQ ID NO: 259) |

TABLE 27
Sequences of heavy chain variable (VH) region and light chain
variable (VL) region of anti-IBP4 antibody 7G11.H4 and variant 7G11.H4.

| | Nucleotide Sequence | Amino Acid Sequence |
|---|---|---|
| Heavy | CAGGTTCAACTGCAGCAGTCTGGGGCT | QVQLQQSGAELVRPGASVTLSCKASGYTFT |
| Chain | GAACTGGTGAGGCCTGGGGCTTCAGTG | DYEMHWVRQTPVHGLEWIGAIDTETGGPAY |
| 7G11.H4 | ACGCTGTCCTGCAAGGCTTCGGGCTAC | NQKFKGKAILTADKSSSTAYMELRSLTSEDS |
| | ACATTTACTGACTATGAAATGCACTGG | AVYFCTRARGSRSVYYFDNWGQGTTLTVSS |
| | GTGAGGCAGACACCTGTGCATGGCCTG | SKTTPPSVYPLAPGSAAQTNSMVTLGCLVKG |
| | GAATGGATTGGAGCTATTGATACTGAA | YFPEPVTVTWNSGSLSSGVHTFPAVLQSDLY |
| | ACTGGTGGTCCTGCCTACAATCAGAAG | TLSSSVTVPSSTWPSQTVTCNVAHPASSTKV |
| | TTCAAGGGCAAGGCCATACTGACTGCA | DKKIVPRDCGCKPCICTVPEVSSVFIFPPKPK |
| | GACAAATCCTCCAGCACAGCCTACATG | DVLTITLTPKVTCVVVDISKDDPEVQFSWFV |
| | GAGCTCCGCAGCCTGACATCTGAGGAC | DDVEVHTAQTKPREEQINSTFRSVSELPIMH |
| | TCTGCCGTCTATTTCTGTACAAGAGCTC | QDWLNGKEFKCRVNSAAFPAPIEKTISKTKG |
| | GCGGTAGTAGGTCCGTGTACTACTTTG | RPKAPQVYTIPPPKEQMAKDKVSLTCMITNF |
| | ACAACTGGGGCCAAGGCACCACTCTCA | FPEDITVEWQWNGQPAENYKNTQPIMDTDG |
| | CAGTCTCCTCATCCAAAACGACACCCC | SYFVYSKLNVQKSNWEAGNTFTCSVLHEGL |
| | CATCTGTCTATCCACTGGCCCCTGGAT | HNHHTEKSLSHSPGK |
| | CTGCTGCCCAAACTAACTCCATGGTGA | (SEQ ID NO: 266) |
| | CCCTGGGATGCCTGGTCAAGGGCTATT | |
| | TCCCTGAGCCAGTGACAGTGACCTGGA | |
| | ACTCTGGATCCCTGTCCAGCGGTGTGC | |
| | ACACCTTCCCAGCTGTCCTGCAGTCTG | |
| | ACCTCTACACTCTGAGCAGCTCAGTGA | |
| | CTGTCCCCTCCAGCACCTGGCCCAGCC | |
| | AGACCGTCACCTGCAACGTTGCCCACC | |
| | CGGCCAGCAGCACCAAGGTGGACAAG | |
| | AAAATTGTGCCCAGGGATTGTGGTTGT | |
| | AAGCCTTGCATATGTACAGTCCCAGAA | |
| | GTATCATCTGTCTTCATCTTCCCCCCAA | |
| | AGCCCAAGGATGTGCTCACCATTACTC | |
| | TGACTCCTAAGGTCACGTGTGTTGTGG | |
| | TAGACATCAGCAAGGATGATCCCGAG | |
| | GTCCAGTTCAGCTGGTTTGTAGATGAT | |
| | GTGGAGGTGCACACAGCTCAGACGAA | |
| | ACCCCGGGAGGAGCAGATCAACAGCA | |
| | CTTTCCGTTCAGTCAGTGAACTTCCCAT | |
| | CATGCACCAGGACTGGCTCAATGGCAA | |
| | GGAGTTCAAATGCAGGGTCAACAGTGC | |
| | AGCTTTCCCTGCCCCCATCGAGAAAAC | |
| | CATCTCCAAAACCAAAGGCAGACCGA | |
| | AGGCTCCACAGGTGTACACCATTCCAC | |
| | CTCCCAAGGAGCAGATGGCCAAGGAT | |
| | AAAGTCAGTCTGACCTGCATGATAACA | |
| | AACTTCTTCCCTGAAGACATTACTGTG | |
| | GAGTGGCAGTGGAATGGGCAGCCAGC | |
| | GGAGAACTACAAGAACACTCAGCCCA | |
| | TCATGGACACAGATGGCTCTTACTTCG | |
| | TCTACAGCAAGCTCAATGTGCAGAAGA | |
| | GCAACTGGGAGGCAGGAAATACTTTCA | |
| | CCTGCTCTGTGTTACATGAGGGCCTGC | |
| | ACAACCACCATACTGAGAAGAGCCTCT | |
| | CCCACTCTCCTGGTAAATGA (SEQ ID | |
| | NO: 265) | |
| Heavy | CAGGTTCAACTGCAGCAGTCTGGGGCT | QVQLQQSGAELVRPGASVTLSCKASGYTFT |
| Chain of | GAACTGGTGAGGCCTGGGGCTTCAGTG | DYEMHWVRQTPVHGLEWIGAIDTETGGPAY |
| variant | ACGCTGTCCTGCAAGGCTTCGGGCTAC | NQKFKGKAILTADKSSSTAYMELRSLTSEDS |
| 7G11.H4 | ACATTTACTGACTATGAAATGCACTGG | AVYFCTRARGSRSVYYFDNWGQGTTLTVSS |
| | GTGAGGCAGACACCTGTGCATGGCCTG | SKTTPPSVYPLAPGSAAQTNSMVTLGCLVKG |
| | GAATGGATTGGAGCTATTGATACTGAA | YFPEPVTVTWNSGSLSSGVHTFPAVLQSDLY |
| | ACTGGTGGTCCTGCCTACAATCAGAAG | TLSSSVTVPSSTWPSQTVTCNVAHPASSTKV |
| | TTCAAGGGCAAGGCCATACTGACTGCA | DKKIVPRDCGCKPCICTVPEVSSVFIFPPKPK |
| | GACAAATCCTCCAGCACAGCCTACATG | DVLTITLTPKVTCVVVDISKDDPEVQFSWFV |
| | GAGCTCCGCAGCCTGACATCTGAGGAC | DDVEVHTAQTKPREEQINSTFRSVSELPIMH |
| | TCTGCCGTCTATTTCTGTACAAGAGCTC | QDWLNGKEFKCRVNSAAFPAPIEKTISKTKG |
| | GCGGTAGTAGGTCCGTGTACTACTTTG | RPKAPQVYTIPPPKEQMAKDKVSLTCMITNF |
| | ACAACTGGGGCCAAGGCACCACTCTCA | FPEDITVEWQWNGQPAENYKNTQPIMDTDG |
| | CAGTCTCCTCATCCAAAACGACACCCC | SYFVYSKLNVQKSNWEAGNTFTCSVLHEGL |
| | CATCTGTCTATCCACTGGCCCCTGGAT | HNHHTEKSLSHSPGK |
| | CTGCTGCCCAAACTAACTCCATGGTGA | (SEQ ID NO: 337) |
| | CCCTGGGATGCCTGGTCAAGGGCTATT | |
| | TCCCTGAGCCAGTGACAGTGACCTGGA | |
| | ACTCTGGATCCCTGTCCAGCGGTGTGC | |
| | ACACCTTCCCAGCTGTCCTGCAGTCTG | |
| | ACCTCTACACTCTGAGCAGCTCAGTGA | |
| | CTGTCCCCTCCAGCACCTGGCCCAGCC | |
| | AGACCGTCACCTGCAACGTTGCCCACC | |
| | CGGCCAGCAGCACCAAGGTGGACAAG | |
| | AAAATTGTGCCCAGGGATTGTGGTTGT | |
| | AAGCCTTGCATATGTACAGTCCCAGAA | |
| | GTATCATCTGTCTTCATCTTCCCCCCAA | |
| | AGCCCAAGGATGTGCTCACCATTACTC | |
| | TGACTCCTAAGGTCACGTGTGTTGTGG | |
| | TAGACATCAGCAAGGATGATCCCGAG | |
| | GTCCAGTTCAGCTGGTTTGTAGATGAT | |
| | GTGGAGGTGCACACAGCTCAGACGAA | |
| | ACCCCGGGAGGAGCAGATCAACAGCA | |
| | CTTTCCGTTCAGTCAGTGAACTTCCCAT | |
| | CATGCACCAGGACTGGCTCAATGGCAA | |
| | GGAGTTCAAATGCAGGGTCAACAGTGC | |
| | AGCTTTCCCTGCCCCCATCGAGAAAAC | |
| | CATCTCCAAAACCAAAGGCAGACCGA | |
| | AGGCTCCACAGGTGTACACCATTCCAC | |
| | CTCCCAAGGAGCAGATGGCCAAGGAT | |
| | AAAGTCAGTCTGACCTGCATGATAACA | |
| | AACTTCTTCCCTGAAGACATTACTGTG | |
| | GAGTGGCAGTGGAATGGGCAGCCAGC | |
| | GGAGAACTACAAGAACACTCAGCCCA | |
| | TCATGGACACAGATGGCTCTTACTTCG | |
| | TCTACAGCAAGCTCAATGTGCAGAAGA | |
| | GCAACTGGGAGGCAGGAAATACTTTCA | |
| | CCTGCTCTGTGTTACATGAGGGCCTGC | |
| | ACAACCACCATACTGAGAAGAGCCTCT | |
| | CCCACTCTCCTGGTTGA | |
| | (SEQ ID NO: 338) | |
| Light | GACATTGTGATGACCCAGTCTCAAAAA | DIVMTQSQKFMSTSVGDRVSVTCKASQNVD |
| Chain | TTCATGTCCACATCAGTGGGAGACAGG | TNIAWYQQKPGHSPKALIYSASYRYSGVPDR |
| 7G11.H4 | GTCAGCGTCACCTGCAAGGCCAGTCAG | FTGSGSGTDFTLTIGNVQSEDLADYFCQQYD |
| | AATGTGGATACTAATATAGCCTGGTAT | TYPLTFGSGTKLEIKRADAAPTVSIFPPSSEQL |
| | CAGCAGAAACCAGGGCACTCTCCTAAA | TSGGASVVCFLNNFYPRDINVKWKIDGSERQ |
| | GCACTGATTTACTCGGCATCCTACCGG | NGVLNSWTDQDSKDSTYSMSSTLTLTKDEY |
| | TACAGTGGAGTCCCTGATCGCTTCACA | ERHNSYTCEATHKTSTSPIVKSFNRNEC |
| | GGCAGTGGATCTGGGACAGATTTCACT | (SEQ ID NO: 268) |
| | CTCACCATCGGCAATGTGCAGTCTGAG | |
| | GACTTGGCAGACTATTTCTGTCAGCAA | |
| | TATGACACCTATCCTCTCACGTTCGGCT | |
| | CGGGGACAAAGTTGGAAATAAAACGG | |
| | GCTGATGCTGCACCAACTGTATCCATC | |
| | TTCCCACCATCCAGTGAGCAGTTAACA | |
| | TCTGGAGGTGCCTCAGTCGTGTGCTTC | |
| | TTGAACAACTTCTACCCCAGAGACATC | |
| | AATGTCAAGTGGAAGATTGATGGCAGT | |
| | GAACGACAAAATGGTGTCCTGAACAGT | |
| | TGGACTGATCAGGACAGCAAAGACAG | |
| | CACCTACAGCATGAGCAGCACCCTCAC | |
| | ATTGACCAAGGACGAGTATGAACGAC | |
| | ATAACAGCTATACCTGTGAGGCCACTC | |
| | ACAAGACATCAACTTCACCCATCGTCA | |
| | AGAGCTTCAACAGGAATGAGTGTTAG | |
| | (SEQ ID NO: 267) | |
| Light | GACATTGTGATGACCCAGTCTCAAAAA | DIVMTQSQKFMSTSVGDRVSVTCKASQNVD |
| Chain of | TTCATGTCCACATCAGTGGGAGACAGG | TNIAWYQQKPGHSPKALIYSASYRYSGVPDR |
| variant | GTCAGCGTCACCTGCAAGGCCAGTCAG | FTGSGSGTDFTLTIGNVQSEDLADYFCQQYD |
| 7G11.H4 | AATGTGGATACTAATATAGCCTGGTAT | TYPLTFGSGTKLEIKRADAAPTVSIFPPSSEQL |
| | CAGCAGAAACCAGGGCACTCTCCTAAA | TSGGASVVCFLNNFYPRDINVKWKIDGSERQ |
| | GCACTGATTTACTCGGCATCCTACCGG | NGVLNSWTDQDSKDSTYSMSSTLTLTKDEY |
| | TACAGTGGAGTCCCTGATCGCTTCACA | ERHNSYTCEATHKTSTSPIVKSFNRNEC (SEQ |
| | GGCAGTGGATCTGGGACAGATTTCACT | ID NO: 268) |
| | CTCACCATCGGCAATGTGCAGTCTGAG | |
| | GACTTGGCAGACTATTTCTGTCAGCAA | |
| | TATGACACCTATCCTCTCACGTTCGGCT | |
| | CGGGGACAAAGTTGGAAATAAAACGG | |
| | GCTGATGCTGCACCAACTGTATCCATC | |
| | TTCCCACCATCCAGTGAGCAGTTAACA | |
| | TCTGGAGGTGCCTCAGTCGTGTGCTTC | |
| | TTGAACAACTTCTACCCCAGAGACATC | |
| | AATGTCAAGTGGAAGATTGATGGCAGT | |
| | GAACGACAAAATGGTGTCCTGAACAGT | |
| | TGGACTGATCAGGACAGCAAAGACAG | |
| | CACCTACAGCATGAGCAGCACCCTCAC | |
| | ATTGACCAAGGACGAGTATGAACGAC | |
| | ATAACAGCTATACCTGTGAGGCCACTC | |
| | ACAAGACATCAACTTCACCCATCGTCA | |
| | AGAGCTTCAACAGGAATGAGTGTTAG | |
| | (SEQ ID NO: 267) | |
| Heavy | CAGGTTCAACTGCAGCAGTCTGGGGCT | QVQLQQSGAELVRPGASVTLSCKASGYTFT |
| Chain | GAACTGGTGAGGCCTGGGGCTTCAGTG | DYEMHWVRQTPVHGLEWIGAIDTETGGPAY |
| Variable | ACGCTGTCCTGCAAGGCTTCGGGCTAC | NQKFKGKAILTADKSSSTAYMELRSLTSEDS |
| Region | ACATTTACTGACTATGAAATGCACTGG | AVYFCTRARGSRSVYYFDNWGQGTTLTVSS |
| 7G11.H4 | GTGAGGCAGACACCTGTGCATGGCCTG | (SEQ ID NO: 241) |
| | GAATGGATTGGAGCTATTGATACTGAA | |
| | ACTGGTGGTCCTGCCTACAATCAGAAG | |
| | TTCAAGGGCAAGGCCATACTGACTGCA | |
| | GACAAATCCTCCAGCACAGCCTACATG | |
| | GAGCTCCGCAGCCTGACATCTGAGGAC | |
| | TCTGCCGTCTATTTCTGTACAAGAGCTC | |
| | GCGGTAGTAGGTCCGTGTACTACTTTG | |
| | ACAACTGGGGCCAAGGCACCACTCTCA | |
| | CAGTCTCCTCAT (SEQ ID NO: 269) | |
| Light | GACATTGTGATGACCCAGTCTCAAAAA | DIVMTQSQKFMSTSVGDRVSVTCKASQNVD |
| Chain | TTCATGTCCACATCAGTGGGAGACAGG | TNIAWYQQKPGHSPKALIYSASYRYSGVPDR |
| Variable | GTCAGCGTCACCTGCAAGGCCAGTCAG | FTGSGSGTDFTLTIGNVQSEDLADYFCQQYD |
| Region | AATGTGGATACTAATATAGCCTGGTAT | TYPLTFGSGTKLEIK (SEQ ID NO: 271) |
| 7G11.H4 | CAGCAGAAACCAGGGCACTCTCCTAAA | |
| | GCACTGATTTACTCGGCATCCTACCGG | |
| | TACAGTGGAGTCCCTGATCGCTTCACA | |
| | GGCAGTGGATCTGGGACAGATTTCACT | |
| | CTCACCATCGGCAATGTGCAGTCTGAG | |
| | GACTTGGCAGACTATTTCTGTCAGCAA | |
| | TATGACACCTATCCTCTCACGTTCGGCT | |
| | CGGGGACAAAGTTGGAAATAAAAC | |
| | (SEQ ID NO: 270) | |

Underlined residues indicate position(s) that were altered in the variant of clone
7G11.H4.

TABLE 28
CDR Sequences of anti-IBP4 antibody 7G11.H4 and variant 7G11.H4.

| | Region | | | |
|---|---|---|---|---|
| | Definition | CDR1 | CDR2 | CDR3 |
| Heavy | IMGT | GYTFTDYE | IDTETGGP | TRARGSRSVYYFDN |
| Chain | | (SEQ ID NO: 244) | (SEQ ID NO: 245) | (SEQ ID NO: 246) |
| | Chothia | GYTFTDYEMH | AIDTETGGPAYNQKFKG | ARGSRSVYYFDN |
| | | (SEQ ID NO: 247) | (SEQ ID NO: 248) | (SEQ ID NO: 249) |
| | AbM | GYTFTDYEMHWVR | IGAIDTETGGPAYNQKFKGKA | RARGSRSVYYFDNW |
| | | (SEQ ID NO: 250) | (SEQ ID NO: 251) | (SEQ ID NO: 252) |
| | Contact | TDYEMHWVR | LEWIGAIDTETGGPAYNQKFKGKA | CTRARGSRSVYYFDN |
| | | (SEQ ID NO: 253) | (SEQ ID NO: 254) | (SEQ ID NO: 255) |
| | Kabat | YEMHWVR | IGAIDTETGGPAYNQKFKGKAILT | ARGSRSVYYFDNW |
| | | (SEQ ID NO: 256) | (SEQ ID NO: 257) | (SEQ ID NO: 258) |
| Light | IMGT | QNVDTN | SAS | QQYDTYPLT |
| Chain | | (SEQ ID NO: 23) | (SEQ ID NO: 24) | (SEQ ID NO: 259) |
| | Chothia | KASQNVDTNIA | SASYRY | QQYDTYPLT |
| | | (SEQ ID NO: 272) | (SEQ ID NO: 273) | (SEQ ID NO: 259) |
| | AbM | TCKASQNVDTNIAWY | SASYRYSGVPD | FCQQYDTYPLTF |
| | | (SEQ ID NO: 274) | (SEQ ID NO: 94) | (SEQ ID NO: 262) |
| | Contact | NVDTNIAWYQQ | ALIYSASYRYSGVP | FCQQYDTYPLT |
| | | (SEQ ID NO: 275) | (SEQ ID NO: 97) | (SEQ ID NO: 264) |
| | Kabat | TCKASQNVDTNIAWY | SASYRYSGVPD | QQYDTYPLT |
| | | (SEQ ID NO: 274) | (SEQ ID NO: 94) | (SEQ ID NO: 259) |

TABLE 29
Sequences of heavy chain variable (VH) region and light chain
variable (VL) region of anti-IBP4 antibody 3D4.E9 and variant 3D4.E9.

| | Nucleotide Sequence | Amino Acid Sequence |
|---|---|---|
| Heavy | CAGGTTCAACTGCAGCAGTCTGGGGCT | QVQLQQSGAELVRPGASVTLSCKASGYTFT |
| Chain | GAGCTGGTGAGGCCTGGGGCTTCAGTG | DYEMHWVKQTPVHGLEWIGAIDPETGGAA |
| 3D4.E9 | ACGCTGTCCTGCAAGGCTTCGGGCTAC | YNQKFKGKAILTADKSSSTAYMELRSLTSED |
| | ACATTTACTGACTATGAAATGCACTGG | SAVYYCTRARGSSSVYYFDYWGQGTTLTVS |
| | GTGAAGCAGACACCTGTGCATGGCCTG | SAKTTPPSVYPLAPGCGDTTGSSVTLGCLVK |
| | GAATGGATTGGAGCTATTGATCCTGAA | GYFPESVTVTWNSGSLSSSVHTFPALLQSGL |
| | ACTGGTGGTGCTGCCTACAATCAGAAG | YTMSSSVTVPSSTWPSQTVTCSVAHPASSTT |
| | TTCAAGGGCAAGGCCATACTGACTGCA | VDKKLEPSGPISTINPCPPCKECHKCPAPNLE |
| | GACAAATCCTCCAGCACAGCCTACATG | GGPSVFIFPPNIKDVLMISLTPKVTCVVVDVS |
| | GAGCTCCGCAGCCTGACATCTGAGGAC | EDDPDVRISWFVNNVEVHTAQTQTHREDYN |
| | TCTGCCGTCTATTATTGTACAAGAGCT | STIRVVSALPIQHQDWMSGKEFKCKVNNKD |
| | CGCGGTAGTAGCTCCGTGTACTACTTT | LPSPIERTISKIKGLVRAPQVYILPPPAEQLSR |
| | GACTACTGGGGCCAAGGCACCACTCTC | KDVSLTCLVVGFNPGDISVEWTSNGHTEENY |
| | ACAGTCTCCTCAGCCAAAACAACACCC | KDTAPVLDSDGSYFIYSKLDIKTSKWEKTDS |
| | CCATCAGTCTATCCACTGGCCCCTGGG | FSCNVRHEGLKNYYLKKTISRSPGK (SEQ ID |
| | TGTGGAGATACAACTGGTTCCTCTGTG | NO: 277) |
| | ACTCTGGGATGCCTGGTCAAGGGCTAC | |
| | TTCCCTGAGTCAGTGACTGTGACTTGG | |
| | AACTCTGGATCCCTGTCCAGCAGTGTG | |
| | CACACCTTCCCAGCTCTCCTGCAGTCT | |
| | GGACTCTACACTATGAGCAGCTCAGTG | |
| | ACTGTCCCCTCCAGCACCTGGCCAAGT | |
| | CAGACCGTCACCTGCAGCGTTGCTCAC | |
| | CCAGCCAGCAGCACCACGGTGGACAA | |
| | AAAACTTGAGCCCAGCGGGCCCATTTC | |
| | AACAATCAACCCCTGTCCTCCATGCAA | |
| | GGAGTGTCACAAATGCCCAGCTCCTAA | |
| | CCTCGAGGGTGGACCATCCGTCTTCAT | |
| | CTTCCCTCCAAATATCAAGGATGTACT | |
| | CATGATCTCCCTGACACCCAAGGTCAC | |
| | GTGTGTGGTGGTGGATGTGAGCGAGGA | |
| | TGACCCAGACGTCCGGATCAGCTGGTT | |
| | TGTGAACAACGTGGAAGTACACACAG | |
| | CTCAGACACAAACCCATAGAGAGGATT | |
| | ACAACAGTACTATCCGGGTGGTCAGTG | |
| | CCCTCCCCATCCAGCACCAGGACTGGA | |
| | TGAGTGGCAAGGAGTTCAAATGCAAG | |
| | GTCAACAACAAAGACCTCCCATCACCC | |
| | ATCGAGAGAACCATCTCAAAAATTAAA | |
| | GGGCTAGTCAGAGCTCCACAAGTATAC | |
| | ATCTTGCCGCCACCAGCAGAGCAGTTG | |
| | TCCAGGAAAGATGTCAGTCTCACTTGC | |
| | CTGGTCGTGGGCTTCAACCCTGGAGAC | |
| | ATCAGTGTGGAGTGGACCAGCAATGG | |
| | GCATACAGAGGAGAACTACAAGGACA | |
| | CCGCACCAGTCCTGGACTCTGACGGTT | |
| | CTTACTTCATATACAGCAAGCTCGATA | |
| | TAAAAACAAGCAAGTGGGAGAAAACA | |
| | GATTCCTTCTCATGCAACGTGAGACAC | |
| | GAGGGTCTGAAAAATTACTACCTGAAG | |
| | AAGACCATCTCCCGGTCTCCGGGTAAA | |
| | TGA (SEQ ID NO: 276) | |
| Heavy | CAGGTTCAACTGCAGCAGTCTGGGGCT | QVQLQQSGAELVRPGASVTLSCKASGYTFT |
| Chain of | GAGCTGGTGAGGCCTGGGGCTTCAGTG | DYEMHWVKQTPVHGLEWIGAIDPETGGAA |
| variant | ACGCTGTCCTGCAAGGCTTCGGGCTAC | YNQKFKGKAILTADKSSSTAYMELRSLTSED |
| 3D4.E9 | ACATTTACTGACTATGAAATGCACTGG | SAVYYCTRARGSSSVYYFDYWGQGTTLTVS |
| | GTGAAGCAGACACCTGTGCATGGCCTG | SAKTTPPSVYPLAPGCGDTTGSSVTLGCLVK |
| | GAATGGATTGGAGCTATTGATCCTGAA | GYFPESVTVTWNSGSLSSSVHTFPALLQSGL |
| | ACTGGTGGTGCTGCCTACAATCAGAAG | YTMSSSVTVPSSTWPSQTVTCSVAHPASSTT |
| | TTCAAGGGCAAGGCCATACTGACTGCA | VDKKLEPSGPISTINPCPPCKECHKCPAPNLE |
| | GACAAATCCTCCAGCACAGCCTACATG | GGPSVFIFPPNIKDVLMISLTPKVTCVVVDVS |
| | GAGCTCCGCAGCCTGACATCTGAGGAC | EDDPDVRISWFVNNVEVHTAQTQTHREDYN |
| | TCTGCCGTCTATTATTGTACAAGAGCT | STIRVVSALPIQHQDWMSGKEFKCKVNNKD |
| | CGCGGTAGTAGCTCCGTGTACTACTTT | LPSPIERTISKIKGLVRAPQVYILPPPAEQLSR |
| | GACTACTGGGGCCAAGGCACCACTCTC | KDVSLTCLVVGFNPGDISVEWTSNGHTEENY |
| | ACAGTCTCCTCAGCCAAAACAACACCC | KDTAPVLDSDGSYFIYSKLDIKTSKWEKTDS |
| | CCATCAGTCTATCCACTGGCCCCTGGG | FSCNVRHEGLKNYYLKKTISRSPG (SEQ ID |
| | TGTGGAGATACAACTGGTTCCTCTGTG | NO: 339) |
| | ACTCTGGGATGCCTGGTCAAGGGCTAC | |
| | TTCCCTGAGTCAGTGACTGTGACTTGG | |
| | AACTCTGGATCCCTGTCCAGCAGTGTG | |
| | CACACCTTCCCAGCTCTCCTGCAGTCT | |
| | GGACTCTACACTATGAGCAGCTCAGTG | |
| | ACTGTCCCCTCCAGCACCTGGCCAAGT | |
| | CAGACCGTCACCTGCAGCGTTGCTCAC | |
| | CCAGCCAGCAGCACCACGGTGGACAA | |
| | AAAACTTGAGCCCAGCGGGCCCATTTC | |
| | AACAATCAACCCCTGTCCTCCATGCAA | |
| | GGAGTGTCACAAATGCCCAGCTCCTAA | |
| | CCTCGAGGGTGGACCATCCGTCTTCAT | |
| | CTTCCCTCCAAATATCAAGGATGTACT | |
| | CATGATCTCCCTGACACCCAAGGTCAC | |
| | GTGTGTGGTGGTGGATGTGAGCGAGGA | |
| | TGACCCAGACGTCCGGATCAGCTGGTT | |
| | TGTGAACAACGTGGAAGTACACACAG | |
| | CTCAGACACAAACCCATAGAGAGGATT | |
| | ACAACAGTACTATCCGGGTGGTCAGTG | |
| | CCCTCCCCATCCAGCACCAGGACTGGA | |
| | TGAGTGGCAAGGAGTTCAAATGCAAG | |
| | GTCAACAACAAAGACCTCCCATCACCC | |
| | ATCGAGAGAACCATCTCAAAAATTAAA | |
| | GGGCTAGTCAGAGCTCCACAAGTATAC | |
| | ATCTTGCCGCCACCAGCAGAGCAGTTG | |
| | TCCAGGAAAGATGTCAGTCTCACTTGC | |
| | CTGGTCGTGGGCTTCAACCCTGGAGAC | |
| | ATCAGTGTGGAGTGGACCAGCAATGG | |
| | GCATACAGAGGAGAACTACAAGGACA | |
| | CCGCACCAGTCCTGGACTCTGACGGTT | |
| | CTTACTTCATATACAGCAAGCTCGATA | |
| | TAAAAACAAGCAAGTGGGAGAAAACA | |
| | GATTCCTTCTCATGCAACGTGAGACAC | |
| | GAGGGTCTGAAAAATTACTACCTGAAG | |
| | AAGACCATCTCCCGGTCTCCGGGTTGA | |
| | (SEQ ID NO: 340) | |
| | GACATTGTGATGACCCAGTCTCAAAAA | DIVMTQSQKFMSTSVGDRVSVTCKASQNVG |
| | TTCATGTCCACATCAGTAGGAGACAGG | TNVAWYQQKPGQSPKALIYSTSYRYSGVPD |
| | GTCAGCGTCACCTGCAAGGCCAGTCAG | RFTGSRSGTDFILTISNVQSEDLAEYFCQQYD |
| Light | AATGTGGGTACTAATGTAGCCTGGTAT | SYPLTFGSGTKLEIKRADAAPTVSIFPPSSEQL |
| Chain | CAACAGAAACCAGGGCAATCTCCTAA | TSGGASVVCFLNNFYPRDINVKWKIDGSERQ |
| 3D4.E9 | AGCACTGATTTACTCGACATCCTACCG | NGVLNSWTDQDSKDSTYSMSSTLTLTKDEY |
| | GTACAGTGGAGTCCCTGATCGCTTCAC | ERHNSYTCEATHKTSTSPIVKSFNRNEC |
| | AGGCAGTAGATCTGGGACAGATTTCAT | (SEQ ID NO: 279) |
| | TCTCACCATCAGCAATGTGCAGTCTGA | |
| | AGACTTGGCAGAGTATTTCTGTCAGCA | |
| | ATATGACAGCTATCCTCTCACGTTCGG | |
| | CTCGGGGACAAAGTTGGAAATAAAAC | |
| | GGGCTGATGCTGCACCAACTGTATCCA | |
| | TCTTCCCACCATCCAGTGAGCAGTTAA | |
| | CATCTGGAGGTGCCTCAGTCGTGTGCT | |
| | TCTTGAACAACTTCTACCCCAGAGACA | |
| | TCAATGTCAAGTGGAAGATTGATGGCA | |
| | GTGAACGACAAAATGGTGTCCTGAACA | |
| | GTTGGACTGATCAGGACAGCAAAGAC | |
| | AGCACCTACAGCATGAGCAGCACCCTC | |
| | ACATTGACCAAGGACGAGTATGAACG | |
| | ACATAACAGCTATACCTGTGAGGCCAC | |
| | TCACAAGACATCAACTTCACCCATCGT | |
| | CAAGAGCTTCAACAGGAATGAGTGTTA | |
| | G (SEQ ID NO: 278) | |
| Light | GACATTGTGATGACCCAGTCTCAAAAA | DIVMTQSQKFMSTSVGDRVSVTCKASQNVG |
| Chain of | TTCATGTCCACATCAGTAGGAGACAGG | TNVAWYQQKPGQSPKALIYSTSYRYSGVPD |
| variant | GTCAGCGTCACCTGCAAGGCCAGTCAG | RFTGSRSGTDFILTISNVQSEDLAEYFCQQYD |
| 3D4.E9 | AATGTGGGTACTAATGTAGCCTGGTAT | SYPLTFGSGTKLEIKRADAAPTVSIFPPSSEQL |
| | CAACAGAAACCAGGGCAATCTCCTAA | TSGGASVVCFLNNFYPRDINVKWKIDGSERQ |
| | AGCACTGATTTACTCGACATCCTACCG | NGVLNSWTDQDSKDSTYSMSSTLTLTKDEY |
| | GTACAGTGGAGTCCCTGATCGCTTCAC | ERHNSYTCEATHKTSTSPIVKSFNRNEC (SEQ |
| | AGGCAGTAGATCTGGGACAGATTTCAT | ID NO: 279) |
| | TCTCACCATCAGCAATGTGCAGTCTGA | |
| | AGACTTGGCAGAGTATTTCTGTCAGCA | |
| | ATATGACAGCTATCCTCTCACGTTCGG | |
| | CTCGGGGACAAAGTTGGAAATAAAAC | |
| | GGGCTGATGCTGCACCAACTGTATCCA | |
| | TCTTCCCACCATCCAGTGAGCAGTTAA | |
| | CATCTGGAGGTGCCTCAGTCGTGTGCT | |
| | TCTTGAACAACTTCTACCCCAGAGACA | |
| | TCAATGTCAAGTGGAAGATTGATGGCA | |
| | GTGAACGACAAAATGGTGTCCTGAACA | |
| | GTTGGACTGATCAGGACAGCAAAGAC | |
| | AGCACCTACAGCATGAGCAGCACCCTC | |
| | ACATTGACCAAGGACGAGTATGAACG | |
| | ACATAACAGCTATACCTGTGAGGCCAC | |
| | TCACAAGACATCAACTTCACCCATCGT | |
| | CAAGAGCTTCAACAGGAATGAGTGTTA | |
| | G (SEQ ID NO: 278) | |
| Heavy | CAGGTTCAACTGCAGCAGTCTGGGGCT | QVQLQQSGAELVRPGASVTLSCKASGYTFT |
| Chain | GAGCTGGTGAGGCCTGGGGCTTCAGTG | DYEMHWVKQTPVHGLEWIGAIDPETGGAA |
| Variable | ACGCTGTCCTGCAAGGCTTCGGGCTAC | YNQKFKGKAILTADKSSSTAYMELRSLTSED |
| Region | ACATTTACTGACTATGAAATGCACTGG | SAVYYCTRARGSSSVYYFDYWGQGTTLTVS |
| 3D4.E9 | GTGAAGCAGACACCTGTGCATGGCCTG | S (SEQ ID NO: 281) |
| | GAATGGATTGGAGCTATTGATCCTGAA | |
| | ACTGGTGGTGCTGCCTACAATCAGAAG | |
| | TTCAAGGGCAAGGCCATACTGACTGCA | |
| | GACAAATCCTCCAGCACAGCCTACATG | |
| | GAGCTCCGCAGCCTGACATCTGAGGAC | |
| | TCTGCCGTCTATTATTGTACAAGAGCT | |
| | CGCGGTAGTAGCTCCGTGTACTACTTT | |
| | GACTACTGGGGCCAAGGCACCACTCTC | |
| | ACAGTCTCCTCAG (SEQ ID NO: 280) | |
| Light | GACATTGTGATGACCCAGTCTCAAAAA | DIVMTQSQKFMSTSVGDRVSVTCKASQNVG |
| Chain | TTCATGTCCACATCAGTAGGAGACAGG | TNVAWYQQKPGQSPKALIYSTSYRYSGVPD |
| Variable | GTCAGCGTCACCTGCAAGGCCAGTCAG | RFTGSRSGTDFILTISNVQSEDLAEYFCQQYD |
| Region | AATGTGGGTACTAATGTAGCCTGGTAT | SYPLTFGSGTKLEIK (SEQ ID NO: 283) |
| 3D4.E9 | CAACAGAAACCAGGGCAATCTCCTAA | |
| | AGCACTGATTTACTCGACATCCTACCG | |
| | GTACAGTGGAGTCCCTGATCGCTTCAC | |
| | AGGCAGTAGATCTGGGACAGATTTCAT | |
| | TCTCACCATCAGCAATGTGCAGTCTGA | |
| | AGACTTGGCAGAGTATTTCTGTCAGCA | |
| | ATATGACAGCTATCCTCTCACGTTCGG | |
| | CTCGGGGACAAAGTTGGAAATAAAAC | |
| | (SEQ ID NO: 282) | |

Underlined residues indicate position(s) that were altered in the variant of clone
3D4.E9.

TABLE 30
CDR Sequences of anti-IBP4 antibody 3D4.E9 and variant 3D4.E9.

| | Region | | | |
|---|---|---|---|---|
| | Definition | CDR1 | CDR2 | CDR3 |
| Heavy | IMGT | GYTFTDYE | IDPETGGA | TRARGSSSVYYFDY |
| | | (SEQ ID NO: 244) | (SEQ ID NO: 284) | (SEQ ID NO: 285) |
| Chain | Chothia | GYTFTDYEMH | AIDPETGGAAYNQKFKG | ARGSSSVYYFDY |
| | | (SEQ ID NO: 247) | (SEQ ID NO: 286) | (SEQ ID NO: 287) |
| | AbM | GYTFTDYEMHWVK | IGAIDPETGGAAYNQKFKGKA | RARGSSSVYYFDYW |
| | | (SEQ ID NO: 288) | (SEQ ID NO: 289) | (SEQ ID NO: 290) |
| | Contact | TDYEMHWVK | LEWIGAIDPETGGAAYNQKFKGKA | CTRARGSSSVYYFDY |
| | | (SEQ ID NO: 291) | (SEQ ID NO: 292) | (SEQ ID NO: 293) |
| | Kabat | YEMHWVK | IGAIDPETGGAAYNQKFKGKAILT | ARGSSSVYYFDYW |
| | | (SEQ ID NO: 294) | (SEQ ID NO: 295) | (SEQ ID NO: 296) |
| Light | IMGT | QNVGTN | STS | QQYDSYPLT |
| Chain | | (SEQ ID NO: 87) | (SEQ ID NO: 297) | (SEQ ID NO: 298) |
| | Chothia | KASQNVGTNVA | STSYRY | QQYDSYPLT |
| | | (SEQ ID NO: 90) | (SEQ ID NO: 299) | (SEQ ID NO: 298) |
| | AbM | TCKASQNVGTNVAWY | STSYRYSGVPD | FCQQYDSYPLTF |
| | | (SEQ ID NO: 93) | (SEQ ID NO: 300) | (SEQ ID NO: 301) |
| | Contact | NVGTNVAWYQQ | ALIYSTSYRYSGVP | FCQQYDSYPLT |
| | | (SEQ ID NO: 96) | (SEQ ID NO: 302) | (SEQ ID NO: 303) |
| | Kabat | TCKASQNVGTNVAWY | STSYRYSGVPD | QQYDSYPLT |
| | | (SEQ ID NO: 93) | (SEQ ID NO: 300) | (SEQ ID NO: 298) |

TABLE 31
Sequences of heavy chain variable (VH) region and light chain
variable (VL) region of anti-IBP4 antibody 1G3.A3 and variant 1G3.A3.

| | Nucleotide Sequence | Amino Acid Sequence |
|---|---|---|
| Heavy | CAGGTTCAACTGCAGCAGTCTGGGGCT | QVQLQQSGAELVRPGASVTLSCKASGYTFT |
| Chain | GAGCTGGTGAGGCCTGGGGCTTCAGTG | DYEMHWVKQTPVHGLEWIGAIDPETGGIAY |
| 1G3.A3 | ACGCTGTCCTGCAAGGCTTCGGGCTAC | NOKFRGKAILTADKSSSTAYMELRSLTSEDS |
| | ACATTTACTGACTATGAAATGCACTGG | AVYYCARNYGISSYYAVDYWGQGTSVTVSS |
| | GTGAAACAGACACCTGTGCATGGCCTG | AKTTPPSVYPLAPGSAAQTNSMVTLGCLVK |
| | GAATGGATTGGAGCTATTGATCCTGAA | GYFPEPVTVTWNSGSLSSGVHTFPAVLQSDL |
| | ACTGGTGGTATTGCCTACAATCAGAAG | YTLSSSVTVPSSTWPSQTVTCNVAHPASSTK |
| | TTCAGGGGCAAGGCCATACTGACTGCA | VDKKIVPRDCGCKPCICTVPEVSSVFIFPPKP |
| | GACAAATCCTCCAGCACAGCCTACATG | KDVLTITLTPKVTCVVVDISKDDPEVQFSWF |
| | GAGCTCCGCAGCCTGACATCTGAGGAC | VDDVEVHTAQTKPREEQINSTFRSVSELPIM |
| | TCTGCCGTCTATTACTGTGCAAGAAAC | HQDWLNGKEFKCRVNSAAFPAPIEKTISKTK |
| | TACGGTATTAGCTCCTACTATGCTGTG | GRPKAPQVYTIPPPKEQMAKDKVSLTCMITN |
| | GACTACTGGGGTCAAGGAACCTCAGTC | FFPEDITVEWQWNGQPAENYKNTQPIMDTD |
| | ACCGTCTCCTCAGCCAAAACGACACCC | GSYFVYSKLNVQKSNWEAGNTFTCSVLHEG |
| | CCATCTGTCTATCCACTGGCCCCTGGA | LHNHHTEKSLSHSPGK |
| | TCTGCTGCCCAAACTAACTCCATGGTG | (SEQ ID NO: 305) |
| | ACCCTGGGATGCCTGGTCAAGGGCTAT | |
| | TTCCCTGAGCCAGTGACAGTGACCTGG | |
| | AACTCTGGATCCCTGTCCAGCGGTGTG | |
| | CACACCTTCCCAGCTGTCCTGCAGTCT | |
| | GACCTCTACACTCTGAGCAGCTCAGTG | |
| | ACTGTCCCCTCCAGCACCTGGCCCAGC | |
| | CAGACCGTCACCTGCAACGTTGCCCAC | |
| | CCGGCCAGCAGCACCAAGGTGGACAA | |
| | GAAAATTGTGCCCAGGGATTGTGGTTG | |
| | TAAGCCTTGCATATGTACAGTCCCAGA | |
| | AGTATCATCTGTCTTCATCTTCCCCCCA | |
| | AAGCCCAAGGATGTGCTCACCATTACT | |
| | CTGACTCCTAAGGTCACGTGTGTTGTG | |
| | GTAGACATCAGCAAGGATGATCCCGA | |
| | GGTCCAGTTCAGCTGGTTTGTAGATGA | |
| | TGTGGAGGTGCACACAGCTCAGACGA | |
| | AACCCCGGGAGGAGCAGATCAACAGC | |
| | ACTTTCCGTTCAGTCAGTGAACTTCCC | |
| | ATCATGCACCAGGACTGGCTCAATGGC | |
| | AAGGAGTTCAAATGCAGGGTCAACAG | |
| | TGCAGCTTTCCCTGCCCCCATCGAGAA | |
| | AACCATCTCCAAAACCAAAGGCAGAC | |
| | CGAAGGCTCCACAGGTGTACACCATTC | |
| | CACCTCCCAAGGAGCAGATGGCCAAG | |
| | GATAAAGTCAGTCTGACCTGCATGATA | |
| | ACAAACTTCTTCCCTGAAGACATTACT | |
| | GTGGAGTGGCAGTGGAATGGGCAGCC | |
| | AGCGGAGAACTACAAGAACACTCAGC | |
| | CCATCATGGACACAGATGGCTCTTACT | |
| | TCGTCTACAGCAAGCTCAATGTGCAGA | |
| | AGAGCAACTGGGAGGCAGGAAATACT | |
| | TTCACCTGCTCTGTGTTACATGAGGGC | |
| | CTGCACAACCACCATACTGAGAAGAGC | |
| | CTCTCCCACTCTCCTGGTAAATGA (SEQ | |
| | ID NO: 304) | |
| Heavy | CAGGTTCAACTGCAGCAGTCTGGGGCT | QVQLQQSGAELVRPGASVTLSCKASGYTFT |
| Chain of | GAGCTGGTGAGGCCTGGGGCTTCAGTG | DYEMHWVKQTPVHGLEWIGAIDPETGGIAY |
| variant | ACGCTGTCCTGCAAGGCTTCGGGCTAC | NOKFRGKAILTADKSSSTAYMELRSLTSEDS |
| 1G3.A3 | ACATTTACTGACTATGAAATGCACTGG | AVYYCARNYGISSYYAVDYWGQGTSVTVSS |
| | GTGAAACAGACACCTGTGCATGGCCTG | AKTTPPSVYPLAPGSAAQTNSMVTLGCLVK |
| | GAATGGATTGGAGCTATTGATCCTGAA | GYFPEPVTVTWNSGSLSSGVHTFPAVLQSDL |
| | ACTGGTGGTATTGCCTACAATCAGAAG | YTLSSSVTVPSSTWPSQTVTCNVAHPASSTK |
| | TTCAGGGGCAAGGCCATACTGACTGCA | VDKKIVPRDCGCKPCICTVPEVSSVFIFPPKP |
| | GACAAATCCTCCAGCACAGCCTACATG | KDVLTITLTPKVTCVVVDISKDDPEVQFSWF |
| | GAGCTCCGCAGCCTGACATCTGAGGAC | VDDVEVHTAQTKPREEQINSTFRSVSELPIM |
| | TCTGCCGTCTATTACTGTGCAAGAAAC | HQDWLNGKEFKCRVNSAAFPAPIEKTISKTK |
| | TACGGTATTAGCTCCTACTATGCTGTG | GRPKAPQVYTIPPPKEQMAKDKVSLTCMITN |
| | GACTACTGGGGTCAAGGAACCTCAGTC | FFPEDITVEWQWNGQPAENYKNTQPIMDTD |
| | ACCGTCTCCTCAGCCAAAACGACACCC | GSYFVYSKLNVQKSNWEAGNTFTCSVLHEG |
| | CCATCTGTCTATCCACTGGCCCCTGGA | LHNHHTEKSLSHSPG |
| | TCTGCTGCCCAAACTAACTCCATGGTG | (SEQ ID NO: 341) |
| | ACCCTGGGATGCCTGGTCAAGGGCTAT | |
| | TTCCCTGAGCCAGTGACAGTGACCTGG | |
| | AACTCTGGATCCCTGTCCAGCGGTGTG | |
| | CACACCTTCCCAGCTGTCCTGCAGTCT | |
| | GACCTCTACACTCTGAGCAGCTCAGTG | |
| | ACTGTCCCCTCCAGCACCTGGCCCAGC | |
| | CAGACCGTCACCTGCAACGTTGCCCAC | |
| | CCGGCCAGCAGCACCAAGGTGGACAA | |
| | GAAAATTGTGCCCAGGGATTGTGGTTG | |
| | TAAGCCTTGCATATGTACAGTCCCAGA | |
| | AGTATCATCTGTCTTCATCTTCCCCCCA | |
| | AAGCCCAAGGATGTGCTCACCATTACT | |
| | CTGACTCCTAAGGTCACGTGTGTTGTG | |
| | GTAGACATCAGCAAGGATGATCCCGA | |
| | GGTCCAGTTCAGCTGGTTTGTAGATGA | |
| | TGTGGAGGTGCACACAGCTCAGACGA | |
| | AACCCCGGGAGGAGCAGATCAACAGC | |
| | ACTTTCCGTTCAGTCAGTGAACTTCCC | |
| | ATCATGCACCAGGACTGGCTCAATGGC | |
| | AAGGAGTTCAAATGCAGGGTCAACAG | |
| | TGCAGCTTTCCCTGCCCCCATCGAGAA | |
| | AACCATCTCCAAAACCAAAGGCAGAC | |
| | CGAAGGCTCCACAGGTGTACACCATTC | |
| | CACCTCCCAAGGAGCAGATGGCCAAG | |
| | GATAAAGTCAGTCTGACCTGCATGATA | |
| | ACAAACTTCTTCCCTGAAGACATTACT | |
| | GTGGAGTGGCAGTGGAATGGGCAGCC | |
| | AGCGGAGAACTACAAGAACACTCAGC | |
| | CCATCATGGACACAGATGGCTCTTACT | |
| | TCGTCTACAGCAAGCTCAATGTGCAGA | |
| | AGAGCAACTGGGAGGCAGGAAATACT | |
| | TTCACCTGCTCTGTGTTACATGAGGGC | |
| | CTGCACAACCACCATACTGAGAAGAGC | |
| | CTCTCCCACTCTCCTGGTTGA | |
| | (SEQ ID NO: 342) | |
| Light | GACATTGTGATGACCCAGTCTCAAAAA | DIVMTQSQKFMSTTVGERVSITCKASQNVGT |
| Chain | TTCATGTCCACAACAGTAGGAGAGAGG | AVAWYQQKPGQSPKLLIYSASNRYTGVPDR |
| 1G3.A3 | GTCAGCATCACCTGCAAGGCCAGTCAG | FTGSGSGTDFTLTISNMQSEDLADYVSQQYS |
| | AATGTGGGTACTGCTGTAGCCTGGTAT | SYPLTFGAGTKLELKRADAAPTVSIFPPSSEQ |
| | CAACAGAAACCAGGACAATCTCCTAA | LTSGGASVVCFLNNFYPRDINVKWKIDGSER |
| | ACTACTGATTTACTCAGCATCCAATCG | QNGVLNSWTDQDSKDSTYSMSSTLTLTKDE |
| | GTACACTGGAGTCCCTGATCGCTTCAC | YERHNSYTCEATHKTSTSPIVKSFNRNEC |
| | AGGCAGTGGATCTGGGACAGATTTCAC | (SEQ ID NO: 307) |
| | TCTCACCATTAGCAATATGCAGTCTGA | |
| | AGACCTGGCAGATTATGTCTCTCAGCA | |
| | ATATAGCAGCTATCCTCTCACGTTCGG | |
| | TGCTGGGACCAAGCTGGAGCTGAAAC | |
| | GGGCTGATGCTGCACCAACTGTATCCA | |
| | TCTTCCCACCATCCAGTGAGCAGTTAA | |
| | CATCTGGAGGTGCCTCAGTCGTGTGCT | |
| | TCTTGAACAACTTCTACCCCAGAGACA | |
| | TCAATGTCAAGTGGAAGATTGATGGCA | |
| | GTGAACGACAAAATGGTGTCCTGAACA | |
| | GTTGGACTGATCAGGACAGCAAAGAC | |
| | AGCACCTACAGCATGAGCAGCACCCTC | |
| | ACATTGACCAAGGACGAGTATGAACG | |
| | ACATAACAGCTATACCTGTGAGGCCAC | |
| | TCACAAGACATCAACTTCACCCATCGT | |
| | CAAGAGCTTCAACAGGAATGAGTGTTA | |
| | G (SEQ ID NO: 306) | |
| Light | GACATTGTGATGACCCAGTCTCAAAAA | DIVMTQSQKFMSTTVGERVSITCKASQNVGT |
| Chain of | TTCATGTCCACAACAGTAGGAGAGAGG | AVAWYQQKPGQSPKLLIYSASNRYTGVPDR |
| variant | GTCAGCATCACCTGCAAGGCCAGTCAG | FTGSGSGTDFTLTISNMQSEDLADYVSQQYS |
| 1G3.A3 | AATGTGGGTACTGCTGTAGCCTGGTAT | SYPLTFGAGTKLELKRADAAPTVSIFPPSSEQ |
| | CAACAGAAACCAGGACAATCTCCTAA | LTSGGASVVCFLNNFYPRDINVKWKIDGSER |
| | ACTACTGATTTACTCAGCATCCAATCG | QNGVLNSWTDQDSKDSTYSMSSTLTLTKDE |
| | GTACACTGGAGTCCCTGATCGCTTCAC | YERHNSYTCEATHKTSTSPIVKSFNRNEC |
| | AGGCAGTGGATCTGGGACAGATTTCAC | (SEQ ID NO: 307) |
| | TCTCACCATTAGCAATATGCAGTCTGA | |
| | AGACCTGGCAGATTATGTCTCTCAGCA | |
| | ATATAGCAGCTATCCTCTCACGTTCGG | |
| | TGCTGGGACCAAGCTGGAGCTGAAAC | |
| | GGGCTGATGCTGCACCAACTGTATCCA | |
| | TCTTCCCACCATCCAGTGAGCAGTTAA | |
| | CATCTGGAGGTGCCTCAGTCGTGTGCT | |
| | TCTTGAACAACTTCTACCCCAGAGACA | |
| | TCAATGTCAAGTGGAAGATTGATGGCA | |
| | GTGAACGACAAAATGGTGTCCTGAACA | |
| | GTTGGACTGATCAGGACAGCAAAGAC | |
| | AGCACCTACAGCATGAGCAGCACCCTC | |
| | ACATTGACCAAGGACGAGTATGAACG | |
| | ACATAACAGCTATACCTGTGAGGCCAC | |
| | TCACAAGACATCAACTTCACCCATCGT | |
| | CAAGAGCTTCAACAGGAATGAGTGTTA | |
| | G (SEQ ID NO: 306) | |
| Heavy | CAGGTTCAACTGCAGCAGTCTGGGGCT | QVQLQQSGAELVRPGASVTLSCKASGYTFT |
| Chain | GAGCTGGTGAGGCCTGGGGCTTCAGTG | DYEMHWVKQTPVHGLEWIGAIDPETGGIAY |
| Variable | ACGCTGTCCTGCAAGGCTTCGGGCTAC | NOKFRGKAILTADKSSSTAYMELRSLTSEDS |
| Region | ACATTTACTGACTATGAAATGCACTGG | AVYYCARNYGISSYYAVDYWGQGTSVTVSS |
| 1G3.A3 | GTGAAACAGACACCTGTGCATGGCCTG | (SEQ ID NO: 309) |
| | GAATGGATTGGAGCTATTGATCCTGAA | |
| | ACTGGTGGTATTGCCTACAATCAGAAG | |
| | TTCAGGGGCAAGGCCATACTGACTGCA | |
| | GACAAATCCTCCAGCACAGCCTACATG | |
| | GAGCTCCGCAGCCTGACATCTGAGGAC | |
| | TCTGCCGTCTATTACTGTGCAAGAAAC | |
| | TACGGTATTAGCTCCTACTATGCTGTG | |
| | GACTACTGGGGTCAAGGAACCTCAGTC | |
| | ACCGTCTCCTCAG (SEQ ID NO: 308) | |
| Light | GACATTGTGATGACCCAGTCTCAAAAA | DIVMTQSQKFMSTTVGERVSITCKASQNVGT |
| Chain | TTCATGTCCACAACAGTAGGAGAGAGG | AVAWYQQKPGQSPKLLIYSASNRYTGVPDR |
| Variable | GTCAGCATCACCTGCAAGGCCAGTCAG | FTGSGSGTDFTLTISNMQSEDLADYVSQQYS |
| Region | AATGTGGGTACTGCTGTAGCCTGGTAT | SYPLTFGAGTKLELK (SEQ ID NO: 311) |
| 1G3.A3 | CAACAGAAACCAGGACAATCTCCTAA | |
| | ACTACTGATTTACTCAGCATCCAATCG | |
| | GTACACTGGAGTCCCTGATCGCTTCAC | |
| | AGGCAGTGGATCTGGGACAGATTTCAC | |
| | TCTCACCATTAGCAATATGCAGTCTGA | |
| | AGACCTGGCAGATTATGTCTCTCAGCA | |
| | ATATAGCAGCTATCCTCTCACGTTCGG | |
| | TGCTGGGACCAAGCTGGAGCTGAAAC | |
| | (SEQ ID NO: 310) | |

Underlined residues indicate position(s) that were altered in the variant of clone
1G3.A3.

TABLE 32
CDR Sequences of anti-IBP4 antibody 1G3.A3 and variant 1G3.A3.

| | Region | | | |
|---|---|---|---|---|
| | Definition | CDR1 | CDR2 | CDR3 |
| Heavy | IMGT | GYTFTDYE | IDPETGGI | ARNYGISSYYAVDY |
| Chain | | (SEQ ID NO: 244) | (SEQ ID NO: 312) | (SEQ ID NO: 313) |
| | Chothia | GYTFTDYEMHW | AIDPETGGIAYNQKFRG | NYGISSYYAVDY |
| | | (SEQ ID NO: 314) | (SEQ ID NO: 315) | (SEQ ID NO: 316) |
| | AbM | GYTFTDYEMHWVK | IGAIDPETGGIAYNQKFRGKA | RNYGISSYYAVDYW |
| | | (SEQ ID NO: 288) | (SEQ ID NO: 317) | (SEQ ID NO: 318) |
| | Contact | TDYEMHWVK | LEWIGAIDPETGGIAYNQKFRGKA | CARNYGISSYYAVDY |
| | | (SEQ ID NO: 291) | (SEQ ID NO: 319) | (SEQ ID NO: 320) |
| | Kabat | YEMHWVK | IGAIDPETGGIAYNQKFRGKAILT | NYGISSYYAVDYW |
| | | (SEQ ID NO: 294) | (SEQ ID NO: 321) | (SEQ ID NO: 322) |
| Light | IMGT | QNVGTA | SAS | QQYSSYPLT |
| Chain | | (SEQ ID NO: 323) | (SEQ ID NO: 24) | (SEQ ID NO: 324) |
| | Chothia | KASQNVGTAVA | SASNRY | QQYSSYPLT |
| | | (SEQ ID NO: 325) | (SEQ ID NO: 326) | (SEQ ID NO: 324) |
| | AbM | TCKASQNVGTAVAWY | SASNRYTGVPD | VSQQYSSYPLTF |
| | | (SEQ ID NO: 327) | (SEQ ID NO: 328) | (SEQ ID NO: 329) |
| | Contact | NVGTAVAWYQQ | LLIYSASNRYTGVP | VSQQYSSYPLT |
| | | (SEQ ID NO: 330) | (SEQ ID NO: 331) | (SEQ ID NO: 332) |
| | Kabat | TCKASQNVGTAVAWY | SASNRYTGVPD | QQYSSYPLT |
| | | (SEQ ID NO: 327) | (SEQ ID NO: 328) | (SEQ ID NO: 324) |

What is claimed is:

1. An isolated anti-sex hormone-binding globulin (SHBG) antibody or a fragment thereof, comprising:
(i) a heavy chain, wherein the heavy chain comprises:
a heavy chain complementarity determining region 1 (HCDR1) comprising an amino acid sequence selected from the group consisting of SEQ ID NOS: 11, 14, 17, 20, 52, and 115; and
a heavy chain complementarity determining region 2 (HCDR2) comprising an amino acid sequence selected from the group consisting of SEQ ID NOS: 12, 15, 18, 21, 44, 47, 50, 53, 116, and 117; and
a heavy chain complementarity determining region 3 (HCDR3) comprising an amino acid sequence selected from the group consisting of SEQ ID NOS: 13, 16, 19, 22, 45, 48, 51, and 54;
or
(ii) a light chain, wherein the light chain comprises:
a light chain complementarity determining region 1 (LCDR1) comprising an amino acid sequence selected from the group consisting of SEQ ID NOS: 23, 26, 29, and 32; and
a light chain complementarity determining region 2 (LCDR2) comprising an amino acid sequence selected from the group consisting of SEQ ID NOS: 24, 27, 30, and 33; and
a light chain complementarity determining region 3 (LCDR3) comprising an amino acid sequence selected from the group consisting of SEQ ID NOS: 25, 31, and 34.

2. The antibody or the fragment thereof of claim 1, wherein the antibody or the fragment thereof comprises the heavy chain.

3. The antibody or the fragment thereof of claim 1, wherein the antibody or the fragment thereof comprises the light chain.

4. The antibody or the fragment thereof of claim 1, wherein the antibody or the fragment thereof comprises the heavy chain and the light chain.

5. The antibody or the fragment thereof of claim 1, wherein:
(i) the HCDR1 comprises the amino acid sequence of SEQ ID NO: 11, the HCDR2 comprises the amino acid sequence of SEQ ID NO: 12, and the HCDR3 comprises the amino acid sequence of SEQ ID NO: 13; and
(ii) the LCDR1 comprises the amino acid sequence of SEQ ID NO: 23, the LCDR2 comprises the amino acid sequence of SEQ ID NO: 24, and the LCDR3 comprises the amino acid sequence of SEQ ID NO: 25.

6. The antibody or the fragment thereof of claim 1, wherein:
(i) the HCDR1 comprises the amino acid sequence of SEQ ID NO: 11, the HCDR2 comprises the amino acid sequence of SEQ ID NO: 44, and the HCDR3 comprises the amino acid sequence of SEQ ID NO: 45; and
(ii) the LCDR1 comprises the amino acid sequence of SEQ ID NO:23, the LCDR2 comprises the amino acid sequence of SEQ ID NO:24, and the LCDR3 comprises the amino acid sequence of SEQ ID NO:25.

7. The antibody or the fragment thereof of claim 1, wherein:
(i) the HCDR1 comprises the amino acid sequence of SEQ ID NO: 14, the HCDR2 comprises the amino acid sequence of SEQ ID NO: 15, and the HCDR3 comprises the amino acid sequence of SEQ ID NO: 16; and
(ii) the LCDR1 comprises the amino acid sequence of SEQ ID NO: 26, the LCDR2 comprises the amino acid sequence of SEQ ID NO: 27, and the LCDR3 comprises the amino acid sequence of SEQ ID NO: 25.

8. The antibody or the fragment thereof of claim 1, wherein:
(i) the HCDR1 comprises the amino acid sequence of SEQ ID NO: 14, the HCDR2 comprises the amino acid sequence of SEQ ID NO: 47, and the HCDR3 comprises the amino acid sequence of SEQ ID NO: 48; and
(ii) the LCDR1 comprises the amino acid sequence of SEQ ID NO: 26, the LCDR2 comprises the amino acid sequence of SEQ ID NO: 27, and the LCDR3 comprises the amino acid sequence of SEQ ID NO: 25.

9. The antibody or the fragment thereof of claim 1, wherein:
(i) the HCDR1 comprises the amino acid sequence of SEQ ID NO: 17, the HCDR2 comprises the amino acid sequence of SEQ ID NO: 18, and the HCDR3 comprises the amino acid sequence of SEQ ID NO: 19; and
(ii) the LCDR1 comprises the amino acid sequence of SEQ ID NO: 29, the LCDR2 comprises the amino acid sequence of SEQ ID NO: 30, and the LCDR3 comprises the amino acid sequence of SEQ ID NO: 31.

10. The antibody or the fragment thereof of claim 1, wherein:
(i) the HCDR1 comprises the amino acid sequence of SEQ ID NO: 17, the HCDR2 comprises the amino acid sequence of SEQ ID NO: 50, and the HCDR3 comprises the amino acid sequence of SEQ ID NO: 51; and
(ii) the LCDR1 comprises the amino acid sequence of SEQ ID NO: 29, the LCDR2 comprises the amino acid sequence of SEQ ID NO: 30, and the LCDR3 comprises the amino acid sequence of SEQ ID NO: 31.

11. The antibody or the fragment thereof of claim 1, wherein:
(i) the HCDR1 comprises the amino acid sequence of SEQ ID NO: 20, the HCDR2 comprises the amino acid sequence of SEQ ID NO: 21, and the HCDR3 comprises the amino acid sequence of SEQ ID NO: 22; and
(ii) the LCDR1 comprises the amino acid sequence of SEQ ID NO: 32, the LCDR2 comprises the amino acid sequence of SEQ ID NO: 33, and the LCDR3 comprises the amino acid sequence of SEQ ID NO: 34.

12. The antibody or the fragment thereof of claim 1, wherein:
(i) the HCDR1 comprises the amino acid sequence of SEQ ID NO: 52, the HCDR2 comprises the amino acid sequence of SEQ ID NO: 53, the HCDR3 comprises the amino acid sequence of SEQ ID NO: 54; and
(ii) the LCDR1 comprises the amino acid sequence of SEQ ID NO: 32, the LCDR2 comprises the amino acid sequence of SEQ ID NO: 33, and the LCDR3 comprises the amino acid sequence of SEQ ID NO: 34.

13. The antibody or the fragment thereof of claim 1, wherein:
(i) the HCDR1 comprises the amino acid sequence of SEQ ID NO: 115, the HCDR2 comprises the amino acid sequence of SEQ ID NO: 116, and the HCDR3 comprises the amino acid sequence of SEQ ID NO: 19; and
(ii) the LCDR1 comprises the amino acid sequence of SEQ ID NO: 29, the LCDR2 comprises the amino acid sequence of SEQ ID NO: 30, and the LCDR3 comprises the amino acid sequence of SEQ ID NO: 31.

14. The antibody or the fragment thereof of claim 1, wherein:
(i) the HCDR1 comprises the amino acid sequence of SEQ ID NO: 115, the HCDR2 comprises the amino acid sequence of SEQ ID NO: 117, and the HCDR3 comprises the amino acid sequence of SEQ ID NO: 51; and
(ii) the LCDR1 comprises the amino acid sequence of SEQ ID NO: 29, the LCDR2 comprises the amino acid sequence of SEQ ID NO: 30, and the LCDR3 comprises the amino acid sequence of SEQ ID NO: 31.

15. An isolated anti-SHBG antibody or a fragment thereof, comprising:
(i) a heavy chain comprising a HCDR1 comprising amino acid sequence GYTFTGYW (SEQ ID NO: 11), a HCDR2 comprising amino acid sequence IDPSX₁X₂YT (SEQ ID NO: 112), wherein X₁ is independently any naturally occurring amino acid residue and X₂ is independently any naturally occurring amino acid residue, and a HCDR3 comprising amino acid sequence TRX₃PLVTADFX₃Y (SEQ ID NO: 113), wherein X₃ is independently any naturally occurring amino acid residue; and
(ii) a light chain comprising a LCDR1 comprising amino acid sequence QNVDTN (SEQ ID NO: 23), a LCDR2 comprising amino acid sequence SAS (SEQ ID NO: 24), and a LCDR3 comprising amino acid sequence QQYDSYPY (SEQ ID NO: 25).

16. The antibody or the fragment thereof of claim 15, wherein X₁ comprises threonine or aspartic acid, X₂ comprises glycine or threonine, and X₃ comprises glutamic acid or aspartic acid.

17. The antibody or the fragment thereof of claim 15, wherein X₁ comprises threonine, X₂ comprises glycine, and X₃ comprises glutamic acid.

18. An isolated anti-SHBG antibody or a fragment thereof, comprising:
(i) a heavy chain comprising an amino acid sequence that has at least 85%, 90%, 95%, 96%, 97%, 98%, 99%, or 100% identity to SEQ ID NO: 4, or a light chain comprising an amino acid sequence that has at least 85%, 90%, 95%, 96%, 97%, 98%, 99%, or 100% identity to SEQ ID NO: 6;
or
(ii) a heavy chain comprising an amino acid sequence that has at least 85%, 90%, 95%, 96%, 97%, 98%, 99%, or 100% identity to SEQ ID NO: 36, or a light chain comprising an amino acid sequence that has at least 85%, 90%, 95%, 96%, 97%, 98%, 99%, or 100% identity to SEQ ID NO: 38.

19. The antibody or the fragment thereof of claim 18, wherein the antibody or the fragment thereof comprises the heavy chain of SEQ ID NO: 4.

20. The antibody or the fragment thereof of claim 18, wherein the antibody or the fragment thereof comprises the light chain of SEQ ID NO: 6.

21. The antibody or the fragment thereof of claim 18, wherein the antibody or the fragment thereof comprises the heavy chain of SEQ ID NO: 4 and the light chain of SEQ ID NO: 6.

22. The antibody or the fragment thereof of claim 18, wherein the antibody or the fragment thereof comprises the heavy chain of SEQ ID NO: 36.

23. The antibody or the fragment thereof of claim 18, wherein the antibody or the fragment thereof comprises the light chain of SEQ ID NO: 38.

24. The antibody or the fragment thereof of claim 18, wherein the antibody or the fragment thereof comprises the heavy chain of SEQ ID NO: 36 and the light chain of SEQ ID NO: 38.

25. The antibody or the fragment thereof of claim 3, wherein the heavy chain and the light chain form an antigen binding domain for binding to an epitope of SHBG.

26. The antibody or the fragment thereof of claim 25, wherein the epitope of SHBG comprises the amino acid sequence of SEQ ID NO: 110.

27. The antibody or the fragment thereof of any one of claims 1-26, wherein the antibody or the fragment thereof has a higher affinity for a biologically active form of SHBG than for a biologically inactive form of SHBG.

28. The antibody or the fragment thereof of any one of claims 1-26, wherein the antibody or the fragment thereof comprises a higher affinity for glycosylated SHBG than for non-glycosylated SHBG.

29. The antibody or the fragment thereof of claim 28, wherein the glycosylated SHBG comprises an epitope comprising a glycosylated amino acid, wherein the glycosylated amino acid comprises a threonine at position 36.

30. The antibody or the fragment thereof of claim 30, wherein the glycosylated amino acid comprises an O-linked glycan.

31. The antibody or the fragment thereof of any one of claims 1-30, wherein the antibody or the fragment thereof has a dissociation constant (K_{D}) of about 1×10⁻⁷ M or less, 1×10⁻⁸ M or less, 1×10⁻⁹ M or less, 1×10⁻¹⁰ M or less, 2×10⁻⁷ M or less, 2×10⁻⁸ M or less, 2×10⁻⁹ M or less, 2×10⁻¹⁰ M or less, 5×10⁻⁷ M or less, 5×10⁻⁸ M or less, 5×10⁻⁹ M or less, or 5×10⁻¹⁰ M or less.

32. An isolated anti-insulin like growth factor binding protein 4 (IBP4) antibody or a fragment thereof, comprising:
(i) a heavy chain, wherein the heavy chain comprises:
a heavy chain complementarity determining region 1 (HCDR1) comprising an amino acid sequence selected from the group consisting of SEQ ID NOS: 75, 78, 81, 84, 118, 244, 247, 250, 253, 256, 288, 291, 294, and 314; and
a heavy chain complementarity determining region 2 (HCDR2) comprising an amino acid sequence selected from the group consisting of SEQ ID NOS: 76, 79, 82, 85, 119, 216, 218, 220, 222, 224, 245, 248, 251, 254, 257, 284, 286, 289, 292, 295, 312, 315, 317, 319, and 321; and
a heavy chain complementarity determining region 3 (HCDR3) comprising an amino acid sequence selected from the group consisting of SEQ ID NOS: 77, 80, 83, 86, 217, 219, 221, 223, 225, 246, 249, 252, 255, 258, 285, 287, 290, 293, 296, 313, 316, 318, 320, 322;
or
(ii) a light chain, wherein the light chain comprises:
a light chain complementarity determining region 1 (LCDR1) comprising an amino acid sequence selected from the group consisting of SEQ ID NOS: 23, 87, 90, 93, 96, 226, 228, 230, 233, 272, 274, 275, 323, 325, 327, and 330; and
a light chain complementarity determining region 2 (LCDR2) comprising an amino acid sequence selected from the group consisting of SEQ ID NOS: 24, 91, 94, 97, 229, 231, 234, 260, 261, 263, 273, 297, 299, 300, 302, 326, 328, and 331; and
a light chain complementarity determining region 3 (LCDR3) comprising an amino acid sequence selected from the group consisting of SEQ ID NOS: 89, 95, 98, 99, 100, 101, 227, 232, 235, 259, 262, 264, 298, 301, 303, 324, 329, and 332.

33. The antibody or the fragment thereof of claim 32, wherein the antibody or the fragment thereof comprises the heavy chain.

34. The antibody or the fragment thereof of claim 32, wherein the antibody or the fragment thereof comprises the light chain.

35. The antibody or the fragment thereof of claim 32, wherein the antibody or the fragment thereof comprises the heavy chain and the light chain.

36. The antibody or the fragment thereof of claim 32, wherein:
(i) the HCDR1 comprises the amino acid sequence of SEQ ID NO: 75, the HCDR2 comprises the amino acid sequence of SEQ ID NO: 76, and the HCDR3 comprises the amino acid sequence of SEQ ID NO: 77; and
(ii) the LCDR1 comprises the amino acid sequence of SEQ ID NO: 87, the LCDR2 comprises the amino acid sequence of SEQ ID NO: 24, and the LCDR3 comprises the amino acid sequence of SEQ ID NO: 99.

37. The antibody or the fragment thereof of claim 32, wherein:
(i) the HCDR1 comprises the amino acid sequence of SEQ ID NO: 75, the HCDR2 comprises the amino acid sequence of SEQ ID NO: 76, and the HCDR3 comprises the amino acid sequence of SEQ ID NO: 77; and
(ii) the LCDR1 comprises the amino acid sequence of SEQ ID NO: 87, the LCDR2 comprises the amino acid sequence of SEQ ID NO: 24, and the LCDR3 comprises the amino acid sequence of SEQ ID NO: 89.

38. The antibody or the fragment thereof of claim 32, wherein:
(i) the HCDR1 comprises the amino acid sequence of SEQ ID NO: 78, the HCDR2 comprises the amino acid sequence of SEQ ID NO: 79, and the HCDR3 comprises the amino acid sequence of SEQ ID NO: 80; and
(ii) the LCDR1 comprises the amino acid sequence of SEQ ID NO: 90, the LCDR2 comprises the amino acid sequence of SEQ ID NO: 91, and the LCDR3 comprises the amino acid sequence of SEQ ID NO: 89.

39. The antibody or the fragment thereof of claim 32, wherein:
(i) the HCDR1 comprises the amino acid sequence of SEQ ID NO: 78, the HCDR2 comprises the amino acid sequence of SEQ ID NO: 79, and the HCDR3 comprises the amino acid sequence of SEQ ID NO: 80; and
(ii) the LCDR1 comprises the amino acid sequence of SEQ ID NO: 90, the LCDR2 comprises the amino acid sequence of SEQ ID NO: 91, and the LCDR3 comprises the amino acid sequence of SEQ ID NO: 99.

40. The antibody or the fragment thereof of claim 32, wherein:
(i) the HCDR1 comprises the amino acid sequence of SEQ ID NO: 81, the HCDR2 comprises the amino acid sequence of SEQ ID NO: 82, and the HCDR3 comprises the amino acid sequence of SEQ ID NO: 83; and
(ii) the LCDR1 comprises the amino acid sequence of SEQ ID NO: 93, the LCDR2 comprises the amino acid sequence of SEQ ID NO: 94, and the LCDR3 comprises the amino acid sequence of SEQ ID NO: 95.

41. The antibody or the fragment thereof of claim 32, wherein:
(i) the HCDR1 comprises the amino acid sequence of SEQ ID NO: 81, the HCDR2 comprises the amino acid sequence of SEQ ID NO: 82, the HCDR3 comprises the amino acid sequence of SEQ ID NO: 83; and
(ii) the LCDR1 comprises the amino acid sequence of SEQ ID NO: 93, the LCDR2 comprises the amino acid sequence of SEQ ID NO: 94, and the LCDR3 comprises the amino acid sequence of SEQ ID NO: 100.

42. The antibody or the fragment thereof of claim 32, wherein:
(i) the HCDR1 comprises the amino acid sequence of SEQ ID NO: 84, the HCDR2 comprises the amino acid sequence of SEQ ID NO: 85, and the HCDR3 comprises the amino acid sequence of SEQ ID NO: 86; and
(ii) the LCDR1 comprises the amino acid sequence of SEQ ID NO: 96, the LCDR2 comprises the amino acid sequence of SEQ ID NO: 97, and the LCDR3 comprises the amino acid sequence of SEQ ID NO: 98.

43. The antibody or the fragment thereof of claim 32, wherein:
(i) the HCDR1 comprises the amino acid sequence of SEQ ID NO: 84, the HCDR2 comprises the amino acid sequence of SEQ ID NO: 85, and the HCDR3 comprises the amino acid sequence of SEQ ID NO: 86; and
(ii) the LCDR1 comprises the amino acid sequence of SEQ ID NO: 96, the LCDR2 comprises the amino acid sequence of SEQ ID NO: 97, and the LCDR3 comprises the amino acid sequence of SEQ ID NO: 101.

44. The antibody or the fragment thereof of claim 32, wherein:
(i) the HCDR1 comprises the amino acid sequence of SEQ ID NO: 118, the HCDR2 comprises the amino acid sequence of SEQ ID NO: 119, and the HCDR3 comprises the amino acid sequence of SEQ ID NO: 83; and
(ii) the LCDR1 comprises the amino acid sequence of SEQ ID NO: 93, the LCDR2 comprises the amino acid sequence of SEQ ID NO: 94, and the LCDR3 comprises the amino acid sequence of SEQ ID NO: 89.

45. The antibody or the fragment thereof of claim 32, wherein:
(i) the HCDR1 comprises the amino acid sequence of SEQ ID NO: 118, the HCDR2 comprises the amino acid sequence of SEQ ID NO: 119, and the HCDR3 comprises the amino acid sequence of SEQ ID NO: 83; and
(ii) the LCDR1 comprises the amino acid sequence of SEQ ID NO: 93, the LCDR2 comprises the amino acid sequence of SEQ ID NO: 94, and the LCDR3 comprises the amino acid sequence of SEQ ID NO: 99.

46. The antibody or the fragment thereof of claim 32, wherein:
(i) the HCDR1 comprises the amino acid sequence of SEQ ID NO: 75, the HCDR2 comprises the amino acid sequence of SEQ ID NO: 216, and the HCDR3 comprises the amino acid sequence of SEQ ID NO: 217; and
(ii) the LCDR1 comprises the amino acid sequence of SEQ ID NO: 226, the LCDR2 comprises the amino acid sequence of SEQ ID NO: 24, and the LCDR3 comprises the amino acid sequence of SEQ ID NO: 227.

47. The antibody or the fragment thereof of claim 32, wherein:
(i) the HCDR1 comprises the amino acid sequence of SEQ ID NO: 78, the HCDR2 comprises the amino acid sequence of SEQ ID NO: 218, and the HCDR3 comprises the amino acid sequence of SEQ ID NO: 219; and
(ii) the LCDR1 comprises the amino acid sequence of SEQ ID NO: 228, the LCDR2 comprises the amino acid sequence of SEQ ID NO: 229, and the LCDR3 comprises the amino acid sequence of SEQ ID NO: 227.

48. The antibody or the fragment thereof of claim 32, wherein:
(i) the HCDR1 comprises the amino acid sequence of SEQ ID NO: 81, the HCDR2 comprises the amino acid sequence of SEQ ID NO: 220, and the HCDR3 comprises the amino acid sequence of SEQ ID NO: 221; and
(ii) the LCDR1 comprises the amino acid sequence of SEQ ID NO: 230, the LCDR2 comprises the amino acid sequence of SEQ ID NO: 231, and the LCDR3 comprises the amino acid sequence of SEQ ID NO: 232.

49. The antibody or the fragment thereof of claim 32, wherein:
(i) the HCDR1 comprises the amino acid sequence of SEQ ID NO: 84, the HCDR2 comprises the amino acid sequence of SEQ ID NO: 222, and the HCDR3 comprises the amino acid sequence of SEQ ID NO: 223; and
(ii) the LCDR1 comprises the amino acid sequence of SEQ ID NO: 233, the LCDR2 comprises the amino acid sequence of SEQ ID NO: 234, and the LCDR3 comprises the amino acid sequence of SEQ ID NO: 235.

50. The antibody or the fragment thereof of claim 32, wherein:
(i) the HCDR1 comprises the amino acid sequence of SEQ ID NO: 118, the HCDR2 comprises the amino acid sequence of SEQ ID NO: 224, and the HCDR3 comprises the amino acid sequence of SEQ ID NO: 225; and
(ii) the LCDR1 comprises the amino acid sequence of SEQ ID NO: 230, the LCDR2 comprises the amino acid sequence of SEQ ID NO: 231, and the LCDR3 comprises the amino acid sequence of SEQ ID NO: 227.

51. The antibody or the fragment thereof of claim 32, wherein:
(i) the HCDR1 comprises the amino acid sequence of SEQ ID NO: 244, the HCDR2 comprises the amino acid sequence of SEQ ID NO: 245, and the HCDR3 comprises the amino acid sequence of SEQ ID NO: 246; and
(ii) the LCDR1 comprises the amino acid sequence of SEQ ID NO: 87, the LCDR2 comprises the amino acid sequence of SEQ ID NO: 24, and the LCDR3 comprises the amino acid sequence of SEQ ID NO: 259.

52. The antibody or the fragment thereof of claim 32, wherein:
(i) the HCDR1 comprises the amino acid sequence of SEQ ID NO: 247, the HCDR2 comprises the amino acid sequence of SEQ ID NO: 248, and the HCDR3 comprises the amino acid sequence of SEQ ID NO: 249; and
(ii) the LCDR1 comprises the amino acid sequence of SEQ ID NO: 90, the LCDR2 comprises the amino acid sequence of SEQ ID NO: 260, and the LCDR3 comprises the amino acid sequence of SEQ ID NO: 259.

53. The antibody or the fragment thereof of claim 32, wherein:
(i) the HCDR1 comprises the amino acid sequence of SEQ ID NO: 250, the HCDR2 comprises the amino acid sequence of SEQ ID NO: 251, and the HCDR3 comprises the amino acid sequence of SEQ ID NO: 252; and
(ii) the LCDR1 comprises the amino acid sequence of SEQ ID NO: 93, the LCDR2 comprises the amino acid sequence of SEQ ID NO: 261, and the LCDR3 comprises the amino acid sequence of SEQ ID NO: 262.

54. The antibody or the fragment thereof of claim 32, wherein:
(i) the HCDR1 comprises the amino acid sequence of SEQ ID NO: 253, the HCDR2 comprises the amino acid sequence of SEQ ID NO: 254, and the HCDR3 comprises the amino acid sequence of SEQ ID NO: 255; and
(ii) the LCDR1 comprises the amino acid sequence of SEQ ID NO: 96, the LCDR2 comprises the amino acid sequence of SEQ ID NO: 263, and the LCDR3 comprises the amino acid sequence of SEQ ID NO: 264.

55. The antibody or the fragment thereof of claim 32, wherein:
(i) the HCDR1 comprises the amino acid sequence of SEQ ID NO: 256, the HCDR2 comprises the amino acid sequence of SEQ ID NO: 257, and the HCDR3 comprises the amino acid sequence of SEQ ID NO: 258; and
(ii) the LCDR1 comprises the amino acid sequence of SEQ ID NO: 93, the LCDR2 comprises the amino acid sequence of SEQ ID NO: 261, and the LCDR3 comprises the amino acid sequence of SEQ ID NO: 259.

56. The antibody or the fragment thereof of claim 32, wherein:
(i) the HCDR1 comprises the amino acid sequence of SEQ ID NO: 244, the HCDR2 comprises the amino acid sequence of SEQ ID NO: 245, and the HCDR3 comprises the amino acid sequence of SEQ ID NO: 246; and
(ii) the LCDR1 comprises the amino acid sequence of SEQ ID NO: 23, the LCDR2 comprises the amino acid sequence of SEQ ID NO: 24, and the LCDR3 comprises the amino acid sequence of SEQ ID NO: 259.

57. The antibody or the fragment thereof of claim 32, wherein:
(i) the HCDR1 comprises the amino acid sequence of SEQ ID NO: 247, the HCDR2 comprises the amino acid sequence of SEQ ID NO: 248, and the HCDR3 comprises the amino acid sequence of SEQ ID NO: 249; and
(ii) the LCDR1 comprises the amino acid sequence of SEQ ID NO: 272, the LCDR2 comprises the amino acid sequence of SEQ ID NO: 273, and the LCDR3 comprises the amino acid sequence of SEQ ID NO: 259.

58. The antibody or the fragment thereof of claim 32, wherein:
(i) the HCDR1 comprises the amino acid sequence of SEQ ID NO: 250, the HCDR2 comprises the amino acid sequence of SEQ ID NO: 251, and the HCDR3 comprises the amino acid sequence of SEQ ID NO: 252; and
(ii) the LCDR1 comprises the amino acid sequence of SEQ ID NO: 274, the LCDR2 comprises the amino acid sequence of SEQ ID NO: 94, and the LCDR3 comprises the amino acid sequence of SEQ ID NO: 262.

59. The antibody or the fragment thereof of claim 32, wherein:
(i) the HCDR1 comprises the amino acid sequence of SEQ ID NO: 253, the HCDR2 comprises the amino acid sequence of SEQ ID NO: 254, and the HCDR3 comprises the amino acid sequence of SEQ ID NO: 255; and
(ii) the LCDR1 comprises the amino acid sequence of SEQ ID NO: 275, the LCDR2 comprises the amino acid sequence of SEQ ID NO: 97, and the LCDR3 comprises the amino acid sequence of SEQ ID NO: 264.

60. The antibody or the fragment thereof of claim 32, wherein:
(i) the HCDR1 comprises the amino acid sequence of SEQ ID NO: 256, the HCDR2 comprises the amino acid sequence of SEQ ID NO: 257, and the HCDR3 comprises the amino acid sequence of SEQ ID NO: 258; and
(ii) the LCDR1 comprises the amino acid sequence of SEQ ID NO: 274, the LCDR2 comprises the amino acid sequence of SEQ ID NO: 94, and the LCDR3 comprises the amino acid sequence of SEQ ID NO: 259.

61. The antibody or the fragment thereof of claim 32, wherein:
(i) the HCDR1 comprises the amino acid sequence of SEQ ID NO: 244, the HCDR2 comprises the amino acid sequence of SEQ ID NO: 284, and the HCDR3 comprises the amino acid sequence of SEQ ID NO: 285; and
(ii) the LCDR1 comprises the amino acid sequence of SEQ ID NO: 87, the LCDR2 comprises the amino acid sequence of SEQ ID NO: 297, and the LCDR3 comprises the amino acid sequence of SEQ ID NO: 298.

62. The antibody or the fragment thereof of claim 32, wherein:
(i) the HCDR1 comprises the amino acid sequence of SEQ ID NO: 247, the HCDR2 comprises the amino acid sequence of SEQ ID NO: 286, and the HCDR3 comprises the amino acid sequence of SEQ ID NO: 287; and
(ii) the LCDR1 comprises the amino acid sequence of SEQ ID NO: 90, the LCDR2 comprises the amino acid sequence of SEQ ID NO: 299, and the LCDR3 comprises the amino acid sequence of SEQ ID NO: 298.

63. The antibody or the fragment thereof of claim 32, wherein:
(i) the HCDR1 comprises the amino acid sequence of SEQ ID NO: 288, the HCDR2 comprises the amino acid sequence of SEQ ID NO: 289, and the HCDR3 comprises the amino acid sequence of SEQ ID NO: 290; and
(ii) the LCDR1 comprises the amino acid sequence of SEQ ID NO: 93, the LCDR2 comprises the amino acid sequence of SEQ ID NO: 300, and the LCDR3 comprises the amino acid sequence of SEQ ID NO: 301.

64. The antibody or the fragment thereof of claim 32, wherein:
(i) the HCDR1 comprises the amino acid sequence of SEQ ID NO: 291, the HCDR2 comprises the amino acid sequence of SEQ ID NO: 292, and the HCDR3 comprises the amino acid sequence of SEQ ID NO: 293; and
(ii) the LCDR1 comprises the amino acid sequence of SEQ ID NO: 96, the LCDR2 comprises the amino acid sequence of SEQ ID NO: 302, and the LCDR3 comprises the amino acid sequence of SEQ ID NO: 303.

65. The antibody or the fragment thereof of claim 32, wherein:
(i) the HCDR1 comprises the amino acid sequence of SEQ ID NO: 294, the HCDR2 comprises the amino acid sequence of SEQ ID NO: 295, and the HCDR3 comprises the amino acid sequence of SEQ ID NO: 296; and
(ii) the LCDR1 comprises the amino acid sequence of SEQ ID NO: 93, the LCDR2 comprises the amino acid sequence of SEQ ID NO: 300, and the LCDR3 comprises the amino acid sequence of SEQ ID NO: 298.

66. The antibody or the fragment thereof of claim 32, wherein:
(i) the HCDR1 comprises the amino acid sequence of SEQ ID NO: 244, the HCDR2 comprises the amino acid sequence of SEQ ID NO: 312, and the HCDR3 comprises the amino acid sequence of SEQ ID NO: 313; and
(ii) the LCDR1 comprises the amino acid sequence of SEQ ID NO: 323, the LCDR2 comprises the amino acid sequence of SEQ ID NO: 24, and the LCDR3 comprises the amino acid sequence of SEQ ID NO: 324.

67. The antibody or the fragment thereof of claim 32, wherein:
(i) the HCDR1 comprises the amino acid sequence of SEQ ID NO: 314, the HCDR2 comprises the amino acid sequence of SEQ ID NO: 315, and the HCDR3 comprises the amino acid sequence of SEQ ID NO: 316; and
(ii) the LCDR1 comprises the amino acid sequence of SEQ ID NO: 325, the LCDR2 comprises the amino acid sequence of SEQ ID NO: 326, and the LCDR3 comprises the amino acid sequence of SEQ ID NO: 324.

68. The antibody or the fragment thereof of claim 32, wherein:
(i) the HCDR1 comprises the amino acid sequence of SEQ ID NO: 288, the HCDR2 comprises the amino acid sequence of SEQ ID NO: 317, and the HCDR3 comprises the amino acid sequence of SEQ ID NO: 318; and
(ii) the LCDR1 comprises the amino acid sequence of SEQ ID NO: 327, the LCDR2 comprises the amino acid sequence of SEQ ID NO: 328, and the LCDR3 comprises the amino acid sequence of SEQ ID NO: 329.

69. The antibody or the fragment thereof of claim 32, wherein:
(i) the HCDR1 comprises the amino acid sequence of SEQ ID NO: 291, the HCDR2 comprises the amino acid sequence of SEQ ID NO: 319, and the HCDR3 comprises the amino acid sequence of SEQ ID NO: 320; and
(ii) the LCDR1 comprises the amino acid sequence of SEQ ID NO: 330, the LCDR2 comprises the amino acid sequence of SEQ ID NO: 331, and the LCDR3 comprises the amino acid sequence of SEQ ID NO: 332.

70. The antibody or the fragment thereof of claim 32, wherein:
(i) the HCDR1 comprises the amino acid sequence of SEQ ID NO: 294, the HCDR2 comprises the amino acid sequence of SEQ ID NO: 321, and the HCDR3 comprises the amino acid sequence of SEQ ID NO: 322; and
(ii) the LCDR1 comprises the amino acid sequence of SEQ ID NO: 327, the LCDR2 comprises the amino acid sequence of SEQ ID NO: 328, and the LCDR3 comprises the amino acid sequence of SEQ ID NO: 324.

71. The antibody or the fragment thereof of claim 35, wherein the heavy chain and the light chain form an antigen binding domain for binding to an epitope of IBP4.

72. The antibody or the fragment thereof of claim 71, wherein the epitope of IBP4 comprises the amino acid sequence of SEQ ID NO: 111.

73. An isolated anti-IBP4 antibody or a fragment thereof, comprising:
(i) a HCDR1 comprising amino acid sequence GYTFTDYY (SEQ ID NO: 75), a HCDR2 comprising amino acid sequence INPNNGGS (SEQ ID NO: 76), and a HCDR3 comprising amino acid sequence ARWDGGFDY (SEQ ID NO: 77); and
(ii) a LCDR1 comprising amino acid sequence QNVGTN (SEQ ID NO: 87), a LCDR2 comprising amino acid sequence SAS (SEQ ID NO: 24), and a LCDR3 comprising amino acid sequence QQYNX₁YPLT (SEQ ID NO: 114) wherein X₁ is any naturally occurring amino acid residue.

74. The antibody or the fragment thereof of claim 73, wherein X₁ comprises isoleucine or serine.

75. An isolated anti-IBP4 antibody or a fragment thereof, comprising:
(i) a heavy chain comprising a HCDR1 comprising amino acid sequence GYTFTYX₁ (SEQ ID NO: 354), a HCDR2 comprising amino acid sequence IX₂X₃X₄X₅GGX₆ (SEQ ID NO: 355), and a HCDR3 comprising amino acid sequence X₇RX₈X₉X₁₀X₁₁X₁₂X₁₃X₁₄X₁₅X₁₆X₁₇DX₁₈ (SEQ ID NO: 356); and
(ii) a light chain comprising a LCDR1 comprising amino acid sequence QNVX₁₉X₂₀X₂₁ (SEQ ID NO: 357), a LCDR2 comprising amino acid sequence SX₂₂S (SEQ ID NO: 358), and a LCDR3 comprising amino acid sequence QQYX₂₃X₂₄YPLX₂₅ (SEQ ID NO: 359),
wherein each of X₁-X₅, X₇, X₁₂-X₁₄, X₁₈-X₂₀, X₂₂, and X₂₄-X₂₅ are independently any naturally occurring amino acid residue, wherein X₆ is independently any naturally occurring amino acid residue except isoleucine, wherein X₈ is independently a deletion or any naturally occurring amino acid residue except asparagine, wherein X₉ is independently any naturally occurring amino acid residue except tyrosine, wherein X₁₀ is independently a deletion or any naturally occurring amino acid residue, wherein X₁₁ is independently a deletion or any naturally occurring amino acid residue except isoleucine, wherein X₁₅ is independently a deletion or any naturally occurring amino acid residue, wherein X₁₆ is independently a deletion or any naturally occurring amino acid residue except alanine, wherein X₁₇ is independently any naturally occurring amino acid residue except valine, wherein X₂₁ is independently any naturally occurring amino acid residue except alanine, and wherein X₂₃ is independently any naturally occurring amino acid residue except serine.

76. The antibody or the fragment thereof of claim 75, wherein
X₁ comprises tyrosine or glutamic acid;
X₂ comprises asparagine or aspartic acid;
X₃ comprises proline or threonine;
X₄ comprises glutamic acid or asparagine;
X₅ comprises threonine or asparagine;
X₆ comprises alanine or proline;
X₇ comprises alanine or threonine;
X₈ comprises alanine;
X₉ comprises tryptophan or arginine;
X₁₀ comprises glycine;
X₁₁ comprises tryptophan or serine;
X₁₂ comprises aspartic acid or arginine or serine;
X₁₃ comprises arginine or serine or glycine;
X₁₄ comprises tyrosine or glycine or valine;
X₁₅ comprises tyrosine;
X₁₆ comprises tyrosine;
X₁₇ comprises phenylalanine;
X₁₈ comprises tyrosine or asparagine;
X₁₉ comprises glycine or aspartic acid;
X₂₀ comprises threonine or isoleucine;
X₂₁ comprises aspartic acid or asparagine;
X₂₂ comprises alanine or threonine;
X₂₃ comprises asparagine or aspartic acid;
X₂₄ comprises isoleucine or serine or threonine;
X₂₅ comprises alanine or threonine,
or a combination thereof.

77. An isolated anti-IBP4 antibody or a fragment thereof, comprising:
(i) a heavy chain comprising a HCDR1 comprising amino acid sequence GYTFTDYY (SEQ ID NO: 75), a HCDR2 comprising amino acid sequence INPNNGGX₁ (SEQ ID NO: 360), and a HCDR3 comprising amino acid sequence ARWX₂X₃DX₄X₅FDY (SEQ ID NO: 361); and
(ii) a light chain comprising a LCDR1 comprising amino acid sequence QNVGX₆X₇ (SEQ ID NO: 362), a LCDR2 comprising amino acid sequence SAS (SEQ ID NO: 24), and a LCDR3 comprising amino acid sequence QQYNX₈YPLX₉ (SEQ ID NO: 363),
wherein X₁ is independently any naturally occurring amino acid residue except isoleucine, wherein each of X₂ and X₃ are independently a deletion or any naturally occurring amino acid residue, wherein X₄ is independently any naturally occurring amino acid residue except serine, wherein each of X₅, X₆, X₈, and X₉ are independently any naturally occurring amino acid residue, and wherein X₇ is independently any naturally occurring amino acid residue except alanine.

78. The antibody or the fragment thereof of claim 77, wherein
X₁ comprises alanine or serine;
X₂ comprises glycine;
X₃ comprises tryptophan;
X₄ comprises arginine or glycine;
X₅ comprises tyrosine or glycine;
X₆ comprises isoleucine or threonine;
X₇ comprises aspartic acid or asparagine;
X₈ comprises serine or isoleucine;
X₉ comprises alanine or threonine;
or a combination thereof.

79. An isolated anti-IBP4 antibody or a fragment thereof, comprising:
(i) a heavy chain comprising a HCDR1 comprising amino acid sequence GYTFTDYE (SEQ ID NO: 244), a HCDR2 comprising amino acid sequence IDX₁ETGGX₂ (SEQ ID NO: 364), and a HCDR3 comprising amino acid sequence TRARGSX₃SVYYFDX₄ (SEQ ID NO: 365); and
(ii) a light chain comprising a LCDR1 comprising amino acid sequence QNVX₅TN (SEQ ID NO: 366), a LCDR2 comprising amino acid sequence SX₆S (SEQ ID NO: 367), and a LCDR3 comprising amino acid sequence QQYDX₇YPLT (SEQ ID NO: 368),
wherein each of X₁, X₃, X₄, X₅, X₆, and X₇ are independently any naturally occurring amino acid residue, and wherein X₂ is independently any naturally occurring amino acid residue except isoleucine.

80. The antibody or the fragment thereof of claim 79, wherein
X₁ comprises proline or threonine;
X₂ comprises proline or alanine;
X₃ comprises serine or arginine;
X₄ comprises tyrosine or asparagine;
X₅ comprises glycine or aspartic acid;
X₆ comprises alanine or threonine;
X₇ comprises serine or threonine;
or a combination thereof.

81. An isolated anti-IBP4 antibody or a fragment thereof, comprising:
(i) a heavy chain comprising an amino acid sequence that has at least 85%, 90%, 95%, 96%, 97%, 98%, or 99% identity to SEQ ID NO: 68, or a light chain comprising an amino acid sequence that has at least 85%, 90%, 95%, 96%, 97%, 98%, or 99% identity to SEQ ID NO: 70;
(ii) a heavy chain comprising an amino acid sequence that has at least 85%, 90%, 95%, 96%, 97%, 98%, or 99% identity to SEQ ID NO: 103, or a light chain comprising an amino acid sequence that has at least 85%, 90%, 95%, 96%, 97%, 98%, or 99% identity to SEQ ID NO: 105;
(iii) a heavy chain comprising an amino acid sequence that has at least 85%, 90%, 95%, 96%, 97%, 98%, or 99% identity to SEQ ID NO: 123, or a light chain comprising an amino acid sequence that has at least 85%, 90%, 95%, 96%, 97%, 98%, or 99% identity to SEQ ID NO: 70;
(iv) a heavy chain comprising an amino acid sequence that has at least 85%, 90%, 95%, 96%, 97%, 98%, or 99% identity to SEQ ID NO: 128, or a light chain comprising an amino acid sequence that has at least 85%, 90%, 95%, 96%, 97%, 98%, or 99% identity to SEQ ID NO: 105;
(v) a heavy chain comprising an amino acid sequence that has at least 85%, 90%, 95%, 96%, 97%, 98%, or 99% identity to SEQ ID NO: 209, or a light chain comprising an amino acid sequence that has at least 85%, 90%, 95%, 96%, 97%, 98%, or 99% identity to SEQ ID NO: 211;
(vi) a heavy chain comprising an amino acid sequence that has at least 85%, 90%, 95%, 96%, 97%, 98%, or 99% identity to SEQ ID NO: 237, or a light chain comprising an amino acid sequence that has at least 85%, 90%, 95%, 96%, 97%, 98%, or 99% identity to SEQ ID NO: 239;
(vii) a heavy chain comprising an amino acid sequence that has at least 85%, 90%, 95%, 96%, 97%, 98%, or 99% identity to SEQ ID NO: 266, or a light chain comprising an amino acid sequence that has at least 85%, 90%, 95%, 96%, 97%, 98%, or 99% identity to SEQ ID NO: 268;
(viii) a heavy chain comprising an amino acid sequence that has at least 85%, 90%, 95%, 96%, 97%, 98%, or 99% identity to SEQ ID NO: 277, or a light chain comprising an amino acid sequence that has at least 85%, 90%, 95%, 96%, 97%, 98%, or 99% identity to SEQ ID NO: 279;
(ix) a heavy chain comprising an amino acid sequence that has at least 85%, 90%, 95%, 96%, 97%, 98%, or 99% identity to SEQ ID NO: 305, or a light chain comprising an amino acid sequence that has at least 85%, 90%, 95%, 96%, 97%, 98%, or 99% identity to SEQ ID NO: 307;
(x) a heavy chain comprising an amino acid sequence that has at least 85%, 90%, 95%, 96%, 97%, 98%, or 99% identity to SEQ ID NO: 333, or a light chain comprising an amino acid sequence that has at least 85%, 90%, 95%, 96%, 97%, 98%, or 99% identity to SEQ ID NO: 211;
(xi) a heavy chain comprising an amino acid sequence that has at least 85%, 90%, 95%, 96%, 97%, 98%, or 99% identity to SEQ ID NO: 335, or a light chain comprising an amino acid sequence that has at least 85%, 90%, 95%, 96%, 97%, 98%, or 99% identity to SEQ ID NO: 239;
(xii) a heavy chain comprising an amino acid sequence that has at least 85%, 90%, 95%, 96%, 97%, 98%, or 99% identity to SEQ ID NO: 337, or a light chain comprising an amino acid sequence that has at least 85%, 90%, 95%, 96%, 97%, 98%, or 99% identity to SEQ ID NO: 268;
(xiii) a heavy chain comprising an amino acid sequence that has at least 85%, 90%, 95%, 96%, 97%, 98%, or 99% identity to SEQ ID NO: 339, or a light chain comprising an amino acid sequence that has at least 85%, 90%, 95%, 96%, 97%, 98%, or 99% identity to SEQ ID NO: 279; or
(xix) a heavy chain comprising an amino acid sequence that has at least 85%, 90%, 95%, 96%, 97%, 98%, or 99% identity to SEQ ID NO: 341, or a light chain comprising an amino acid sequence that has at least 85%, 90%, 95%, 96%, 97%, 98%, or 99% identity to SEQ ID NO: 307.

82. The antibody or the fragment thereof of claim 81, wherein the antibody or fragment thereof comprises the heavy chain of SEQ ID NO: 68.

83. The antibody or the fragment thereof of claim 81, wherein the antibody or fragment thereof comprises the light chain of SEQ ID NO: 70.

84. The antibody or the fragment thereof of claim 81, wherein the antibody or fragment thereof comprises the heavy chain of SEQ ID NO: 68 and the light chain of SEQ ID NO: 70.

85. The antibody or the fragment thereof of claim 81, wherein the antibody or fragment thereof comprises the heavy chain of SEQ ID NO: 103.

86. The antibody or the fragment thereof of claim 81, wherein the antibody or fragment thereof comprises the light chain of SEQ ID NO: 105.

87. The antibody or the fragment thereof of claim 81, wherein the antibody or fragment thereof comprises the heavy chain of SEQ ID NO: 103 and the light chain of SEQ ID NO: 105.

88. The antibody or the fragment thereof of claim 81, wherein the antibody or fragment thereof comprises the heavy chain of SEQ ID NO: 123.

89. The antibody or the fragment thereof of claim 81, wherein the antibody or fragment thereof comprises the light chain of SEQ ID NO: 70.

90. The antibody or the fragment thereof of claim 81, wherein the antibody or fragment thereof comprises the heavy chain of SEQ ID NO: 123 and the light chain of SEQ ID NO: 70.

91. The antibody or the fragment thereof of claim 81, wherein the antibody or fragment thereof comprises the heavy chain of SEQ ID NO: 128.

92. The antibody or the fragment thereof of claim 81, wherein the antibody or fragment thereof comprises the light chain of SEQ ID NO: 105.

93. The antibody or the fragment thereof of claim 81, wherein the antibody or fragment thereof comprises the heavy chain of SEQ ID NO: 128 and the light chain of SEQ ID NO: 105.

94. The antibody or the fragment thereof of claim 81, wherein the antibody or fragment thereof comprises the heavy chain of SEQ ID NO: 209.

95. The antibody or the fragment thereof of claim 81, wherein the antibody or fragment thereof comprises the light chain of SEQ ID NO: 211.

96. The antibody or the fragment thereof of claim 81, wherein the antibody or fragment thereof comprises the heavy chain of SEQ ID NO: 209 and the light chain of SEQ ID NO: 211.

97. The antibody or the fragment thereof of claim 81, wherein the antibody or fragment thereof comprises the heavy chain of SEQ ID NO: 237.

98. The antibody or the fragment thereof of claim 81, wherein the antibody or fragment thereof comprises the light chain of SEQ ID NO: 239.

99. The antibody or the fragment thereof of claim 81, wherein the antibody or fragment thereof comprises the heavy chain of SEQ ID NO: 237 and the light chain of SEQ ID NO: 239.

100. The antibody or the fragment thereof of claim 81, wherein the antibody or fragment thereof comprises the heavy chain of SEQ ID NO: 266.

101. The antibody or the fragment thereof of claim 81, wherein the antibody or fragment thereof comprises the light chain of SEQ ID NO: 268.

102. The antibody or the fragment thereof of claim 81, wherein the antibody or fragment thereof comprises the heavy chain of SEQ ID NO: 266 and the light chain of SEQ ID NO: 268.

103. The antibody or the fragment thereof of claim 81, wherein the antibody or fragment thereof comprises the heavy chain of SEQ ID NO: 277.

104. The antibody or the fragment thereof of claim 81, wherein the antibody or fragment thereof comprises the light chain of SEQ ID NO: 279.

105. The antibody or the fragment thereof of claim 81, wherein the antibody or fragment thereof comprises the heavy chain of SEQ ID NO: 277 and the light chain of SEQ ID NO: 279.

106. The antibody or the fragment thereof of claim 81, wherein the antibody or fragment thereof comprises the heavy chain of SEQ ID NO: 305.

107. The antibody or the fragment thereof of claim 81, wherein the antibody or fragment thereof comprises the light chain of SEQ ID NO: 307.

108. The antibody or the fragment thereof of claim 81, wherein the antibody or fragment thereof comprises the heavy chain of SEQ ID NO: 305 and the light chain of SEQ ID NO: 307.

109. The antibody or the fragment thereof of claim 81, wherein the antibody or fragment thereof comprises the heavy chain of SEQ ID NO: 333.

110. The antibody or the fragment thereof of claim 81, wherein the antibody or fragment thereof comprises the light chain of SEQ ID NO: 211.

111. The antibody or the fragment thereof of claim 81, wherein the antibody or fragment thereof comprises the heavy chain of SEQ ID NO: 333 and the light chain of SEQ ID NO: 211.

112. The antibody or the fragment thereof of claim 81, wherein the antibody or fragment thereof comprises the heavy chain of SEQ ID NO: 335.

113. The antibody or the fragment thereof of claim 81, wherein the antibody or fragment thereof comprises the light chain of SEQ ID NO: 239.

114. The antibody or the fragment thereof of claim 81, wherein the antibody or fragment thereof comprises the heavy chain of SEQ ID NO: 335 and the light chain of SEQ ID NO: 239.

115. The antibody or the fragment thereof of claim 81, wherein the antibody or fragment thereof comprises the heavy chain of SEQ ID NO: 337.

116. The antibody or the fragment thereof of claim 81, wherein the antibody or fragment thereof comprises the light chain of SEQ ID NO: 268.

117. The antibody or the fragment thereof of claim 81, wherein the antibody or fragment thereof comprises the heavy chain of SEQ ID NO: 337 and the light chain of SEQ ID NO: 268.

118. The antibody or the fragment thereof of claim 81, wherein the antibody or fragment thereof comprises the heavy chain of SEQ ID NO: 339.

119. The antibody or the fragment thereof of claim 81, wherein the antibody or fragment thereof comprises the light chain of SEQ ID NO: 279.

120. The antibody or the fragment thereof of claim 81, wherein the antibody or fragment thereof comprises the heavy chain of SEQ ID NO: 339 and the light chain of SEQ ID NO: 279.

121. The antibody or the fragment thereof of claim 81, wherein the antibody or fragment thereof comprises the heavy chain of SEQ ID NO: 341.

122. The antibody or the fragment thereof of claim 8177, wherein the antibody or fragment thereof comprises the light chain of SEQ ID NO: 307.

123. The antibody or the fragment thereof of claim 81, wherein the antibody or fragment thereof comprises the heavy chain of SEQ ID NO: 341 and the light chain of SEQ ID NO: 307.

124. The antibody or the fragment thereof of any one of claims 32-123, wherein the antibody or the fragment thereof has a dissociation constant (K_{D}) of about 1×10⁻⁷ M or less, 1×10⁻⁸ M or less, 1×10⁻⁹ M or less, 1×10⁻¹⁰ M or less, 2×10⁻⁷ M or less, 2×10⁻⁸ M or less, 2×10⁻⁹ M or less, 2×10⁻¹⁰ M or less, 5×10⁻⁷ M or less, 5×10⁻⁸ M or less, 5×10⁻⁹ M or less, or 5×10⁻¹⁰ M or less.

125. The antibody or the fragment thereof of any one of claims 1 to 124, wherein the antibody or the fragment thereof is a FAB, Fab′, F(ab′)₂, Fv, scFv, (scFv)₂, single chain antibody molecule, dual variable region antibody, single variable region antibody, linear antibody, or a V region formed from antibody fragments.

126. The antibody or the fragment thereof of any one of claims 1 to 125, wherein the antibody or the fragment thereof is humanized, partially humanized, chimeric, or chimeric humanized.

127. The antibody or the fragment thereof of any one of claims 1 to 126, wherein the antibody or the fragment thereof is conjugated or recombinantly fused to a diagnostic or detectable agent.

128. The antibody or the fragment thereof of claim 127, wherein the antibody or fragment thereof is bound to a solid support.

129. The antibody or the fragment thereof of claim 128, wherein the solid support comprises a bead.

130. A nucleic acid molecule encoding the heavy chain of the antibody or the fragment thereof of any one of claims 1 to 126.

131. A nucleic acid molecule encoding the light chain of the antibody or the fragment thereof of any one of claims 1 to 126.

132. A nucleic acid molecule encoding the heavy chain and light chain of the antibody or the fragment thereof of any one of claims 1 to 126.

133. The nucleic acid molecule of any one of claims 130-132, wherein:
(i) the nucleic acid molecule encoding the heavy chain has at least 85%, 90%, 95%, 96%, 97%, 98%, or 99% identity to at least one of SEQ ID NOS: 3, 35, 200, or 202; or
(ii) the nucleic acid molecule encoding the light chain has at least 85%, 90%, 95%, 96%, 97%, 98%, or 99% identity to SEQ ID NO: 5, 37, 201, or 203.

134. The nucleic acid molecule of any one of claims 130-132, wherein:
(i) the nucleic acid molecule encoding the heavy chain has at least 85%, 90%, 95%, 96%, 97%, 98%, or 99% identity to at least one of SEQ ID NOS: 67, 102, 204,206, 208, 236, 265, 276, 304, 334, 336, 338, 340, or 342; or
(ii) the nucleic acid molecule encoding the light chain has at least 85%, 90%, 95%, 96%, 97%, 98%, or 99% identity to SEQ ID NO: 69, 104, 205, 207, 210, 238, 267, 278 or 306.

135. A vector comprising the nucleic acid molecule of any one of claims 130 to 134.

136. An expression vector comprising the nucleic acid molecule of any one of claims 130 to 134.

137. A cell comprising the nucleic acid molecule, vector, or expression vector of any one of claims 130 to 136.

138. A method for detecting one or more biomarkers in a biological sample comprising:
(i) contacting a biological sample comprising one or more biomarkers to the antibody or the fragment thereof of any one of claims 1 to 129; and
(ii) detecting binding of the antibody or the fragment thereof to the one or more biomarkers.

139. The method of claim 138, wherein detecting the binding comprises a proteomics workflow that comprises mass spectrometry quantification.

140. The method of claim 138 or 139, wherein the one or more biomarkers comprises SHBG, and wherein the antibody or the fragment thereof is any one of claims 1 to 31.

141. The method of claim 138 or 139, wherein the one or more biomarkers comprises IBP4, and wherein the antibody or the fragment thereof is any one of claims 32 to 131.

142. The method of claim 138 or 139, wherein the one or more biomarkers comprises SHBG and IBP4, and wherein the antibody or the fragment thereof comprises any one of claims 1 to 31 and any one of claims 32 to 129.

143. The method of any one of claims 138 to 142, wherein the biological sample comprises blood or serum.

144. The method of any one of claims 138 to 145, wherein the detecting comprises separating the one or more biomarkers bound to the antibody or antibody fragment thereof from the biological sample.

145. The method of any one of claims 138 to 144, wherein detecting comprises an immunoassay selected from the group consisting of sandwich immunoassay, immunohistochemistry, enzyme-linked immunosorbent assay (ELISA), Western blotting, nephelometry, turbidimetry, immunoradiometric assay, lateral flow, and immunehisto/cyto-chemistry.

146. A method for assessing or predicting a health status of a subject, the method comprising:
(i) obtaining a biological sample comprising one or more biomarkers from a subject;
(ii) contacting the antibody or the fragment thereof of any one of claims 1 to 129 to the biological sample under conditions suitable for the antibody or the fragment thereof to bind to the one or more biomarkers; and
(iii) detecting the binding of the antibody or the fragment thereof to the one or more biomarker,
wherein detecting the binding of the antibody to the one or more biomarkers indicates the presence of the one or more biomarkers in the biological sample and not detecting the binding of the antibody to the one or more biomarkers indicates the absence of the one or more biomarkers in the biological sample, and where the presence or absence of the one or more biomarker in the biological sample is used to assess or predict the health status of the subject.

147. The method of claim 146, the method further comprises quantitating an amount of the one or more biomarkers bound to the antibody or the fragment thereof in the biological sample, wherein the amount of the one or more biomarker is used to assess or predict the health status of the subject.

148. The method of claim 146 or 147, wherein the subject is a pregnant female.

149. The method of any one of claims 146 to 148, wherein the health status comprises any one of abnormal glucola, gestational diabetes, hypertension, preeclampsia, intrauterine growth restriction, stillbirth, fetal growth restriction, HELLP syndrome, oligohyramnios, chorioamnionitis, chorioamnionitis, placental previa, placental acreta, abruption, abruptio placenta, placental hemorrhage, preterm premature rupture of membranes, preterm labor, unfavorable cervix, postterm pregnancy, cholelithiasis, or uterine over distention.

150. The method of claim 146 to 149, wherein the detecting comprises separating the one or more biomarkers from the biological sample with the antibody or antibody fragment thereof.

151. The method of any one of claims 146 to 150, wherein detecting the binding comprises a proteomics workflow that comprises mass spectrometry quantification.

152. The method of any one of claims 146 to 150, wherein detecting comprises an immunoassay selected from the group consisting of sandwich immunoassay, immunohistochemistry, enzyme-linked immunosorbent assay (ELISA), Western blotting, nephelometry, turbidimetry, immunoradiometric assay, lateral flow, and immunohisto/cyto-chemistry.

153. The method of any one of claims 146 to 152, wherein the one or more biomarkers comprises SHBG, and wherein the antibody or the fragment thereof is any one of claims 1 to 31 or 125 to 129.

154. The method of any one of claims 146 to 152, wherein the one or more biomarkers comprises IBP4, and wherein the antibody or the fragment thereof is any one of claims 32 to 129.

155. The method of any one of claims 146 to 154, wherein the one or more biomarkers comprises SHBG and IBP4, and wherein the antibody or the fragment thereof comprises any one of claims 1 to 129.

156. A method of treating a subject comprising: administering an effective amount of the antibody or the fragment thereof of any one of claims 1 to 126 to the subject.

157. The method of claim 156, wherein the subject is afflicted with a SHBG-associated disease, disorder, or condition, and wherein the subject is administered the antibody or the fragment thereof of any one of claims 1 to 31 or 125 to 126.

158. The method of claim 157, wherein the SHBG-associated disease, disorder, or condition comprises abnormal glucola, gestational diabetes, hypertension, preeclampsia, intrauterine growth restriction, stillbirth, fetal growth restriction, HELLP syndrome, oligohyramnios, chorioamnionitis, chorioamnionitis, placental previa, placental acreta, abruption, abruptio placenta, placental hemorrhage, preterm premature rupture of membranes, preterm labor, unfavorable cervix, postterm pregnancy, cholelithiasis, placental dysfunction, premature cervical shortening, microbial dysbiosis, or uterine over distention.

159. The method of claim 158, wherein the subject is afflicted with a IBP4-associated disease, disorder, or condition, and wherein the subject is administered the antibody or the fragment thereof of any one of claims 32 to 126.

160. The method of claim 159, wherein the IBP4-associated disease, disorder, or condition comprises abnormal glucola, gestational diabetes, hypertension, preeclampsia, intrauterine growth restriction, stillbirth, fetal growth restriction, HELLP syndrome, oligohyramnios, chorioamnionitis, chorioamnionitis, placental previa, placental acreta, abruption, abruptio placenta, placental hemorrhage, preterm premature rupture of membranes, preterm labor, unfavorable cervix, postterm pregnancy, cholelithiasis, placental dysfunction, premature cervical shortening, microbial dysbiosis, or uterine over distention.

161. A composition comprising:
the anti-SHBG antibody or the fragment thereof of any one of claims 1 to 31 or 125 to 129, or the anti-IBP4 antibody or the fragment thereof of any one of claims 32 to 129; and
a carrier.

162. The composition of claim 161, wherein the carrier comprises a solid support.

163. The composition of claim 162, wherein the solid support comprises a bead.

164. The composition of claim 163, wherein the carrier comprises a pharmaceutically acceptable carrier.

165. A kit comprising:
an anti-SHBG antibody or fragment thereof of any one of claims 1 to 31 or 125 to 129, or an anti-IBP4 antibody or fragment thereof of any one of claims 32 to 129;
one or more ancillary reagents; and
packaging for the same.

166. The kit of claim 165, wherein the anti-SHBG antibody or fragment thereof or the anti-IBP4 antibody or fragment thereof is attached to a solid support.

167. The kit of claim 165, wherein the solid support comprises a bead.

168. The kit of claim 165, wherein the kit comprises the anti-IBP4 antibody or the fragment thereof and the anti-SHBG antibody or the fragment thereof.


SEQUENCE LISTING (371 sequences)

SEQ ID NO: 1 - protein, 402 aa, synthetic construct
MESRGPLATSRLLLLLLLLLLRHTRQGWALRPVLPTQSAHDPPAVHLSNGPGQEPIAVMTFDLTKITKTSSSFEVRTWDPEGVIFYGDTNPKDDWFMLGLRDGRPEIQLHNHWAQLTVGAGPRLDDGRWHQVEVKMEGDSVLLEVDGEEVLRLRQVSGPLTSKRHPIMRIALGGLLFPASNLRLPLVPALDGCLRRDSWLDKQAEISASAPTSLRSCDVESNPGIFLPPGTQAEFNLRDIPQPHAEPWAFSLDLGLKQAAGSGHLLALGTPENPSWLSLHLQDQKVVLSSGSGPGLDLPLVLGLPLQLKLSMSRVVLSQGSKMKALALPPLGLAPLLNLWAKPQGRLFLGALPGEDSSTSFCLNGLWAQGQRLDVDQALNRSHEIWTHSCPQSPGNGTDASH

SEQ ID NO: 2 - protein, 258 aa, synthetic construct
MLPLCLVAALLLAAGPGPSLGDEAIHCPPCSEEKLARCRPPVGCEELVREPGCGCCATCALGLGMPCGVYTPRCGSGLRCYPPRGVEKPLHTLMHGQGVCMELAEIEAIQESLQPSDKDEGDHPNNSFSPCSAHDRRCLQKHFAKIRDRSTSGGKMKVNGAPREDARPVPQGSCQSELHRALERLAASQSRTHEDLYIIPIPNCDRNGNFHPKQCHPALDGQRGKCWCVDRKTGVKLPGGLEPKGELDCHQLADSFRE

SEQ ID NO: 3 - DNA, 1350 bp, synthetic construct
  source 1..1350: mol_type = other DNA
caggtccaactgcagcagcctggggctgagctggtgaagcctggggcttcagtgaagatgtcttgtaaggcttctggctacaccttcaccggctactggatgcactgggtgaagcagaggcctggacaaggccttgagtggatcggagtaattgatccttctactggctatactacctacaatgaaaaattcaagggcaaggccacattgactgtagacacatcctccaacacagcctacatgcagctcagcagcctgacctctgaggactctgcggtctattactgtacaagagagccgttagtcactgcggactttgagtattggggccaaggcaccactctcacagtctcctcagccaaaacaacagccccatcggtctatccactggcccctgtgtgtggagatacaactggctcctcggtgactctgggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 4 - protein, 449 aa, synthetic construct
QVQLQQPGAELVKPGASVKMSCKASGYTFTGYWMHWVKQRPGQGLEWIGVIDPSTGYTTYNEKFKGKATLTVDTSSNTAYMQLSSLTSEDSAVYYCTREPLVTADFEYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 5 - DNA, 648 bp, synthetic construct
  source 1..648: mol_type = other DNA
gacattgtgatgacccagtctcaaaaattcatgtccacatcagttggagacaggatcagcgtcacctgcaaggccagtcagaatgtggatactaatgtagcctggtatcgacagaaaccagggcaatctcctaaagcactgatttactcggcatcctaccgcttcagtggagtccctgatcgcttcacaggcagtggatctgggacagatttcactctcaccatcagcactttccagtctgaagacttggcagaatatttctgtcagcaatatgacagctatccttacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgtaaatag

SEQ ID NO: 6 - protein, 215 aa, synthetic construct
DIVMTQSQKFMSTSVGDRISVTCKASQNVDTNVAWYRQKPGQSPKALIYSASYRFSGVPDRFTGSGSGTDFTLTISTFQSEDLAEYFCQQYDSYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECK

SEQ ID NO: 7 - DNA, 358 bp, synthetic construct
  source 1..358: mol_type = other DNA
caggtccaactgcagcagcctggggctgagctggtgaagcctggggcttcagtgaagatgtcttgtaaggcttctggctacaccttcaccggctactggatgcactgggtgaagcagaggcctggacaaggccttgagtggatcggagtaattgatccttctactggctatactacctacaatgaaaaattcaagggcaaggccacattgactgtagacacatcctccaacacagcctacatgcagctcagcagcctgacctctgaggactctgcggtctattactgtacaagagagccgttagtcactgcggactttgagtattggggccaaggcaccactctcacagtctcctcag

SEQ ID NO: 8 - protein, 119 aa, synthetic construct
QVQLQQPGAELVKPGASVKMSCKASGYTFTGYWMHWVKQRPGQGLEWIGVIDPSTGYTTYNEKFKGKATLTVDTSSNTAYMQLSSLTSEDSAVYYCTREPLVTADFEYWGQGTTLTVSS

SEQ ID NO: 9 - DNA, 322 bp, synthetic construct
  source 1..322: mol_type = other DNA
gacattgtgatgacccagtctcaaaaattcatgtccacatcagttggagacaggatcagcgtcacctgcaaggccagtcagaatgtggatactaatgtagcctggtatcgacagaaaccagggcaatctcctaaagcactgatttactcggcatcctaccgcttcagtggagtccctgatcgcttcacaggcagtggatctgggacagatttcactctcaccatcagcactttccagtctgaagacttggcagaatatttctgtcagcaatatgacagctatccttacacgttcggaggggggaccaagctggaaataaaac

SEQ ID NO: 10 - protein, 107 aa, synthetic construct
DIVMTQSQKFMSTSVGDRISVTCKASQNVDTNVAWYRQKPGQSPKALIYSASYRFSGVPDRFTGSGSGTDFTLTISTFQSEDLAEYFCQQYDSYPYTFGGGTKLEIK

SEQ ID NO: 11 - protein, 8 aa, synthetic construct
GYTFTGYW

SEQ ID NO: 12 - protein, 8 aa, synthetic construct
IDPSTGYT

SEQ ID NO: 13 - protein, 12 aa, synthetic construct
TREPLVTADFEY

SEQ ID NO: 14 - protein, 10 aa, synthetic construct
GYTFTGYWMH

SEQ ID NO: 15 - protein, 17 aa, synthetic construct
VIDPSTGYTTYNEKFKG

SEQ ID NO: 16 - protein, 10 aa, synthetic construct
EPLVTADFEY

SEQ ID NO: 17 - protein, 13 aa, synthetic construct
GYTFTGYWMHWVK

SEQ ID NO: 18 - protein, 21 aa, synthetic construct
IGVIDPSTGYTTYNEKFKGKA

SEQ ID NO: 19 - protein, 12 aa, synthetic construct
REPLVTADFEYW

SEQ ID NO: 20 - protein, 9 aa, synthetic construct
TGYWMHWVK

SEQ ID NO: 21 - protein, 24 aa, synthetic construct
LEWIGVIDPSTGYTTYNEKFKGKA

SEQ ID NO: 22 - protein, 13 aa, synthetic construct
CTREPLVTADFEY

SEQ ID NO: 23 - protein, 6 aa, synthetic construct
QNVDTN

SEQ ID NO: 24 - skipped

SEQ ID NO: 25 - protein, 8 aa, synthetic construct
QQYDSYPY

SEQ ID NO: 26 - protein, 11 aa, synthetic construct
KASQNVDTNVA

SEQ ID NO: 27 - protein, 7 aa, synthetic construct
SASYRFS

SEQ ID NO: 28 - skipped

SEQ ID NO: 29 - protein, 15 aa, synthetic construct
TCKASQNVDTNVAWY

SEQ ID NO: 30 - protein, 11 aa, synthetic construct
SASYRFSGVPD

SEQ ID NO: 31 - protein, 12 aa, synthetic construct
FCQQYDSYPYTF

SEQ ID NO: 32 - protein, 11 aa, synthetic construct
NVDTNVAWYRQ

SEQ ID NO: 33 - protein, 14 aa, synthetic construct
ALIYSASYRFSGVP

SEQ ID NO: 34 - protein, 11 aa, synthetic construct
FCQQYDSYPYT

SEQ ID NO: 35 - DNA, 1332 bp, synthetic construct
  source 1..1332: mol_type = other DNA
cagatccaactgcagcagcctggggctgagctggtgaagcctggggcttcagtgaagatgtcctgcaaggcttctggctacaccttcaccggctactggatgcactgggtgaagcagaggcctggacaaggccttgagtggatcggagtgattgatccttctgatacttatactaactacaatcaaaaattcaggggcaaggccacattgactgtagacacatcctccagcacagcctacatgcagctcagcggcctgacctctgaggactctgcggtctattactgtacaagagatccgttagtaactgcggactttgactactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 36 - protein, 443 aa, synthetic construct
QIQLQQPGAELVKPGASVKMSCKASGYTFTGYWMHWVKQRPGQGLEWIGVIDPSDTYTNYNQKFRGKATLTVDTSSSTAYMQLSGLTSEDSAVYYCTRDPLVTADFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 37 - DNA, 648 bp, synthetic construct
  source 1..648: mol_type = other DNA
gacattgtgatgacccagtctcaaaaattcatgtccacatcagttggagacagggtcagcgtcacctgcaaggccagtcagaatgtggatactaatgtagcctggtatcgacagaaaccagggcaatctcctaaagcgctgatttactcggcatcctaccgattcagtggagtccctgatcgcttcacaggcagtggatctgggacagatttcactctcaccatcagcaatttccagtctgaggacttggcagaatatttctgtcagcaatatgacagctatccttacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgtaaatag

SEQ ID NO: 38 - protein, 215 aa, synthetic construct
DIVMTQSQKFMSTSVGDRVSVTCKASQNVDTNVAWYRQKPGQSPKALIYSASYRFSGVPDRFTGSGSGTDFTLTISNFQSEDLAEYFCQQYDSYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECK

SEQ ID NO: 39 - DNA, 358 bp, synthetic construct
  source 1..358: mol_type = other DNA
cagatccaactgcagcagcctggggctgagctggtgaagcctggggcttcagtgaagatgtcctgcaaggcttctggctacaccttcaccggctactggatgcactgggtgaagcagaggcctggacaaggccttgagtggatcggagtgattgatccttctgatacttatactaactacaatcaaaaattcaggggcaaggccacattgactgtagacacatcctccagcacagcctacatgcagctcagcggcctgacctctgaggactctgcggtctattactgtacaagagatccgttagtaactgcggactttgactactggggccaaggcaccactctcacagtctcctcag

SEQ ID NO: 40 - protein, 119 aa, synthetic construct
QIQLQQPGAELVKPGASVKMSCKASGYTFTGYWMHWVKQRPGQGLEWIGVIDPSDTYTNYNQKFRGKATLTVDTSSSTAYMQLSGLTSEDSAVYYCTRDPLVTADFDYWGQGTTLTVSS

SEQ ID NO: 41 - DNA, 322 bp, synthetic construct
  source 1..322: mol_type = other DNA
gacattgtgatgacccagtctcaaaaattcatgtccacatcagttggagacagggtcagcgtcacctgcaaggccagtcagaatgtggatactaatgtagcctggtatcgacagaaaccagggcaatctcctaaagcgctgatttactcggcatcctaccgattcagtggagtccctgatcgcttcacaggcagtggatctgggacagatttcactctcaccatcagcaatttccagtctgaggacttggcagaatatttctgtcagcaatatgacagctatccttacacgttcggaggggggaccaagctggaaataaaac

SEQ ID NO: 42 - protein, 107 aa, synthetic construct
DIVMTQSQKFMSTSVGDRVSVTCKASQNVDTNVAWYRQKPGQSPKALIYSASYRFSGVPDRFTGSGSGTDFTLTISNFQSEDLAEYFCQQYDSYPYTFGGGTKLEIK

SEQ ID NO: 43 - skipped

SEQ ID NO: 44 - protein, 8 aa, synthetic construct
IDPSDTYT

SEQ ID NO: 45 - protein, 12 aa, synthetic construct
TRDPLVTADFDY

SEQ ID NO: 46 - skipped

SEQ ID NO: 47 - protein, 17 aa, synthetic construct
VIDPSDTYTNYNQKFRG

SEQ ID NO: 48 - protein, 10 aa, synthetic construct
DPLVTADFDY

SEQ ID NO: 49 - skipped

SEQ ID NO: 50 - protein, 21 aa, synthetic construct
IGVIDPSDTYTNYNQKFRGKA

SEQ ID NO: 51 - protein, 12 aa, synthetic construct
RDPLVTADFDYW

SEQ ID NO: 52 - protein, 8 aa, synthetic construct
TGYWMHVK

SEQ ID NO: 53 - protein, 24 aa, synthetic construct
LEWIGVIDPSDTYTNYNQKFRGKA

SEQ ID NO: 54 - protein, 13 aa, synthetic construct
CTRDPLVTADFDY

SEQ ID NO: 55-66 - skipped (12 entries)

SEQ ID NO: 67 - DNA, 1323 bp, synthetic construct
  source 1..1323: mol_type = other DNA
gaggtccagctgcaacaatctggacctgagctggtgaagcctggggcttcagcgaagatatcctgtaaggcttctggatacacgttcactgactactacatgaactgggtgaagcagagccatggaaagagccttgagtggattggagatattaatcctaacaatggtggttctgcctacaaccagaagttcaagggcaaggccacattgactgtagacaagtcctccagcacagcctacatggagctccgcagcctgacatctgaggactctgcagtctattactgtgcaagatgggatggaggctttgactactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 68 - protein, 440 aa, synthetic construct
EVQLQQSGPELVKPGASAKISCKASGYTFTDYYMNWVKQSHGKSLEWIGDINPNNGGSAYNQKFKGKATLTVDKSSSTAYMELRSLTSEDSAVYYCARWDGGFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 69 - DNA, 648 bp, synthetic construct
  source 1..648: mol_type = other DNA
gacattgtgatgacccagtctcaaaaattcatgtccacatcagtaagagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagctttcactctcaccatcagcaatgtgcagtctgaagacttggcagagtatttctgtcagcaatataacatctatcctctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccagagacatcaatgtcaagtggaagattgatggcagtgaacgacaaaatggtgtcctgaacagttggactgatcaggacagcaaagacagcacctacagcatgagcagcaccctcacattgaccaaggacgagtatgaacgacataacagctatacctgtgaggccactcacaagacatcaacttcacccatcgtcaagagcttcaacaggaatgagtgtaaatag

SEQ ID NO: 70 - protein, 215 aa, synthetic construct
DIVMTQSQKFMSTSVRDRVSVTCKASQNVGTNVAWYQQKPGQSPKALIYSASYRYSGVPDRFTGSGSGTAFTLTISNVQSEDLAEYFCQQYNIYPLTFGAGTKLELKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNECK

SEQ ID NO: 71 - DNA, 349 bp, synthetic construct
  source 1..349: mol_type = other DNA
gaggtccagctgcaacaatctggacctgagctggtgaagcctggggcttcagcgaagatatcctgtaaggcttctggatacacgttcactgactactacatgaactgggtgaagcagagccatggaaagagccttgagtggattggagatattaatcctaacaatggtggttctgcctacaaccagaagttcaagggcaaggccacattgactgtagacaagtcctccagcacagcctacatggagctccgcagcctgacatctgaggactctgcagtctattactgtgcaagatgggatggaggctttgactactggggccaaggcaccactctcacagtctcctcag

SEQ ID NO: 72 - protein, 116 aa, synthetic construct
EVQLQQSGPELVKPGASAKISCKASGYTFTDYYMNWVKQSHGKSLEWIGDINPNNGGSAYNQKFKGKATLTVDKSSSTAYMELRSLTSEDSAVYYCARWDGGFDYWGQGTTLTVSS

SEQ ID NO: 73 - DNA, 322 bp, synthetic construct
  source 1..322: mol_type = other DNA
gacattgtgatgacccagtctcaaaaattcatgtccacatcagtaagagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagctttcactctcaccatcagcaatgtgcagtctgaagacttggcagagtatttctgtcagcaatataacatctatcctctcacgttcggtgctgggaccaagctggagctgaaac

SEQ ID NO: 74 - protein, 107 aa, synthetic construct
DIVMTQSQKFMSTSVRDRVSVTCKASQNVGTNVAWYQQKPGQSPKALIYSASYRYSGVPDRFTGSGSGTAFTLTISNVQSEDLAEYFCQQYNIYPLTFGAGTKLELK

SEQ ID NO: 75 - protein, 8 aa, synthetic construct
GYTFTDYY

SEQ ID NO: 76 - protein, 8 aa, synthetic construct
INPNNGGS

SEQ ID NO: 77 - protein, 9 aa, synthetic construct
ARWDGGFDY

SEQ ID NO: 78 - protein, 10 aa, synthetic construct
GYTFTDYYMN

SEQ ID NO: 79 - protein, 17 aa, synthetic construct
DINPNNGGSAYNQKFKG

SEQ ID NO: 80 - protein, 7 aa, synthetic construct
WDGGFDY

SEQ ID NO: 81 - protein, 13 aa, synthetic construct
GYTFTDYYMNWVK

SEQ ID NO: 82 - protein, 21 aa, synthetic construct
IGDINPNNGGSAYNQKFKGKA

SEQ ID NO: 83 - protein, 9 aa, synthetic construct
RWDGGFDYW

SEQ ID NO: 84 - protein, 9 aa, synthetic construct
TDYYMNWVK

SEQ ID NO: 85 - protein, 24 aa, synthetic construct
LEWIGDINPNNGGSAYNQKFKGKA

SEQ ID NO: 86 - protein, 10 aa, synthetic construct
CARWDGGFDY

SEQ ID NO: 87 - protein, 6 aa, synthetic construct
QNVGTN

SEQ ID NO: 88 - skipped

SEQ ID NO: 89 - protein, 9 aa, synthetic construct
QQYNIYPLT

SEQ ID NO: 90 - protein, 11 aa, synthetic construct
KASQNVGTNVA

SEQ ID NO: 91 - protein, 7 aa, synthetic construct
SASYRYS

SEQ ID NO: 92 - skipped

SEQ ID NO: 93 - protein, 15 aa, synthetic construct
TCKASQNVGTNVAWY

SEQ ID NO: 94 - protein, 11 aa, synthetic construct
SASYRYSGVPD

SEQ ID NO: 95 - protein, 12 aa, synthetic construct
FCQQYNIYPLTF

SEQ ID NO: 96 - protein, 11 aa, synthetic construct
NVGTNVAWYQQ

SEQ ID NO: 97 - protein, 14 aa, synthetic construct
ALIYSASYRYSGVP

SEQ ID NO: 98 - protein, 11 aa, synthetic construct
FCQQYNIYPLT

SEQ ID NO: 99 - skipped

SEQ ID NO: 100 - protein, 12 aa, synthetic construct
FCQQYNSYPLTF

SEQ ID NO: 101 - protein, 11 aa, synthetic construct
FCQQYNSYPLT

SEQ ID NO: 102 - DNA, 1323 bp, synthetic construct
  source 1..1323: mol_type = other DNA
gaggtccagctgcaacaatctggacctgagctggtgaagcctggggcttcagtgaagatatcctgtaaggcttctggatacacgttcactgactactacatgaactgggtgaagcagagccatggaaagagccttgagtggattggagatattaatcctaacaatggtggttctgcctacaaccagaagttcaagggcaaggccacattgactgtagacaagtcctccagcacagcctacatggagctccgcagcctgacatctgaggactctgcagtctattactgtgcaagatgggatggaggctttgactactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 103 - protein, 440 aa, synthetic construct
EVQLQQSGPELVKPGASVKISCKASGYTFTDYYMNWVKQSHGKSLEWIGDINPNNGGSAYNQKFKGKATLTVDKSSSTAYMELRSLTSEDSAVYYCARWDGGFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 104 - DNA, 648 bp, synthetic construct
  source 1..648: mol_type = other DNA
gacattgtgatgacccagtctcaaaaattcatgtccacatcagtaagagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcagcaatataacagctatcctctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccagagacatcaatgtcaagtggaagattgatggcagtgaacgacaaaatggtgtcctgaacagttggactgatcaggacagcaaagacagcacctacagcatgagcagcaccctcacattgaccaaggacgagtatgaacgacataacagctatacctgtgaggccactcacaagacatcaacttcacccatcgtcaagagcttcaacaggaatgagtgtaaatag

SEQ ID NO: 105 - protein, 215 aa, synthetic construct
DIVMTQSQKFMSTSVRDRVSVTCKASQNVGTNVAWYQQKPGQSPKALIYSASYRYSGVPDRFTGSGSGTDFTLTISNVQSEDLAEYFCQQYNSYPLTFGAGTKLELKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNECK

SEQ ID NO: 106 - DNA, 349 bp, synthetic construct
  source 1..349: mol_type = other DNA
gaggtccagctgcaacaatctggacctgagctggtgaagcctggggcttcagtgaagatatcctgtaaggcttctggatacacgttcactgactactacatgaactgggtgaagcagagccatggaaagagccttgagtggattggagatattaatcctaacaatggtggttctgcctacaaccagaagttcaagggcaaggccacattgactgtagacaagtcctccagcacagcctacatggagctccgcagcctgacatctgaggactctgcagtctattactgtgcaagatgggatggaggctttgactactggggccaaggcaccactctcacagtctcctcag

SEQ ID NO: 107 - protein, 116 aa, synthetic construct
EVQLQQSGPELVKPGASVKISCKASGYTFTDYYMNWVKQSHGKSLEWIGDINPNNGGSAYNQKFKGKATLTVDKSSSTAYMELRSLTSEDSAVYYCARWDGGFDYWGQGTTLTVSS

SEQ ID NO: 108 - DNA, 322 bp, synthetic construct
  source 1..322: mol_type = other DNA
gacattgtgatgacccagtctcaaaaattcatgtccacatcagtaagagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcagcaatataacagctatcctctcacgttcggtgctgggaccaagctggagctgaaac

SEQ ID NO: 109 - protein, 107 aa, synthetic construct
DIVMTQSQKFMSTSVRDRVSVTCKASQNVGTNVAWYQQKPGQSPKALIYSASYRYSGVPDRFTGSGSGTDFTLTISNVQSEDLAEYFCQQYNSYPLTFGAGTKLELK

SEQ ID NO: 110 - protein, 23 aa, synthetic construct
LRPVLPTQSAHDPPAVHLSNGPG

SEQ ID NO: 111 - protein, 17 aa, synthetic construct
KQCHPALDGQRGKCWCV

SEQ ID NO: 112 - protein, 8 aa, synthetic construct
  VARIANT 5: note = X can be any Amino Acid
  VARIANT 6: note = X can be any Amino Acid
IDPSXXYT

SEQ ID NO: 113 - protein, 12 aa, synthetic construct
  VARIANT 3: note = X can be any Amino Acid
  VARIANT 11: note = X can be any Amino Acid
TRXPLVTADFXY

SEQ ID NO: 114 - protein, 9 aa, synthetic construct
  VARIANT 5: note = X can be any Amino Acid
QQYNXYPLT

SEQ ID NO: 115 - protein, 7 aa, synthetic construct
YWMHWVK

SEQ ID NO: 116 - protein, 24 aa, synthetic construct
IGVIDPSTGYTTYNEKFKGKATLT

SEQ ID NO: 117 - protein, 24 aa, synthetic construct
IGVIDPSDTYTNYNQKFRGKATLT

SEQ ID NO: 118 - protein, 7 aa, synthetic construct
YYMNWVK

SEQ ID NO: 119 - protein, 24 aa, synthetic construct
IGDINPNNGGSAYNQKFKGKATLT

SEQ ID NO: 120 - protein, 402 aa, synthetic construct; identical to SEQ ID NO: 1

SEQ ID NO: 121 - protein, 402 aa, synthetic construct; identical to SEQ ID NO: 1

SEQ ID NO: 122 - protein, 214 aa, synthetic construct
DIVMTQSQKFMSTSVRDRVSVTCKASQNVGTNVAWYQQKPGQSPKALIYSASYRYSGVPDRFTGSGSGTAFTLTISNVQSEDLAEYFCQQYNIYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 123 - protein, 439 aa, synthetic construct
EVQLQQSGPELVKPGASAKISCKASGYTFTDYYMNWVKQSHGKSLEWIGDINPNNGGSAYNQKFKGKATLTVDKSSSTAYMELRSLTSEDSAVYYCARWDGGFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPG

SEQ ID NO: 124 - protein, 448 aa, synthetic construct
QVQLQQPGAELVKPGASVKMSCKASGYTFTGYWMHWVKQRPGQGLEWIGVIDPSTGYTTYNEKFKGKATLTVDTSSNTAYMQLSSLTSEDSAVYYCTREPLVTADFEYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 125 - protein, 214 aa, synthetic construct
DIVMTQSQKFMSTSVGDRISVTCKASQNVDTNVAWYRQKPGQSPKALIYSASYRFSGVPDRFTGSGSGTDFTLTISTFQSEDLAEYFCQQYDSYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 126 - protein, 442 aa, synthetic construct
QIQLQQPGAELVKPGASVKMSCKASGYTFTGYWMHWVKQRPGQGLEWIGVIDPSDTYTNYNQKFRGKATLTVDTSSSTAYMQLSGLTSEDSAVYYCTRDPLVTADFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 127 - protein, 214 aa, synthetic construct
DIVMTQSQKFMSTSVGDRVSVTCKASQNVDTNVAWYRQKPGQSPKALIYSASYRFSGVPDRFTGSGSGTDFTLTISNFQSEDLAEYFCQQYDSYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 128 - protein, 439 aa, synthetic construct
EVQLQQSGPELVKPGASVKISCKASGYTFTDYYMNWVKQSHGKSLEWIGDINPNNGGSAYNQKFKGKATLTVDKSSSTAYMELRSLTSEDSAVYYCARWDGGFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPG

SEQ ID NO: 129 - protein, 214 aa, synthetic construct
DIVMTQSQKFMSTSVRDRVSVTCKASQNVGTNVAWYQQKPGQSPKALIYSASYRYSGVPDRFTGSGSGTDFTLTISNVQSEDLAEYFCQQYNSYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 130 - skipped

SEQ ID NO: 131 - protein, 22 aa, synthetic construct
CHPALDGQRGKCWCVDRKTGVK

SEQ ID NO: 132-199 - skipped (68 entries)

SEQ ID NO: 200 - DNA, 1347 bp, synthetic construct
  source 1..1347: mol_type = other DNA
caggtccaactgcagcagcctggggctgagctggtgaagcctggggcttcagtgaagatgtcttgtaaggcttctggctacaccttcaccggctactggatgcactgggtgaagcagaggcctggacaaggccttgagtggatcggagtaattgatccttctactggctatactacctacaatgaaaaattcaagggcaaggccacattgactgtagacacatcctccaacacagcctacatgcagctcagcagcctgacctctgaggactctgcggtctattactgtacaagagagccgttagtcactgcggactttgagtattggggccaaggcaccactctcacagtctcctcagccaaaacaacagccccatcggtctatccactggcccctgtgtgtggagatacaactggctcctcggtgactctgggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggttga

SEQ ID NO: 201 - DNA, 645 bp, synthetic construct
  source 1..645: mol_type = other DNA
gacattgtgatgacccagtctcaaaaattcatgtccacatcagttggagacaggatcagcgtcacctgcaaggccagtcagaatgtggatactaatgtagcctggtatcgacagaaaccagggcaatctcctaaagcactgatttactcggcatcctaccgcttcagtggagtccctgatcgcttcacaggcagtggatctgggacagatttcactctcaccatcagcactttccagtctgaagacttggcagaatatttctgtcagcaatatgacagctatccttacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 202 - DNA, 1329 bp, synthetic construct
  source 1..1329: mol_type = other DNA
cagatccaactgcagcagcctggggctgagctggtgaagcctggggcttcagtgaagatgtcctgcaaggcttctggctacaccttcaccggctactggatgcactgggtgaagcagaggcctggacaaggccttgagtggatcggagtgattgatccttctgatacttatactaactacaatcaaaaattcaggggcaaggccacattgactgtagacacatcctccagcacagcctacatgcagctcagcggcctgacctctgaggactctgcggtctattactgtacaagagatccgttagtaactgcggactttgactactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggttga

SEQ ID NO: 203 - DNA, 645 bp, synthetic construct
  source 1..645: mol_type = other DNA
gacattgtgatgacccagtctcaaaaattcatgtccacatcagttggagacagggtcagcgtcacctgcaaggccagtcagaatgtggatactaatgtagcctggtatcgacagaaaccagggcaatctcctaaagcgctgatttactcggcatcctaccgattcagtggagtccctgatcgcttcacaggcagtggatctgggacagatttcactctcaccatcagcaatttccagtctgaggacttggcagaatatttctgtcagcaatatgacagctatccttacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 204 - DNA, 1132 bp, synthetic construct
  source 1..1132: mol_type = other DNA
gttcaagggcaaggccacattgactgtagacaagtcctccagcacagcctacatggagctccgcagcctgacatctgaggactctgcagtctattactgtgcaagatgggatggaggctttgactactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggttga

SEQ ID NO: 205 - DNA, 645 bp, synthetic construct
  source 1..645: mol_type = other DNA
gacattgtgatgacccagtctcaaaaattcatgtccacatcagtaagagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagctttcactctcaccatcagcaatgtgcagtctgaagacttggcagagtatttctgtcagcaatataacatctatcctctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccagagacatcaatgtcaagtggaagattgatggcagtgaacgacaaaatggtgtcctgaacagttggactgatcaggacagcaaagacagcacctacagcatgagcagcaccctcacattgaccaaggacgagtatgaacgacataacagctatacctgtgaggccactcacaagacatcaacttcacccatcgtcaagagcttcaacaggaatgagtgttag

SEQ ID NO: 206 - DNA, 1320 bp, synthetic construct
  source 1..1320: mol_type = other DNA
gaggtccagctgcaacaatctggacctgagctggtgaagcctggggcttcagtgaagatatcctgtaaggcttctggatacacgttcactgactactacatgaactgggtgaagcagagccatggaaagagccttgagtggattggagatattaatcctaacaatggtggttctgcctacaaccagaagttcaagggcaaggccacattgactgtagacaagtcctccagcacagcctacatggagctccgcagcctgacatctgaggactctgcagtctattactgtgcaagatgggatggaggctttgactactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggttga

SEQ ID NO: 207 - DNA, 645 bp, synthetic construct
  source 1..645: mol_type = other DNA
gacattgtgatgacccagtctcaaaaattcatgtccacatcagtaagagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcagcaatataacagctatcctctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccagagacatcaatgtcaagtggaagattgatggcagtgaacgacaaaatggtgtcctgaacagttggactgatcaggacagcaaagacagcacctacagcatgagcagcaccctcacattgaccaaggacgagtatgaacgacataacagctatacctgtgaggccactcacaagacatcaacttcacccatcgtcaagagcttcaacaggaatgagtgttag

SEQ ID NO: 208 - DNA, 1329 bp, synthetic construct
  source 1..1329: mol_type = other DNA
gaggtccagctgcaacaatctggacctgagctggtgaagcctggggcttcagtgaagatatcctgtaagacttctggatacacgttcactgactactacatgaactgggtgaagcagagccatggaaagagccttgagtggattggagatattaatcctaacaatggtggtgctggctacaaccagaagttcaagggcaaggccacattgactgtagacaagtcctccagcacagcctacatggagctccgcagcctgacatctgaggactctgcagtctattactgtgcaagatggggctgggaccggtactttgactactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 209 - protein, 442 aa, synthetic construct
EVQLQQSGPELVKPGASVKISCKTSGYTFTDYYMNWVKQSHGKSLEWIGDINPNNGGAGYNQKFKGKATLTVDKSSSTAYMELRSLTSEDSAVYYCARWGWDRYFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 210 - DNA, 645 bp, synthetic construct
  source 1..645: mol_type = other DNA
gacattgtgatgacccagtctcaaaaattcatgtccacttcagtaggagacagggtcagcgtcacctgcaaggccagtcagaatgtgggtattgatgtagcctggtatcaacagaaaccaggacaatctcctaaagcactgatttactcggcatcctaccggtccagtggagtccctgatcgcttcacaggcagtggatctgggacagatttcactctcaccatcaacaatgtgcagtctgaagacttggcagagtatttctgtcagcaatataacagctatcctctagcgttcggctcggggacaaagttggaaataaaacgggctgatgctgcaccaactgtatccatcttcccaccatccagtgagcagttaacatctggaggtgcctcagtcgtgtgcttcttgaacaacttctaccccagagacatcaatgtcaagtggaagattgatggcagtgaacgacaaaatggtgtcctgaacagttggactgatcaggacagcaaagacagcacctacagcatgagcagcaccctcacattgaccaaggacgagtatgaacgacataacagctatacctgtgaggccactcacaagacatcaacttcacccatcgtcaagagcttcaacaggaatgagtgttag

SEQ ID NO: 211 - protein, 214 aa, synthetic construct
DIVMTQSQKFMSTSVGDRVSVTCKASQNVGIDVAWYQQKPGQSPKALIYSASYRSSGVPDRFTGSGSGTDFTLTINNVQSEDLAEYFCQQYNSYPLAFGSGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 212 - DNA, 355 bp, synthetic construct
  source 1..355: mol_type = other DNA
gaggtccagctgcaacaatctggacctgagctggtgaagcctggggcttcagtgaagatatcctgtaagacttctggatacacgttcactgactactacatgaactgggtgaagcagagccatggaaagagccttgagtggattggagatattaatcctaacaatggtggtgctggctacaaccagaagttcaagggcaaggccacattgactgtagacaagtcctccagcacagcctacatggagctccgcagcctgacatctgaggactctgcagtctattactgtgcaagatggggctgggaccggtactttgactactggggccaaggcaccactctcacagtctcctcag

SEQ ID NO: 213 - protein, 118 aa, synthetic construct
EVQLQQSGPELVKPGASVKISCKTSGYTFTDYYMNWVKQSHGKSLEWIGDINPNNGGAGYNQKFKGKATLTVDKSSSTAYMELRSLTSEDSAVYYCARWGWDRYFDYWGQGTTLTVSS

SEQ ID NO: 214 - DNA, 322 bp, synthetic construct
  source 1..322: mol_type = other DNA
gacattgtgatgacccagtctcaaaaattcatgtccacttcagtaggagacagggtcagcgtcacctgcaaggccagtcagaatgtgggtattgatgtagcctggtatcaacagaaaccaggacaatctcctaaagcactgatttactcggcatcctaccggtccagtggagtccctgatcgcttcacaggcagtggatctgggacagatttcactctcaccatcaacaatgtgcagtctgaagacttggcagagtatttctgtcagcaatataacagctatcctctagcgttcggctcggggacaaagttggaaataaaac

SEQ ID NO: 215 - protein, 107 aa, synthetic construct
DIVMTQSQKFMSTSVGDRVSVTCKASQNVGIDVAWYQQKPGQSPKALIYSASYRSSGVPDRFTGSGSGTDFTLTINNVQSEDLAEYFCQQYNSYPLAFGSGTKLEIK

SEQ ID NO: 216 - protein, 8 aa, synthetic construct
INPNNGGA

SEQ ID NO: 217 - protein, 11 aa, synthetic construct
ARWGWDRYFDY

SEQ ID NO: 218 - protein, 17 aa, synthetic construct
DINPNNGGAGYNQKFKG

SEQ ID NO: 219 - protein, 9 aa, synthetic construct
WGWDRYFDY

SEQ ID NO: 220 - protein, 21 aa, synthetic construct
IGDINPNNGGAGYNQKFKGKA

SEQ ID NO: 221 - protein, 11 aa, synthetic construct
RWGWDRYFDYW

SEQ ID NO: 222 - protein, 24 aa, synthetic construct
LEWIGDINPNNGGAGYNQKFKGKA

SEQ ID NO: 223 - protein, 12 aa, synthetic construct
CARWGWDRYFDY

SEQ ID NO: 224 - protein, 24 aa, synthetic construct
IGDINPNNGGAGYNQKFKGKATLT

SEQ ID NO: 225 - protein, 10 aa, synthetic construct
WGWDRYFDYW

SEQ ID NO: 226 - protein, 6 aa, synthetic construct
QNVGID

SEQ ID NO: 227 - protein, 9 aa, synthetic construct
QQYNSYPLA

SEQ ID NO: 228 - protein, 11 aa, synthetic construct
KASQNVGIDVA

SEQ ID NO: 229 - protein, 6 aa, synthetic construct
SASYRS

SEQ ID NO: 230 - protein, 15 aa, synthetic construct
TCKASQNVGIDVAWY

SEQ ID NO: 231 - protein, 11 aa, synthetic construct
SASYRSSGVPD

SEQ ID NO: 232 - protein, 12 aa, synthetic construct
FCQQYNSYPLAF

SEQ ID NO: 233 - protein, 11 aa, synthetic construct
NVGIDVAWYQQ

SEQ ID NO: 234 - protein, 14 aa, synthetic construct
ALIYSASYRSSGVP

SEQ ID NO: 235 - protein, 11 aa, synthetic construct
FCQQYNSYPLA

SEQ ID NO: 236 - DNA, 1338 bp, synthetic construct
  source 1..1338: mol_type = other DNA
caggttcaactgcagcagtctggggctgaactggtgaggcctggggcttcagtgacgctgtcctgcaaggcttcgggctacacatttactgactatgaaatgcactgggtgaggcagacacctgtgcatggcctggaatggattggagctattgatactgaaactggtggtcctgcctacaatcagaagttcaagggcaaggccatactgactgcagacaaatcctccagcacagcctacatggagctccgcagcctgacatctgaggactctgccgtctatttctgtacaagagctcgcggtagtaggtccgtgtactactttgacaactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 237 - protein, 445 aa, synthetic construct
QVQLQQSGAELVRPGASVTLSCKASGYTFTDYEMHWVRQTPVHGLEWIGAIDTETGGPAYNQKFKGKAILTADKSSSTAYMELRSLTSEDSAVYFCTRARGSRSVYYFDNWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 238 - DNA, 645 bp, synthetic construct
  source 1..645: mol_type = other DNA
gacattgtgatgacccagtctcaaaaattcatgtccacatcagtgggagacagggtcagcgtcacctgcaaggccagtcagaatgtgggtactaatgtagcctggtatcagcagaaaccagggcactctcctaaagtactgatttactcggcatcctaccggcacagtggagtccctgatcgcttcacaggcagtggatctgggacagatttcactctcaccatcggcaatgtgcagtctgaggacttggcagactatttctgtcagcaatatgacacctatcctctcacgttcggctcggggacaaagttggaaataaaacgggctgatgctgcaccaactgtatccatcttcccaccatccagtgagcagttaacatctggaggtgcctcagtcgtgtgcttcttgaacaacttctaccccagagacatcaatgtcaagtggaagattgatggcagtgaacgacaaaatggtgtcctgaacagttggactgatcaggacagcaaagacagcacctacagcatgagcagcaccctcacattgaccaaggacgagtatgaacgacataacagctatacctgtgaggccactcacaagacatcaacttcacccatcgtcaagagcttcaacaggaatgagtgttag

SEQ ID NO: 239 - protein, 214 aa, synthetic construct
DIVMTQSQKFMSTSVGDRVSVTCKASQNVGTNVAWYQQKPGHSPKVLIYSASYRHSGVPDRFTGSGSGTDFTLTIGNVQSEDLADYFCQQYDTYPLTFGSGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 240 - DNA, 364 bp, synthetic construct
  source 1..364: mol_type = other DNA
caggttcaactgcagcagtctggggctgaactggtgaggcctggggcttcagtgacgctgtcctgcaaggcttcgggctacacatttactgactatgaaatgcactgggtgaggcagacacctgtgcatggcctggaatggattggagctattgatactgaaactggtggtcctgcctacaatcagaagttcaagggcaaggccatactgactgcagacaaatcctccagcacagcctacatggagctccgcagcctgacatctgaggactctgccgtctatttctgtacaagagctcgcggtagtaggtccgtgtactactttgacaactggggccaaggcaccactctcacagtctcctcag

SEQ ID NO: 241 - protein, 121 aa, synthetic construct
QVQLQQSGAELVRPGASVTLSCKASGYTFTDYEMHWVRQTPVHGLEWIGAIDTETGGPAYNQKFKGKAILTADKSSSTAYMELRSLTSEDSAVYFCTRARGSRSVYYFDNWGQGTTLTVSS

SEQ ID NO: 242 - DNA, 322 bp, synthetic construct
  source 1..322: mol_type = other DNA
gacattgtgatgacccagtctcaaaaattcatgtccacatcagtgggagacagggtcagcgtcacctgcaaggccagtcagaatgtgggtactaatgtagcctggtatcagcagaaaccagggcactctcctaaagtactgatttactcggcatcctaccggcacagtggagtccctgatcgcttcacaggcagtggatctgggacagatttcactctcaccatcggcaatgtgcagtctgaggacttggcagactatttctgtcagcaatatgacacctatcctctcacgttcggctcggggacaaagttggaaataaaac

SEQ ID NO: 243 - protein, 107 aa, synthetic construct
DIVMTQSQKFMSTSVGDRVSVTCKASQNVGTNVAWYQQKPGHSPKVLIYSASYRHSGVPDRFTGSGSGTDFTLTIGNVQSEDLADYFCQQYDTYPLTFGSGTKLEIK

SEQ ID NO: 244 - protein, 8 aa, synthetic construct
GYTFTDYE

SEQ ID NO: 245 - protein, 8 aa, synthetic construct
IDTETGGP

SEQ ID NO: 246 - protein, 14 aa, synthetic construct
TRARGSRSVYYFDN

SEQ ID NO: 247 - protein, 10 aa, synthetic construct
GYTFTDYEMH

SEQ ID NO: 248 - protein, 17 aa, synthetic construct
AIDTETGGPAYNQKFKG

SEQ ID NO: 249 - protein, 12 aa, synthetic construct
ARGSRSVYYFDN

SEQ ID NO: 250 - protein, 13 aa, synthetic construct
GYTFTDYEMHWVR

SEQ ID NO: 251 - protein, 21 aa, synthetic construct
IGAIDTETGGPAYNQKFKGKA

SEQ ID NO: 252 - protein, 14 aa, synthetic construct
RARGSRSVYYFDNW

SEQ ID NO: 253 - protein, 9 aa, synthetic construct
TDYEMHWVR

SEQ ID NO: 254 - protein, 24 aa, synthetic construct
LEWIGAIDTETGGPAYNQKFKGKA

SEQ ID NO: 255 - protein, 15 aa, synthetic construct
CTRARGSRSVYYFDN

SEQ ID NO: 256 - protein, 7 aa, synthetic construct
YEMHWVR

SEQ ID NO: 257 - protein, 24 aa, synthetic construct
IGAIDTETGGPAYNQKFKGKAILT

SEQ ID NO: 258 - protein, 13 aa, synthetic construct
ARGSRSVYYFDNW

SEQ ID NO: 259 - protein, 9 aa, synthetic construct
QQYDTYPLT

SEQ ID NO: 260 - protein, 6 aa, synthetic construct
SASYRH

SEQ ID NO: 261 - protein, 11 aa, synthetic construct
SASYRHSGVPD

SEQ ID NO: 262 - protein, 12 aa, synthetic construct
FCQQYDTYPLTF

SEQ ID NO: 263 - protein, 14 aa, synthetic construct
VLIYSASYRHSGVP

SEQ ID NO: 264 - protein, 11 aa, synthetic construct
FCQQYDTYPLT

SEQ ID NO: 265 - DNA, 1338 bp, synthetic construct
  source 1..1338: mol_type = other DNA
caggttcaactgcagcagtctggggctgaactggtgaggcctggggcttcagtgacgctgtcctgcaaggcttcgggctacacatttactgactatgaaatgcactgggtgaggcagacacctgtgcatggcctggaatggattggagctattgatactgaaactggtggtcctgcctacaatcagaagttcaagggcaaggccatactgactgcagacaaatcctccagcacagcctacatggagctccgcagcctgacatctgaggactctgccgtctatttctgtacaagagctcgcggtagtaggtccgtgtactactttgacaactggggccaaggcaccactctcacagtctcctcatccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 266 - protein, 445 aa, synthetic construct
QVQLQQSGAELVRPGASVTLSCKASGYTFTDYEMHWVRQTPVHGLEWIGAIDTETGGPAYNQKFKGKAILTADKSSSTAYMELRSLTSEDSAVYFCTRARGSRSVYYFDNWGQGTTLTVSSSKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 267 - DNA, 645 bp, synthetic construct
  source 1..645: mol_type = other DNA
gacattgtgatgacccagtctcaaaaattcatgtccacatcagtgggagacagggtcagcgtcacctgcaaggccagtcagaatgtggatactaatatagcctggtatcagcagaaaccagggcactctcctaaagcactgatttactcggcatcctaccggtacagtggagtccctgatcgcttcacaggcagtggatctgggacagatttcactctcaccatcggcaatgtgcagtctgaggacttggcagactatttctgtcagcaatatgacacctatcctctcacgttcggctcggggacaaagttggaaataaaacgggctgatgctgcaccaactgtatccatcttcccaccatccagtgagcagttaacatctggaggtgcctcagtcgtgtgcttcttgaacaacttctaccccagagacatcaatgtcaagtggaagattgatggcagtgaacgacaaaatggtgtcctgaacagttggactgatcaggacagcaaagacagcacctacagcatgagcagcaccctcacattgaccaaggacgagtatgaacgacataacagctatacctgtgaggccactcacaagacatcaacttcacccatcgtcaagagcttcaacaggaatgagtgttag

SEQ ID NO: 268 - protein, 214 aa, synthetic construct
DIVMTQSQKFMSTSVGDRVSVTCKASQNVDTNIAWYQQKPGHSPKALIYSASYRYSGVPDRFTGSGSGTDFTLTIGNVQSEDLADYFCQQYDTYPLTFGSGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 269 - DNA, 364 bp, synthetic construct
  source 1..364: mol_type = other DNA
caggttcaactgcagcagtctggggctgaactggtgaggcctggggcttcagtgacgctgtcctgcaaggcttcgggctacacatttactgactatgaaatgcactgggtgaggcagacacctgtgcatggcctggaatggattggagctattgatactgaaactggtggtcctgcctacaatcagaagttcaagggcaaggccatactgactgcagacaaatcctccagcacagcctacatggagctccgcagcctgacatctgaggactctgccgtctatttctgtacaagagctcgcggtagtaggtccgtgtactactttgacaactggggccaaggcaccactctcacagtctcctcat

SEQ ID NO: 270 - DNA, 322 bp, synthetic construct
  source 1..322: mol_type = other DNA
gacattgtgatgacccagtctcaaaaattcatgtccacatcagtgggagacagggtcagcgtcacctgcaaggccagtcagaatgtggatactaatatagcctggtatcagcagaaaccagggcactctcctaaagcactgatttactcggcatcctaccggtacagtggagtccctgatcgcttcacaggcagtggatctgggacagatttcactctcaccatcggcaatgtgcagtctgaggacttggcagactatttctgtcagcaatatgacacctatcctctcacgttcggctcggggacaaagttggaaataaaac

SEQ ID NO: 271 - protein, 107 aa, synthetic construct
DIVMTQSQKFMSTSVGDRVSVTCKASQNVDTNIAWYQQKPGHSPKALIYSASYRYSGVPDRFTGSGSGTDFTLTIGNVQSEDLADYFCQQYDTYPLTFGSGTKLEIK

SEQ ID NO: 272 - protein, 11 aa, synthetic construct
KASQNVDTNIA

SEQ ID NO: 273 - protein, 6 aa, synthetic construct
SASYRY

SEQ ID NO: 274 - protein, 15 aa, synthetic construct
TCKASQNVDTNIAWY

SEQ ID NO: 275 - protein, 11 aa, synthetic construct
NVDTNIAWYQQ

SEQ ID NO: 276 - DNA, 1374 bp, synthetic construct
  source 1..1374: mol_type = other DNA
caggttcaactgcagcagtctggggctgagctggtgaggcctggggcttcagtgacgctgtcctgcaaggcttcgggctacacatttactgactatgaaatgcactgggtgaagcagacacctgtgcatggcctggaatggattggagctattgatcctgaaactggtggtgctgcctacaatcagaagttcaagggcaaggccatactgactgcagacaaatcctccagcacagcctacatggagctccgcagcctgacatctgaggactctgccgtctattattgtacaagagctcgcggtagtagctccgtgtactactttgactactggggccaaggcaccactctcacagtctcctcagccaaaacaacacccccatcagtctatccactggcccctgggtgtggagatacaactggttcctctgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaatga

SEQ ID NO: 277 - protein, 457 aa, synthetic construct
QVQLQQSGAELVRPGASVTLSCKASGYTFTDYEMHWVKQTPVHGLEWIGAIDPETGGAAYNQKFKGKAILTADKSSSTAYMELRSLTSEDSAVYYCTRARGSSSVYYFDYWGQGTTLTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 278 - DNA, 645 bp, synthetic construct
  source 1..645: mol_type = other DNA
gacattgtgatgacccagtctcaaaaattcatgtccacatcagtaggagacagggtcagcgtcacctgcaaggccagtcagaatgtgggtactaatgtagcctggtatcaacagaaaccagggcaatctcctaaagcactgatttactcgacatcctaccggtacagtggagtccctgatcgcttcacaggcagtagatctgggacagatttcattctcaccatcagcaatgtgcagtctgaagacttggcagagtatttctgtcagcaatatgacagctatcctctcacgttcggctcggggacaaagttggaaataaaacgggctgatgctgcaccaactgtatccatcttcccaccatccagtgagcagttaacatctggaggtgcctcagtcgtgtgcttcttgaacaacttctaccccagagacatcaatgtcaagtggaagattgatggcagtgaacgacaaaatggtgtcctgaacagttggactgatcaggacagcaaagacagcacctacagcatgagcagcaccctcacattgaccaaggacgagtatgaacgacataacagctatacctgtgaggccactcacaagacatcaacttcacccatcgtcaagagcttcaacaggaatgagtgttag

SEQ ID NO: 279 - protein, 214 aa, synthetic construct
DIVMTQSQKFMSTSVGDRVSVTCKASQNVGTNVAWYQQKPGQSPKALIYSTSYRYSGVPDRFTGSRSGTDFILTISNVQSEDLAEYFCQQYDSYPLTFGSGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 280 - DNA, 364 bp, synthetic construct
  source 1..364: mol_type = other DNA
caggttcaactgcagcagtctggggctgagctggtgaggcctggggcttcagtgacgctgtcctgcaaggcttcgggctacacatttactgactatgaaatgcactgggtgaagcagacacctgtgcatggcctggaatggattggagctattgatcctgaaactggtggtgctgcctacaatcagaagttcaagggcaaggccatactgactgcagacaaatcctccagcacagcctacatggagctccgcagcctgacatctgaggactctgccgtctattattgtacaagagctcgcggtagtagctccgtgtactactttgactactggggccaaggcaccactctcacagtctcctcag

SEQ ID NO: 281 - protein, 121 aa, synthetic construct
QVQLQQSGAELVRPGASVTLSCKASGYTFTDYEMHWVKQTPVHGLEWIGAIDPETGGAAYNQKFKGKAILTADKSSSTAYMELRSLTSEDSAVYYCTRARGSSSVYYFDYWGQGTTLTVSS

SEQ ID NO: 282 - DNA, 322 bp, synthetic construct
  source 1..322: mol_type = other DNA
gacattgtgatgacccagtctcaaaaattcatgtccacatcagtaggagacagggtcagcgtcacctgcaaggccagtcagaatgtgggtactaatgtagcctggtatcaacagaaaccagggcaatctcctaaagcactgatttactcgacatcctaccggtacagtggagtccctgatcgcttcacaggcagtagatctgggacagatttcattctcaccatcagcaatgtgcagtctgaagacttggcagagtatttctgtcagcaatatgacagctatcctctcacgttcggctcggggacaaagttggaaataaaac

SEQ ID NO: 283 - protein, 107 aa, synthetic construct
DIVMTQSQKFMSTSVGDRVSVTCKASQNVGTNVAWYQQKPGQSPKALIYSTSYRYSGVPDRFTGSRSGTDFILTISNVQSEDLAEYFCQQYDSYPLTFGSGTKLEIK

SEQ ID NO: 284 - protein, 8 aa, synthetic construct
IDPETGGA

SEQ ID NO: 285 - protein, 14 aa, synthetic construct
TRARGSSSVYYFDY

SEQ ID NO: 286 - protein, 17 aa, synthetic construct
AIDPETGGAAYNQKFKG

SEQ ID NO: 287 - protein, 12 aa, synthetic construct
ARGSSSVYYFDY

SEQ ID NO: 288 - protein, 13 aa, synthetic construct
GYTFTDYEMHWVK

SEQ ID NO: 289 - protein, 21 aa, synthetic construct
IGAIDPETGGAAYNQKFKGKA

SEQ ID NO: 290 - protein, 14 aa, synthetic construct
RARGSSSVYYFDYW

SEQ ID NO: 291 - protein, 9 aa, synthetic construct
TDYEMHWVK

SEQ ID NO: 292 - protein, 24 aa, synthetic construct
LEWIGAIDPETGGAAYNQKFKGKA

SEQ ID NO: 293 - protein, 15 aa, synthetic construct
CTRARGSSSVYYFDY

SEQ ID NO: 294 - protein, 7 aa, synthetic construct
YEMHWVK

SEQ ID NO: 295 - protein, 24 aa, synthetic construct
IGAIDPETGGAAYNQKFKGKAILT

SEQ ID NO: 296 - protein, 13 aa, synthetic construct
ARGSSSVYYFDYW

SEQ ID NO: 297 - skipped

SEQ ID NO: 298 - protein, 9 aa, synthetic construct
QQYDSYPLT

SEQ ID NO: 299 - protein, 6 aa, synthetic construct
STSYRY

SEQ ID NO: 300 - protein, 11 aa, synthetic construct
STSYRYSGVPD

SEQ ID NO: 301 - protein, 12 aa, synthetic construct
FCQQYDSYPLTF

SEQ ID NO: 302 - protein, 14 aa, synthetic construct
ALIYSTSYRYSGVP

SEQ ID NO: 303 - protein, 11 aa, synthetic construct
FCQQYDSYPLT

SEQ ID NO: 304 - DNA, 1338 bp, synthetic construct
  source 1..1338: mol_type = other DNA
caggttcaactgcagcagtctggggctgagctggtgaggcctggggcttcagtgacgctgtcctgcaaggcttcgggctacacatttactgactatgaaatgcactgggtgaaacagacacctgtgcatggcctggaatggattggagctattgatcctgaaactggtggtattgcctacaatcagaagttcaggggcaaggccatactgactgcagacaaatcctccagcacagcctacatggagctccgcagcctgacatctgaggactctgccgtctattactgtgcaagaaactacggtattagctcctactatgctgtggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 305 - protein, 445 aa, synthetic construct
QVQLQQSGAELVRPGASVTLSCKASGYTFTDYEMHWVKQTPVHGLEWIGAIDPETGGIAYNQKFRGKAILTADKSSSTAYMELRSLTSEDSAVYYCARNYGISSYYAVDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 306 - DNA, 645 bp, synthetic construct
  source 1..645: mol_type = other DNA
gacattgtgatgacccagtctcaaaaattcatgtccacaacagtaggagagagggtcagcatcacctgcaaggccagtcagaatgtgggtactgctgtagcctggtatcaacagaaaccaggacaatctcctaaactactgatttactcagcatccaatcggtacactggagtccctgatcgcttcacaggcagtggatctgggacagatttcactctcaccattagcaatatgcagtctgaagacctggcagattatgtctctcagcaatatagcagctatcctctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccagagacatcaatgtcaagtggaagattgatggcagtgaacgacaaaatggtgtcctgaacagttggactgatcaggacagcaaagacagcacctacagcatgagcagcaccctcacattgaccaaggacgagtatgaacgacataacagctatacctgtgaggccactcacaagacatcaacttcacccatcgtcaagagcttcaacaggaatgagtgttag

SEQ ID NO: 307 - protein, 183 aa, synthetic construct
AVAWYQQKPGQSPKLLIYSASNRYTGVPDRFTGSGSGTDFTLTISNMQSEDLADYVSQQYSSYPLTFGAGTKLELKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 308 - DNA, 364 bp, synthetic construct
  source 1..364: mol_type = other DNA
caggttcaactgcagcagtctggggctgagctggtgaggcctggggcttcagtgacgctgtcctgcaaggcttcgggctacacatttactgactatgaaatgcactgggtgaaacagacacctgtgcatggcctggaatggattggagctattgatcctgaaactggtggtattgcctacaatcagaagttcaggggcaaggccatactgactgcagacaaatcctccagcacagcctacatggagctccgcagcctgacatctgaggactctgccgtctattactgtgcaagaaactacggtattagctcctactatgctgtggactactggggtcaaggaacctcagtcaccgtctcctcag

SEQ ID NO: 309 - protein, 121 aa, synthetic construct
QVQLQQSGAELVRPGASVTLSCKASGYTFTDYEMHWVKQTPVHGLEWIGAIDPETGGIAYNQKFRGKAILTADKSSSTAYMELRSLTSEDSAVYYCARNYGISSYYAVDYWGQGTSVTVSS

SEQ ID NO: 310 - DNA, 322 bp, synthetic construct
  source 1..322: mol_type = other DNA
gacattgtgatgacccagtctcaaaaattcatgtccacaacagtaggagagagggtcagcatcacctgcaaggccagtcagaatgtgggtactgctgtagcctggtatcaacagaaaccaggacaatctcctaaactactgatttactcagcatccaatcggtacactggagtccctgatcgcttcacaggcagtggatctgggacagatttcactctcaccattagcaatatgcagtctgaagacctggcagattatgtctctcagcaatatagcagctatcctctcacgttcggtgctgggaccaagctggagctgaaac

SEQ ID NO: 311 - protein, 107 aa, synthetic construct
DIVMTQSQKFMSTTVGERVSITCKASQNVGTAVAWYQQKPGQSPKLLIYSASNRYTGVPDRFTGSGSGTDFTLTISNMQSEDLADYVSQQYSSYPLTFGAGTKLELK

SEQ ID NO: 312 - protein, 8 aa, synthetic construct
IDPETGGI

SEQ ID NO: 313 - protein, 14 aa, synthetic construct
ARNYGISSYYAVDY

SEQ ID NO: 314 - protein, 11 aa, synthetic construct
GYTFTDYEMHW

SEQ ID NO: 315 - protein, 17 aa, synthetic construct
AIDPETGGIAYNQKFRG

SEQ ID NO: 316 - protein, 12 aa, synthetic construct
NYGISSYYAVDY

SEQ ID NO: 317 - protein, 21 aa, synthetic construct
IGAIDPETGGIAYNQKFRGKA

SEQ ID NO: 318 - protein, 14 aa, synthetic construct
RNYGISSYYAVDYW

SEQ ID NO: 319 - protein, 24 aa, synthetic construct
LEWIGAIDPETGGIAYNQKFRGKA

SEQ ID NO: 320 - protein, 15 aa, synthetic construct
CARNYGISSYYAVDY

SEQ ID NO: 321 - protein, 24 aa, synthetic construct
IGAIDPETGGIAYNQKFRGKAILT

SEQ ID NO: 322 - protein, 13 aa, synthetic construct
NYGISSYYAVDYW

SEQ ID NO: 323 - protein, 6 aa, synthetic construct
QNVGTA

SEQ ID NO: 324 - protein, 9 aa, synthetic construct
QQYSSYPLT

SEQ ID NO: 325 - protein, 11 aa, synthetic construct
KASQNVGTAVA

SEQ ID NO: 326 - protein, 6 aa, synthetic construct
SASNRY

SEQ ID NO: 327 - protein, 15 aa, synthetic construct
TCKASQNVGTAVAWY

SEQ ID NO: 328 - protein, 11 aa, synthetic construct
SASNRYTGVPD

SEQ ID NO: 329 - protein, 12 aa, synthetic construct
VSQQYSSYPLTF

SEQ ID NO: 330 - protein, 11 aa, synthetic construct
NVGTAVAWYQQ

SEQ ID NO: 331 - protein, 14 aa, synthetic construct
LLIYSASNRYTGVP

SEQ ID NO: 332 - protein, 11 aa, synthetic construct
VSQQYSSYPLT

SEQ ID NO: 333 - protein, 441 aa, synthetic construct
EVQLQQSGPELVKPGASVKISCKTSGYTFTDYYMNWVKQSHGKSLEWIGDINPNNGGAGYNQKFKGKATLTVDKSSSTAYMELRSLTSEDSAVYYCARWGWDRYFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPG

SEQ ID NO: 334 - DNA, 1326 bp, synthetic construct
  source 1..1326: mol_type = other DNA
gaggtccagctgcaacaatctggacctgagctggtgaagcctggggcttcagtgaagatatcctgtaagacttctggatacacgttcactgactactacatgaactgggtgaagcagagccatggaaagagccttgagtggattggagatattaatcctaacaatggtggtgctggctacaaccagaagttcaagggcaaggccacattgactgtagacaagtcctccagcacagcctacatggagctccgcagcctgacatctgaggactctgcagtctattactgtgcaagatggggctgggaccggtactttgactactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggttga

SEQ ID NO: 335 - protein, 444 aa, synthetic construct
QVQLQQSGAELVRPGASVTLSCKASGYTFTDYEMHWVRQTPVHGLEWIGAIDTETGGPAYNQKFKGKAILTADKSSSTAYMELRSLTSEDSAVYFCTRARGSRSVYYFDNWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPG

SEQ ID NO: 336 - DNA, 1335 bp, synthetic construct
  source 1..1335: mol_type = other DNA
caggttcaactgcagcagtctggggctgaactggtgaggcctggggcttcagtgacgctgtcctgcaaggcttcgggctacacatttactgactatgaaatgcactgggtgaggcagacacctgtgcatggcctggaatggattggagctattgatactgaaactggtggtcctgcctacaatcagaagttcaagggcaaggccatactgactgcagacaaatcctccagcacagcctacatggagctccgcagcctgacatctgaggactctgccgtctatttctgtacaagagctcgcggtagtaggtccgtgtactactttgacaactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggttga

SEQ ID NO: 337 - protein, 445 aa, synthetic construct; identical to SEQ ID NO: 266

SEQ ID NO: 338 - DNA, 1335 bp, synthetic construct
  source 1..1335: mol_type = other DNA
caggttcaactgcagcagtctggggctgaactggtgaggcctggggcttcagtgacgctgtcctgcaaggcttcgggctacacatttactgactatgaaatgcactgggtgaggcagacacctgtgcatggcctggaatggattggagctattgatactgaaactggtggtcctgcctacaatcagaagttcaagggcaaggccatactgactgcagacaaatcctccagcacagcctacatggagctccgcagcctgacatctgaggactctgccgtctatttctgtacaagagctcgcggtagtaggtccgtgtactactttgacaactggggccaaggcaccactctcacagtctcctcatccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggttga

SEQ ID NO: 339 - protein, 456 aa, synthetic construct
QVQLQQSGAELVRPGASVTLSCKASGYTFTDYEMHWVKQTPVHGLEWIGAIDPETGGAAYNQKFKGKAILTADKSSSTAYMELRSLTSEDSAVYYCTRARGSSSVYYFDYWGQGTTLTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPG

SEQ ID NO: 340 - DNA, 1371 bp, synthetic construct
  source 1..1371: mol_type = other DNA
caggttcaactgcagcagtctggggctgagctggtgaggcctggggcttcagtgacgctgtcctgcaaggcttcgggctacacatttactgactatgaaatgcactgggtgaagcagacacctgtgcatggcctggaatggattggagctattgatcctgaaactggtggtgctgcctacaatcagaagttcaagggcaaggccatactgactgcagacaaatcctccagcacagcctacatggagctccgcagcctgacatctgaggactctgccgtctattattgtacaagagctcgcggtagtagctccgtgtactactttgactactggggccaaggcaccactctcacagtctcctcagccaaaacaacacccccatcagtctatccactggcccctgggtgtggagatacaactggttcctctgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggttga

SEQ ID NO: 341 - protein, 444 aa, synthetic construct
QVQLQQSGAELVRPGASVTLSCKASGYTFTDYEMHWVKQTPVHGLEWIGAIDPETGGIAYNQKFRGKAILTADKSSSTAYMELRSLTSEDSAVYYCARNYGISSYYAVDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPG

SEQ ID NO: 342 - DNA, 1335 bp, synthetic construct
  source 1..1335: mol_type = other DNA
caggttcaactgcagcagtctggggctgagctggtgaggcctggggcttcagtgacgctgtcctgcaaggcttcgggctacacatttactgactatgaaatgcactgggtgaaacagacacctgtgcatggcctggaatggattggagctattgatcctgaaactggtggtattgcctacaatcagaagttcaggggcaaggccatactgactgcagacaaatcctccagcacagcctacatggagctccgcagcctgacatctgaggactctgccgtctattactgtgcaagaaactacggtattagctcctactatgctgtggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggttga

SEQ ID NO: 343 - protein, 24 aa, synthetic construct
CVDRKTGVKLPGGLEPKGELDCHQ

SEQ ID NO: 344 - protein, 15 aa, synthetic construct
HPKQCHPALDGQRGK

SEQ ID NO: 345 - protein, 15 aa, synthetic construct
KQCHPALDGQRGKCW

SEQ ID NO: 346 - protein, 15 aa, synthetic construct
CHPALDGQRGKCWCV

SEQ ID NO: 347 - protein, 15 aa, synthetic construct
PALDGQRGKCWCVDR

SEQ ID NO: 348 - protein, 15 aa, synthetic construct
LDGQRGKCWCVDRKT

SEQ ID NO: 349 - protein, 15 aa, synthetic construct
GQRGKCWCVDRKTGV

SEQ ID NO: 350 - protein, 15 aa, synthetic construct
RGKCWCVDRKTGVKL

SEQ ID NO: 351 - protein, 15 aa, synthetic construct
KCWCVDRKTGVKLPG

SEQ ID NO: 352 - protein, 15 aa, synthetic construct
WCVDRKTGVKLPGGL

SEQ ID NO: 353 - protein, 30 aa, synthetic construct
VDRKTGVKLPGGLEPRKTGVKLPGGLEPKG

SEQ ID NO: 354 - protein, 7 aa, synthetic construct
  VARIANT 7: note = X can be Y or E
GYTFTYX

SEQ ID NO: 355-358 - skipped (4 entries)

SEQ ID NO: 359 - protein, 9 aa, synthetic construct
  VARIANT 4: note = X can be N,D or S
  VARIANT 5: note = X can be I,S or T
  VARIANT 9: note = X can be A or T
QQYXXYPLX

SEQ ID NO: 360 - protein, 8 aa, synthetic construct
  VARIANT 8: note = X can be A,S or I
INPNNGGX

SEQ ID NO: 361 - protein, 11 aa, synthetic construct
  VARIANT 4: note = X can be G
  VARIANT 5: note = X can be W
  VARIANT 7: note = X can be R,G or S
  VARIANT 8: note = X can be G or Y
ARWXXDXXFDY

SEQ ID NO: 362 - protein, 6 aa, synthetic construct
  VARIANT 5: note = X can be I or T
  VARIANT 6: note = X can be D, N or A
QNVGXX

SEQ ID NO: 363 - protein, 9 aa, synthetic construct
  VARIANT 5: note = X can be I or S
  VARIANT 9: note = X can be A or T
QQYNXYPLX

SEQ ID NO: 364 - protein, 8 aa, synthetic construct
  VARIANT 3: note = X can be P or T
  VARIANT 8: note = X can be I,P or A
IDXETGGX

SEQ ID NO: 365 - protein, 14 aa, synthetic construct
  VARIANT 7: note = X can be S orR
  VARIANT 14: note = X can be Y or N
TRARGSXSVYYFDX

SEQ ID NO: 366 - protein, 6 aa, synthetic construct
  VARIANT 4: note = X can be G or D
QNVXTN

SEQ ID NO: 367 - skipped

SEQ ID NO: 368 - protein, 9 aa, synthetic construct
  VARIANT 5: note = X can be S or T
QQYDXYPLT

SEQ ID NO: 369 - protein, 102 aa, synthetic construct
KVNGAPREDARPVPQGSCQSELHRALERLAASQSRTHEDLYIIPIPNCDRNGNFHPKQCHPALDGQRGKCWCVDRKTGVKLPGGLEPKGELDCHQLADSFRE

SEQ ID NO: 370 - protein, 10 aa, synthetic construct
QCHPALDGQR

SEQ ID NO: 371 - protein, 14 aa, synthetic construct
IALGGLLFPASNLR